(19) United States
(12) Patent Application Publication
(10) Pub. No.: US 2025/0387499 A1
(43) Pub. Date: Dec. 25, 2025

(54) LIGAND-PAYLOAD CONJUGATES

(71) Applicant: Pyxis Oncology, Inc., Boston, MA (US)

(72) Inventors: Ravi Chari, Needham, MA (US); Arnab Ghatak, Basking Ridge, NJ (US); David Pedersen, Chelmsford, MA (US); Jianwen Feng, Newton, MA (US); Chuan Shen, Boston, MA (US)

(73) Assignee: Pyxis Oncology, Inc., Boston, MA (US)

(21) Appl. No.: 18/850,092

(22) PCT Filed: Mar. 30, 2023
(86) PCT No.: PCT/US2023/065173

§ 371 Date: Sep. 24, 2024

Related U.S. Application Data
(60) Provisional application No. 63/362,145, filed on Mar. 30, 2022.
(60) Provisional application No. 63/362,149, filed on Mar. 30, 2022.

(51) Int. Cl. A61K 47/68; C07K 16/32

(52) U.S. Cl. CPC A61K 47/68037; A61K 47/6855; A61K 47/6889; C07K 16/32

(57) ABSTRACT

The present disclosure relates to ligand-payload conjugates, and compositions and use thereof for treating diseases, disorders, or conditions, such as cancers, autoimmune diseases, or infectious diseases. The present disclosure also provides methods of synthesizing ligand-payload conjugates, and related intermediates.

### CROSS-REFERENCE TO RELATED APPLICATIONS

This application claims priority under 35 U.S.C. § 119(e) to U.S. Provisional Application No. 63/362,145, filed Mar. 30, 2022 and U.S. Provisional Application No. 63/362,149, filed Mar. 30, 2022, the disclosures of which are incorporated herein by reference in their entireties.

### INCORPORATION-BY-REFERENCE OF SEQUENCE LISTING

The contents of the text file named “PYX002-US1_Sequence_Listing_ST26”, which was created Jul. 25, 2025, and is 35.1 KB in size, are hereby incorporated by reference in their entireties.

### BACKGROUND

Ligand-payload conjugates can provide targeted therapeutic treatment in patients with various disease and disorders. For example, an antibody-drug conjugate (ADC) having a drug with cytotoxicity conjugated to an antibody, whose antigen is expressed on a surface of cancer cells or extracellular matrix (ECM), can deliver the drug selectively to tumor microenvironment (TME). Comprising highly hydrophobic payload molecules (such as MMAE), ADCs with two to four drug molecules per antibody have been reported to be generally superior to more heavily loaded conjugates (e.g., greater than four drugs per antibody) in terms of ADC stability, in vivo efficacy, tolerability, and pharmacokinetics, and thus resulting in higher therapeutic index. See, e.g., Hamblett et al., Clinical Cancer Research, 10: 7063-7070 (2004). However, for less hydrophobic payload molecules (such as Dxd), higher DAR (8 and above) are preferred.

There is a need in the art for novel ligand-payload conjugates, which in certain embodiments can be used to treat, ameliorate, and/or prevent a disease or disorder in a subject.

### SUMMARY

In one aspect, the present disclosure relates to ligand-payload conjugates, wherein payloads are attached to a ligand, optionally via branched linkers, and uses thereof for treating, ameliorating, and/or preventing certain diseases or disorders, such as but not limited to cancers and immunological diseases. The disclosure also provides methods for synthesizing the ligand-payload conjugates, and related intermediate compounds.

The ligand-payload conjugates of the present disclosure comprise specifically designed linkers to attach payloads to ligands, which provide an enhanced linker-payload stability and an increased therapeutic index.

In one aspect, the instant disclosure provides a ligand-payload conjugate of formula (I):

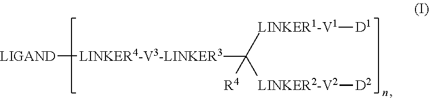

or a pharmaceutically acceptable salt thereof, wherein each variable is independently as defined and described herein.

In one aspect, the instant invention provides a ligand-payload conjugate of formula (I*):

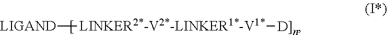

or a pharmaceutically acceptable salt thereof, wherein each variable is independently as defined and described herein.

In another aspect, the instant disclosure provides a linker-payload compound of formula (II):

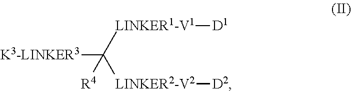

or a pharmaceutically acceptable salt thereof, wherein each variable is independently as defined and described herein.

In another aspect, the instant invention provides a linker-payload compound of formula (II*):

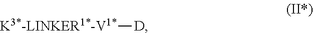

or a pharmaceutically acceptable salt thereof, wherein each variable is independently as defined and described herein.

In another aspect, the instant disclosure provides a ligand of formula (III):

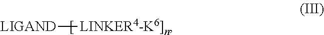

or a pharmaceutically acceptable salt thereof, wherein each variable is independently as defined and described herein.

In another aspect, the instant invention provides a ligand of formula (III*):

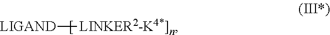

or a pharmaceutically acceptable salt thereof, wherein each variable is independently as defined and described herein.

In another aspect, the instant disclosure provides a method of synthesizing a ligand-payload conjugate of formula (I), comprising reacting a linker-payload compound of formula (II) with a ligand of formula (III).

In another aspect, the instant invention provides a method of synthesizing a ligand-payload conjugate of formula (I*), comprising reacting a linker-payload compound of formula (II*) with a ligand of formula (III*).

In another aspect, the instant disclosure provides a method of treating a disease or a disorder in a subject in need thereof, comprising administering to the subject a ligand-payload conjugate of formula (I), or a pharmaceutically acceptable salt thereof. In some embodiments, a disease or a disorder is selected from the diseases or disorders as described herein.

In another aspect, the instant invention provides a method of treating a disease or a disorder in a subject in need thereof, comprising administering to the subject a ligand-payload conjugate of formula (I*), or a pharmaceutically acceptable salt thereof. In some embodiments, a disease or a disorder is selected from the diseases or disorders as described herein.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1B illustrates FACS binding curves for certain ADCs of the disclosure in certain cell lines.

FIG. 2 illustrates selected results from FACS binding studies.

FIG. 3 illustrates selected results from plasma stability studies of ADCs of the disclosure.

FIG. 4 illustrates cytotoxicity curves obtained for certain ADCs of the disclosure.

FIG. 5 illustrates cytotoxicity results obtained for certain ADCs of the disclosure.

FIG. 6 illustrates in vivo tumor volume results obtained in a xenograft model using certain ADCs of the disclosure.

FIG. 7 illustrates rat body weight obtained in a xenograft model using certain ADCs of the disclosure.

FIG. 8 illustrates in vivo tumor volume results obtained in a xenograft model using certain ADCs of the disclosure.

FIG. 9 illustrates rat body weight obtained in a xenograft model using certain ADCs of the disclosure.

### DETAILED DESCRIPTION OF CERTAIN EMBODIMENTS

Ligand-payload conjugates of the present disclosure, and pharmaceutical compositions thereof, provide targeted therapeutic treatment for diseases and disorders, such as those as described herein. Without wishing to be bound by any particular theory, it is believed that the linkers of the present disclosure, which attach payloads to ligands, enhance the linker-payload stability for specific conjugation chemistries, and the therapeutic index of the ligand-payload conjugates across a number of payload classes and payload-ligand ratios.

### 1. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₈ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

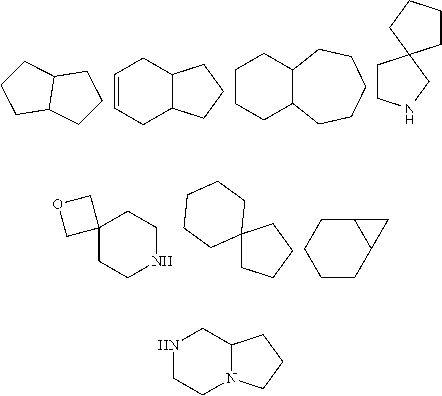

Exemplary bridged bicyclics include:

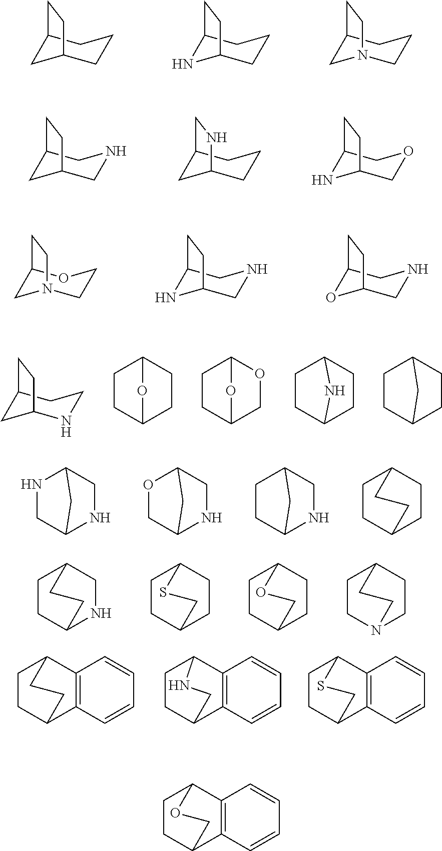

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

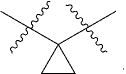 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄₀S(O)₂R^{o}; —S(O)₂NR^{o}₂; —S(O)(NR^{o})R^{o}; —S(O)₂N═C(NR^{o}₂)₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂.

Each R^{o} is independently hydrogen, C₁₋₆ aliphatic, —CH₂Ph, —O(CH₂)₀₋₁Ph, —CH₂— (5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{o} selected from ═O and ═S; or each R^{o} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR•, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR•, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R†, —NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(R†)S(O)₂R†; wherein each R† is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R† is C₁₋₆ aliphatic, R† is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example, to prevent or delay their recurrence.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15th Ed., Mack Publ. Co., Easton, PA [1975].

As used herein, the term “glutamine-containing tag”, “glutamine tag”, “Q-containing tag” “Q-tag”, or “transglutaminase tag” refers to a polypeptide or a protein containing one or more Gln residue(s) that acts as an amine acceptor or acyl donor in the transglutaminase reaction.

As used herein, the term “amine donor agent” or “acyl acceptor” refers to an agent containing one or more reactive amines (e.g., primary amines). For example, the amine donor agent can comprise an amine donor unit (e.g., primary amine NH₂), a linker (e.g., a molecule that is linked to an amine donor unit and contains additional functionality for attachment to a payload such as a small molecule, a polypeptide, or a biocompatible polymer), and an agent moiety (e.g., a payload such as a small molecule). The amine donor agent can also be a polypeptide (e.g., an antibody) or a biocompatible polymer containing one or more reactive lysine, N-termini, or reactive amines.

As used herein, the term “site specificity”, “site-specifically conjugated”, or “site-specifically crosslinked” refers to the specific conjugation or crosslinking of the amine donor agent to the antibody at a specific site (e.g. at various positions listed in Table 1) via a glutamine-containing tag, endogenous glutamine, and/or an endogenous glutamine made reactive by the antibody engineering with an engineered transglutaminase. Site specificity can be measured by various techniques, including, but not limited to, mass spectrometry (e.g., matrix-assisted laser-desorption ionization mass spectrometry (MALDI-MS), electrospray ionization mass spectrometry (ESI-MS), tandem mass spectrometry (MS-MS), and time-of-flight mass spectrometry (TOF-MS), hydrophobic interaction chromatography, ion exchange chromatography, site-directed mutagenesis, fluorescence-labeling, size exclusion chromatography, peptide digest, and X-ray crystallography.

As used herein, the term “an endogenous glutamine (Q made reactive” refers to an endogenous glutamine that has been made accessible, exposed, or reactive to the amine donor agent in the presence of a transglutaminase by antibody engineering (e.g., enzymatic deglycosylation and/or amino acid modification) or by an engineered transglutaminase.

As used herein, the term “biocompatible polymer” refers to a polymer (e.g., repeating monomeric or structural units) that is suitable for therapeutic or medical treatment in a recipient (e.g., human) without elicit any undesirable local or systemic effects in the recipient. A biocompatible polymer (synthetic, recombinant, or native) can be a water soluble or water insoluble polymer. A biocompatible polymer can also be a linear or a branched polymer.

As used herein, the term “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, unless otherwise indicated by context, the term is intended to encompass not only intact polyclonal or monoclonal antibodies, but also fragments thereof (such as Fab, Fab′, F(ab′)₂, Fv), single chain (ScFv) and domain antibodies, including shark and camelid antibodies), and fusion proteins comprising an antibody portion, multivalent antibodies (e.g., COVX-BODY™), multispecific antibodies (e.g., biparatopic antibodies, bispecific antibodies so long as they exhibit the desired biological activity) and antibody fragments as described herein, and any other modified configuration of the immunoglobulin molecule that comprises ai antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. On one aspect, the immunoglobulin is a human, murine, monkey, or rabbit immunoglobulin.

As used herein, the term “Fab containing polypeptide” refers to a polypeptide comprising a Fab fragment, Fab′ fragment, or “(Fab′)2 fragment.” A Fab-containing polypeptide may comprise part or all of a wild-type hinge seqluence (generally at the carboxyl terminus of the Fab portion of the polypeptide). A Fab-containing polypeptide may be obtained or derived from any suitable immunoglobulin, such as from at least one of the various IgG1, IgG2, IgG3, or IgG4 subtypes, or from IgA, IgE, IgD or IgM. A Fab-containing polypeptide may be a Fab-containing fusion polypeptide, wherein one or more polypeptides are linked to a Fab-containing polypeptide. A Fab fusion combines the Fab polypeptide of an immunoglobulin with a fusion partner, which in general may be any protein, polypeptide, or small molecule. Virtually any protein or small molecule may be linked to the Fab polypeptide to generate a Fab-containing fusion polypeptide. Fab-containing fusion partners may include, but are not limited to, the target-binding region of a receptor, an adhesion molecule, a ligand, an enzyme, a cytokine, a chemokine, or some other protein or protein domain.

A “Fab fragment” is comprised of one light chain and the CH 1 and variable regions of one heavy chain. The heavy chain of a Fab molecule cannot form a disulfide bond with another heavy chain molecule.

A “Fab′ fragment” contains one light chain and a portion of one heavy chain that contains the VH domain and the CH 1 domain and also the region between the CH 1 and CH2 domains, such that an interchain disulfide bond can be formed between the two heavy chains of two Fab′ fragments to form a F(ab′)2 molecule.

A “F(ab′)2 fragment” contains two light chains and two heavy chains containing a portion of the constant region between the CH 1 and CH2 domains, such that an interchain disulfide bond is formed between the two heavy chains. A F(ab′)2 fragment thus is composed of two Fab′ fragments that are held together by a disulfide bond between the two heavy chains.

As used herein, “antibody fragments” comprise only a portion of an intact antibody, wherein the portion preferably retains at least one, preferably most or all, of the functions normally associated with that portion when present in an intact antibody.

A “multispecific antibody” is one that targets more than one antigen or epitope. A “biparatopic” antibody binds two different epitopes of the same antigen. A “bispecific,” “dual-specific” or “Afunctional” antibody is a hybrid antibody having two different antigen binding sites. Bispecific antibodies are a species of multispecific antibody and may be produced by a variety of methods including, but not limited to, fusion of hybridomas, linking of Fab′ fragments, or mutations at the antibody hinge and CH3 domains. See, e.g., Songsivilai & Lachmann, Clin. Exp. Immunol. 79:315-321 (1990); Kostelny et al., J. Immunol. 148: 1547-1553 (1992); and Strop et al., J. Mol. Biol. 420(3):204-219 (2012). The two binding sites of a bispecific antibody will bind to two different epitopes, which may reside on the same or different protein targets.

The term “monoclonal antibody” as used herein refers to an antibody obtained from a population of substantially homogeneous antibodies. i.e., the individual antibodies comprising the population are identical except for possible naturally occurring mutations that may be present in minor amounts. Monoclonal antibodies are highly specific, being directed against a single antigen. Further, in contrast to polyclonal antibody preparations that typically include different antibodies directed against different determinants (epitopes), each monoclonal antibody is directed against a single determinant on the antigen.

The monoclonal antibodies herein may, in certain embodiments, specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Nail. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Humanized antibodies may, moreover, comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-1 15 (1998); Harris. Biochem. Soc. Transactions 23: 1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

The “hinge region.” “hinge sequence,” and variation thereof, as used herein, includes the meaning known in the art, which is illustrated, for example. Janeway et al., ImmunoBiology: the immune system in health and disease. (Elsevier Science Ltd., NY) (4^{th} ed., 1999); Bloom et al., Protein Science (1997), 6:407-415; Humphreys et al., J. Immunol. Methods (1997), 209: 193-202.

The term “Fc-containing polypeptide” as used herein refers to a polypeptide (e.g., an antibody or an immunoadhesin) comprising the carboxyl terminal polypeptide sequences of an immunoglobulin heavy chain. The Fc-containing polypeptide may comprise native or variant Fc regions (i.e., sequences). The Fe region of an immunoglobulin generally comprises two constant domains, a CH2 domain and a CH3 domain, and optionally comprises a CH4 domain. An Fc-containing polypeptide may comprise part or all of a wild-type hinge sequence (generally at amino terminus of the Fc-containing polypeptide). An Fc-containing polypeptide may also be a dimer. An Fc-containing polypeptide may be obtained or derived from any suitable immunoglobulin, such as from at least one of the various IgG1, IgG2, IgG3, or IgG4 subtypes, or from IgA, IgE, IgD or IgM. Although the boundaries of the Fc region of an immunoglobulin heavy chain might vary, for example, the human IgG heavy chain Fc region is usually defined to stretch from an amino acid residue at position Glu216, or from Ala231, to the carboxyl-terminus thereof. The numbering of the residues in the Fe region is that of the EU index as in Kabat. Kabat et al., Sequences of Proteins of Immunological Interest, 5th Ed. Public Health Service, National Institutes of Health, Bethesda, Md., 1991.

An Fc-containing polypeptide may be an Fe-containing fusion polypeptide, wherein one or more polypeptides are linked to an Fe-containing polypeptide. An Fc fusion combines the Fc polypeptide of an immunoglobulin with a fusion partner, which in general may be any protein, polypeptide, or small molecule. Virtually any protein or small molecule may be linked to the Fe region to generate an Fc-containing fusion polypeptide. Fc-containing fusion partners may include, but are not limited to, the target-binding region of a receptor, an adhesion molecule, a ligand, an enzyme, a cytokine, a chemokine, or some other protein or protein domain.

As used herein, the term “immunoadhesin” designates antibody-like or immunoglobulin-like molecules which combine the “binding domain” of a heterologous protein (an “adhesin”, e.g. a receptor, ligand or enzyme) with the effector component of immunoglobulin constant domains (i.e., Fc domain). Structurally, the imnunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG1, IgG2, IgG3, or IgG4 subtypes, IgA, IgE, IgD or IgM.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the term “wild-type amino acid,” “wild-type IgG,” or “wild-type mAb” refers to a sequence of amino acids or nucleic acids that occurs naturally within a certain population (e.g., human, mice, rats, cells, etc.).

As used herein, the term “conjugation efficiency” or “crosslinking efficiency” is the ratio between the experimentally measured amounts of the ADC as described herein divided by the maximum expected ADC amount. Conjugation efficiency or crosslinking efficiency can be measured by various techniques well known to persons skilled in the art, such as hydrophobic interaction chromatography. Conjugation efficiency can also be measured at different temperature, such as room temperature or 37° C.

The term “effector function” refers to the biological activities attributable to the Fc region of an antibody. Examples of antibody effector functions include, but are not limited to, antibody-dependent cell-mediated cytotoxicity (ADCC), Fc receptor binding, complement dependent cytotoxicity (CDC), phagocytosis, C1 q binding, and down regulation of cell surface receptors (e.g., B cell receptor; BCR). See, e.g., U.S. Pat. No. 6,737,056. Such effector functions generally require the Fc region to be combined with a binding domain (e.g., an antibody variable domain) and can be assessed using various assays known in the art for evaluating such antibody effector functions. An exemplary measurement of effector function is through Fcv3 and/or C1 q binding.

As used herein “antibody-dependent cell-mediated cytotoxicity” or “ADCC” refers to a cell-mediated reaction in which nonspecific cytotoxic cells that express Fe receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” or “CDC” refers to the lysing of a target in the presence of complement. The complement activation pathway is initiated by the binding of the first component of the complement system (C1 q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcvRI, FcvRII, FcvRIII, and FcyRIV subclasses, including allelic variants and alternatively spliced forms of these receptors. FcyRII receptors include FcyRIIA (an “activating receptor”) and FcvRIIB (an “inhibiting receptor”), which have similar amino acid sequences that differ primarily in the cytoplasmic domains thereof. FcRs are reviewed in Ravetch and Kinet. 1991, Ann. Rev. Immunol., 9:457-92; Capel et al., 1994, Immunomethods, 4:25-34; de Haas et al., 1995. J. Lab. Clin. Med., 126:330-41; Nimmerjahn et al., 2005, Immunity 23:2-4. “FcR” also includes the neonatal receptor, FcRn, which is responsible for the transfer of maternal IgGs to the fetus (Guyer et al., 1976. J. Immunol., 1 17:587; and Kim et al., 1994, J. Immunol., 24:249).

### 2. Description of Exemplary Embodiments of Ligand-Payload Conjugates

In one aspect, the present disclosure provides a ligand-payload conjugate of Formula (I):

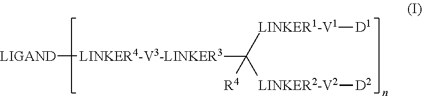

or a pharmaceutically acceptable salt thereof, wherein:
LIGAND is a ligand;
each of V¹, V², and V³ is independently a linker moiety formed by a coupling reaction;
each of LINKER¹, LINKER², LINKER³, and LINKER⁴ is independently a bivalent linker;
each of D¹ and D² is independently a payload;
each R⁴ is independently R, —N(R)₂, or —N(R)C(O)—R;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur; and
n is 2, 3, 4, 5, or 6.

In one aspect, the present invention provides a ligand-payload conjugate of Formula (I*):

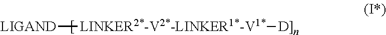

or a pharmaceutically acceptable salt thereof, wherein:
LIGAND is a ligand;
each of V¹* and V²* is independently a linker moiety formed by a coupling reaction;
each of LINKER¹* and LINKER²* is independently a bivalent linker;
each D is independently a payload;
n is 2, 3, 4, 5, 6, 7, 8, 9, 10, 11, or 12.

### 2.1. Ligand

As defined generally above, LIGAND is a ligand. A ligand includes within its scope any unit of a ligand that binds or reactively associates or complexes with a receptor, antigen or other receptive moiety associated with a given target-cell population and/or tissue. A ligand can be any molecule that binds to, complexes with or reacts with a moiety of a cell population and/or tissue sought to be therapeutically or otherwise biologically modified. A ligand acts to deliver a payload to the particular target cell population with which the ligand moiety reacts.

In some embodiments, a ligand is selected from large molecular weight proteins such as, for example, full-length antibodies, antibody fragments, smaller molecular weight proteins, polypeptide or peptides, and lectins.

In some embodiments, a ligand comprises a full-length antibody heavy chain and an antibody light chain. In some embodiments, a ligand is a monoclonal antibody, a polyclonal antibody, a human antibody, a humanized antibody, a chimeric antibody, a bispecific antibody, a minibody, a diabody, or an antibody fragment.

In some embodiments, an antibody is an IgG. In some embodiments, the IgG is selected from the group consisting of IgG1, IgG2, IgG3, and IgG4.

In some embodiments, an antibody is an IgA, IgE, IgD, or IgM.

In some embodiments, an antibody is an anti-A33 antibody, an anti-B7-H3 antibody, an anti-CanAg antibody, an anti-CD20 antibody, an anti-CD22 antibody, an anti-CD30 antibody, an anti-CD33 antibody, an anti-CD56 antibody, an anti-CD70 antibody, an anti-CEA antibody, an anti-Cripto antibody, an anti-EphA2 antibody, an anti-G250 antibody, anti-CRTAM antibody, an anti-MUC1 antibody, an anti-GPNMB antibody, an anti-integrin antibody, an anti-PSMA antibody, an anti-tenascin-C antibody, an anti-SLC44A4 antibody, or an anti-mesothelin antibody. In some embodiments, an anti-mesothelin antibody is selected from the group consisting of DLK1, EDB, EDA, Nectin4, SLITRK6, SLC7A11, MUC16, TEM8, FAP, LRRC15, Notch 3, Ephrin A4, LIV-1, ROR1, Tissue Factor, Claudin 6, Glypican 3, and FGFR3.

In some embodiments, an antibody is selected from: trastuzumab, trastuzumab mutants (such as the trastuzumab mutants disclosed in WO2014068443 or WO2013068946), oregovomab, edrecolomab, cetuximab, a humanized monoclonal antibody to the vitronectin receptor (θvβ3), alemtuzumab, anti-HLA-DR antibodies including a humanized anti-HLA-DR antibody for the treatment of non-Hodgkin's lymphoma, 1311 Lym-1, anti-cd33 antibodies, anti-cd22 antibodies including a humanized anti-CD22 mAb for the treatment of Hodgkin's Disease or non-Hodgkin's lymphoma, labetuzumab, bevacizumab, ibritumomab tiuxetan, ofatumumab, panitumumab, rituximab, tositumomab, ipilimumab, and gemtuzumab.

In some embodiments, an antibody, or antigen-binding fragment thereof, is selected from the group consisting of an anti-HER2 (ErbB2) antibody, an anti-EGFR antibody, an anti-B7H3 antibody, anti-c-Met antibody, anti-HER3 (ErbB3) antibody, anti-HER4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-CRTAM antibody, anti-MUCl antibody, anti-Lewis Y antibody, anti-VEGFR antibody, anti-GPNMB antibody, anti-Integrin Antibodies, anti-PSMA antibodies, anti-Tenascin-C antibodies, anti-SLC44A4 antibodies, anti-Mesothelin antibodies, anti-SLC39A6 antibody, anti-Nectin4 antibody, Anti-ANTXR1 antibody, anti-TROP2 antibody, Anti-CDCP1 antibody, anti-FAP antibody, anti-ROR1 antibody, anti-TM4SF1 antibody, anti-CDH6 antibody, anti-SLC7A11 antibody, anti-LRRC15 antibody, anti-EPCAM antibody, anti-tissue factor antibody, anti-Folate Receptor antibody, anti-CEACAM6 antibody, anti-claudin 6 antibody, anti-TNFRSF17 antibody, anti-DLK1 antibody, anti-CD19 antibody, anti-TNFRSF13B antibody, anti-CD274 antibody, anti-Notch3 antibody, anti-CLEC12A antibody, anti-CD38 antibody, anti-CD79b antibody, anti-DLL3 antibody, anti-SLAMF7 antibody, anti-ITGB1 antibody, anti-cadherin 11 antibody, anti-claudin 11 antibody, anti-SLC16A1 antibody, anti-SLC45A2 antibody, anti-SLC6A3 antibody, anti-SLC1A6 antibody, anti-SLC6A2 antibody, anti-SLC26A3 antibody, anti-SLC26A3 antibody, anti-SLC26A9 antibody, anti-TRPM1 antibody, anti-NCAN antibody, anti-CACNG7 antibody, anti-SYT4 antibody, anti-NPSR1 antibody, anti-Cadherin19 antibody, anti-LRP8 antibody, anti-GPC3 antibody, anti-TPBG antibody, anti-Claudin18 antibody, anti-FOLH1 antibody, anti-SLC34A2 antibody, anti-FGFR2 antibody, anti-FGFR3 antibody, anti-CCR antibody, anti-MUC16 antibody, anti-CD93 antibody, anti-LGALS3BP antibody, anti-EDB antibody, anti-EDA antibody, anti-COL11A1 antibody, anti-SLITRK6 antibody or antigen-binding fragments thereof.

In some embodiments, an antibody or antigen-binding fragment thereof is selected from the group consisting of Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96, and Glembatumumab, or antigen-binding fragments thereof.

In some embodiments, the number of the

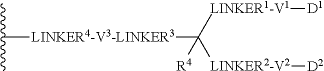

moiety attached to a ligand is n, which is 2, 3, 4, 5, or 6. In some embodiments, n is 2, which means a payload-to-ligand ratio of 4. In some embodiments, n is 3, which means a payload-to-ligand ratio of 6. In some embodiments, n is 4, which means a payload-to-ligand ratio of 8. In some embodiments, n is 5, which means a payload-to-ligand ratio of 10. In some embodiments, n is 6, which means a payload-to-ligand ratio of 12.

In some embodiments, a ligand is an antibody, which is site-specifically conjugated to each of the

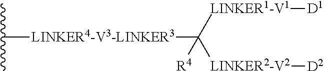

moiety, for example, at a site selected from any of the amino acid positions as listed in Table 1 below.

In some embodiments, a ligand is an antibody, and the number of the

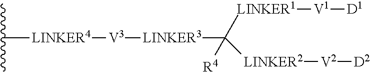

moiety attached to an antibody is n, which is 2, 3, 4, 5, or 6. In some embodiments, n is 2, which means a drug-to-antibody ratio of 4 (DAR4). In some embodiments, n is 3, which means a drug-to-antibody ratio of 6 (DAR6). In some embodiments, n is 4, which means a drug-to-antibody ratio of 8 (DAR8). In some embodiments, n is 5, which means a drug-to-antibody ratio of 10 (DAR10). In some embodiments, n is 6, which means a drug-to-antibody ratio of 12 (DAR12).

In some embodiments, R⁴ is H. In some embodiments, R⁴ is —CH₃. In some embodiments, R⁴ is

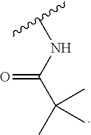 [SMILES: *NC(=O)C(C)(C)C]

In some embodiments, R⁴ is

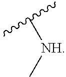 [SMILES: *NC]

In some embodiments, R⁴ is

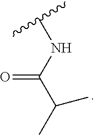 [SMILES: *NC(=O)C(C)C]

In some embodiments, R⁴ is

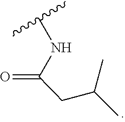 [SMILES: *NC(=O)CC(C)C]

In some embodiments, R⁴ is

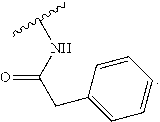 [SMILES: *NC(=O)Cc1ccccc1]

In some embodiments, R⁴ is

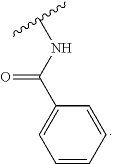 [SMILES: *NC(=O)c1ccccc1]

In some embodiments, R⁴ is

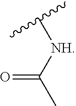 [SMILES: *NC(C)=O]

In some embodiments, R⁴ is

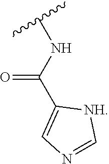 [SMILES: *NC(=O)c1cnc[nH]1]

In some embodiments, R⁴ is

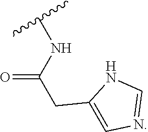 [SMILES: *NC(=O)Cc1cnc[nH]1]

In some embodiments, R⁴ is

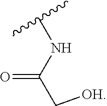 [SMILES: *NC(=O)CO]

In some embodiments, the number of the

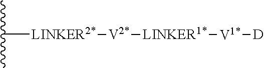

moiety attached to a ligand is n, which is 2, 3, 4, 5, 6, 7, 8, 9, 10, 11, or 12. In some embodiments, n is 2, which means a payload-to-ligand ratio of 2. In some embodiments, n is 3, which means a payload-to-ligand ratio of 3. In some embodiments, n is 4, which means a payload-to-ligand ratio of 4. In some embodiments, n is 5, which means a payload-to-ligand ratio of 5. In some embodiments, n is 6, which means a payload-to-ligand ratio of 6. In some embodiments, n is 7, which means a payload-to-ligand ratio of 7. In some embodiments, n is 8, which means a payload-to-ligand ratio of 8. In some embodiments, n is 9, which means a payload-to-ligand ratio of 9. In some embodiments, n is 10, which means a payload-to-ligand ratio of 10. In some embodiments, n is 11, which means a payload-to-ligand ratio of 11. In some embodiments, n is 12, which means a payload-to-ligand ratio of 12.

In some embodiments, a ligand is an antibody, which is site-specifically conjugated to each of the

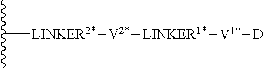

moiety, for example, at a site selected from any of the amino acid positions as listed in Table 1 below.

In some embodiments, a ligand is an antibody, and the number of the

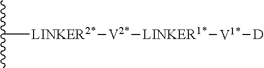

moiety attached to an antibody is n, which is 2, 3, 4, 5, 6, 7, 8, 9, 10, 11, or 12. In some embodiments, n is 2, which means a drug-to-antibody ratio of 2 (DAR2). In some embodiments, n is 3, which means a drug-to-antibody ratio of 4 (DAR3). In some embodiments, n is 4, which means a drug-to-antibody ratio of 4 (DAR4). In some embodiments, n is 5, which means a drug-to-antibody ratio of 5 (DAR5). In some embodiments, n is 6, which means a drug-to-antibody ratio of 6 (DAR6). In some embodiments, n is 7, which means a drug-to-antibody ratio of 7 (DAR7). In some embodiments, n is 8, which means a drug-to-antibody ratio of 8 (DAR8). In some embodiments, n is 9, which means a drug-to-antibody ratio of 9 (DAR9). In some embodiments, n is 10, which means a drug-to-antibody ratio of 10 (DAR10). In some embodiments, n is 11, which means a drug-to-antibody ratio of 11 (DAR11). In some embodiments, n is 12, which means a drug-to-antibody ratio of 12 (DAR12).
2.2. Bivalent Linker LINKER⁴ and LINKER²*

As defined generally above, each LINKER⁴ is independently a bivalent linker.

In some embodiments, LINKER⁴ is S⁴, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

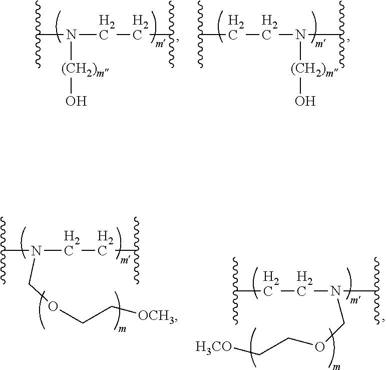

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein:
each -Cy- is independently an optionally substituted 3-8 membered bivalent saturated, partially unsaturated, or aromatic ring having 0-4 heteroatoms independently selected from nitrogen, oxygen, or sulfur;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

As defined generally above, each S⁴ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

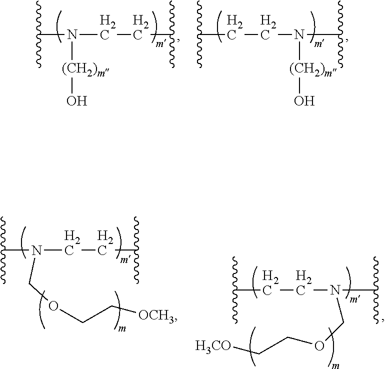

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S⁴ is a covalent bond.

In some embodiments, S⁴ is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ—,

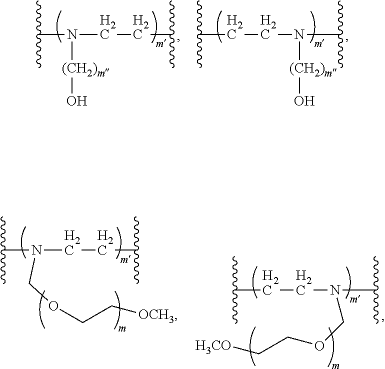

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S⁴ is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

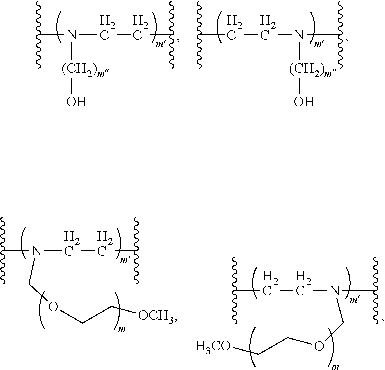

—S—, —N(R)—, —O—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S⁴ is a bivalent C₁₋₈ saturated or unsaturated, straight or branched, hydrocarbon chain wherein three methylene units of the chain are optionally and independently replaced by -Cy-, —C(R)₂—, —(OCH₂CH₂)ₘ—, —(CH₂CH₂O)ₘ,

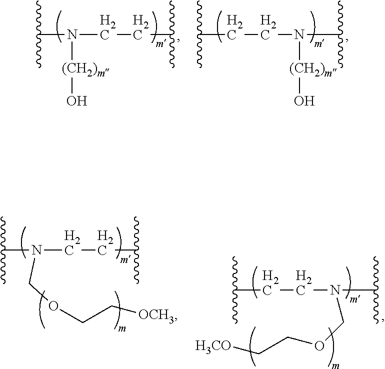

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

As defined generally above, each -Cy- is independently an optionally substituted 3-8 membered bivalent saturated, partially unsaturated, or aromatic ring having 0-4 heteroatoms independently selected from nitrogen, oxygen, or sulfur.

In some embodiments, -Cy- is an optionally substituted 3, 4, 5, 6, 7, or 8 membered bivalent saturated ring having 0, 1, 2, 3, or 4 heteroatoms independently selected from nitrogen, oxygen, or sulfur.

In some embodiments, -Cy- is an optionally substituted 3, 4, 5, 6, 7, or 8 membered bivalent unsaturated ring having 0, 1, 2, 3, or 4 heteroatoms independently selected from nitrogen, oxygen, or sulfur.

In some embodiments, -Cy- is an optionally substituted 3, 4, 5, 6, 7, or 8 membered bivalent aromatic ring having 0, 1, 2, 3, or 4 heteroatoms independently selected from nitrogen, oxygen, or sulfur.

As defined generally above, each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur.

In some embodiments, R is H. In some embodiments, R is optionally substituted C₁₋₆ aliphatic. In some embodiments, R is an optionally substituted 3-8 membered saturated or partially unsaturated monocyclic carbocyclic ring. In some embodiments, R is an optionally substituted 3-8 membered saturated or partially unsaturated monocyclic heterocyclic ring having 1-4 heteroatoms independently selected from nitrogen, oxygen, or sulfur. In some embodiments, R is optionally substituted phenyl. In some embodiments, R is an optionally substituted 5-6 membered monocyclic heteroaromatic ring having 1-4 heteroatoms independently selected from nitrogen, oxygen, or sulfur. In some embodiments, R is an optionally substituted 8-10 membered bicyclic aromatic carbocyclic ring. In some embodiments, R is an optionally substituted 8-10 membered bicyclic heteroaromatic ring having 1-5 heteroatoms independently selected from nitrogen, oxygen, or sulfur.

As defined generally above, each m is independently 0, 1, 2, 3, 4, 5, 6, 7, 8, 9, 10, 11, 12, 13, 14, 15, 16, 17, 18, 19, 20, 21, 22, 23, or 24. In some embodiments, m is 0. In some embodiments, m is 1, 2, 3, 4, or 5. In some embodiments, m is 6, 7, 8, 9, or 10. In some embodiments, m is 11, 12, 13, 14, or 15. In some embodiments, m is 16, 17, 18, 19, or 20. In some embodiments, m is 21, 22, 23, or 24. In some embodiments, m is 2, 4, 8, 12, 16, 20, or 24.

As defined generally above, each m′ is independently 0, 1, 2, 3, 4, 5, 6, 7, 8, 9, 10, 11, 12, 13, 14, 15, 16, 17, 18, 19, 20, 21, 22, 23, or 24. In some embodiments, m′ is 0. In some embodiments, m′ is 1, 2, 3, 4, or 5. In some embodiments, m′ is 6, 7, 8, 9, or 10. In some embodiments, m′ is 11, 12, 13, 14, or 15. In some embodiments, m′ is 16, 17, 18, 19, or 20. In some embodiments, m′ is 21, 22, 23, or 24. In some embodiments, m′ is 2, 4, 8, 12, 16, 20, or 24.

As defined generally above, each m″ is independently 1, 2, or 3. In some embodiments, m″ is 1. In some embodiments, m″ is 2. In some embodiments, m″ is 3.

In some embodiments, LINKER⁴ is S⁴, which is a glutamine-containing tag engineered at a specific site, 2) an endogenous glutamine, and/or 3) an endogenous glutamine made reactive by antibody engineering with an engineered transglutaminase, and V³ is —C(O)N(R)—, and wherein the carbonyl moiety of V³ is the carbonyl moiety of the γ-carboxamide groups (—(C═O)NH₂) of glutamine residue of S⁴.

In some embodiments, the present disclosure provides an antibody-drug conjugate (ADC), which is a ligand-payload conjugate of Formula (I), wherein LIGAND is an antibody; each LINKER⁴ is independently S⁴, which is a glutamine-containing tag engineered at a specific site, 2) an endogenous glutamine, and/or 3) an endogenous glutamine made reactive by antibody engineering or an engineered transglutaminase, and V³ is —C(O)N(R)—, and wherein the carbonyl moiety of V³ is the carbonyl moiety of the γ-carboxamide groups (—(C═O)NH₂) of glutamine residue of S⁴.

In some embodiments, S⁴ is a glutamine-containing tag comprising, or consisting of, an amino acid sequence XXQX (SEQ ID NO:24), and V³ is —C(O)N(R)—, wherein the carbonyl moiety of V³ is the carbonyl moiety of the γ-carboxamide groups (—(C═O)NH₂) of glutamine residue of S⁴, wherein X can be a conventional or nonconventional amino acid, as described herein. For example, in some embodiments. X is L (Leu), A (Ala), G (Gly), S (Ser), V (Val), F (Phe), Y (Tyr), H (His), R (Arg), N (Asn), E (Glu), D (Asp), C (Cys), Q (Gln), I (lie), M (Met), P (Pro), T (Thr), K (Lys), or W (Trp). In some embodiments, S⁴ is a glutamine-containing tag, comprising, or consisting of, an amino acid sequence selected from the group consisting of Q, LQG, LLQGG (SEQ ID NO:1), LLQG (SEQ ID NO:2), LSLSQG (SEQ ID NO:3), GGGLLQGG (SEQ ID NO:4), GLLQG (SEQ ID NO:5), LLQ, GSPLAQSHGG (SEQ ID NO:6), GLLQGGG (SEQ ID NO:7), GLLQGG (SEQ ID NO:8), GLLQ (SEQ ID NO:9), LLQLLQGA (SEQ ID NO: 10), LLQGA (SEQ ID NO:11), LLQYQGA (SEQ ID NO:12), LLQGSG (SEQ ID NO:13), LLQYQG (SEQ ID NO:14), LLQLLQG (SEQ ID NO:15), SLLQG (SEQ ID NO:16), LLQLQ (SEQ ID NO:17). LLQLLQ (SEQ ID NO:18), LLQGR (SEQ ID NO:19), LLQGPP (SEQ ID NO:20), LLQG PA (SEQ ID NO:21), GGLLQGPP (SEQ ID NO:22), GGLLQGA (SEQ ID NO:23), LLQGPGK (SEQ ID NO:25), LLQGPG (SEQ ID NO:26), LLQGP (SEQ ID NO:27), LLQP (SEQ ID NO:28), LLQPGK (SEQ ID NO:29), LLQAPGK (SEQ ID NO:30), LLQGAPG (SEQ ID NO:31), LLQGAP (SEQ ID NO:32), and LLQLQG (SEQ ID NO:33), and V³ is —C(O)N(R)—, wherein the carbonyl moiety of V³ is the carbonyl moiety of the γ-carboxamide groups (—(C═O)NH₂) of glutamine residue of S⁴.

In some embodiments, S⁴ is a glutamine-containing tag selected from those listed in Table 1 below, and V³ is —C(O)N(R)—, wherein the carbonyl moiety of V³ is the carbonyl moiety of the γ-carboxamide groups (—(C═O)NH₂) of glutamine residue of S⁴.

As defined generally above, each LINKER²* is independently a bivalent linker.

In some embodiments, LINKER²* is S²*, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ, —(CH₂CH₂O)ₘ,

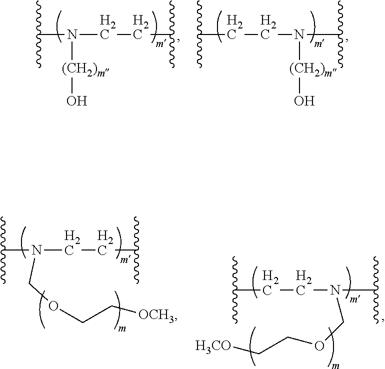

C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein:
each -Cy- is independently an optionally substituted 3-8 membered bivalent saturated, partially unsaturated, or aromatic ring having 0-4 heteroatoms independently selected from nitrogen, oxygen, or sulfur;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

As defined generally above, each S²* is independently a covalent bond or a bivalent C₁₋₈ saturated or unsaturated, straight or branched, hydrocarbon chain wherein one, two or three methylene units of the chain are optionally and independently replaced by -Cy-,

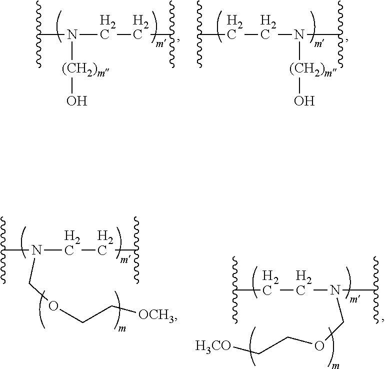

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S²* is a covalent bond.

In some embodiments, S²* is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ—,

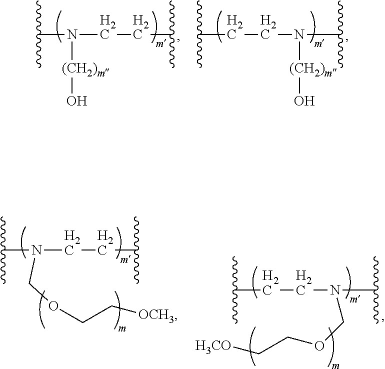

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S²* is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ,

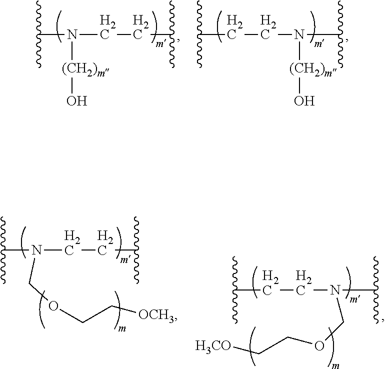

—S—, —N(R)—, —O—, —C(O)—, —OC(O)—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S²* is a bivalent C₁₋₈ saturated or unsaturated, straight or branched, hydrocarbon chain wherein three methylene units of the chain are optionally and independently replaced by -Cy-, —C(R)₂—, —(OCH₂CH₂)ₘ—, —(CH₂CH₂O)ₘ—,

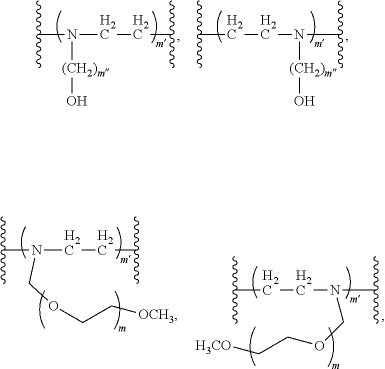

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

As defined generally above, each -Cy- is independently an optionally substituted 3-8 membered bivalent saturated, partially unsaturated, or aromatic ring having 0-4 heteroatoms independently selected from nitrogen, oxygen, or sulfur.

In some embodiments, -Cy- is an optionally substituted 3, 4, 5, 6, 7, or 8 membered bivalent saturated ring having 0, 1, 2, 3, or 4 heteroatoms independently selected from nitrogen, oxygen, or sulfur.

In some embodiments, -Cy- is an optionally substituted 3, 4, 5, 6, 7, or 8 membered bivalent unsaturated ring having 0, 1, 2, 3, or 4 heteroatoms independently selected from nitrogen, oxygen, or sulfur.

In some embodiments, -Cy- is an optionally substituted 3, 4, 5, 6, 7, or 8 membered bivalent aromatic ring having 0, 1, 2, 3, or 4 heteroatoms independently selected from nitrogen, oxygen, or sulfur.

As defined generally above, each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur.

In some embodiments, R is H. In some embodiments, R is optionally substituted C₁₋₆ aliphatic. In some embodiments, R is an optionally substituted 3-8 membered saturated or partially unsaturated monocyclic carbocyclic ring. In some embodiments, R is an optionally substituted 3-8 membered saturated or partially unsaturated monocyclic heterocyclic ring having 1-4 heteroatoms independently selected from nitrogen, oxygen, or sulfur. In some embodiments, R is optionally substituted phenyl. In some embodiments, R is an optionally substituted 5-6 membered monocyclic heteroaromatic ring having 1-4 heteroatoms independently selected from nitrogen, oxygen, or sulfur. In some embodiments, R is an optionally substituted 8-10 membered bicyclic aromatic carbocyclic ring. In some embodiments, R is an optionally substituted 8-10 membered bicyclic heteroaromatic ring having 1-5 heteroatoms independently selected from nitrogen, oxygen, or sulfur.

As defined generally above, each m is independently 0, 1, 2, 3, 4, 5, 6, 7, 8, 9, 10, 11, 12, 13, 14, 15, 16, 17, 18, 19, 20, 21, 22, 23, or 24. In some embodiments, m is 0. In some embodiments, m is 1, 2, 3, 4, or 5. In some embodiments, m is 6, 7, 8, 9, or 10. In some embodiments, m is 11, 12, 13, 14, or 15. In some embodiments, m is 16, 17, 18, 19, or 20. In some embodiments, m is 21, 22, 23, or 24. In some embodiments, m is 4, 8, 12, 16, 20, or 24.

As defined generally above, each m′ is independently 0, 1, 2, 3, 4, 5, 6, 7, 8, 9, 10, 11, 12, 13, 14, 15, 16, 17, 18, 19, 20, 21, 22, 23, or 24. In some embodiments, m′ is 0. In some embodiments, m′ is 1, 2, 3, 4, or 5. In some embodiments, m′ is 6, 7, 8, 9, or 10. In some embodiments, m′ is 11, 12, 13, 14, or 15. In some embodiments, m′ is 16, 17, 18, 19, or 20. In some embodiments, m′ is 21, 22, 23, or 24. In some embodiments, m′ is 4, 8, 12, 16, 20, or 24.

As defined generally above, each m″ is independently 1, 2, or 3. In some embodiments, m″ is 1. In some embodiments, m″ is 2. In some embodiments, m″ is 3.

In some embodiments, LINKER²* is S²*, which is a glutamine-containing tag engineered at a specific site, 2) an endogenous glutamine, and/or 3) an endogenous glutamine made reactive by antibody engineering or an engineered transglutaminase, and V²* is —C(O)N(R)—, and wherein the carbonyl moiety of V²* is the carbonyl moiety of the γ-carboxamide groups (—(C═O)NH₂) of glutamine residue of S²*.

In some embodiments, the present invention provides an antibody-drug conjugate (ADC), which is a ligand-payload conjugate of Formula (I*), wherein LIGAND is an antibody; each LINKER²* is independently S²*, which is a glutamine-containing tag engineered at a specific site, 2) an endogenous glutamine, and/or 3) an endogenous glutamine made reactive by antibody engineering or an engineered transglutaminase, and V²* is —C(O)N(R)—, and wherein the carbonyl moiety of V²* is the carbonyl moiety of the γ-carboxamide groups (—(C═O)NH₂) of glutamine residue of S²*.

In some embodiments, S²* is a glutamine-containing tag comprising, or consisting of an amino acid sequence XXQX (SEQ ID NO:24), and V²* is —C(O)N(R)—, wherein the carbonyl moiety of V²* is the carbonyl moiety of the γ-carboxamide groups (—(C═O)NH₂) of glutamine residue of S²*, wherein X can be a conventional or nonconventional amino acid, as described herein. For example, in some embodiments, X is L (Lei), A (Ala), G (Gly) S (Ser), V (Val), F (Phe), Y (Tyr), H (His), R (Arg), N (Asn), E (Glu), D (Asp), C (Cys), Q (Gln), I (lie), M (Met), P (Pro), T (Thr), K (Lys), or W (Trp). In some embodiments. S²* is a glutamine-containing tag, comprising, or consisting of, an amino acid sequence selected from the group consisting of Q, LQG, LLQGG (SEQ ID NO:1), LLQG (SEQ ID NO:2), LSLSQG (SEQ ID NO:3), GGGLLQGG (SEQ ID NO:4), GLLQG (SEQ ID NO:5), LLQ, GSPLAQSHGG (SEQ ID NO:6), GLLQGGG (SEQ ID NO:7), GLLQGG (SEQ ID NO:8), GLLQ (SEQ ID NO:9). LLQLLQGA (SEQ ID NO:10). LLQGA (SEQ ID NO: 11), LLQYQGA (SEQ II) NO:12), LLQGSG (SEQ II) NO:13), LLQYQG (SEQ II) NO:14), LLQLLQG (SEQ ID NO: 15). SLLQG (SEQ ID NO:16), LLQLQ (SEQ ID NO:17), LLQLLQ (SEQ ID NO:18). LLQGR (SEQ ID NO:19), LLQGPP (SEQ ID NO:20), LLQG PA (SEQ ID NO:21), GGLLQGPP (SEQ ID NO:22), GGLLQGA (SEQ ID NO:23), LLQGPGK (SEQ ID NO:25), LLQGPG (SEQ ID NO:26), LLQGP (SEQ ID NO:27), LLQP (SEQ ID NO:28), LLQPGK (SEQ ID NO:29), LLQAPGK (SEQ ID NO:30), LLQGAPG (SEQ ID NO:31), LLQGAP (SEQ ID NO:32), and LLQLQG (SEQ ID NO:33), and V², is —C(O)N(R)—, wherein the carbonyl moiety of V²* is the carbonyl moiety of the γ-carboxamide groups (—(C═O)NH₂) of glutamine residue of S²*.

In some embodiments, S²* is a glutamine-containing tag selected from those listed in Table 1 below, and V²* is —C(O)N(R)—, wherein the carbonyl moiety of V²* is the carbonyl moiety of the γ-carboxamide groups (—(C═O)NH₂) of glutamine residue of S²*.

TABLE 1
Examples of glutamine-containing tags and corresponding engineered
positions.

| Glutamine- | | |
|---|---|---|
| containing | | |
| Tag Name | Sequence | Amino Acid Positions |
| TG6 | LLQGA (SEQ ID NO: 11) | C-terminus of the antibody heavy chain (e.g., |
| | | K447) |
| LCQ04 | GGLLQGA (SEQ ID NO: 23) | C-terminus of the antibody light chain |
| LCQ05 | GGLLQGPP (SEQ ID NO: 22) | C-terminus of the antibody light chain |
| H7c | LLQG (SEQ ID NO: 2) | Insertion after residue T135 in the antibody heavy |
| | | chain |
| L11b | LLQG (SEQ ID NO: 2) | Replacement of residues G200-S202 with the |
| | | glutamine-containing tag in the antibody light |
| | | chain |
| H1 | LLQGSG (SEQ ID NO: 13) | N-terminus |
| H8a | LLQG (SEQ ID NO: 2) | Insertion after residue S160 in the antibody heavy |
| | | chain |
| H10 | LLQG (SEQ ID NO: 2) | Replacement of residues S190-S192 with the |
| | | glutamine-containing tag in the antibody heavy |
| | | chain |
| H10a | LLQYQG (SEQ ID NO: 14) | Replacement of residues P189-S192 with the |
| | | glutamine-containing tag in the antibody heavy |
| | | chain |
| H10b | LLQLLQG (SEQ ID NO: 15) | Replacement of residues P189-S192 with the |
| | | glutamine-containing tag in the antibody heavy |
| | | chain |
| H12 | LLQG (SEQ ID NO: 2) | Replacement of residues K222-T225 with the |
| | | glutamine-containing tag in the antibody heavy |
| | | chain |
| H12c | LLQG (SEQ ID NO: 2) | Replacement of residues K222-T223 with the |
| | | glutamine-containing tag in the antibody heavy |
| | | chain |
| H12d | LLQG (SEQ ID NO: 2) | Insertion after residue T223 in the antibody heavy |
| | | chain |
| H13a | SLLQG (SEQ ID NO: 16) | Replacement of residues L251-S254 with the |
| | | glutamine-containing tag in the antibody heavy |
| | | chain |
| H13b | LQG | Replacement of residues M252-1253 with the |
| | | glutamine-containing tag in the antibody heavy |
| | | chain |
| H16 | LLQG (SEQ ID NO: 2) | Replacement of residues E294-N297 with the |
| | | glutamine-containing tag in the antibody heavy |
| | | chain |
| H16a | LLQLQG (SEQ ID NO: 33) | Replacement of residues E293-N297 with the |
| | | glutamine-containing tag in the antibody heavy |
| | | chain |
| H16b | LLQLLQG (SEQ ID NO: 15) | Replacement of residues E293-N297 with the |
| | | glutamine-containing tag in the antibody heavy |
| | | chain |
| H16c | LLQLQ (SEQ ID NO: 17) | Replacement of residues E294-N297 with the |
| | | glutamine-containing tag in the antibody heavy |
| | | chain |
| H16d | LLQLLQ (SEQ ID NO: 18) | Replacement of residues E294-N297 with the |
| | | glutamine-containing tag in the antibody heavy |
| | | chain |
| N297A | A | Replacement of residue N297 with amino acid A, |
| | | resulting in aglycosylation at position 297 and |
| | | accessible/reactive endogenous glutamine at |
| | | position 295 |
| H21a | LLQG (SEQ ID NO: 2) | Insertion after residue G385 in the antibody heavy |
| | | chain |
| L2 | LLQG (SEQ ID NO: 2) | Replacement of residues S60-R61 with the |
| | | glutamine-containing tag in the antibody light |
| | | chain |
| L4b | LLQG (SEQ ID NO: 2) | Insertion after residue R108 in the antibody light |
| | | chain |
| L8a | LLQG (SEQ ID NO: 2) | Insertion after residue S168 in the antibody light |
| | | chain |
| L11b | LLQG (SEQ ID NO: 2) | Replacement of residues G200-S202 with the |
| | | glutamine-containing tag in the antibody light |
| | | chain |
| L11c | LLQGR (SEQ ID NO: 19) | Replacement of residues G200-S202 with the |
| | | glutamine-containing tag in the antibody light |
| | | chain |
| TG4 | LLQYQGA (SEQ ID NO: 12) | C terminus of the antibody heavy chain (e.g., |
| | | K447) |
| TG5 | LLQLLQGA (SEQ ID NO: | C-terminus of the antibody heavy chain (e.g., |
| | 10) | K447) |
| TG17 | LLQGPP (SEQ ID NO: 20) | C-terminus of the antibody heavy chain (e.g., |
| | | K447) |

In some embodiments, S⁴ is a glutamine-containing tag selected from those described in US 2013/0230543, US2013/0122020, and US 20170/0043033, each of which is incorporated by reference herein in its entirety, and V³ is —C(O)N(R)—, wherein the carbonyl moiety of V³ is the carbonyl moiety of the γ-carboxamide groups (—(C═O)NH₂) of glutamine residue of S⁴.

In some embodiments, S²* is a glutamine-containing tag selected from those described in US2013/0230543, US2013/0122020, and US2017/0043033, each of which is incorporated by reference herein in its entirety, and V²* is —C(O)N(R)—, wherein the carbonyl moiety of V²* is the carbonyl moiety of the γ-carboxamide groups (—(C═O)NH₂) of glutamine residue of S²*

2.3. Linker Moiety V¹, V², V³, V¹*, and V²*

As defined generally above, each of V¹, V², and V³ is independently a linker moiety formed by a coupling reaction. In some embodiments, each of V¹, V², and V³ is independently a linker moiety formed by a coupling reaction selected from those listed in Table 2. In some embodiments, each of V¹, V², and V³ is independently a linker moiety formed by a coupling reaction between a thiol group and a maleimide group, wherein the maleimide group is selected from those as described in U.S. Pat. No. 9,504,756, and Lyon, et al., Nature Biotechnology 2014, doi:10.1038/nbt.2968, each of which is incorporated herein by reference in its entirety. In some embodiments, each of V¹, V², and V³ is independently a linker moiety formed by a coupling reaction selected from those as described in Agarwal et al., Bioconjugate Chem. 2015, 26, 176-192, and Dolan et al., Antibody Therapeutics 2020, Vol. 3, No. 4, 271-284, each of which is incorporated herein by reference in its entirety.

As defined generally above, each of V¹* and V²* is independently a linker moiety formed by a coupling reaction. In some embodiments, each of V¹* and V²* is independently a linker moiety formed by a coupling reaction selected from those listed in Table 2. In some embodiments, each of V¹* and V²* is independently a linker moiety formed by a coupling reaction between a thiol group and a maleimide group, wherein the maleimide group is selected from those as described in U.S. Pat. No. 9,504,756, and Lyon, et al., Nature Biotechnology 2014, doi:10.1038/nbt.2968, each of which is incorporated herein by reference in its entirety. In some embodiments, each of V¹* and V²* is independently a linker moiety formed by a coupling reaction selected from those as described in Agarwal et al., Bioconjugate Chem. 2015, 26, 176-192, and Dolan et al., Antibody Therapeutics 2020, Vol. 3, No. 4, 271-284, each of which is incorporated herein by reference in its entirety.

TABLE 2
Exemplary Cross-linking Technologies

| Coupling | | |
|---|---|---|
| Reaction | | |
| Type | Functional Groups | Linker Moiety Formed |
| Thiol-yne | 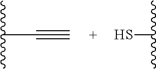 | 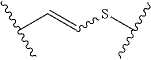 [SMILES: *C=CS*] |
| NHS ester | 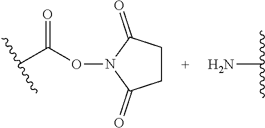 | 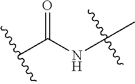 [SMILES: *NC(*)=O] |
| Thiol-ene | 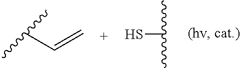 | 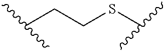 [SMILES: *CCS*] |
| Isocyanate | 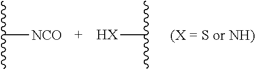 | 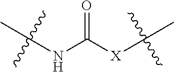 |
| Epoxide or aziridine | 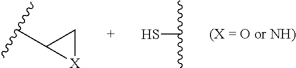 | 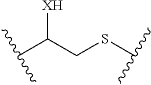 |
| Aldehyde- aminoxy | 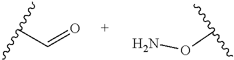 | 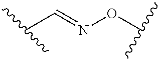 [SMILES: */C=N/O*] |
| Ketone- aminoxy | 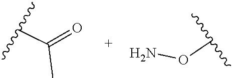 | 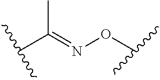 [SMILES: *O/N=C(/*)C] |
| Ketone- hydrazide | 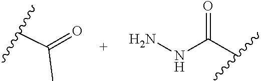 | 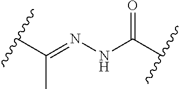 [SMILES: *C(=O)N/N=C(/*)C] |
| Cu- catalyzed- azide-alkyne cycloaddition | 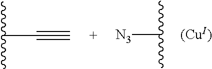 | 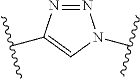 [SMILES: *c1cn(*)nn1] |
| Strain- promoted cycloaddition | Cyclooctyne cycloaddition (with azide, nitrile, or nitrone) 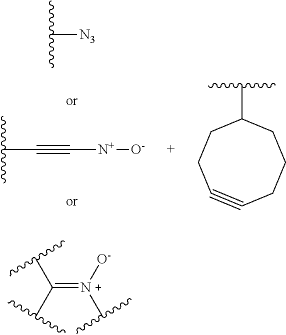 | 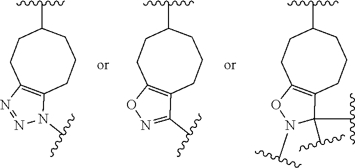 [SMILES: *C1CCCc2c(nnn2*)CC1 ; *c1noc2c1CCCC(*)CC2 ; *C1CCCC2=C(CC1)ON(*)C2(*)*] |
| | Norbornene cycloaddition (with azide, nitrile oxide, or nitrone 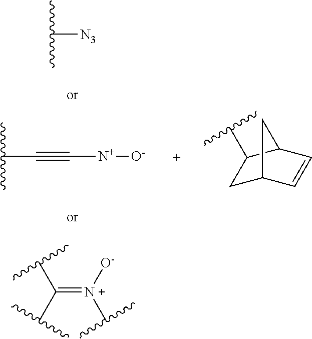 | 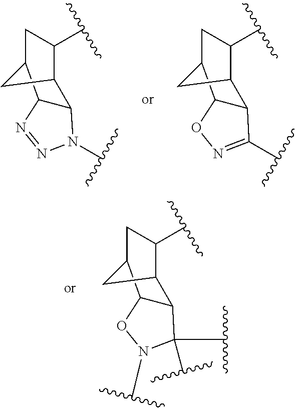 [SMILES: *C1CC2CC1C1C2N=NN1* ; *C1=NOC2C3CC(*)C(C3)C12 ; *C1CC2CC1C1C2ON(*)C1(*)*] |
| | Oxanorbornadiene cycloaddition 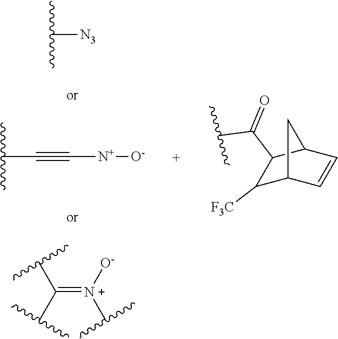 | 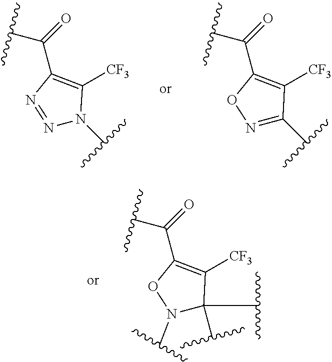 [SMILES: *C(=O)c1nnn(*)c1C(F)(F)F ; *C(=O)c1onc(*)c1C(F)(F)F ; *C(=O)C1=C(C(F)(F)F)C(*)(*)N(*)O1] |
| Staudinger ligation | 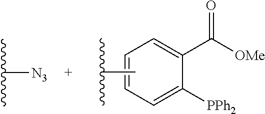 | 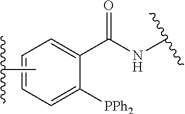 |
| Tetrazine ligation | 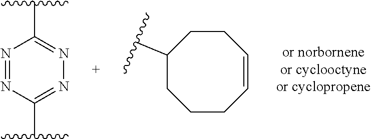 | 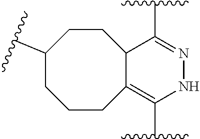 [SMILES: *C1=NNC(*)=C2CCCC(*)CCC12] |
| Photo- induced tetrazole- alkene cycloaddition | 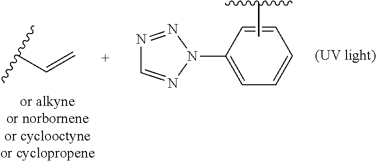 | 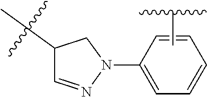 |
| [4 + 1] cycloaddition | 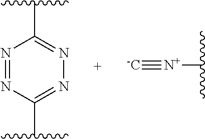 | 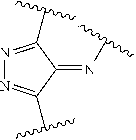 [SMILES: *N=C1C(*)=NN=C1*] |
| Quadricyclane ligation | 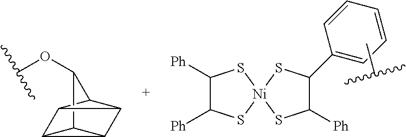 | 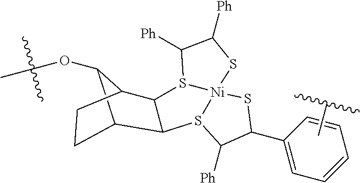 |

In some embodiments, V³ is

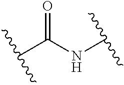 [SMILES: *NC(*)=O]

formed by a transglutaminase mediated acyl addition, wherein the γ-carboxamide group (—(C═O)NH₂) of a glutamine residue of LINKER⁴ is the acyl donor, and the primary amine —NH₂ attaching to LINKER³ is the acyl acceptor and the anime donor.

In some embodiments, V³ is

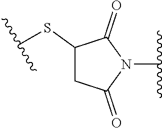 [SMILES: *SC1CC(=O)N(*)C1=O]

formed by a thiol group —SH attached to LINKER⁴ and a maleimide group

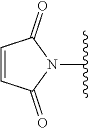 [SMILES: *N1C(=O)C=CC1=O]

attached to LINKER³. In some embodiments, V³ is

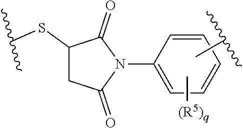

formed by a thiol group —SH attached to LINKER⁴ and a N-aryl maleimide group

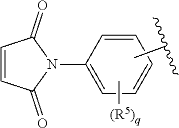

attached to LINKER³, wherein each R⁵ is independently R, Halogen, —OR, —SR, or —N(R)₂; q is 0, 1, 2, 3, or 4; and each R is independently as described herein. In some embodiments, V³ is

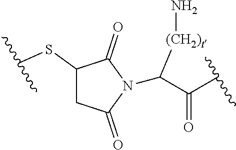

formed by a thiol group —SH attached to LINKER⁴ and a maleimide group

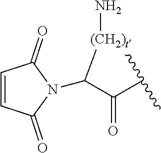

attached to LINKER³, wherein t′ is an integer from 0-20. In some embodiments, t′ is 0, 1, 2, 3, 4, 5, 6, 7, 8, 9, 10, 11, 12, 13, 14, 15, 16, 17, 18, 19, or 20. In some embodiments, t′ is 1.

In some embodiments, V³ is

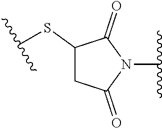 [SMILES: *SC1CC(=O)N(*)C1=O]

formed by a thiol group —SH of a cysteine residue of LINKER⁴ and a maleimide group

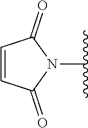 [SMILES: *N1C(=O)C=CC1=O]

attached to LINKER³. In some embodiments, V³ is

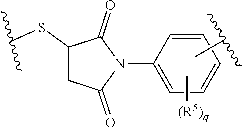

formed by a thiol group —SH of a cysteine residue of LINKER⁴ and a N-aryl maleimide group

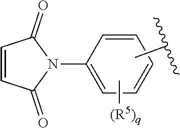

attached to LINKER³, wherein each of R⁵ and q is independently as described herein. In some embodiments, V³ is

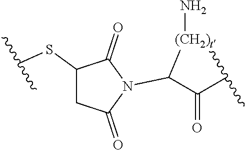

formed by a thiol group —SH of a cysteine residue of LINKER⁴ and a maleimide group

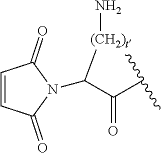

attached to LINKER³, wherein each t′ is independently as described herein.

In some embodiments, V³ is

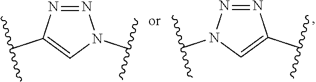 [SMILES: *c1cn(*)nn1 ; *c1cn(*)nn1]

formed by an azide-alkyne cycloaddition as shown in Table 2.

In some embodiments, V³ is selected from

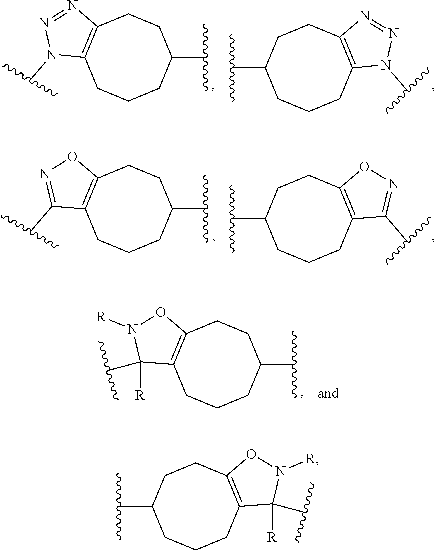

formed by a strain-promoted cycloaddition as shown in Table 2.

In some embodiments, V¹ is

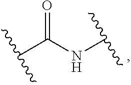 [SMILES: *NC(*)=O]

which is linked to LINKER¹ via the carbon atom, and linked to D¹ via the nitrogen atom.

In some embodiments, V² is

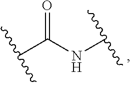 [SMILES: *NC(*)=O]

which is linked to LINKER² via the carbon atom, and linked to D² via the nitrogen atom.

In some embodiments, V²* is

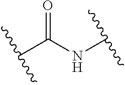 [SMILES: *NC(*)=O]

formed by a transglutaminase mediated acyl addition, wherein the γ-carboxamide group (—(C═O)NH₂) of a glutamine residue of LINKER²* is the acyl donor, and the primary amine —NH₂ attaching to LINKER¹* is the acyl acceptor and the anime donor.

In some embodiments, V²* is

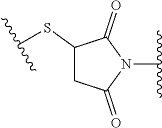 [SMILES: *SC1CC(=O)N(*)C1=O]

formed by a thiol group —SH attached to LINKER²* and a maleimide group

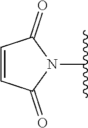 [SMILES: *N1C(=O)C=CC1=O]

attached to LINKER¹*. In some embodiments, V²* is

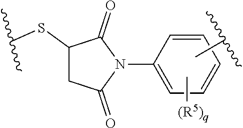

formed by a thiol group —SH attached to LINKER²* and a N-aryl maleimide group

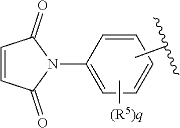

attached to LINKER¹*, wherein each R⁵* is independently R, Halogen, —OR, —SR, or —N(R)₂; q is 0, 1, 2, 3, or 4; and each R is independently as described herein. In some embodiments, V²* is

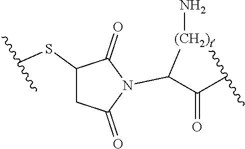

formed by a thiol group —SH attached to LINKER²* and a maleimide group

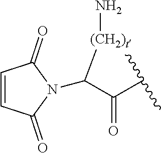

attached to LINKER*, wherein t′ is an integer from 0-20. In some embodiments, t′ is 0, 1, 2, 3, 4, 5, 6, 7, 8, 9, 10, 11, 12, 13, 14, 15, 16, 17, 18, 19, or 20. In some embodiments, t′ is 1.

In some embodiments, V²* is

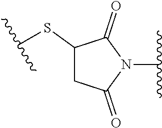 [SMILES: *SC1CC(=O)N(*)C1=O]

formed by a thiol group —SH of a cysteine residue of LINKER²* and a maleimide group

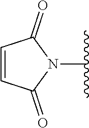 [SMILES: *N1C(=O)C=CC1=O]

attached to LINKER¹*. In some embodiments, V² is

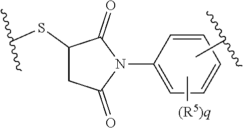

formed by a thiol group —SH of a cysteine residue of LINKER²* and a N-aryl maleimide group

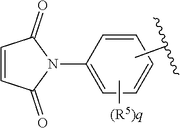

attached to LINKER¹*, wherein each of R¹* and q is independently as described herein. In some embodiments, V²* is

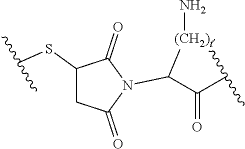

formed by a thiol group —SH of a cysteine residue of LINKER²* and a maleimide group

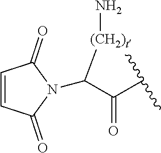

attached to LINKER¹*, wherein each t′ is independently as described herein.

In some embodiments, V²* is

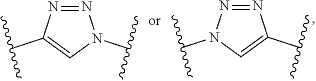 [SMILES: *c1cn(*)nn1 ; *c1cn(*)nn1]

formed by an azide-alkyne cycloaddition as shown in Table 2.

In some embodiments, V²* is selected from

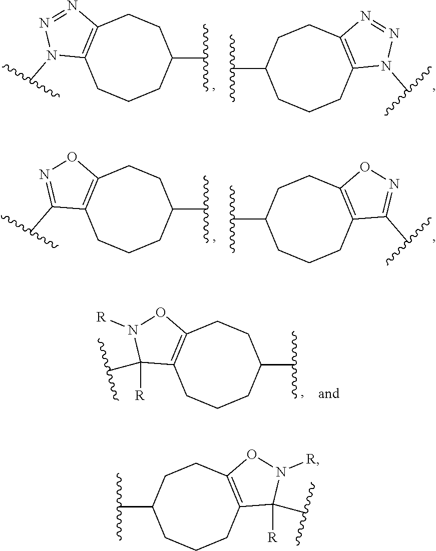

formed by a strain-promoted cycloaddition as shown in Table 2.

In some embodiments, V¹* is

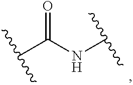 [SMILES: *NC(*)=O]

which is linked to LINKER¹* via the carbon atom, and linked to D via the nitrogen atom.

### 2.4. Bivalent Linker LINKER³

As defined generally above, each LINKER³ is independently a bivalent linker. In some embodiments, LINKER³ comprises a hydrocarbon chain unit T³. In some embodiments, LINKER³ comprises an amide moiety W³. In some embodiments, LINKER³ comprises a hydrocarbon chain unit S³.

In some embodiments, LINKER³ is

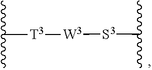

wherein:
each of S³ and T³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

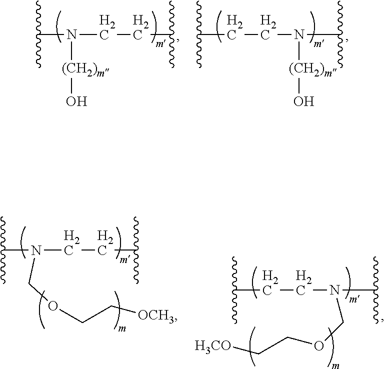

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each W³ is independently —C(O)—N(R³)— or —N(R³)—C(O)—;
each -Cy- is independently an optionally substituted 3-8 membered bivalent saturated, partially unsaturated, or aromatic ring having 0-4 heteroatoms independently selected from nitrogen, oxygen, or sulfur;
each R³ is independently H or optionally substituted C₁₋₆ aliphatic;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently an integer from 0-24.

As defined generally above, each S³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

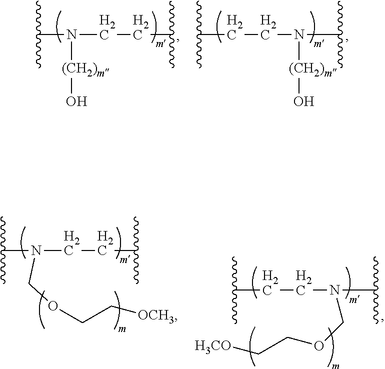

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S³ is a covalent bond.

In some embodiments, S³ is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ—,

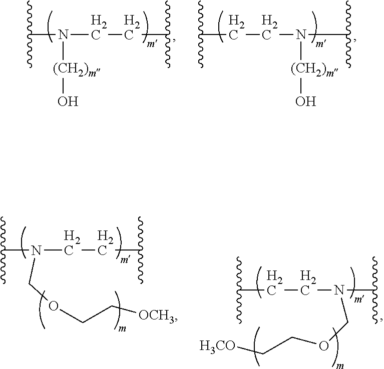

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S³ is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

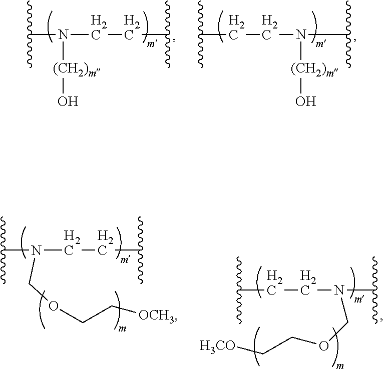

—S—, —N(R)—, —O—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S³ is a bivalent C₁₋₈ saturated or unsaturated, straight or branched, hydrocarbon chain wherein three methylene units of the chain are optionally and independently replaced by -Cy-, —C(R)₂—, —(OCH₂CH₂)ₘ—, —(CH₂CH₂O)ₘ—,

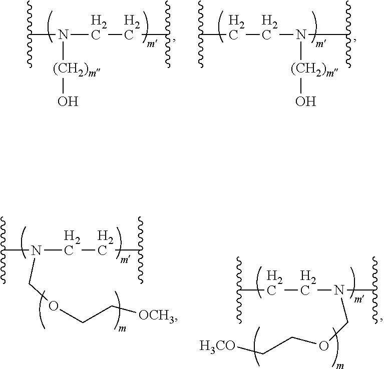

—S—, —N(R)—, —O—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

As defined generally above, each W³ is independently —C(O)—N(R³)— or —N(R³)—C(O)—, wherein each R³ is independently as described herein.

In some embodiments, W³ is —C(O)—N(R³)—, wherein R³ is H or optionally substituted C₁₋₆ aliphatic. In some embodiments, R³ is H. In some embodiments, R³ is optionally substituted C₁₋₆ aliphatic. In some embodiments, R³ is optionally substituted C₁₋₆ alkyl. In some embodiments, R³ is methyl, ethyl, propyl, isopropyl, butyl, isobutyl, or tert-butyl. In some embodiments, W³ is —C(O)—NH—.

In some embodiments, W³ is —N(R³)—C(O)—, wherein R³ is H or optionally substituted C₁₋₆ aliphatic. In some embodiments, W³ is —N(R³)—C(O)—, wherein R³ is as described above. In some embodiments, W³ is —NH—C(O)—.

As defined generally above, each T³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

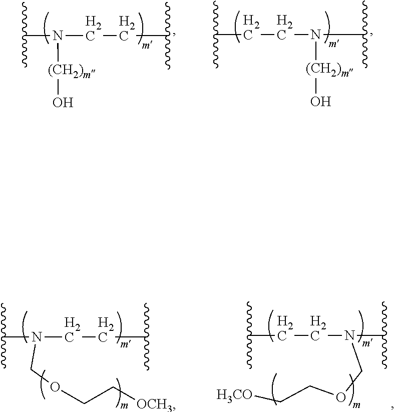

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, T³ is a covalent bond.

In some embodiments, T³ is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, (CH₂CH₂O)ₘ—,

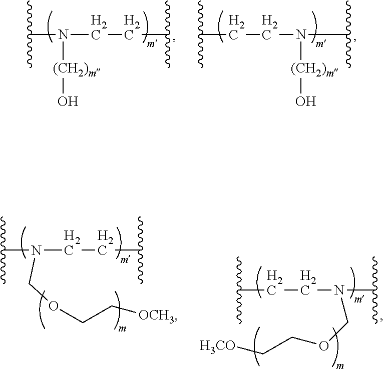

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, T³ is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

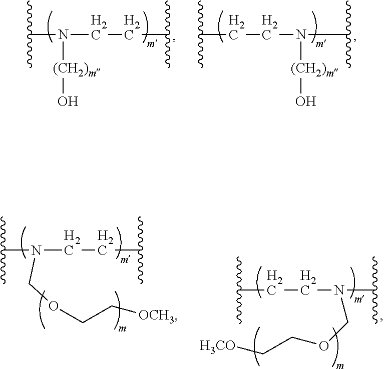

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, T³ is a bivalent C₁₋₈ saturated or unsaturated, straight or branched, hydrocarbon chain wherein three methylene units of the chain are optionally and independently replaced by -Cy-, —C(R)₂—, —(OCH₂CH₂)ₘ—, —(CH₂CH₂O)ₘ—,

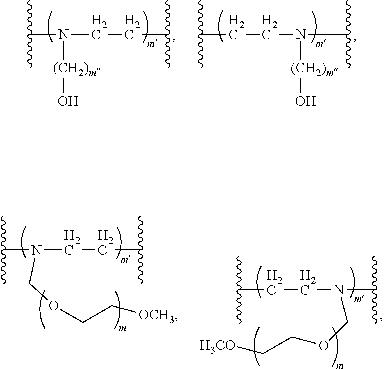

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, T³ is

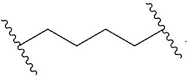 [SMILES: *CCCC*]

In some embodiments, T³ is

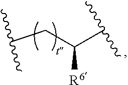

wherein R^{6′} is R, —N(R)₂, or —N(R)C(O)—R, t″ is an integer from 0-20, and each R is independently as described herein. In some embodiments, t″ is 0, 1, 2, 3, 4, 5, 6, 7, 8, 9, 10, 11, 12, 13, 14, 15, 16, 17, 18, 19, or 20. In some embodiments, R^{6′} is H. In some embodiments, R^{6′} is —CH₃. In some embodiments, R^{6′} is

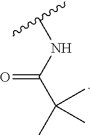 [SMILES: *NC(=O)C(C)(C)C]

In some embodiments, R^{6′} is

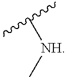 [SMILES: *NC]

In some embodiments, R^{6′} is

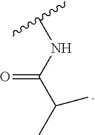 [SMILES: *NC(=O)C(C)C]

In some embodiments, R^{6′} is

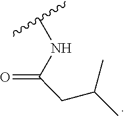 [SMILES: *NC(=O)CC(C)C]

In some embodiments, R^{6′} is

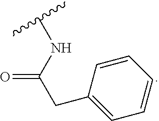 [SMILES: *NC(=O)Cc1ccccc1]

In some embodiments, R^{6′} is

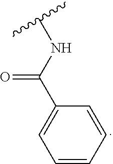 [SMILES: *NC(=O)c1ccccc1]

In some embodiments, R^{6′} is

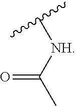 [SMILES: *NC(C)=O]

In some embodiments, R^{6′} is

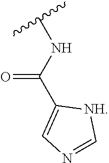 [SMILES: *NC(=O)c1cnc[nH]1]

In some embodiments, R^{6′} is

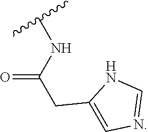 [SMILES: *NC(=O)Cc1cnc[nH]1]

In some embodiments, R^{6′} is

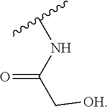 [SMILES: *NC(=O)CO]

In some embodiments, T³ is

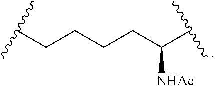 [SMILES: *CCCC[C@@H](*)NC(C)=O]

In some embodiments, R³ is H. In some embodiments, R³ is optionally substituted C₁₋₆ aliphatic. In some embodiments, R³ is optionally substituted C₁₋₆ alkyl. In some embodiments, R³ is methyl, ethyl, propyl, isopropyl, butyl, isobutyl, or tert-butyl.

In some embodiments, is

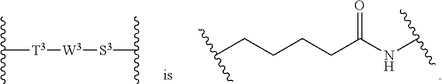

### 2.5. Bivalent Linker LINKER¹

As defined generally above, each LINKER¹ is independently a bivalent linker. In some embodiments, LINKER¹ comprises a hydrocarbon chain unit S¹. In some embodiments, LINKER¹ comprises an amide moiety W¹. In some embodiments, LINKER¹ comprises a PEG unit T¹. In some embodiments, LINKER¹ comprises a peptide unit P¹. In some embodiments, LINKER¹ comprises a cleavable linker L¹.

In some embodiments, LINKER¹ is

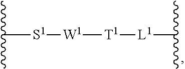

wherein:
S¹ is a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

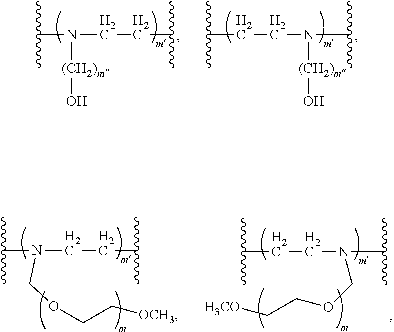

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

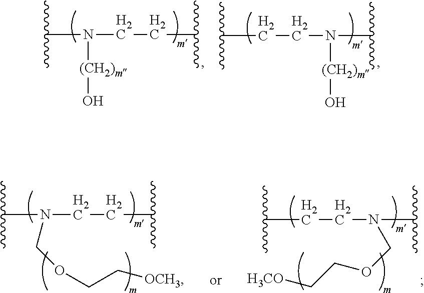

or W¹ and T¹ are absent;
L¹ is —C(O)-P¹-X¹— or —N(R¹)-P¹-X¹—, wherein each P¹ is absent or a peptide comprising 1-20 amino acids, and each X¹ is a cleavable linker;
each of R¹ and R³ is independently H or optionally substituted C₁₋₆ aliphatic;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently an integer from 0-24.

As defined generally above, each S¹ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

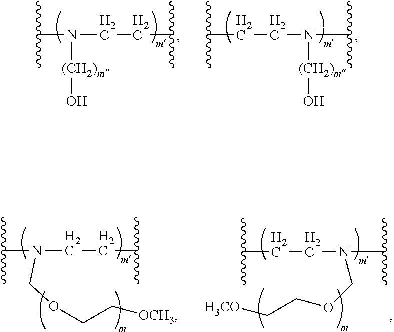

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹ is a covalent bond.

In some embodiments, S¹ is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ,

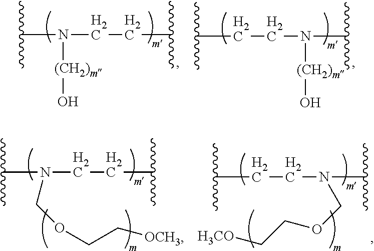

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹ is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

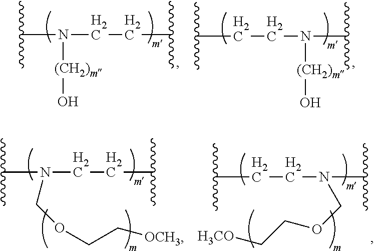

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹ is a bivalent C₁₋₈ saturated or unsaturated, straight or branched, hydrocarbon chain wherein three methylene units of the chain are optionally and independently replaced by -Cy, —C(R)₂—, —(CH₂CH₂)ₘ, (CH₂CH₂O)ₘ—,

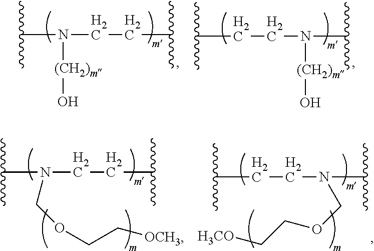

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹ is —CH₂—CH₂—.

In some embodiments, S¹ is

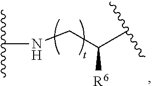

wherein R⁶ is R, —N(R)₂, or —N(R)C(O)—R, each t is independently an integer from 0-20, and each R is independently as described herein. In some embodiments, t is 0, 1, 2, 3, 4, 5, 6, 7, 8, 9, 10, 11, 12, 13, 14, 15, 16, 17, 18, 19, or 20. In some embodiments, R⁶ is H. In some embodiments, R⁶ is —CH₃. In some embodiments, R⁶ is

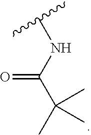 [SMILES: *NC(=O)C(C)(C)C]

In some embodiments, R⁶ is

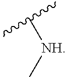 [SMILES: *NC]

In some embodiments, R⁶ is

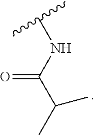 [SMILES: *NC(=O)C(C)C]

In some embodiments, R⁶ is

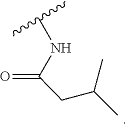 [SMILES: *NC(=O)CC(C)C]

In some embodiments, R⁶ is

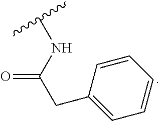 [SMILES: *NC(=O)Cc1ccccc1]

In some embodiments, R⁶ is

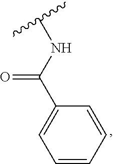 [SMILES: *NC(=O)c1ccccc1]

In some embodiments, R⁶ is

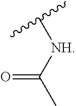 [SMILES: *NC(C)=O]

In some embodiments, R⁶

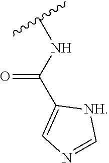 [SMILES: *NC(=O)c1cnc[nH]1]

In some embodiments, R⁶ is

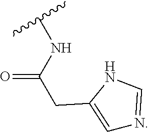 [SMILES: *NC(=O)Cc1cnc[nH]1]

In some embodiments, R⁶ is

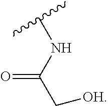 [SMILES: *NC(=O)CO]

In some embodiments, S¹ is

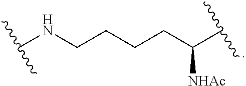 [SMILES: *NCCCC[C@@H](*)NC(C)=O]

As defined generally above, each W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

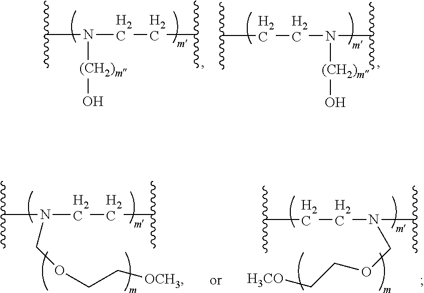

or W¹ and T¹ are absent, wherein each of R³, m, m′, and m″ is independently as described herein.

In some embodiments, W¹ is —C(O)—N(R³)—, wherein R³ is H or optionally substituted C₁₋₆ aliphatic. In some embodiments, W¹ is —C(O)—N(R³)—, wherein R³ is as described herein. In some embodiments, W¹ is —C(O)—NH—.

In some embodiments, W¹ is —N(R³)—C(O)—, wherein R³ is H or optionally substituted C₁₋₆ aliphatic. In some embodiments, W¹ is —N(R³)—C(O)—, wherein R³ is as described herein. In some embodiments, W¹ is —NH—C(O)—.

As defined generally above, each T¹ is independently a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

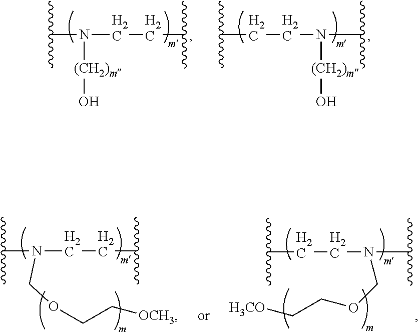

wherein each of m, m′, and m″ is independently as described herein.

In some embodiments, T¹ is a bivalent C₁₋₈ saturated or unsaturated, straight or branched, hydrocarbon chain wherein one methylene unit of the chain is replaced by —(OCH₂CH₂)ₘ—, wherein m is as described herein.

In some embodiments, T¹ is a bivalent C₁₋₈ saturated or unsaturated, straight or branched, hydrocarbon chain wherein one methylene unit of the chain is replaced by —(CH₂CH₂O)ₘ—, wherein m is as described herein.

In some embodiments, T¹ is a bivalent C₁₋₈ saturated or unsaturated, straight or branched, hydrocarbon chain wherein one methylene unit of the chain is replaced by

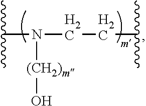

wherein each of m′ and m″ is as described herein.

In some embodiments, T¹ is a bivalent C₁₋₈ saturated or unsaturated, straight or branched, hydrocarbon chain wherein one methylene unit of the chain is replaced by

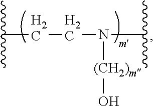

wherein each of m′ and m″ is as described herein.

In some embodiments, T¹ is a bivalent C₁₋₈ saturated or unsaturated, straight or branched, hydrocarbon chain wherein one methylene unit of the chain is replaced by

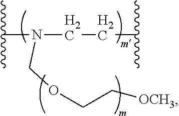

wherein each of m and m′ is as described herein.

In some embodiments, T¹ is a bivalent C₁₋₈ saturated or unsaturated, straight or branched, hydrocarbon chain wherein one methylene unit of the chain is replaced by

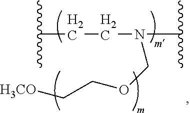

wherein each of m and m′ is as described herein.

In some embodiments, T¹ is

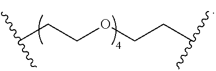

In some embodiments, T¹ and W¹ are absent.

As defined generally above, each L¹ is independently —C(O)-P¹-X¹— or —N(R¹)-P¹—X¹—, wherein P¹ is absent or a peptide comprising 1-20 amino acids, X¹ is a cleavable linker, and R¹ is H or optionally substituted C₁₋₆ aliphatic.

In some embodiments, L¹ is —C(O)-P¹-X¹— or —N(R¹)-P¹-X¹—, wherein R¹ is H or optionally substituted C₁₋₆ aliphatic, P¹ is a peptide comprising 1-20 amino acids, and X¹ is a cleavable linker. In some embodiments, L¹ is —C(O)-P¹-X¹—, wherein each of P¹ and X¹ is independently as described herein. In some embodiments, L¹ is —N(R¹)-P¹-X¹—, wherein each of R¹, P¹, and X¹ is independently as described herein.

As defined generally above, each R¹ is independently H or optionally substituted C₁₋₆ aliphatic. In some embodiments, R¹ is H. In some embodiments, R¹ is optionally substituted C₁₋₆ aliphatic. In some embodiments, R¹ is optionally substituted C₁₋₆ alkyl. In some embodiments, R¹ is methyl, ethyl, propyl, isopropyl, butyl, isobutyl, or tert-butyl.

As defined generally above, each P¹ is independently absent or a peptide comprising 1-20 amino acids. In some embodiments, P¹ is absent. In some embodiments, P¹ is a peptide comprising 1-20 amino acids. In some embodiments, P¹ is a peptide having 1, 2, 3, 4, or 5 amino acids. In some embodiments, P¹ is a peptide having 6, 7, 8, 9, or 10 amino acids. In some embodiments, P¹ is a peptide having 11, 12, 13, 14, or 15 amino acids. In some embodiments, P¹ is a peptide having 16, 17, 18, 19, or 20 amino acids. In some embodiments, each amino acids of P¹ is a L-amino acid. In some embodiments, P¹ is a dipeptide selected from -(L-ala)-(L-ala)-, -Val-Cit-, and -Val-Ala-. In some embodiments, P¹ is a tripeptide -Glu-Val-Cit-. In some embodiments, P¹ is a tripeptide -AcLys-Val-Cit-. In some embodiments, P¹ is a tetrapeptide -Gly-Gly-Phe-Gly-.

As defined generally above, each X¹ is independently a cleavable linker. In some embodiments, X¹ is a self-immolative linker capable of releasing -D¹ without the need for a separate hydrolysis step. In some embodiments X¹ is a p-amino-benzyloxy (PAB) group, which is linked to P¹ via the amino nitrogen atom of the PAB group, and linked to V¹ via the oxygen atom of the PAB group. In some embodiments, X¹ is a PAB group

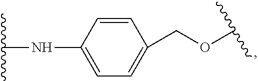 [SMILES: *Nc1ccc(CO*)cc1]

which is linked to P¹ via the amino nitrogen atom of the PAB group, and linked to V¹ via the oxygen atom of the PAB group. In some embodiments X¹ is a substituted p-amino-benzyl (PAB) group, which is linked to P¹ via the amino nitrogen atom of the PAB group, and linked to V¹ via the oxygen atom of the PAB group. In some embodiments X¹ is a p-amino-benzyl (PAB) group substituted with a phosphate group, including but not limited to —OPO³H, wherein the PAB group is linked to P¹ via the amino nitrogen atom of the PAB group, and linked to V¹ via the oxygen atom of the PAB group. In some embodiments, X¹ is

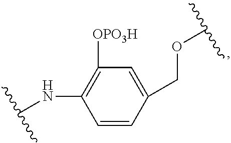

which is linked to P¹ via the amino nitrogen atom, and linked to V¹ via the oxygen atom. In some embodiments, X¹ is

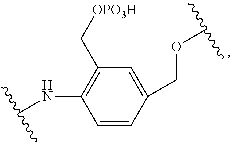

which is linked to P¹ via the amino nitrogen atom, and linked to V¹ via the oxygen atom. In some embodiments, X¹ is

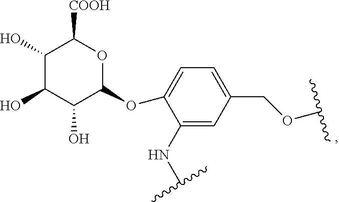

which is linked to P¹ via the amino nitrogen atom, and linked to V¹ via the oxygen atom.

In some embodiments, L¹ is —N(R¹)-P¹-X¹—, wherein each of R¹, P¹, and X¹ is independently as described herein.

In some embodiments, L¹ is

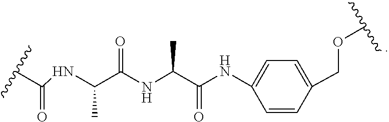 [SMILES: *OCc1ccc(NC(=O)[C@H](C)NC(=O)[C@H](C)NC(*)=O)cc1]

In some embodiments, L¹ is

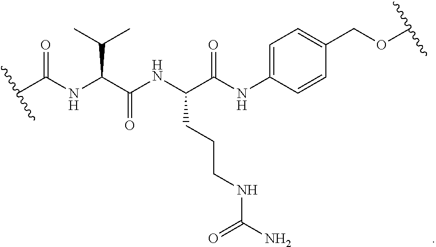 [SMILES: *OCc1ccc(NC(=O)[C@H](CCCNC(N)=O)NC(=O)[C@@H](NC(*)=O)C(C)C)cc1]

In some embodiments, L¹ is

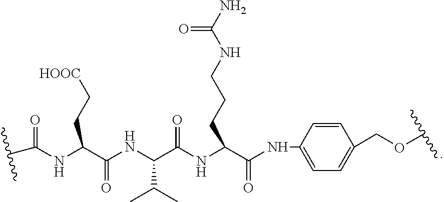 [SMILES: *OCc1ccc(NC(=O)[C@H](CCCNC(N)=O)NC(=O)[C@@H](NC(=O)[C@H](CCC(=O)O)NC(*)=O)C(C)C)cc1]

In some embodiments,

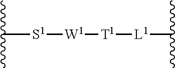

is

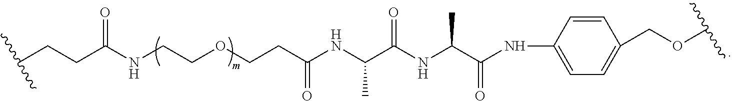

In some embodiments,

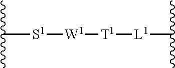

is

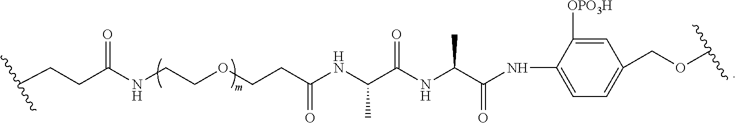

In some embodiments,

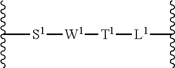

is

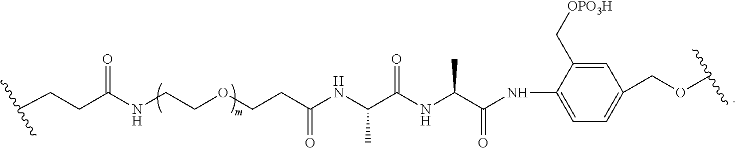

In some embodiments,

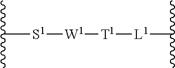

is

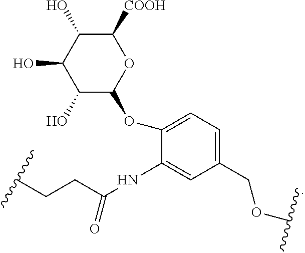

In some embodiments,

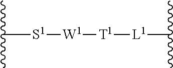

is

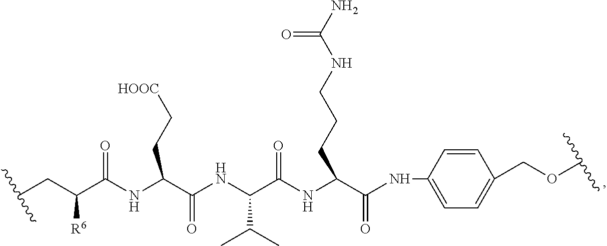

wherein each R⁶ is independently as described herein.

In some embodiments,

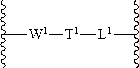

is

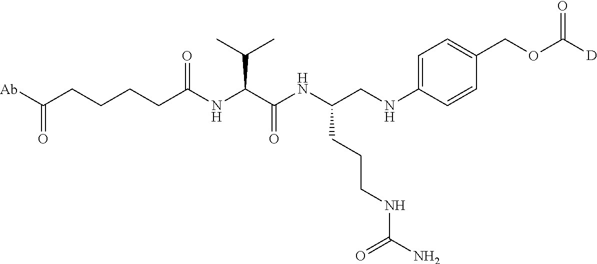

wherein each R⁶ is independently as described herein.

In some embodiments,

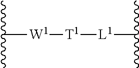

is

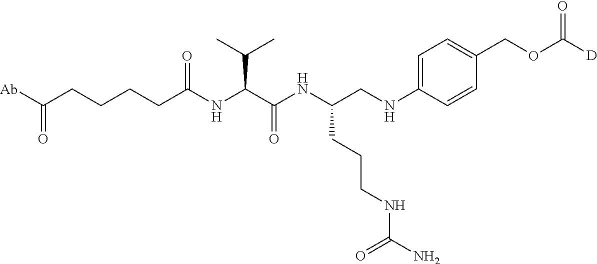

In some embodiments,

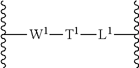

is

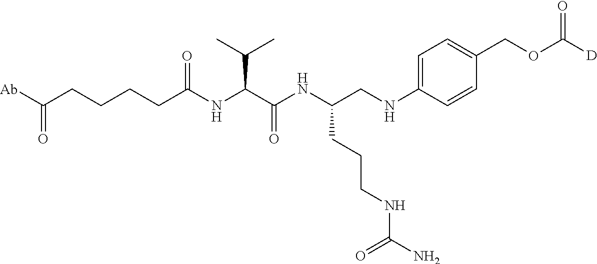

In some embodiments,

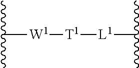

is

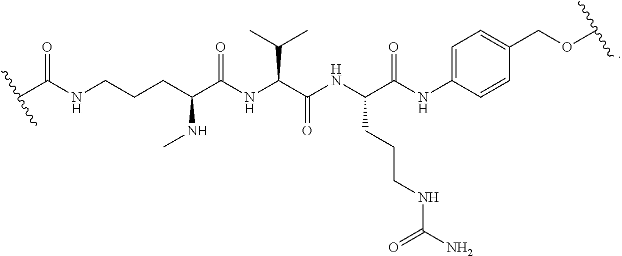 [SMILES: *OCc1ccc(NC(=O)[C@H](CCCNC(N)=O)NC(=O)[C@@H](NC(=O)[C@H](CCCNC(*)=O)NC)C(C)C)cc1]

In some embodiments,

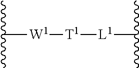

is

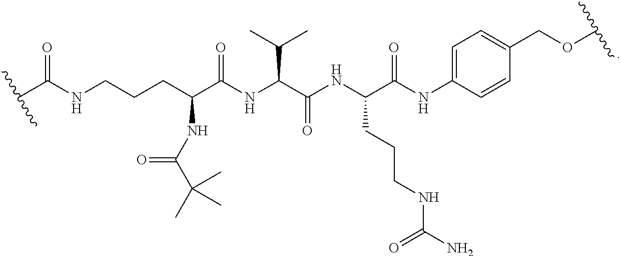 [SMILES: *OCc1ccc(NC(=O)[C@H](CCCNC(N)=O)NC(=O)[C@@H](NC(=O)[C@H](CCCNC(*)=O)NC(=O)C(C)(C)C)C(C)C)cc1]

In some embodiments,

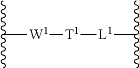

is

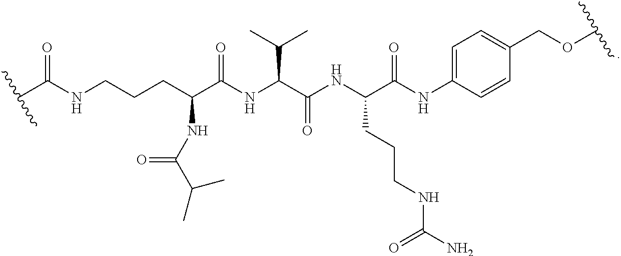 [SMILES: *OCc1ccc(NC(=O)[C@H](CCCNC(N)=O)NC(=O)[C@@H](NC(=O)[C@H](CCCNC(*)=O)NC(=O)C(C)C)C(C)C)cc1]

In some embodiments,

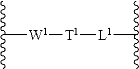

is

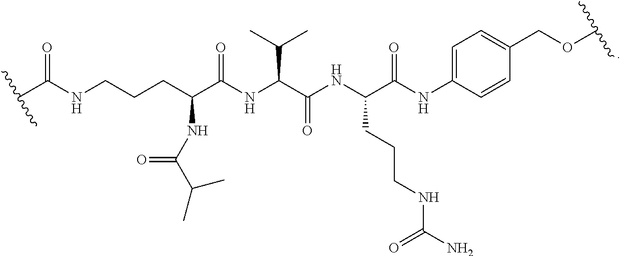 [SMILES: *OCc1ccc(NC(=O)[C@H](CCCNC(N)=O)NC(=O)[C@@H](NC(=O)[C@H](CCCNC(*)=O)NC(=O)C(C)C)C(C)C)cc1]

In some embodiments,

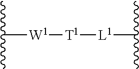

is

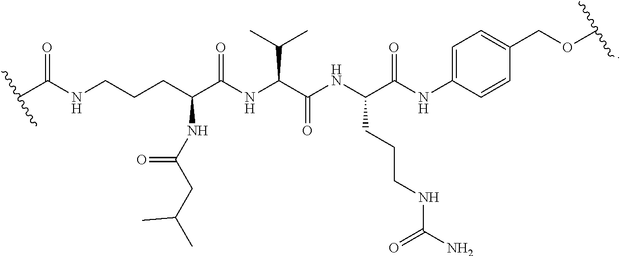 [SMILES: *OCc1ccc(NC(=O)[C@H](CCCNC(N)=O)NC(=O)[C@@H](NC(=O)[C@H](CCCNC(*)=O)NC(=O)CC(C)C)C(C)C)cc1]

In some embodiments,

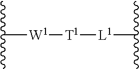

is

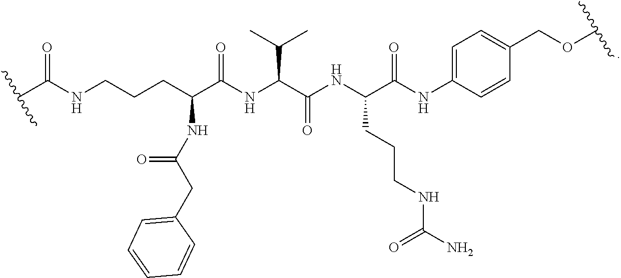 [SMILES: *OCc1ccc(NC(=O)[C@H](CCCNC(N)=O)NC(=O)[C@@H](NC(=O)[C@H](CCCNC(*)=O)NC(=O)Cc2ccccc2)C(C)C)cc1]

In some embodiments,

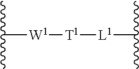

is

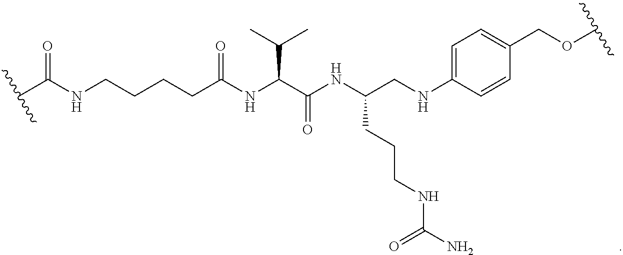 [SMILES: *OCc1ccc(NC[C@H](CCCNC(N)=O)NC(=O)[C@@H](NC(=O)CCCCNC(*)=O)C(C)C)cc1]

In some embodiments,

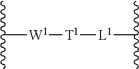

is

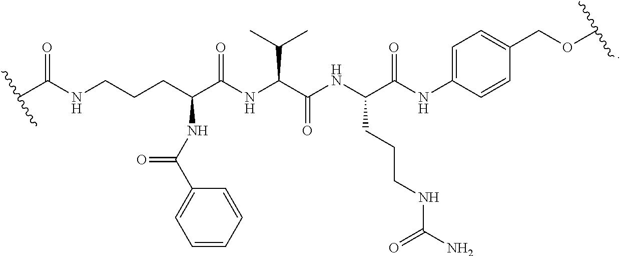 [SMILES: *OCc1ccc(NC(=O)[C@H](CCCNC(N)=O)NC(=O)[C@@H](NC(=O)[C@H](CCCNC(*)=O)NC(=O)c2ccccc2)C(C)C)cc1]

In some embodiments,

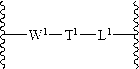

is

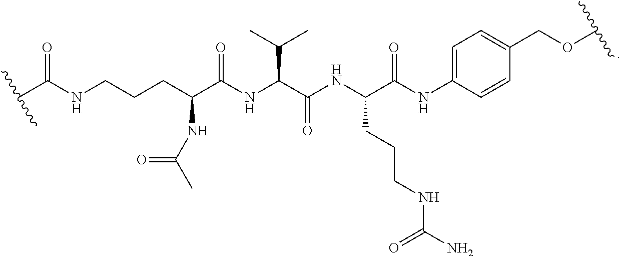 [SMILES: *OCc1ccc(NC(=O)[C@H](CCCNC(N)=O)NC(=O)[C@@H](NC(=O)[C@H](CCCNC(*)=O)NC(C)=O)C(C)C)cc1]

In some embodiments,

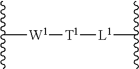

is

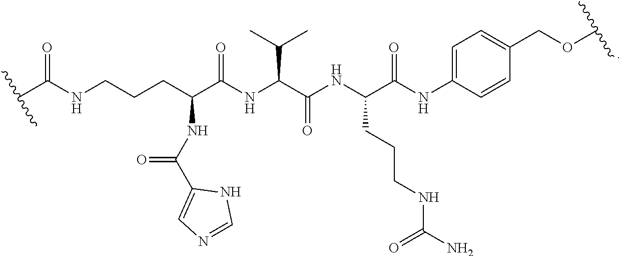 [SMILES: *OCc1ccc(NC(=O)[C@H](CCCNC(N)=O)NC(=O)[C@@H](NC(=O)[C@H](CCCNC(*)=O)NC(=O)c2cnc[nH]2)C(C)C)cc1]

In some embodiments,

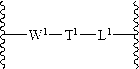

is

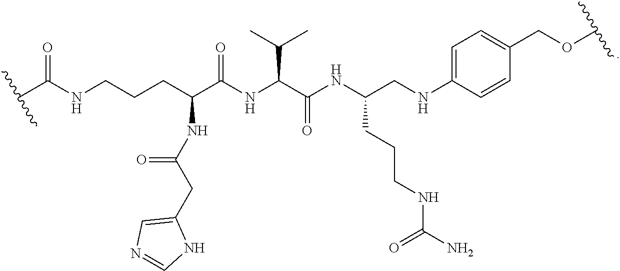 [SMILES: *OCc1ccc(NC[C@H](CCCNC(N)=O)NC(=O)[C@@H](NC(=O)[C@H](CCCNC(*)=O)NC(=O)Cc2cnc[nH]2)C(C)C)cc1]

In some embodiments,

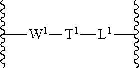

is

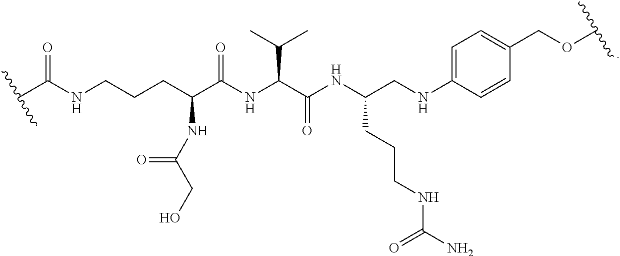 [SMILES: *OCc1ccc(NC[C@H](CCCNC(N)=O)NC(=O)[C@@H](NC(=O)[C@H](CCCNC(*)=O)NC(=O)CO)C(C)C)cc1]

In some embodiments,

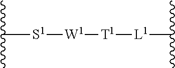

is

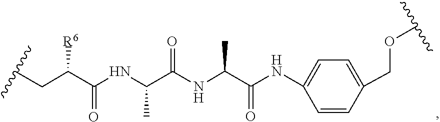

wherein each R⁶ is independently as described herein.

### 2.6. Bivalent Linker LINKER¹*

As defined generally above, each LINKER¹* is independently a bivalent linker. In some embodiments, LINKER¹* comprises a hydrocarbon chain unit S¹*. In some embodiments, LINKER¹* comprises a PEG unit T¹*. In some embodiments, LINKER¹* comprises a peptide unit P¹*. In some embodiments, LINKER¹* comprises a cleavable linker X¹*.

In some embodiments, LINKER¹* is

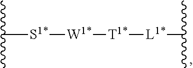

wherein:
S¹* is a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ,

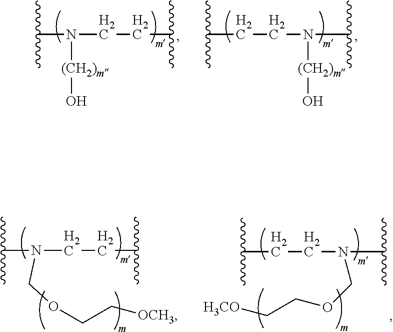

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each W¹* is independently —C(O)—N(R³*)— or —N(R³*)—C(O)—, and each T¹* is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

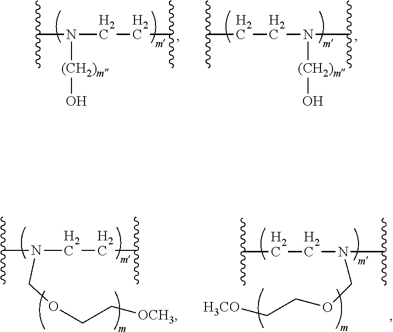

or W¹* and T¹* are absent;
each -Cy- is independently an optionally substituted 3-8 membered bivalent saturated, partially unsaturated, or aromatic ring having 0-4 heteroatoms independently selected from nitrogen, oxygen, or sulfur;
each R³* is independently H or optionally substituted C₁₋₆ aliphatic; L¹* is —C(O)-P¹*—X¹*— or —N(R¹*)-P¹*—X¹*—, wherein each P¹* is absent or a peptide comprising 1-20 amino acids, and each X¹* is a cleavable linker;
each R¹* is independently H or optionally substituted C₁₋₆ aliphatic;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

As defined generally above, each S¹* is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

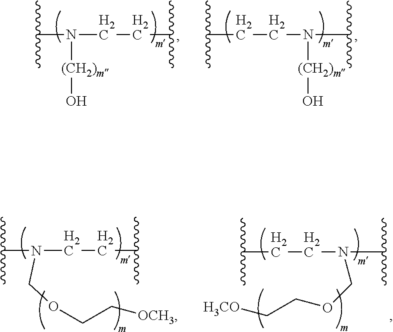

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹* is a covalent bond.

In some embodiments, S¹* is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ—,

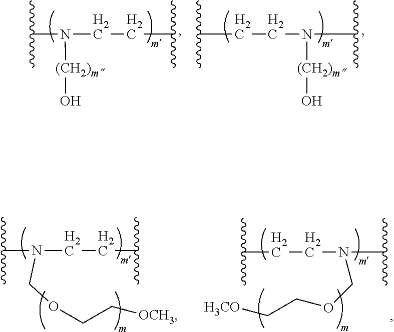

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹* is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

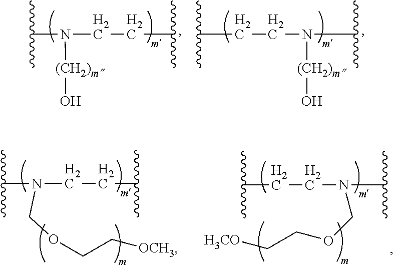

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹* is a bivalent C₁₋₈ saturated or unsaturated, straight or branched, hydrocarbon chain wherein three methylene units of the chain are optionally and independently replaced by -Cy-, —C(R)₂—, —(OCH₂CH₂)ₘ—, —(CH₂CH₂O)ₘ—,

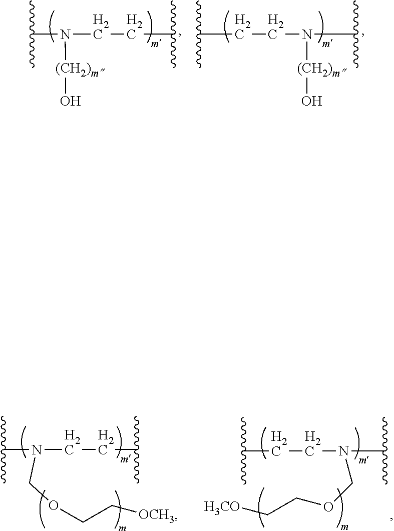

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S¹* is

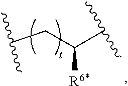

wherein R⁶* is R, —N(R)₂, or —N(R)C(O)—R, t is an integer from 0-20, and each R is independently as described herein. In some embodiments, t is 0, 1, 2, 3, 4, 5, 6, 7, 8, 9, 10, 11, 12, 13, 14, 15, 16, 17, 18, 19, or 20. In some embodiments, R⁶* is H. In some embodiments, R⁶* is —CH₃. In some embodiments, R⁶* is

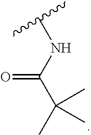 [SMILES: *NC(=O)C(C)(C)C]

In some embodiments, R⁶* is

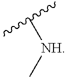 [SMILES: *NC]

In some embodiments, R⁶* is

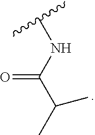 [SMILES: *NC(=O)C(C)C]

In some embodiments, R⁶* is

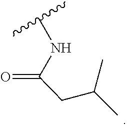 [SMILES: *NC(=O)CC(C)C]

In some embodiments, R⁶* is

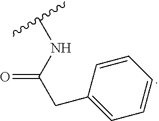 [SMILES: *NC(=O)Cc1ccccc1]

In some embodiments, R⁶* is

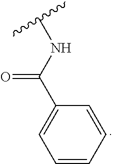 [SMILES: *NC(=O)c1ccccc1]

In some embodiments, R⁶* is

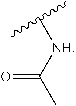 [SMILES: *NC(C)=O]

In some embodiments, R⁶* is

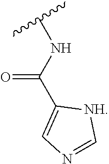 [SMILES: *NC(=O)c1cnc[nH]1]

In some embodiments, R⁶* is

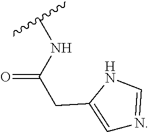 [SMILES: *NC(=O)Cc1cnc[nH]1]

In some embodiments, R⁶* is

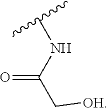 [SMILES: *NC(=O)CO]

In some embodiments, S¹* is

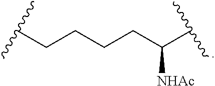 [SMILES: *CCCC[C@@H](*)NC(C)=O]

As defined generally above, each W¹* is independently —C(O)—N(R³*)— or —N(R³*)—C(O)—, wherein each R³* is independently as described herein.

In some embodiments, W¹* is —C(O)—N(R³*)—, wherein R³* is H or optionally substituted C₁₋₆ aliphatic. In some embodiments, R³* is H. In some embodiments, R³* is optionally substituted C₁₋₆ aliphatic. In some embodiments, R³* is optionally substituted C₁₋₆ alkyl. In some embodiments, R³* is methyl, ethyl, propyl, isopropyl, butyl, isobutyl, or tert-butyl. In some embodiments, W¹* is —C(O)—NH—.

In some embodiments, W¹* is —N(R³*)—C(O)—, wherein R³* is H or optionally substituted C₁₋₆ aliphatic. In some embodiments, W¹* is —N(R³*)—C(O)—, wherein R³* is as described above. In some embodiments, W¹* is —NH—C(O)—.

In some embodiments, -S¹*-W¹*- is

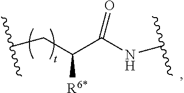

wherein R⁶* is as described above. In some embodiments, -S¹*-W¹*- is

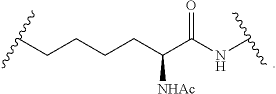 [SMILES: *CCCC[C@H](NC(C)=O)C(=O)N*]

As defined generally above, each T¹* is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

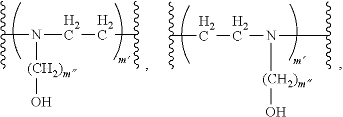

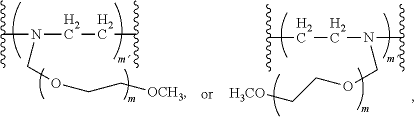

wherein each of m, m′, and m″ is independently as described herein.

In some embodiments, T¹* is a bivalent C₁₋₈ saturated or unsaturated, straight or branched, hydrocarbon chain wherein one methylene unit of the chain is replaced by —(OCH₂CH₂)ₘ—, wherein m is as described herein.

In some embodiments, T¹* is a bivalent C₁₋₈ saturated or unsaturated, straight or branched, hydrocarbon chain wherein one methylene unit of the chain is replaced by —(CH₂CH₂O)ₘ—, wherein m is as described herein.

In some embodiments, T¹* is a bivalent C₁₋₈ saturated or unsaturated, straight or branched, hydrocarbon chain wherein one methylene unit of the chain is replaced by

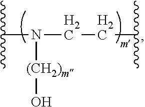

wherein each of m′ and m″ is as described herein.

In some embodiments, T¹* is a bivalent C₁₋₈ saturated or unsaturated, straight or branched, hydrocarbon chain wherein one methylene unit of the chain is replaced by

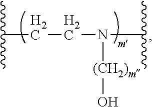

wherein each of m′ and m″ is as described herein.

In some embodiments, T¹* is a bivalent C₁₋₈ saturated or unsaturated, straight or branched, hydrocarbon chain wherein one methylene unit of the chain is replaced by

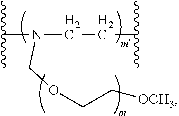

wherein each of m and m′ is independently as described herein.

In some embodiments, T¹* is a bivalent C₁₋₈ saturated or unsaturated, straight or branched, hydrocarbon chain wherein one methylene unit of the chain is replaced by

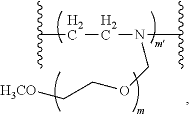

wherein each of m and m′ is independently as described herein.

In some embodiments, T¹* is

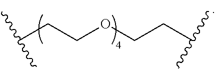

In some embodiments, W¹* and T¹* are absent.

As defined generally above, each L¹* is independently —C(O)-P¹*-X¹*- or —N(R¹*)-P¹*-X¹*—, wherein P¹* is absent or a peptide comprising 1-20 amino acids, X¹* is a cleavable linker, and R¹* is H or optionally substituted C₁₋₆ aliphatic.

In some embodiments, L¹* is —C(O)-P¹*-X¹*- or —N(R¹*)-P¹*-X¹*—, wherein R¹* is H or optionally substituted C₁₋₆ aliphatic, P¹* is a peptide comprising 1-20 amino acids, and X¹* is a cleavable linker. In some embodiments, L¹* is —C(O)-P¹*-X¹*—, wherein each of P¹* and X¹* is independently as described herein. In some embodiments, L¹* is —N(R¹*)-P¹*-X¹*- wherein each of R¹*, P¹*, and X¹* is independently as described herein.

As defined generally above, each R¹* is independently H or optionally substituted C₁₋₆ aliphatic. In some embodiments, R¹* is H. In some embodiments, R¹* is optionally substituted C₁₋₆ aliphatic. In some embodiments, R¹* is optionally substituted C₁₋₆ alkyl. In some embodiments, R¹* is methyl, ethyl, propyl, isopropyl, butyl, isobutyl, or tert-butyl.

As defined generally above, each P¹* is independently absent or a peptide comprising 1-20 amino acids. In some embodiments, P¹* is absent. In some embodiments, P¹* is a peptide comprising 1-20 amino acids. In some embodiments, P¹* is a peptide having 1, 2, 3, 4, or 5 amino acids. In some embodiments, P¹* is a peptide having 6, 7, 8, 9, or 10 amino acids. In some embodiments, P¹* is a peptide having 11, 12, 13, 14, or 15 amino acids. In some embodiments, P¹* is a peptide having 16, 17, 18, 19, or 20 amino acids. In some embodiments, each amino acids of P¹* is a L-amino acid. In some embodiments, P¹* is a dipeptide selected from -(L-ala)-(L-ala)-, -Val-Cit-, and -Val-Ala-. In some embodiments, P¹* is a tripeptide -Glu-Val-Cit-. In some embodiments, P¹* is a tripeptide -AcLys-Val-Cit-. In some embodiments, P¹* is a tetrapeptide -Gly-Gly-Phe-Gly-.

As defined generally above, each X¹* is independently a cleavable linker. In some embodiments, X¹* is a self-immolative linker capable of releasing -D¹* without the need for a separate hydrolysis step. In some embodiments X¹* is a p-amino-benzyloxy (PAB) group, which is linked to P¹* via the amino nitrogen atom of the PAB group, and linked to V¹* via the oxygen atom of the PAB group. In some embodiments, X¹* is a PAB group

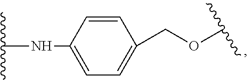 [SMILES: *Nc1ccc(CO*)cc1]

which is linked to P¹* via the amino nitrogen atom of the PAB group, and linked to V¹* via the oxygen atom of the PAB group. In some embodiments, X¹* is a substituted p-amino-benzyloxy (PAB) group, which is linked to P¹* via the amino nitrogen atom of the PAB group, and linked to V¹* via the oxygen atom of the PAB group. In some embodiments X¹*is a p-amino-benzyl (PAB) group substituted with a phosphate group, including but not limited to —OPO³H, wherein the PAB group is linked to P¹* via the amino nitrogen atom of the PAB group, and linked to V¹* via the oxygen atom of the PAB group. In some embodiments, X¹* is

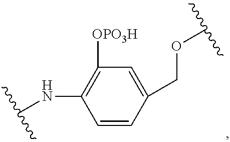

which is linked to P¹* via the amino nitrogen atom, and linked to V¹* via the oxygen atom. In some embodiments, X¹* is

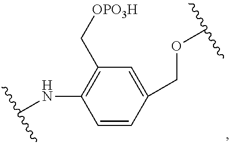

which is linked to P¹* via the amino nitrogen atom, and linked to V¹* via the oxygen atom. In some embodiments, X¹* is

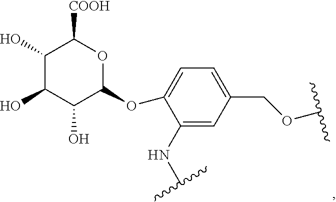

which is linked to P¹* via the amino nitrogen atom, and linked to V¹* via the oxygen atom.

In some embodiments, L¹* is —N(R¹*)-P¹*-X¹*—, wherein each of R¹*, P¹*, and X¹* is independently as described herein.

In some embodiments, L¹* is

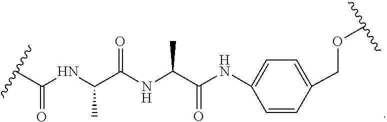 [SMILES: *OCc1ccc(NC(=O)[C@H](C)NC(=O)[C@H](C)NC(*)=O)cc1]

In some embodiments, L¹* is

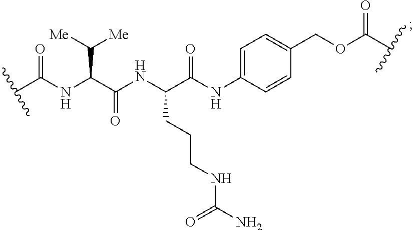 [SMILES: *C(=O)N[C@H](C(=O)N[C@@H](CCCNC(N)=O)C(=O)Nc1ccc(COC(*)=O)cc1)C(C)C]

In some embodiments, L¹* is

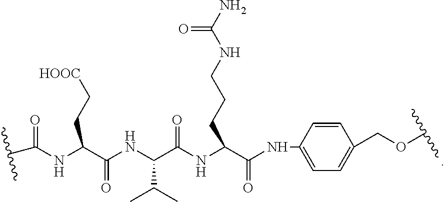 [SMILES: *OCc1ccc(NC(=O)[C@H](CCCNC(N)=O)NC(=O)[C@@H](NC(=O)[C@H](CCC(=O)O)NC(*)=O)C(C)C)cc1]

In some embodiments,

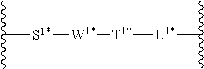

is

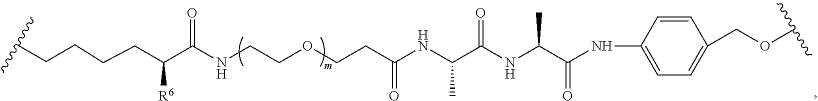

wherein each of R⁶* and m is independently as described herein.

In some embodiments,

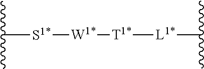

is

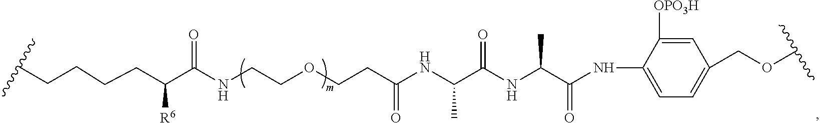

wherein each of R⁶* and m is independently as described herein. In some embodiments,

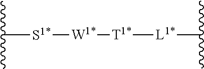

is

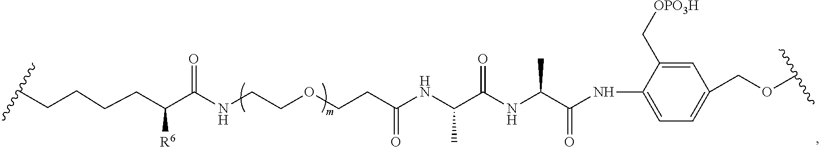

wherein each of R⁶* and m is independently as described herein.

In some embodiments,

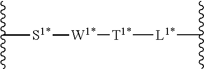

is

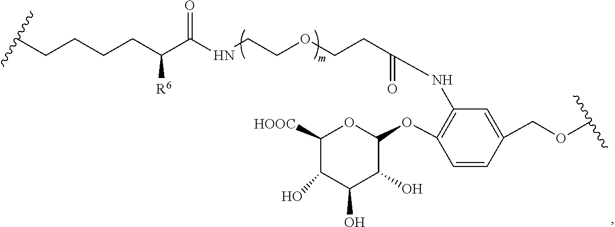

wherein each of R⁶* and m is independently as described herein.

In some embodiments,

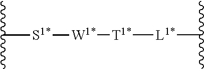

is

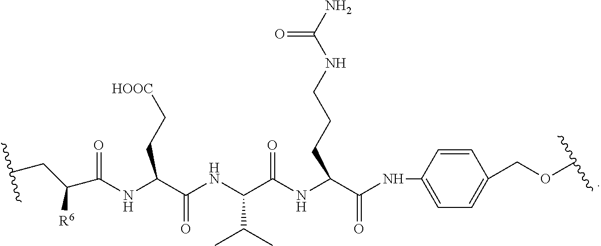

wherein each of R⁶* is independently as described herein.

In some embodiments,

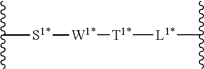

is

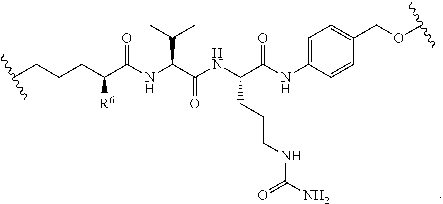

wherein each R⁶* is independently as described herein.

In some embodiments,

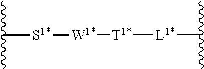

is

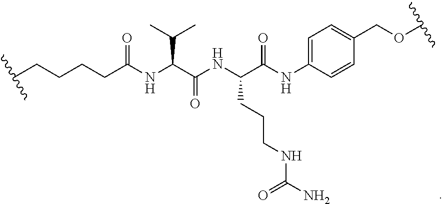 [SMILES: *CCCCC(=O)N[C@H](C(=O)N[C@@H](CCCNC(N)=O)C(=O)Nc1ccc(CO*)cc1)C(C)C]

In some embodiments,

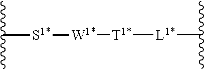

is

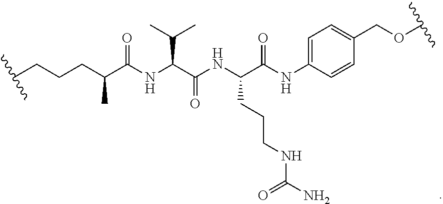 [SMILES: *CCC[C@H](C)C(=O)N[C@H](C(=O)N[C@@H](CCCNC(N)=O)C(=O)Nc1ccc(CO*)cc1)C(C)C]

In some embodiments,

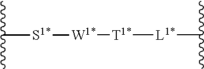

is

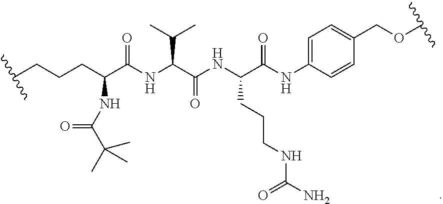 [SMILES: *CCC[C@H](NC(=O)C(C)(C)C)C(=O)N[C@H](C(=O)N[C@@H](CCCNC(N)=O)C(=O)Nc1ccc(CO*)cc1)C(C)C]

In some embodiments,

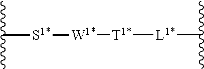

is

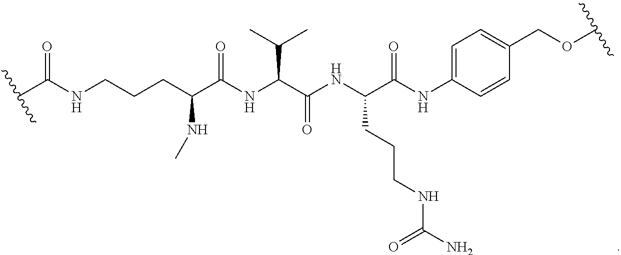 [SMILES: *OCc1ccc(NC(=O)[C@H](CCCNC(N)=O)NC(=O)[C@@H](NC(=O)[C@H](CCCNC(*)=O)NC)C(C)C)cc1]

In some embodiments,

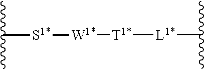

is

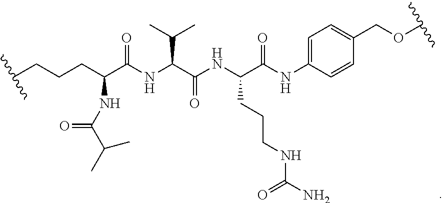 [SMILES: *CCC[C@H](NC(=O)C(C)C)C(=O)N[C@H](C(=O)N[C@@H](CCCNC(N)=O)C(=O)Nc1ccc(CO*)cc1)C(C)C]

In some embodiments,

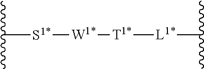

is

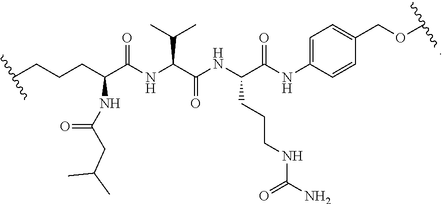 [SMILES: *CCC[C@H](NC(=O)CC(C)C)C(=O)N[C@H](C(=O)N[C@@H](CCCNC(N)=O)C(=O)Nc1ccc(CO*)cc1)C(C)C]

In some embodiments,

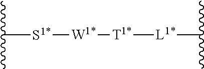

is

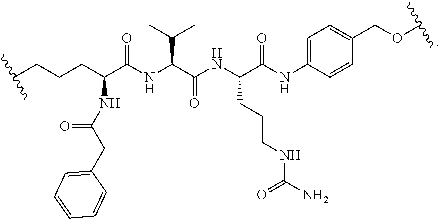 [SMILES: *CCC[C@H](NC(=O)Cc1ccccc1)C(=O)N[C@H](C(=O)N[C@@H](CCCNC(N)=O)C(=O)Nc1ccc(CO*)cc1)C(C)C]

In some embodiments,

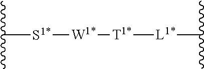

is

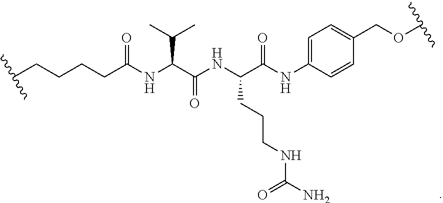 [SMILES: *CCCCC(=O)N[C@H](C(=O)N[C@@H](CCCNC(N)=O)C(=O)Nc1ccc(CO*)cc1)C(C)C]

In some embodiments,

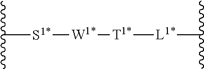

is

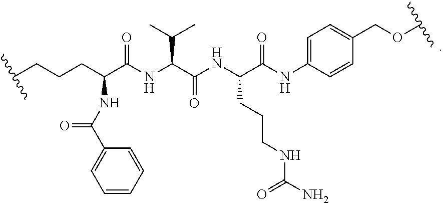 [SMILES: *CCC[C@H](NC(=O)c1ccccc1)C(=O)N[C@H](C(=O)N[C@@H](CCCNC(N)=O)C(=O)Nc1ccc(CO*)cc1)C(C)C]

In some embodiments,

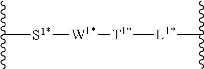

is

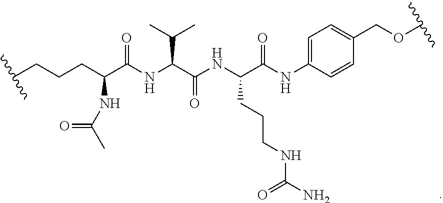 [SMILES: *CCC[C@H](NC(C)=O)C(=O)N[C@H](C(=O)N[C@@H](CCCNC(N)=O)C(=O)Nc1ccc(CO*)cc1)C(C)C]

In some embodiments,

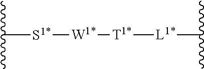

is

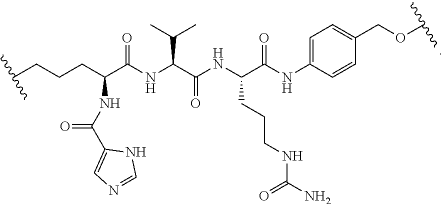 [SMILES: *CCC[C@H](NC(=O)c1cnc[nH]1)C(=O)N[C@H](C(=O)N[C@@H](CCCNC(N)=O)C(=O)Nc1ccc(CO*)cc1)C(C)C]

In some embodiments,

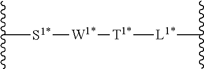

is

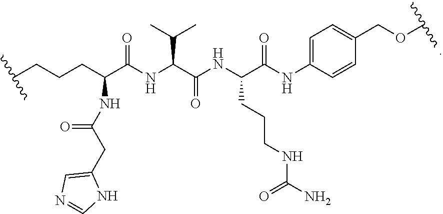 [SMILES: *CCC[C@H](NC(=O)Cc1cnc[nH]1)C(=O)N[C@H](C(=O)N[C@@H](CCCNC(N)=O)C(=O)Nc1ccc(CO*)cc1)C(C)C]

In some embodiments

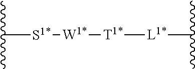

is

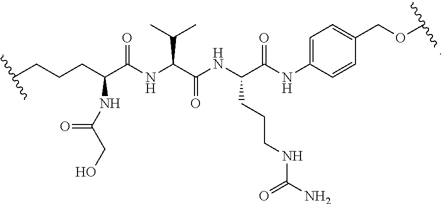 [SMILES: *CCC[C@H](NC(=O)CO)C(=O)N[C@H](C(=O)N[C@@H](CCCNC(N)=O)C(=O)Nc1ccc(CO*)cc1)C(C)C]

In some embodiments,

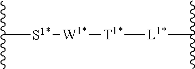

is

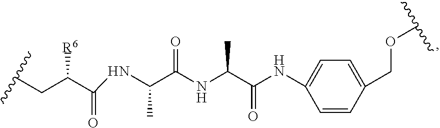

wherein each R⁶* is independently as described herein.

### 2.7. Bivalent Linker LINKER²

As defined generally above, each LINKER² is independently a bivalent linker. In some embodiments, LINKER² comprises a hydrocarbon chain unit S². In some embodiments, LINKER² comprises an amide moiety W². In some embodiments, LINKER² comprises a PEG unit T². In some embodiments, LINKER² comprises a peptide unit P². In some embodiments, LINKER² comprises a cleavable linker L².

In some embodiments, LINKER² is

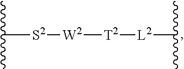

wherein:
S² is a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

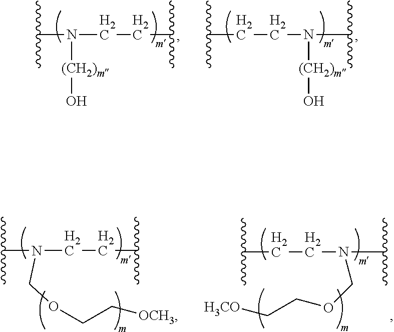

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

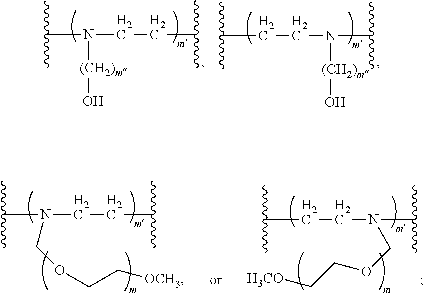

or W² and T² are absent;
L² is —C(O)-P²-X²— or —N(R²)-P²-X²—, wherein each P² is absent or a peptide comprising 1-20 amino acids, and each X² is a cleavable linker;
each of R² and R³ is independently H or optionally substituted C₁₋₆ aliphatic;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently an integer from 0-24.

As defined generally above, each S² is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

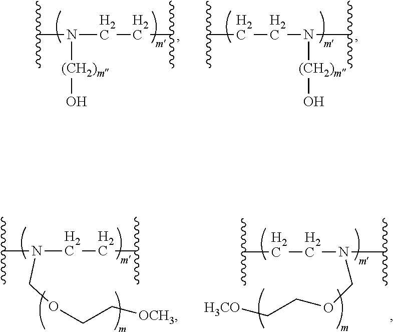

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S² is a covalent bond.

In some embodiments, S² is a bivalent C₁₋₈ saturated or unsaturated, straight or branched, hydrocarbon chain wherein one methylene unit of the chain is optionally and independently replaced by -Cy-, —C(R)₂—, —(OCH₂CH₂)ₘ—, —(CH₂CH₂O)ₘ,

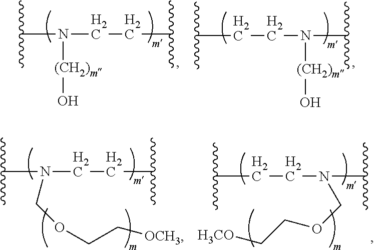

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S² is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

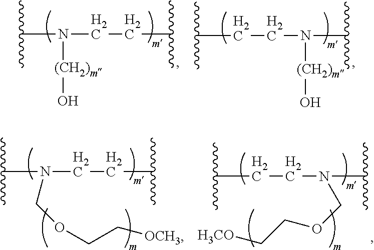

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

In some embodiments, S² is a bivalent C₁₋₈ saturated or unsaturated, straight or branched, hydrocarbon chain wherein two methylene units of the chain are optionally and independently replaced by -Cy-, —C(R)₂—, —(OCH₂CH₂)ₘ—, —(CH₂CH₂O)ₘ—,

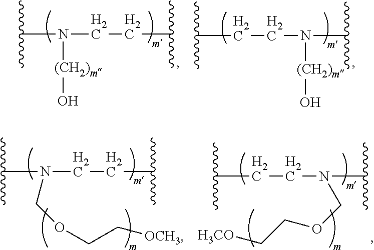

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein each of -Cy-, R, m, m′, and m″ is independently as described herein.

As defined generally above, each W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

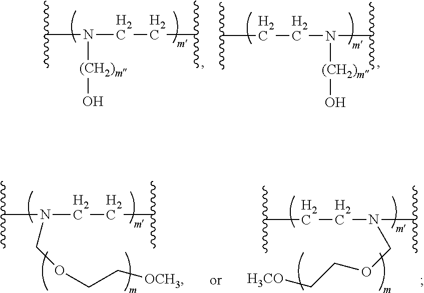

or W² and T² are absent, wherein each of R³, m, m′, and m″ is independently as described herein.

In some embodiments, W² is —C(O)—N(R³)—, wherein R³ is H or optionally substituted C₁₋₆ aliphatic. In some embodiments, W² is —C(O)—N(R³)—, wherein R³ is as described herein. In some embodiments, W² is —C(O)—NH—.

In some embodiments, W² is —N(R³)—C(O)—, wherein R³ is H or optionally substituted C₁₋₆ aliphatic. In some embodiments, W² is —N(R³)—C(O)—, wherein R³ is as described herein. In some embodiments, W² is —NH—C(O)—.

As defined generally above, each T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

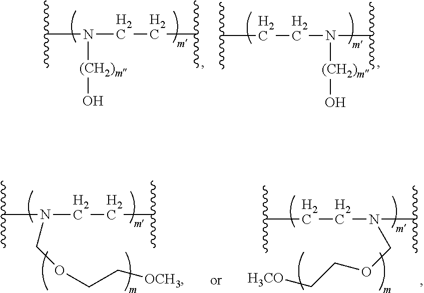

wherein each of m, m′, and m″ is independently as described herein.

In some embodiments, T² is a bivalent C₁₋₈ saturated or unsaturated, straight or branched, hydrocarbon chain wherein one methylene unit of the chain is replaced by —(OCH₂CH₂)ₘ—, wherein m is as described herein.

In some embodiments, T² is a bivalent C₁₋₈ saturated or unsaturated, straight or branched, hydrocarbon chain wherein one methylene unit of the chain is replaced by —(CH₂CH₂O)ₘ—, wherein m is as described herein.

In some embodiments, T² is a bivalent C₁₋₈ saturated or unsaturated, straight or branched, hydrocarbon chain wherein one methylene unit of the chain is replaced by

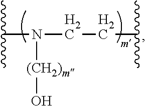

wherein each of m′ and m″ is as described herein.

In some embodiments, T² is a bivalent C₁₋₈ saturated or unsaturated, straight or branched, hydrocarbon chain wherein one methylene unit of the chain is replaced by

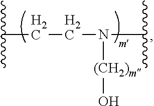

wherein each of m′ and m″ is as described herein.

In some embodiments, T² is a bivalent C₁₋₈ saturated or unsaturated, straight or branched, hydrocarbon chain wherein one methylene unit of the chain is replaced by

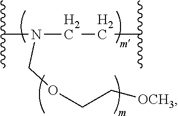

wherein each of m and m′ is as described herein.

In some embodiments, T² is a bivalent C₁₋₈ saturated or unsaturated, straight or branched, hydrocarbon chain wherein one methylene unit of the chain is replaced by

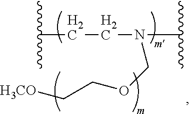

wherein each of m and m′ is as described herein.

In some embodiments, T² is

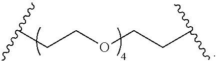

In some embodiments, T² and W² are absent.

As defined generally above, each L² is independently —C(O)-P²-X²— or —N(R²)-P²-X²—, wherein P² is absent or a peptide comprising 1-20 amino acids, X² is a cleavable linker, and R² is H or optionally substituted C₁₋₆ aliphatic.

In some embodiments, L² is —C(O)-P²-X²— or —N(R²)-P²-X²—, wherein R² is H or optionally substituted C₁₋₆ aliphatic, P² is a peptide comprising 1-20 amino acids, and X² is a cleavable linker. In some embodiments, L² is —C(O)-P²-X²—, wherein each of P² and X² is independently as described herein. In some embodiments, L² is —N(R²)-P²-X²—, wherein each of R², P², and X² is independently as described herein.

As defined generally above, each R² is independently H or optionally substituted C₁₋₆ aliphatic. In some embodiments, R² is H. In some embodiments, R¹ is optionally substituted C₁₋₆ aliphatic. In some embodiments, R² is optionally substituted C₁₋₆ alkyl. In some embodiments, R² is methyl, ethyl, propyl, isopropyl, butyl, isobutyl, or tert-butyl.

As defined generally above, each P² is independently absent or a peptide comprising 1-20 amino acids. In some embodiments, P¹ is absent. In some embodiments, P² is a peptide comprising 1-20 amino acids. In some embodiments, P² is a peptide having 1, 2, 3, 4, or 5 amino acids. In some embodiments, P² is a peptide having 6, 7, 8, 9, or 10 amino acids. In some embodiments, P² is a peptide having 11, 12, 13, 14, or 15 amino acids. In some embodiments, P² is a peptide having 16, 17, 18, 19, or 20 amino acids. In some embodiments, each amino acids of P² is a L-amino acid. In some embodiments, P² is a dipeptide selected from -(L-ala)-(L-ala)-, -Val-Cit-, and -Val-Ala-. In some embodiments, P² is a tripeptide -Glu-Val-Cit-. In some embodiments, P² is a tripeptide -AcLys-Val-Cit-. In some embodiments, P² is a tetrapeptide -Gly-Gly-Phe-Gly-.

As defined generally above, each X² is independently a cleavable linker. In some embodiments, X² is a self-immolative linker capable of releasing -D² without the need for a separate hydrolysis step. In some embodiments X² is a p-amino-benzyloxy (PAB) group, which is linked to P² via the amino nitrogen atom of the PAB group, and linked to V² via the oxygen atom of the PAB group. In some embodiments, X² is a PAB group

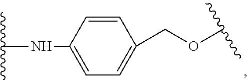 [SMILES: *Nc1ccc(CO*)cc1]

which is linked to P² via the amino nitrogen atom of the PAB group, and linked to V² via the oxygen atom of the PAB group. In some embodiments, X² is a substituted p-amino-benzyloxy (PAB) group, which is linked to P² via the amino nitrogen atom of the PAB group, and linked to V² via the oxygen atom of the PAB group. In some embodiments X² is a p-amino-benzyl (PAB) group substituted with a phosphate group, including but not limited to —OPO3H, wherein the PAB group is linked to P² via the amino nitrogen atom of the PAB group, and linked to V² via the oxygen atom of the PAB group. In some embodiments, X² is

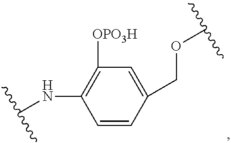

which is linked to P² via the amino nitrogen atom, and linked to V² via the oxygen atom. In some embodiments, X² is

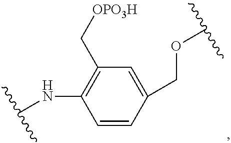

which is linked to P² via the amino nitrogen atom, and linked to V² via the oxygen atom. In some embodiments, X² is

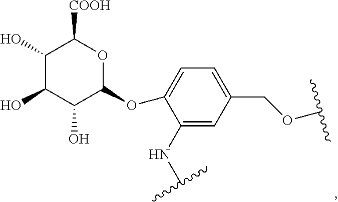

which is linked to P² via the amino nitrogen atom, and linked to V² via the oxygen atom.

In some embodiments, L² is —N(R²)-P²-X²—, wherein each of R², P², and X² is independently as described herein.

In some embodiments, L² is

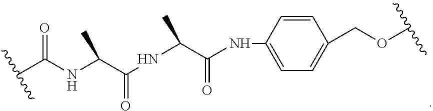 [SMILES: *OCc1ccc(NC(=O)[C@H](C)NC(=O)[C@H](C)NC(*)=O)cc1]

In some embodiments, L² is

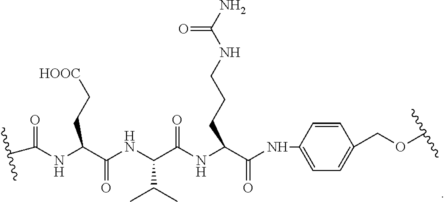 [SMILES: *OCc1ccc(NC(=O)[C@H](CCCNC(N)=O)NC(=O)[C@@H](NC(=O)[C@H](CCC(=O)O)NC(*)=O)C(C)C)cc1]

In some embodiments,

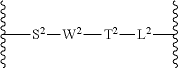

is

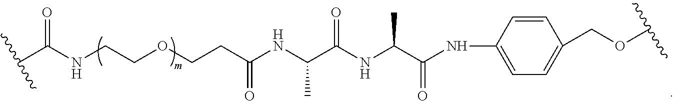

In some embodiments,

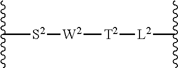

is

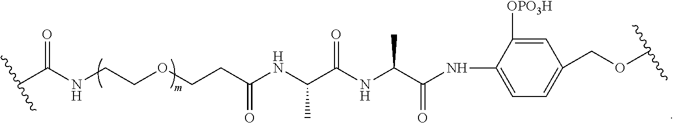

In some embodiments,

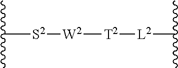

is

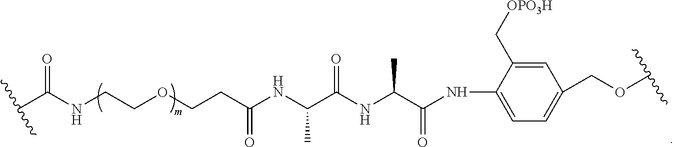

In some embodiments,

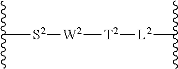

is

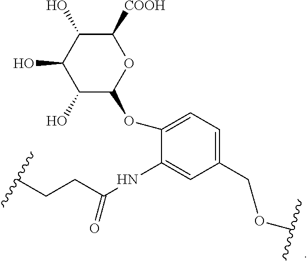

In some embodiments,

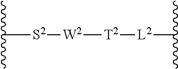

is

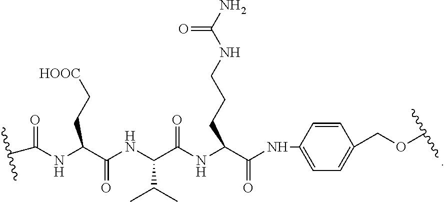 [SMILES: *OCc1ccc(NC(=O)[C@H](CCCNC(N)=O)NC(=O)[C@@H](NC(=O)[C@H](CCC(=O)O)NC(*)=O)C(C)C)cc1]

2.8. Payload D, D¹, and D²

As defined generally above, each of D, D¹, and D² is independently a payload. A payload can be a small molecule, a protein or polypeptide, or a biocompatible polymer.

In some embodiments, each of D, D¹, and D² is independently a cytotoxic agent, an immunosuppressive agent, or an imaging agent (e.g., a fluorophore). In some embodiments, a cytotoxic agent is a chemotherapeutic agent.

In some embodiments, each of D, D¹, and D² is independently a NAMPT inhibitor.

In some embodiments, each of D, D¹, and D² is independently a PRMT5 inhibitor.

In some embodiments, each of D, D¹, and D² is independently an Alpha-Amanitin.

In some embodiments, each of D, D¹, and D² is independently a PNU anthracycline. In some embodiments, each of D, D¹, and D² is independently a HSP90 inhibitor.

In some embodiments, each of D, D¹, and D² is independently a cytotoxic agent, selected from anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof.

In some embodiments, each of D, D¹, and D² is independently an anthracycline or a derivative thereof, including, but not limited to, daunorubicin, doxorubicin (i.e., adriamycin), epirubicin, idarubicin, valrubicin, and mitoxantrone.

In some embodiments, each of D, D¹, and D² is independently a dolastatin or auristatin, or a derivative thereof, including, but not limited to, dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), and the cytotoxic pentapeptides described in U.S. Pat. No. 8,828,401, including but not limited to Aur0101 (PF06380101), which is hereby incorporated by reference in its entirety.

In some embodiments, each of D, D¹, and D² is independently a camptothecin or a derivative thereof, including, but not limited to, topotecan and irinotecan, or a metabolite thereof, such as SN-38. In some embodiments, each of D, D¹, and D² is independently exatecan

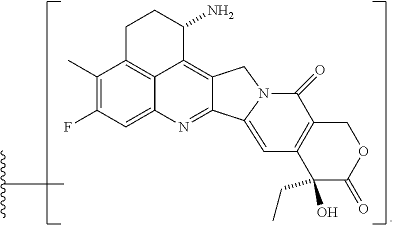

In some embodiments, D, D¹, and D² are

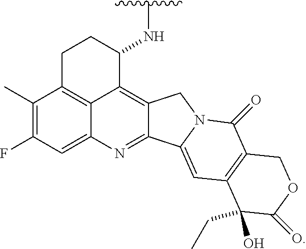 [SMILES: *N[C@H]1CCc2c(C)c(F)cc3nc4c(c1c23)Cn1c-4cc2c(c1=O)COC(=O)[C@]2(O)CC]

In some embodiments, D, D¹, and D² are

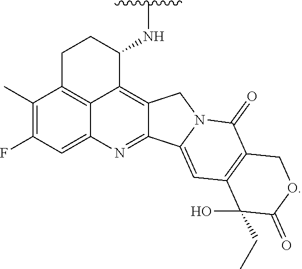 [SMILES: *N[C@H]1CCc2c(C)c(F)cc3nc4c(c1c23)Cn1c-4cc2c(c1=O)COC(=O)[C@]2(O)CC]

In some embodiments, D¹ is

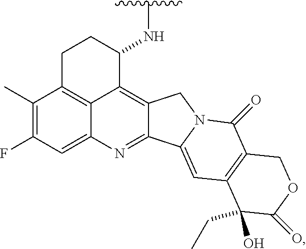 [SMILES: *N[C@H]1CCc2c(C)c(F)cc3nc4c(c1c23)Cn1c-4cc2c(c1=O)COC(=O)[C@]2(O)CC]

### and D² is

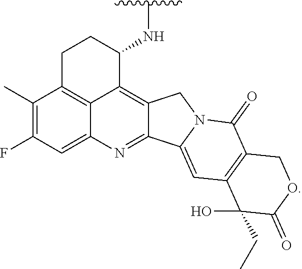 [SMILES: *N[C@H]1CCc2c(C)c(F)cc3nc4c(c1c23)Cn1c-4cc2c(c1=O)COC(=O)[C@]2(O)CC]

In some embodiments, D¹ is

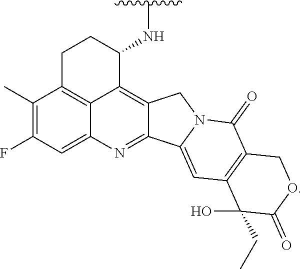 [SMILES: *N[C@H]1CCc2c(C)c(F)cc3nc4c(c1c23)Cn1c-4cc2c(c1=O)COC(=O)[C@]2(O)CC]

### and D² is

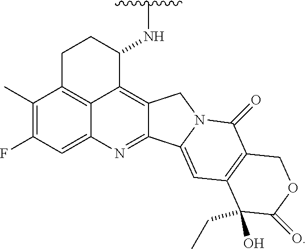 [SMILES: *N[C@H]1CCc2c(C)c(F)cc3nc4c(c1c23)Cn1c-4cc2c(c1=O)COC(=O)[C@]2(O)CC]

In some embodiments, D is

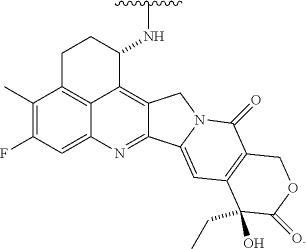 [SMILES: *N[C@H]1CCc2c(C)c(F)cc3nc4c(c1c23)Cn1c-4cc2c(c1=O)COC(=O)[C@]2(O)CC]

In some embodiments, D is

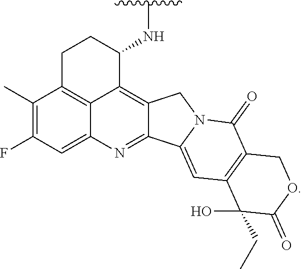 [SMILES: *N[C@H]1CCc2c(C)c(F)cc3nc4c(c1c23)Cn1c-4cc2c(c1=O)COC(=O)[C@]2(O)CC]

In some embodiments, each of D, D¹, and D² is independently a combretastatin or a derivative thereof, including, but not limited to, combretastatin A-4 (CA-4) and ombrabulin.

In some embodiments, each of D, D¹, and D² is independently an enediyne or a derivative thereof, including, but not limited to, calicheamicin, esperamicin, uncialamicin, dynemicin, and their derivatives.

In some embodiments, each of D, D¹, and D² is independently a geldanamycin or a derivative thereof, including, but not limited to, 17-AAG (17-N-Allylamino-17-Demethoxygeldanamycin) and 17-DMAG (17-Dimethylaminoethylamino-17-demethoxygeldanamycin).

In some embodiments, each of D, D¹, nd D² is independently a hemiasterlin or a derivative thereof, including, but not limited to, HTI-286.

In some embodiments, each of D, D¹, and D² is independently a maytansine or a maytansinoid, or a derivative thereof, including, but not limited to, mertansine (DM1) and its derivatives as well as ansamitocin.

In some embodiments, each of D, D¹, and D² is independently a pyrrolobenzodiazepine dimer (PBD) or an indolino-benzodiazepine dimer (IGN), or a derivative thereof, including, but not limited to, talirine and tesirine.

In some embodiments, each of D, D¹, and D² is independently a spliceostatin or a pladienolide, or a derivative thereof, including, but not limited to, spliceostatin A, FR901464, Pladienolide B, Pladienolide D, and E7107.

In some embodiments, each of D, D¹, and D² is independently a taxane or a derivative thereof, including, but not limited to, paclitaxel (e.g. TAXOL®) and docetaxel (TAXOTERE®).

In some embodiments, each of D, D¹, and D² is independently a tubulysin or a derivative thereof, including, but not limited to, tubulysin A, tubulysin B, and tubulysin D.

In some embodiments, each of D, D¹, and D² is independently a vinca alkyloid or a derivative thereof, including, but not limited to, vincristine, vinblastine, vindesine, and vinorelbine.

In some embodiments, each of D, D¹, and D² is independently an immunosuppressive agent, including, but not limited to, ganciclovir, etanercept, tacrolimus, sirolimus, voclosporin, cyclosporine, rapamycin, cyclophosphamide, azathioprine, mycophenolgate mofetil, methotrextrate, and glucocorticoids and their analogs and derivatives.

In some embodiments, each of D, D¹, and D² is independently an imaging agent (e.g., a fluorophore or a chelator), such as fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5-TAMRA), tetramethylrhodamine (TMR), and sulforhodamine (SR) (e.g., SR101). Examples of chelators include, but are not limited to, 1,4,7,10-tetraazacyclododecane-N,N′,N″,N′″-tetraacetic acid (DOT A), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

In some embodiments, each of D, D¹, and D² is independently a polypeptide. In some embodiments, a polypeptide is an antibody, such as a humanized, human, chimeric, or murine monoclonal antibody.

In some embodiments, each of D, D¹, and D² is independently a toxin polypeptide (or a toxin protein), including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPI I, and PAP-S), Momordica charantia inhibitor, curcin, crotin, Sapaonaria officinalis inhibitor, gelonin, mitogellin, restrictocin, phenomycin, enomycin, tricothecenes, inhibitor cystine knot (ICK) peptides (e.g., ceratotoxins), and conotoxin (e.g., KIIIA or SIIIA).

In some embodiments, each of D, D¹, and D² is independently a payload as described in WO2015162563, which is incorporated herein by reference in its entirety.

In some embodiments, the present disclosure provides a ligand-payload conjugate of Formula (I-1):

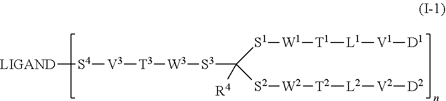

or a pharmaceutically acceptable salt thereof, wherein each variable is independently as described herein.

In some embodiments, the present disclosure provides a ligand-payload conjugate of Formula (I-2):

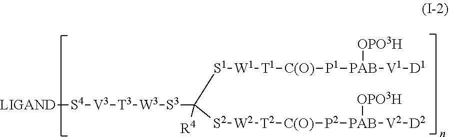

or a pharmaceutically acceptable salt thereof, wherein each variable is independently as described herein.

In some embodiments, the present disclosure provides a ligand-payload conjugate of Formula (I-3):

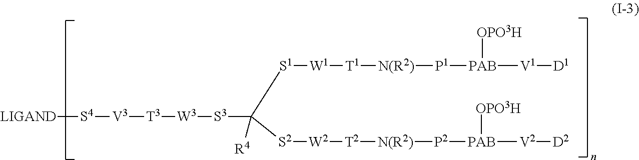

or a pharmaceutically acceptable salt thereof, wherein each variable is independently as described herein.

In some embodiments, the present disclosure provides a ligand-payload conjugate of Formula (I-4):

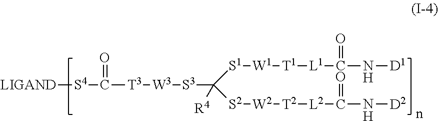

or a pharmaceutically acceptable salt thereof, wherein each variable is independently as described herein.

In some embodiments, the present disclosure provides a ligand-payload conjugate of Formula (I-5):

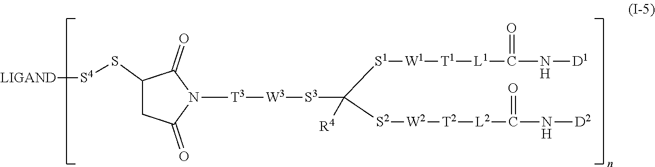

or a pharmaceutically acceptable salt thereof, wherein each variable is independently as described herein.

In some embodiments, the present disclosure provides a ligand-payload conjugate of Formula (I-6):

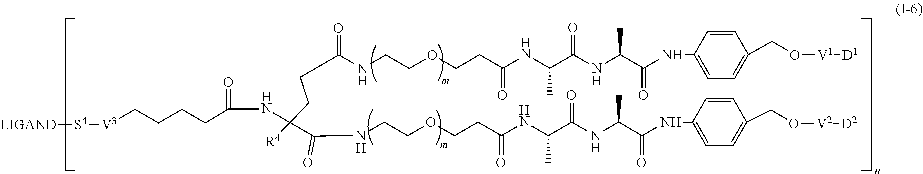

or a pharmaceutically acceptable salt thereof, wherein each variable is independently as described herein.

In some embodiments, the present disclosure provides a ligand-payload conjugate of Formula (I-7):

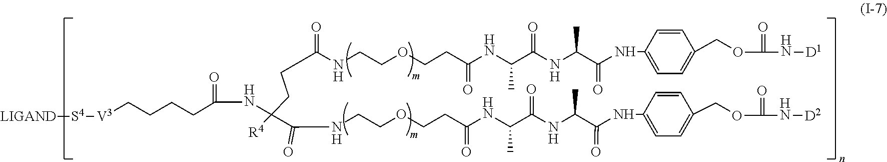

or a pharmaceutically acceptable salt thereof, wherein each variable is independently as described herein.

In some embodiments, the present disclosure provides a ligand-payload conjugate of Formula (I-8):

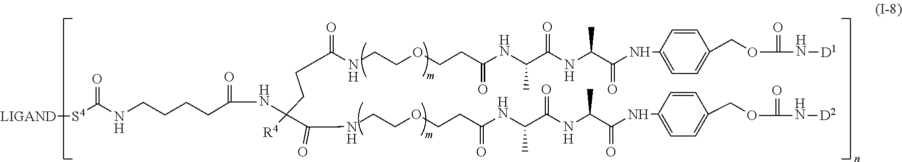

or a pharmaceutically acceptable salt thereof, wherein each variable is independently as described herein.

In some embodiments, the present disclosure provides a ligand-payload conjugate of Formula (I-9):

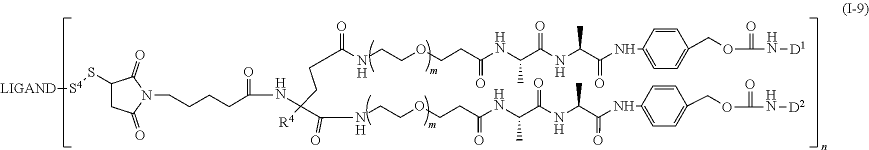

or a pharmaceutically acceptable salt thereof, wherein each variable is independently as described herein.

In some embodiments, the present disclosure provides a ligand-payload conjugate of Formula (I-10):

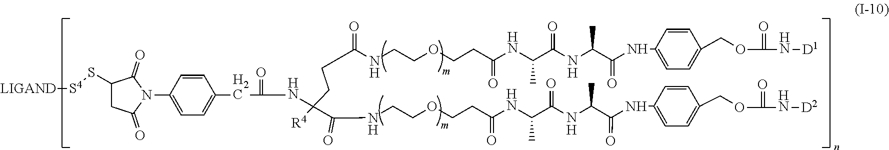

or a pharmaceutically acceptable salt thereof, wherein each t is independently an integer from 0-20, and each variable is independently as described herein. In some embodiments, t is 0, 1, 2, 3, 4, 5, 6, 7, 8, 9, 10, 11, 12, 13, 14, 15, 16, 17, 18, 19, or 20.

In some embodiments, the present disclosure provides a ligand-payload conjugate of Formula (I-11):

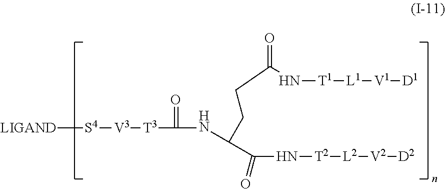

or a pharmaceutically acceptable salt thereof, wherein each variable is independently as described herein.

In some embodiments, the present disclosure provides a ligand-payload conjugate of Formula (I-12):

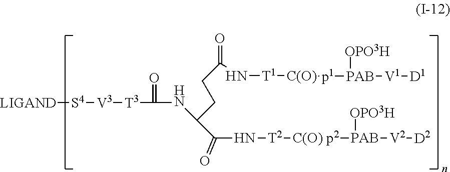

or a pharmaceutically acceptable salt thereof, wherein each variable is independently as described herein.

In some embodiments, the present disclosure provides a ligand-payload conjugate of Formula (I-13):

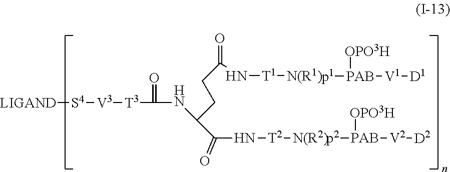

or a pharmaceutically acceptable salt thereof, wherein each variable is independently as described herein.

In some embodiments, the present disclosure provides a ligand-payload conjugate of Formula (I-14):

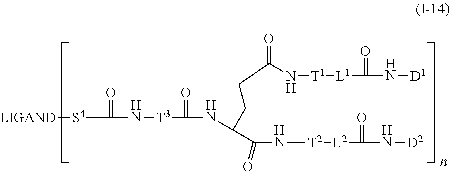

or a pharmaceutically acceptable salt thereof, wherein each variable is independently as described herein.

In some embodiments, the present disclosure provides a ligand-payload conjugate of Formula (I-15):

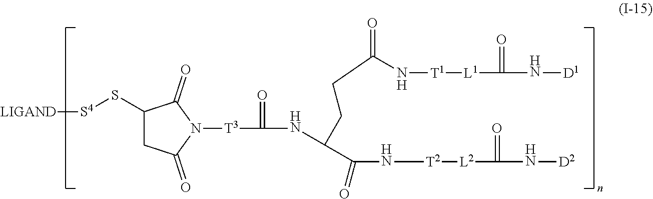

or a pharmaceutically acceptable salt thereof, wherein each variable is independently as described herein.

In some embodiments, the present disclosure provides a ligand-payload conjugate of Formula (I-16):

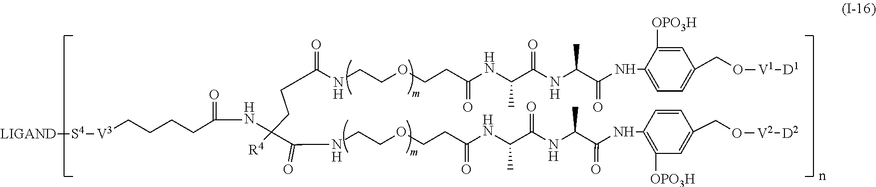

or a pharmaceutically acceptable salt thereof, wherein each variable is independently as described herein.

In some embodiments, the present disclosure provides a ligand-payload conjugate of Formula (I-17):

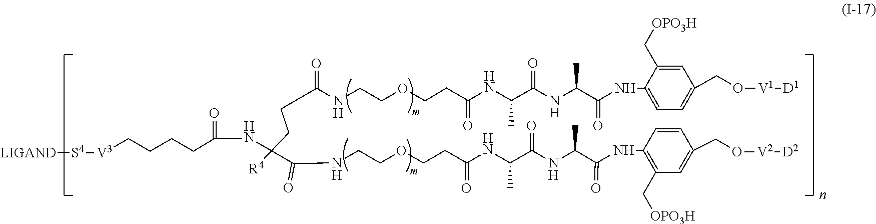

or a pharmaceutically acceptable salt thereof, wherein each variable is independently as described herein.

In some embodiments, the present disclosure provides a ligand-payload conjugate of Formula (I-18):

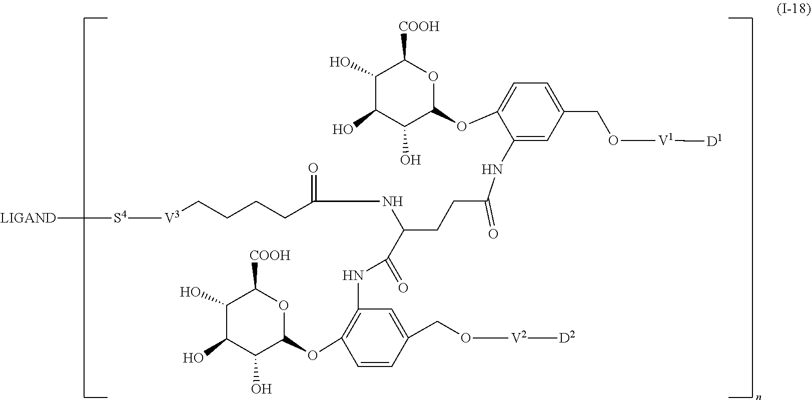

or a pharmaceutically acceptable salt thereof, wherein each variable is independently as described herein.

In some embodiments, the present invention provides a ligand-payload conjugate of Formula (I-1*):

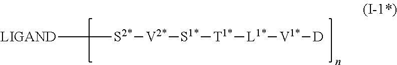

or a pharmaceutically acceptable salt thereof, wherein each variable is independently as described herein.

In some embodiments, the present invention provides a ligand-payload conjugate of Formula (I-2*):

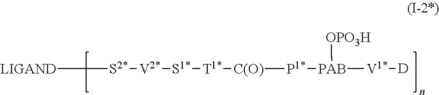

or a pharmaceutically acceptable salt thereof, wherein each variable is independently as described herein.

In some embodiments, the present invention provides a ligand-payload conjugate of Formula (I-3*):

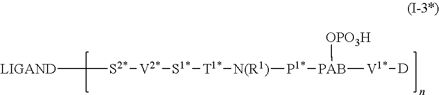

or a pharmaceutically acceptable salt thereof, wherein each variable is independently as described herein.

In some embodiments, the present invention provides a ligand-payload conjugate of Formula (I-4*):

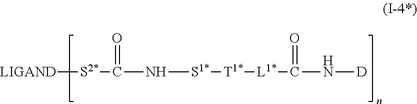

or a pharmaceutically acceptable salt thereof, wherein each variable is independently as described herein.

In some embodiments, the present invention provides a ligand-payload conjugate of Formula (I-5*):

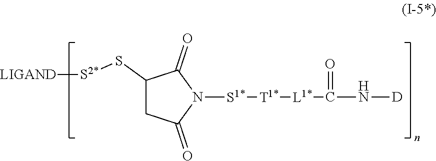

or a pharmaceutically acceptable salt thereof, wherein each variable is independently as described herein.

In some embodiments, the present invention provides a ligand-payload conjugate of Formula (I-6*):

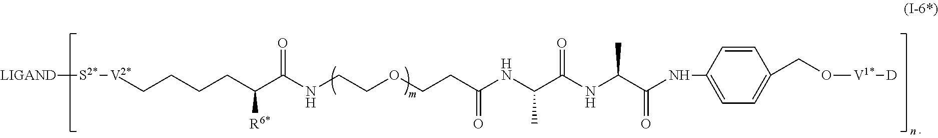

or a pharmaceutically acceptable salt thereof, wherein each variable is independently as described herein.

In some embodiments, the present invention provides a ligand-payload conjugate of Formula (I-7*):

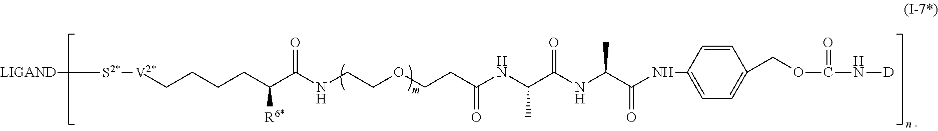

or a pharmaceutically acceptable salt thereof, wherein each variable is independently as described herein.

In some embodiments, the present invention provides a ligand-payload conjugate of Formula (I-8*):

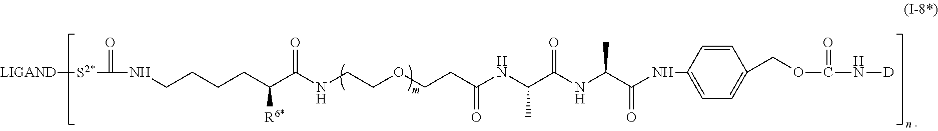

or a pharmaceutically acceptable salt thereof, wherein each variable is independently as described herein.

In some embodiments, the present invention provides a ligand-payload conjugate of Formula (I-9*):

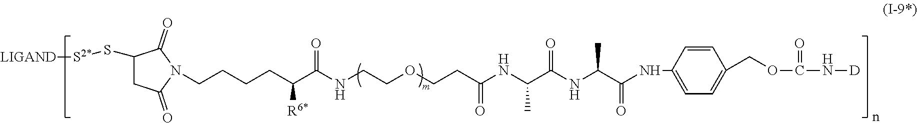

or a pharmaceutically acceptable salt thereof, wherein each variable is independently as described herein.

In some embodiments, the present invention provides a ligand-payload conjugate of Formula (I-10*):

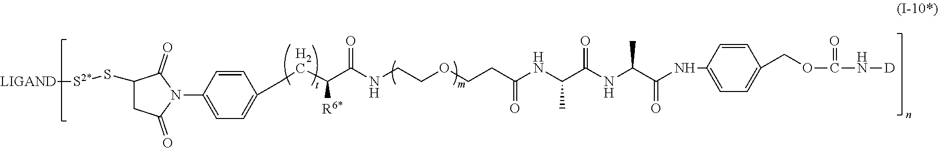

or a pharmaceutically acceptable salt thereof, wherein t is an integer from 0-20, and each variable is independently as described herein. In some embodiments, t is 0, 1, 2, 3, 4, 5, 6, 7, 8,9, 10, 11, 12, 13, 14, 15, 16, 17, 18, 19, or 20.

In some embodiments, the present invention provides a ligand-payload conjugate of Formula (I-11*):

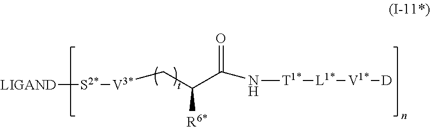

or a pharmaceutically acceptable salt thereof, wherein each variable is independently as described herein.

In some embodiments, the present invention provides a ligand-payload conjugate of Formula (I-12*):

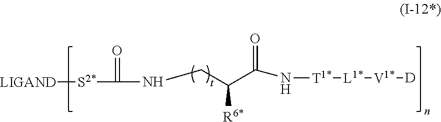

or a pharmaceutically acceptable salt thereof, wherein each variable is independently as described herein.

In some embodiments, the present invention provides a ligand-payload conjugate of Formula (I-13*)

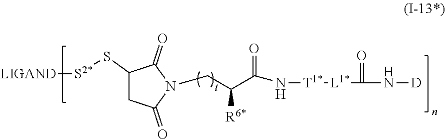

or a pharmaceutically acceptable salt thereof, wherein each variable is independently as described herein.

### 3. General Methods of Providing the Present Compounds and Related Intermediates

In one aspect, the instant disclosure provides a linker-payload compound of formula (II):

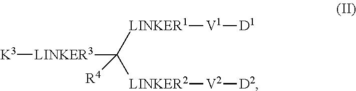

or a pharmaceutically acceptable salt thereof, wherein K³ is a functional group capable of forming a linker moiety by a coupling reaction with a different functional group, and each of LINKER¹, LINKER², LINKER³, V¹, V², R⁴, D¹, and D² is independently as defined and described herein.

As defined generally above, each K³ is independently a functional group capable of forming a linker moiety by a coupling reaction with a different functional group. In some embodiments, each K³ is independently a functional group selected from those listed in Table 2.

In some embodiments, K³ is a functional group capable of forming a linker moiety by a Michael addition with a different functional group. In some embodiments, K³ is a Michael acceptor. In some embodiments, K³ is a maleimide group

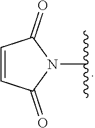 [SMILES: *N1C(=O)C=CC1=O]

attached to LINKER³. In some embodiments, K³ is a N-aryl maleimide group

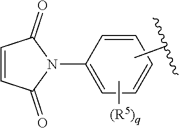

attached to LINKER³, wherein each of R⁵ and q is independently as described herein. In some embodiments, K³ is a N-aryl maleimide group

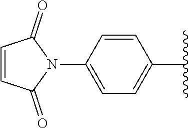 [SMILES: *c1ccc(N2C(=O)C=CC2=O)cc1]

attached to LINKER³. In some embodiments, K³ is a maleimide group

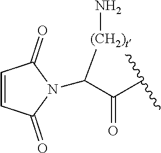

attached to LINKER³, wherein each t′ is independently as described herein. In some embodiments, K³ is a maleimide group

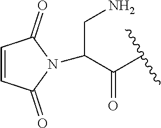 [SMILES: *C(=O)C(CN)N1C(=O)C=CC1=O]

attached to LINKER³. In some embodiments, K³ is a Michael donor. In some embodiments, K³ is a thiol group —SH attached to LINKER³.

In some embodiments, K³ is a functional group capable of forming a linker moiety by a leaving group displacement reaction with a different functional group. In some embodiments, K³ is an amine group or an amine donor attached to LINKER³. In some embodiments, K³ is —NH₂. In some embodiments, K³ is a leaving group attached to LINKER³. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K³ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K³ is an activated ester selected from N-hydroxysuccinimde, pentafluorophenyl, and p-nitrophenyl esters.

In some embodiments, K³ is a functional group capable of forming a linker moiety by an azide-alkyne cycloaddition. In some embodiments, K³ is an azide group —N₃ attached to LINKER³. In some embodiments, K³ is an alkyne group

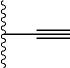 [SMILES: *C#C]

attached to LINKER³. In some embodiments, K³ is a cyclooctyne group

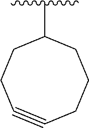 [SMILES: *C1CCC#CCCC1]

attached to LINKER³.

In some embodiments, K³ is a functional group capable of forming a linker moiety by a transglutaminase mediated acyl addition. In some embodiments, K³ is an acyl acceptor and the anime donor. In some embodiments, K³ is —NH₂. In some embodiments, K³ is an acyl donor. In some embodiments, K³ is the γ-carboxamide group (—(C═O)NH₂) of a glutamine residue of LINKER³.

In one aspect, the instant disclosure provides a ligand of formula (III):

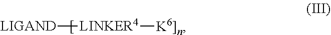

or a pharmaceutically acceptable salt thereof, wherein each K⁶ is independently a functional group capable of forming a linker moiety by a coupling reaction with a different functional group, and each of LIGAND, LINKER⁴, and n is independently as defined and described herein.

As defined generally above, each K⁶ is independently a functional group capable of forming a linker moiety by a coupling reaction with a different functional group. In some embodiments, each K⁶ is independently a functional group selected from those listed in Table 2.

In some embodiments, K⁶ is a functional group capable of forming a linker moiety by a Michael addition with a different functional group. In some embodiments, K⁶ is a Michael acceptor. In some embodiments, K⁶ is a maleimide group

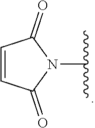 [SMILES: *N1C(=O)C=CC1=O]

attached to LINKER⁴. In some embodiments, K⁶ is a N-aryl maleimide group

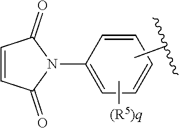

attached to LINKER⁴, wherein each of R⁵ and q is independently as described herein. In some embodiments, K⁶ is a N-aryl maleimide group

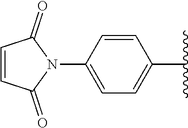 [SMILES: *c1ccc(N2C(=O)C=CC2=O)cc1]

attached to LINKER⁴. In some embodiments, K⁶ is a maleimide group

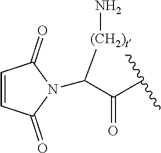

attached to LINKER⁴, wherein each t′ is independently as described herein. In some embodiments, K⁶ is a maleimide group

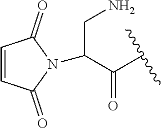 [SMILES: *C(=O)C(CN)N1C(=O)C=CC1=O]

attached to LINKER⁴. In some embodiments, K⁶ is a Michael donor. In some embodiments, K⁶ is a thiol group —SH attached to LINKER⁴.

In some embodiments, K⁶ is a functional group capable of forming a linker moiety by a leaving group displacement reaction with a different functional group. In some embodiments, K⁶ is an amine group or an amine donor attached to LINKER⁴. In some embodiments, K⁶ is —NH₂. In some embodiments, K⁶ is a leaving group attached to LINKER⁴. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁶ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁶ is an activated ester selected from N-hydroxysuccinimde, pentafluorophenyl, and p-nitrophenyl esters.

In some embodiments, K⁶ is a functional group capable of forming a linker moiety by an azide-alkyne cycloaddition. In some embodiments, K⁶ is an azide group —N₃ attached to LINKER⁴. In some embodiments, K⁶ is an alkyne group

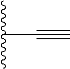 [SMILES: *C#C]

attached to LINKER⁴. In some embodiments, K⁶ is a cyclooctyne group

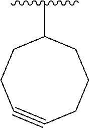 [SMILES: *C1CCC#CCCC1]

attached to LINKER⁴.

In some embodiments, K⁶ is a functional group capable of forming a linker moiety by a transglutaminase mediated acyl addition. In some embodiments, K⁶ is an acyl acceptor and the anime donor. In some embodiments, K⁶ is an acyl donor. In some embodiments, K⁶ is the γ-carboxamide group (—(C═O)NH₂) of a glutamine residue of LINKER⁴.

In one aspect, the present disclosure provides a method for synthesizing a ligand-payload conjugate of Formula (I):

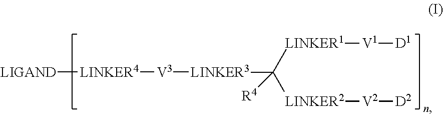

by a coupling reaction between a linker-payload compound of formula (II):

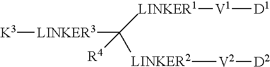

and a ligand of formula (III):

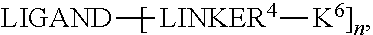

wherein each variable is independently as defined and described herein, and the functional groups K³ and K⁶ form a linker moiety V³ by a coupling reaction.

In some embodiments, K³ and K⁶ form a linker moiety V³ by a Michael addition, wherein K³ is a maleimide group

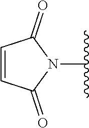 [SMILES: *N1C(=O)C=CC1=O]

and K⁶ is a thiol group —SH, or wherein K³ is a thiol group —SH, and K⁶ is a maleimide group

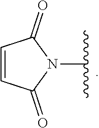 [SMILES: *N1C(=O)C=CC1=O]

In some embodiments, K³ and K⁶ form a linker moiety V³ by a Michael addition, wherein K³ is a maleimide group

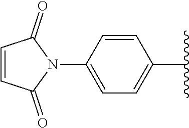 [SMILES: *c1ccc(N2C(=O)C=CC2=O)cc1]

and K⁶ is a thiol group —SH, or wherein K³ is a thiol group —SH, and K⁶ is a maleimide group

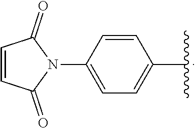 [SMILES: *c1ccc(N2C(=O)C=CC2=O)cc1]

In some embodiments, K³ and K⁶ form a linker moiety V³ by a Michael addition, wherein K³ is a maleimide group

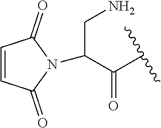 [SMILES: *C(=O)C(CN)N1C(=O)C=CC1=O]

and K⁶ is a thiol group —SH, or wherein K³ is a thiol group —SH, and K⁶ is a maleimide group

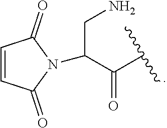 [SMILES: *C(=O)C(CN)N1C(=O)C=CC1=O]

In some embodiments, K³ and K⁶ form a linker moiety V³ by a leaving group displacement reaction, wherein K³ is —NH₂ and K⁶ is a leaving group, or wherein K³ is a leaving group, and K⁶ is —NH₂. In some embodiments, K³ and K⁶ form a linker moiety V³ by a leaving group displacement reaction, wherein K³ is —NH₂ and K⁶ is an activated ester —C(O)-LG, or wherein K³ is an activated ester —C(O)-LG, and K⁶ is —NH₂.

In some embodiments, K³ and K⁶ form a linker moiety V³ an azide-alkyne cycloaddition, wherein K³ is —N₃ and K⁶ is

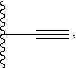 [SMILES: *C#C]

or wherein K³ is

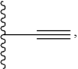 [SMILES: *C#C]

and K⁶ is —N₃. In some embodiments, K³ and K⁶ form a linker moiety V³ by an azide-alkyne cycloaddition, wherein K³ is —N₃ and K⁶ is

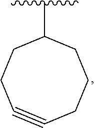 [SMILES: *C1CCC#CCCC1]

or wherein K³ is

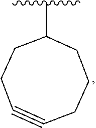 [SMILES: *C1CCC#CCCC1]

and K⁶ is —N₃.

In some embodiments, K³ and K⁶ form a linker moiety V³ by a transglutaminase mediated acyl addition, wherein K³ is —NH₂ and K⁶ is γ-carboxamide group (—(C═O)NH₂) of a glutamine residue of LINKER⁴, or K³ is γ-carboxamide group (—(C═O)NH₂) of a glutamine residue of LINKER³ and K⁶ is —NH₂.

In one aspect, the instant disclosure provides a linker-payload compound of formula (IV-1):

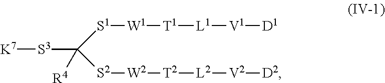

or a pharmaceutically acceptable salt thereof, wherein each K⁷ is independently a functional group capable of forming a carboxamide linker moiety by a coupling reaction with a different functional group, each K⁷ optionally comprising a protecting group, and each of L¹, L², S¹, S², S³, R⁴, W¹, W², T¹, T², V¹, V², D¹, and D² is independently as defined and described herein.

As defined generally above, each K⁷ is independently a functional group capable of forming a carboxamide linker moiety by a coupling reaction with a different functional group. In some embodiments, each K⁷ is independently a functional group selected from those listed in Table 2.

In some embodiments, K⁷ is a functional group capable of forming a carboxamide linker moiety by a leaving group displacement reaction with a different functional group. In some embodiments, K⁷ is an amine group or an amine. In some embodiments, K⁷ is —NH₂. In some embodiments, K⁷ comprises a leaving group. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁷ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁷ is an activated ester selected from N-hydroxysuccinimde, pentafluorophenyl, and p-nitrophenyl esters.

In some embodiments, K⁷ is a functional group capable of forming a carboxamide linker moiety by an acyl addition. In some embodiments, K⁷ is an acyl acceptor and the anime donor. In some embodiments, K⁷ is —NH₂. In some embodiments, K⁷ is an acyl donor. In some embodiments, K⁷ is —COOH.

In some embodiments, K⁷ is —NH-PG, wherein PG is a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. In some embodiments, a suitable amino protecting group PG is selected from t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

In one aspect, the instant disclosure provides a linker of formula (V):

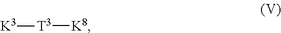

or a pharmaceutically acceptable salt thereof, wherein each K⁸ is independently a functional group capable of forming a carboxamide linker moiety by a coupling reaction with a different functional group, and each of K³ and T³ is independently as defined and described herein.

As defined generally above, each K⁸ is independently a functional group capable of forming a carboxamide linker moiety by a coupling reaction with a different functional group. In some embodiments, each K⁸ is independently a functional group selected from those listed in Table 2.

In some embodiments, K⁸ is a functional group capable of forming a carboxamide linker moiety by a leaving group displacement reaction with a different functional group. In some embodiments, K⁸ is an amine group or an anine. In some embodiments, K⁸ is —NH₂. In some embodiments, K⁸ comprises a leaving group. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁸ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁸ is an activated ester selected from N-hydroxysuccinimde, pentafluorophenyl, and p-nitrophenyl esters.

In some embodiments, K⁸ is a functional group capable of forming a carboxamide linker moiety by an acyl addition. In some embodiments, K⁸ is an acyl acceptor and the anime donor. In some embodiments, K⁸ is —NH₂. In some embodiments, K⁸ is an acyl donor. In some embodiments, K⁸ is —COOH.

In one aspect, the present disclosure provides a method for synthesizing a linker-payload compound of Formula (II-1):

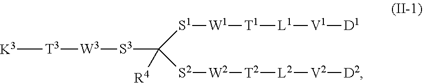

by a coupling reaction between a linker-payload compound of formula (IV-1):

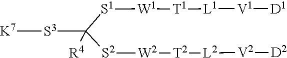

and a linker of formula (V): K³-T³-K⁸, wherein each variable is independently as defined and described herein, and the functional groups K⁷ and K⁸ form a linker moiety W³ by a coupling reaction.

In some embodiments, the functional groups K⁷ and K⁸ form a carboxamide linker W³ by a coupling reaction.

In some embodiments, K⁷ is an amine group or an amine, and K⁸ comprises a leaving group as described herein. In some embodiments, K⁷ comprises a leaving group as described herein, and K⁸ is an amine group or an amine. In some embodiments, K⁷ is —NH₂, and K⁸ is an activated ester —C(O)-LG as described herein. In some embodiments, K⁷ is an activated ester —C(O)-LG as described herein, and K⁸ is —NH₂. In some embodiments, K⁷ is —NH₂, and K⁸ is —COOH. In some embodiments, K⁷ is —COOH, and K⁸ is —NH₂.

In one aspect, the instant disclosure provides a linker of formula (VI-1):

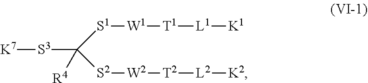

or a pharmaceutically acceptable salt thereof, wherein each K¹ and K² is independently a functional group capable of forming a linker moiety by a coupling reaction with a different functional group, and each of L¹, L², S¹, S², S³, R⁴, T¹, T², S³, W¹, W², and K⁷ is independently as defined and described herein.

As defined generally above, each K¹ is independently a functional group capable of forming a linker moiety by a coupling reaction with a different functional group. In some embodiments, each K¹ is independently a functional group selected from those listed in Table 2.

In some embodiments, K¹ is a functional group capable of forming a linker moiety by a Michael addition with a different functional group. In some embodiments, K¹ is a Michael acceptor. In some embodiments, K¹ is a maleimide group

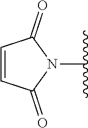 [SMILES: *N1C(=O)C=CC1=O]

attached to LINKER¹. In some embodiments, K¹ is a N-aryl maleimide group

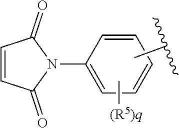

attached to LINKER¹, wherein each of R⁵ and q is independently as described herein. In some embodiments, K¹ is a N-aryl maleimide group

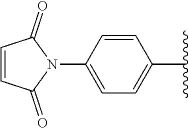 [SMILES: *c1ccc(N2C(=O)C=CC2=O)cc1]

attached to LINKER¹. In some embodiments, K¹ is a maleimide group

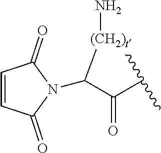

attached to LINKER¹, wherein each t′ is independently as described herein. In some embodiments, K¹ is a maleimide group

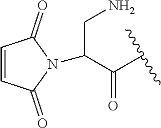 [SMILES: *C(=O)C(CN)N1C(=O)C=CC1=O]

attached to LINKER¹. In some embodiments, K¹ is a Michael donor. In some embodiments, K¹ is a thiol group —SH attached to LINKER¹.

In some embodiments, K¹ is a functional group capable of forming a linker moiety by a leaving group displacement reaction with a different functional group. In some embodiments, K¹ is an amine group or an amine donor attached to LINKER¹. In some embodiments, K¹ is —NH₂. In some embodiments, K¹ is a leaving group attached to LINKER¹. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K¹ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K¹ is an activated ester selected from N-hydroxysuccinimde, pentafluorophenyl, and p-nitrophenyl esters.

In some embodiments, K¹ is a functional group capable of forming a linker moiety by an azide-alkyne cycloaddition. In some embodiments, K¹ is an azide group —N₃ attached to LINKER¹. In some embodiments, K¹ is an alkyne group

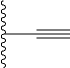 [SMILES: *C#C]

attached to LINKER¹. In some embodiments, K¹ is a cyclooctyne group

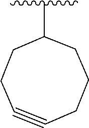 [SMILES: *C1CCC#CCCC1]

attached to LINKER¹.

In some embodiments, K¹ is a functional group capable of forming a linker moiety by an acyl addition. In some embodiments, K¹ is an acyl acceptor and the anime donor. In some embodiments, K¹ is —NH₂. In some embodiments, K¹ is an acyl donor. In some embodiments, K¹ is —COOH.

As defined generally above, each K² is independently a functional group capable of forming a linker moiety by a coupling reaction with a different functional group. In some embodiments, each K² is independently a functional group selected from those listed in Table 2.

In some embodiments, K² is a functional group capable of forming a linker moiety by a Michael addition with a different functional group. In some embodiments, K² is a Michael acceptor. In some embodiments, K² is a maleimide group

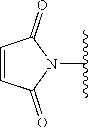 [SMILES: *N1C(=O)C=CC1=O]

attached to LINKER². In some embodiments, K² is a N-aryl maleimide group

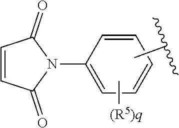

attached to LINKER², wherein each of R⁵ and q is independently as described herein. In some embodiments, K² is a N-aryl maleimide group

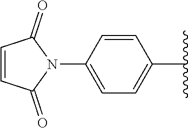 [SMILES: *c1ccc(N2C(=O)C=CC2=O)cc1]

attached to LINKER². In some embodiments, K² is a maleimide group

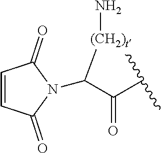

attached to LINKER², wherein each t′ is independently as described herein. In some embodiments, K² is a maleimide group

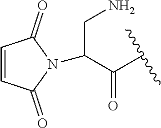 [SMILES: *C(=O)C(CN)N1C(=O)C=CC1=O]

attached to LINKER². In some embodiments, K² is a Michael donor. In some embodiments, K² is a thiol group —SH attached to LINKER².

In some embodiments, K² is a functional group capable of forming a linker moiety by a leaving group displacement reaction with a different functional group. In some embodiments, K² is an amine group or an amine donor attached to LINKER². In some embodiments, K² is —NH₂. In some embodiments, K² is a leaving group attached to LINKER². A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K² is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K² is an activated ester selected from N-hydroxysuccinimde, pentafluorophenyl, and p-nitrophenyl esters.

In some embodiments, K² is a functional group capable of forming a linker moiety by an azide-alkyne cycloaddition. In some embodiments, K² is an azide group —N₃ attached to LINKER². In some embodiments, K² is an alkyne group

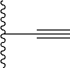 [SMILES: *C#C]

attached to LINKER². In some embodiments, K² is a cyclooctyne group

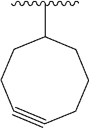 [SMILES: *C1CCC#CCCC1]

attached to LINKER².

In some embodiments, K² is a functional group capable of forming a linker moiety by an acyl addition. In some embodiments, K² is an acyl acceptor and the anime donor. In some embodiments, K² is —NH₂. In some embodiments, K² is an acyl donor. In some embodiments, K² is —COOH.

In another aspect, the instant disclosure provides a payload of formula (VII):

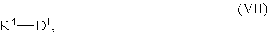

or a pharmaceutically acceptable salt thereof, wherein each K⁴ is a functional group capable of forming a linker moiety by a coupling reaction with a different functional group, and each D¹ is independently as defined and described herein.

As defined generally above, each K⁴ is independently a functional group capable of forming a linker moiety by a coupling reaction with a different functional group. In some embodiments, each K⁴ is independently a functional group selected from those listed in Table 2.

In some embodiments, K⁴ is a functional group capable of forming a linker moiety by a Michael addition with a different functional group. In some embodiments, K⁴ is a Michael acceptor. In some embodiments, K⁴ is a maleimide group

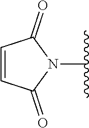 [SMILES: *N1C(=O)C=CC1=O]

attached to D¹. In some embodiments, K⁴ is a N-aryl maleimide group

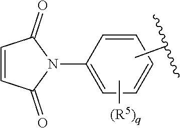

attached to D¹, wherein each of R⁵ and q is independently as described herein. In some embodiments, K⁴ is a N-aryl maleimide group

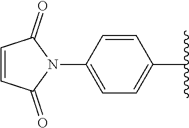 [SMILES: *c1ccc(N2C(=O)C=CC2=O)cc1]

attached to D¹. In some embodiments, K⁴ is a maleimide group

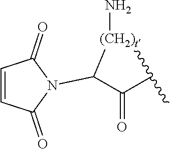

attached to D¹, wherein each t′ is independently as described herein. In some embodiments, K⁴ is a maleimide group

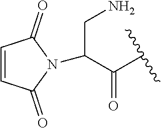 [SMILES: *C(=O)C(CN)N1C(=O)C=CC1=O]

attached to D¹. In some embodiments, K⁴ is a Michael donor. In some embodiments, K⁴ is a thiol group —SH attached to D¹.

In some embodiments, K⁴ is a functional group capable of forming a linker moiety by a leaving group displacement reaction with a different functional group. In some embodiments, K⁴ is an amine group or an amine donor attached to D¹. In some embodiments, K⁴ is —NH₂. In some embodiments, K⁴ is a leaving group attached to D¹. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁴ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁴ is an activated ester selected from N-hydroxysuccinimde, pentafluorophenyl, and p-nitrophenyl esters.

In some embodiments, K⁴ is a functional group capable of forming a linker moiety by an azide-alkyne cycloaddition. In some embodiments, K⁴ is an azide group —N₃ attached to D¹. In some embodiments, K⁴ is an alkyne group

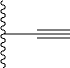 [SMILES: *C#C]

attached to D¹. In some embodiments, K⁴ is a cyclooctyne group

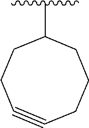 [SMILES: *C1CCC#CCCC1]

attached to D¹.

In some embodiments, K⁴ is a functional group capable of forming a linker moiety by an acyl addition. In some embodiments, K⁴ is an acyl acceptor and the anime donor. In some embodiments, K⁴ is —NH₂. In some embodiments, K⁴ is an acyl donor. In some embodiments, K⁴ is —COOH.

In another aspect, the instant disclosure provides a payload of formula (VIII):

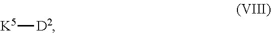

or a pharmaceutically acceptable salt thereof, wherein each K⁵ is a functional group capable of forming a linker moiety by a coupling reaction with a different functional group, and each D² is independently as defined and described herein.

As defined generally above, each K⁵ is independently a functional group capable of forming a linker moiety by a coupling reaction with a different functional group. In some embodiments, each K⁵ is independently a functional group selected from those listed in Table 2.

In some embodiments, K⁵ is a functional group capable of forming a linker moiety by a Michael addition with a different functional group. In some embodiments, K⁵ is a Michael acceptor. In some embodiments, K⁵ is a maleimide group

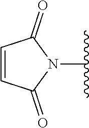 [SMILES: *N1C(=O)C=CC1=O]

attached to D². In some embodiments, K⁵ is a N-aryl maleimide group

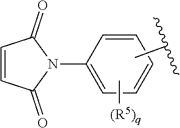

attached to D², wherein each of R⁵ and q is independently as described herein. In some embodiments, K⁵ is a N-aryl maleimide group

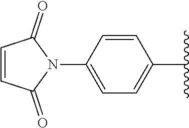 [SMILES: *c1ccc(N2C(=O)C=CC2=O)cc1]

attached to D². In some embodiments, K⁵ is a maleimide group

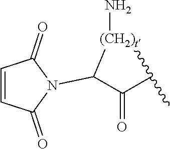

attached to D², wherein each t′ is independently as described herein. In some embodiments, K⁵ is a maleimide group

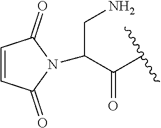 [SMILES: *C(=O)C(CN)N1C(=O)C=CC1=O]

attached to D². In some embodiments, K⁵ is a Michael donor. In some embodiments, K⁵ is a thiol group —SH attached to D².

In some embodiments, K⁵ is a functional group capable of forming a linker moiety by a leaving group displacement reaction with a different functional group. In some embodiments, K⁵ is an amine group or an amine donor attached to D². In some embodiments, K⁵ is —NH₂. In some embodiments, K⁵ is a leaving group attached to D². A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁵ is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁵ is an activated ester selected from N-hydroxysuccinimde, pentafluorophenyl, and p-nitrophenyl esters.

In some embodiments, K⁵ is a functional group capable of forming a linker moiety by an azide-alkyne cycloaddition. In some embodiments, K⁵ is an azide group —N₃ attached to D². In some embodiments, K⁵ is an alkyne group

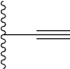 [SMILES: *C#C]

attached to D². In some embodiments, K⁵ is a cyclooctyne group

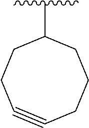 [SMILES: *C1CCC#CCCC1]

attached to D².

In some embodiments, K⁵ is a functional group capable of forming a linker moiety by an acyl addition. In some embodiments, K⁵ is an acyl acceptor and the anime donor. In some embodiments, K⁵ is —NH₂. In some embodiments, K⁵ is an acyl donor. In some embodiments, K⁵ is —COOH.

In one aspect, the present disclosure provides a method for synthesizing a linker-payload of Formula (IV-1):

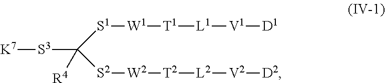

by a coupling reaction between a linker of formula (VI-1):

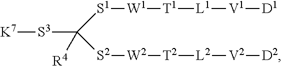

a payload of formula (VII): K⁴-D¹, and a payload of formula (VIII): K⁵-D², wherein the functional groups K¹ and K⁴ form a linker moiety V¹ by a coupling reaction, the functional groups K² and K⁵ form a linker moiety V² by a coupling reaction, and each variable is as defined and described herein.

In some embodiments, K¹ and K⁴ form a linker moiety V¹ by a Michael addition, wherein K¹ is a maleimide group

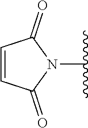 [SMILES: *N1C(=O)C=CC1=O]

and K⁴ is a thiol group —SH, or wherein K¹ is a thiol group —SH, and K⁴ is a maleimide group

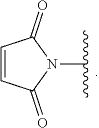 [SMILES: *N1C(=O)C=CC1=O]

In some embodiments, K¹ and K⁴ form a linker moiety V¹ by a Michael addition, wherein K¹ is a maleimide group

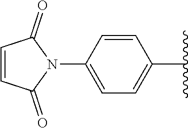 [SMILES: *c1ccc(N2C(=O)C=CC2=O)cc1]

and K⁴ is a thiol group —SH, or wherein K¹ is a thiol group —SH, and K⁴ is a maleimide group

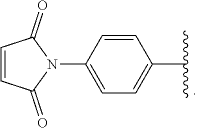 [SMILES: *c1ccc(N2C(=O)C=CC2=O)cc1]

In some embodiments, K¹ and K⁴ form a linker moiety V¹ by a Michael addition, wherein K¹ is a maleimide group

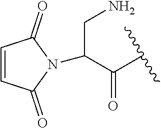 [SMILES: *C(=O)C(CN)N1C(=O)C=CC1=O]

and K⁴ is a thiol group —SH, or wherein K¹ is a thiol group —SH, and K⁴ is a maleimide group

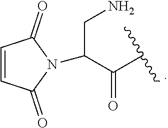 [SMILES: *C(=O)C(CN)N1C(=O)C=CC1=O]

In some embodiments, K¹ and K⁴ form a linker moiety V¹ by a leaving group displacement reaction, wherein K¹ is —NH₂ and K⁴ is a leaving group, or wherein K¹ is a leaving group, and K⁴ is —NH₂. In some embodiments, K¹ and K⁴ form a linker moiety V¹ by a leaving group displacement reaction, wherein K¹ is —NH₂ and K⁴ is an activated ester —C(O)-LG, or wherein K¹ is an activated ester —C(O)-LG, and K⁴ is —NH₂.

In some embodiments, K¹ and K⁴ form a linker moiety V¹ by an azide-alkyne cycloaddition, wherein K¹ is —N₃ and K⁴ is

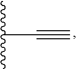 [SMILES: *C#C]

or wherein K¹ is

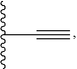 [SMILES: *C#C]

and K⁴ is —N₃. In some embodiments, K¹ and K⁴ form a linker moiety V¹ by an azide-alkyne cycloaddition, wherein K¹ is —N₃ and K⁴ is

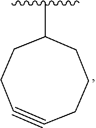 [SMILES: *C1CCC#CCCC1]

or wherein K¹ is

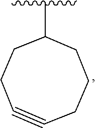 [SMILES: *C1CCC#CCCC1]

and K⁴ is —N₃.

In some embodiments, K¹ and K⁴ form a linker moiety V¹ by an acyl addition, wherein K¹ is —NH₂ and K⁴ is —COOH, or K¹ is —COOH and K⁴ is —NH₂.

In some embodiments, K² and K⁵ form a linker moiety V² by a Michael addition, wherein K² is a maleimide group

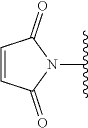 [SMILES: *N1C(=O)C=CC1=O]

and K⁵ is a thiol group —SH, or wherein K² is a thiol group —SH, and K⁵ is a maleimide group

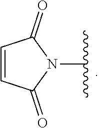 [SMILES: *N1C(=O)C=CC1=O]

In some embodiments, K² and K⁵ form a linker moiety V² by a Michael addition, wherein K² is a maleimide group

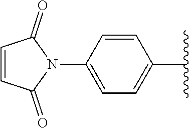 [SMILES: *c1ccc(N2C(=O)C=CC2=O)cc1]

and K⁵ is a thiol group —SH, or wherein K² is a thiol group —SH, and K⁵ is a maleimide group

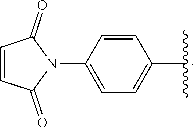 [SMILES: *c1ccc(N2C(=O)C=CC2=O)cc1]

In some embodiments, K² and K⁵ form a linker moiety V² by a Michael addition, wherein K² is a maleimide group

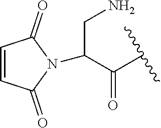 [SMILES: *C(=O)C(CN)N1C(=O)C=CC1=O]

and K⁵ is a thiol group —SH, or wherein K² is a thiol group —SH, and K⁵ is a maleimide group

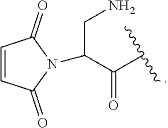 [SMILES: *C(=O)C(CN)N1C(=O)C=CC1=O]

In some embodiments, K² and K⁵ form a linker moiety V² by a leaving group displacement reaction, wherein K² is —NH₂ and K⁵ is a leaving group, or wherein K² is a leaving group, and K⁵ is —NH₂. In some embodiments, K² and K⁵ form a linker moiety V² by a leaving group displacement reaction, wherein K² is —NH₂ and K⁵ is an activated ester —C(O)-LG, or wherein K² is an activated ester —C(O)-LG, and K⁵ is —NH₂.

In some embodiments K² and K⁵ form a linker moiety V² by an azide-alkyne cycloaddition, wherein K² is —N₃ and K⁵ is

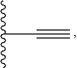 [SMILES: *C#C]

or wherein K² is

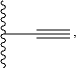 [SMILES: *C#C]

and K⁵ is —N₃. In some embodiments, K² and K⁵ form a linker moiety V² by an azide-alkyne cycloaddition, wherein K² is —N₃ and K⁵ is

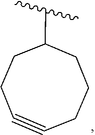 [SMILES: *C1CCC#CCCC1]

or wherein K² is

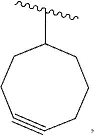 [SMILES: *C1CCC#CCCC1]

and K⁵ is —N₃.

In some embodiments, K² and K⁵ form a linker moiety V² by a transglutaminase mediated acyl addition, wherein K² is —NH₂ and K⁵ is —COOH, or K² is —COOH and K⁵ is —NH₂.

In some embodiments, the instant disclosure provides a linker-payload compound of formula (II-1):

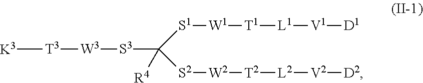

or a pharmaceutically acceptable salt thereof, wherein each variable is independently as described herein.

In some embodiments, the present disclosure provides a linker-payload compound of Formula (II-2):

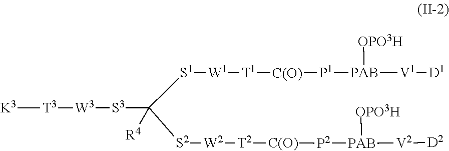

or a pharmaceutically acceptable salt thereof, wherein each variable is independently as described herein.

In some embodiments, the present disclosure provides a linker-payload compound of Formula (II-3):

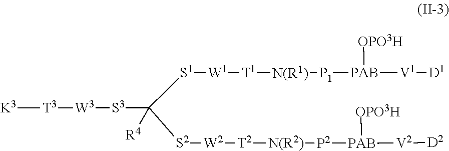

or a pharmaceutically acceptable salt thereof, wherein each variable is independently as described herein.

In some embodiments, the present disclosure provides a linker-payload compound of Formula (II-4):

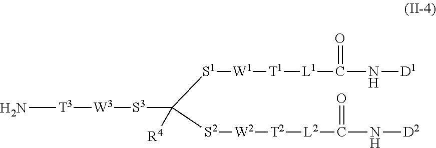

or a pharmaceutically acceptable salt thereof, wherein each variable is independently as described herein.

In some embodiments, the present disclosure provides a linker-payload compound of Formula (II-5):

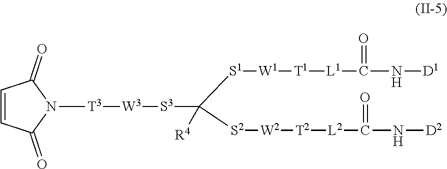

or a pharmaceutically acceptable salt thereof, wherein each variable is independently as described herein.

In some embodiments, the present disclosure provides a linker-payload compound of Formula (II-6):

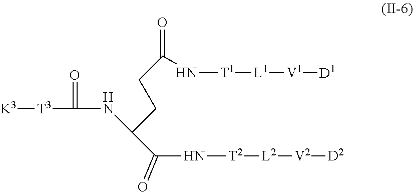

or a pharmaceutically acceptable salt thereof, wherein each variable is independently as described herein.

In some embodiments, the present disclosure provides a linker-payload compound of Formula (II-7):

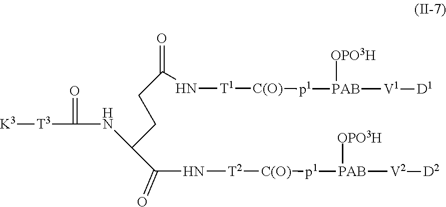

or a pharmaceutically acceptable salt thereof, wherein each variable is independently as described herein.

In some embodiments, the present disclosure provides a linker-payload compound of Formula (II-8):

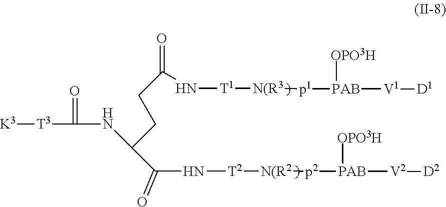

or a pharmaceutically acceptable salt thereof, wherein each variable is independently as described herein.

In some embodiments, the present disclosure provides a linker-payload compound of Formula (II-9a):

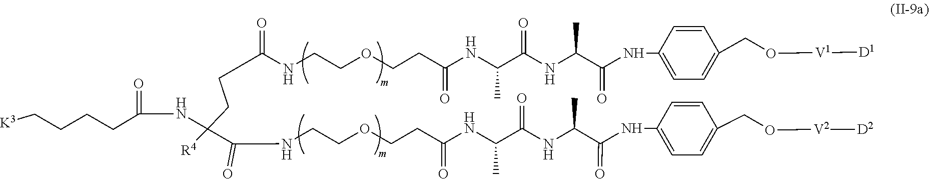

or a pharmaceutically acceptable salt thereof, wherein each variable is independently as described herein.

In some embodiments, the present disclosure provides a linker-payload compound of Formula (II-9b):

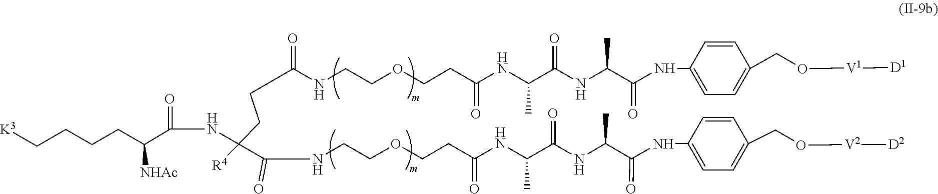

or a pharmaceutically acceptable salt thereof, wherein each variable is independently as described herein.

In some embodiments, the present disclosure provides a linker-payload compound of Formula (II-10a):

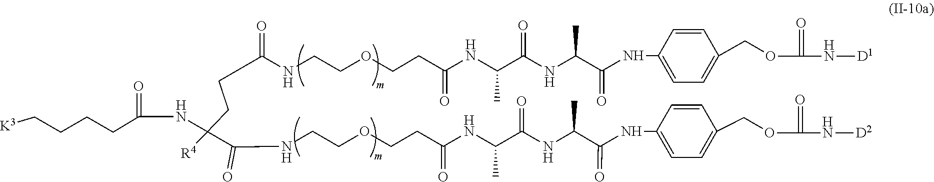

or a pharmaceutically acceptable salt thereof, wherein each variable is independently as described herein.

In some embodiments, the present disclosure provides a linker-payload compound of Formula (II-10b):

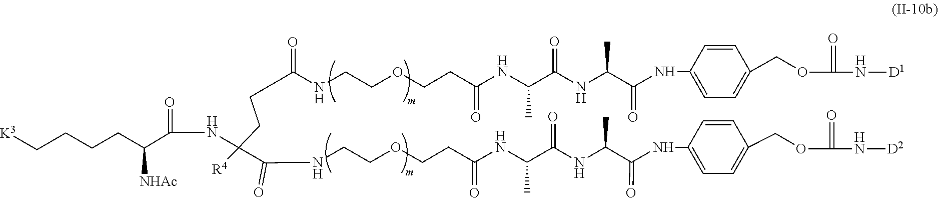

or a pharmaceutically acceptable salt thereof, wherein each variable is independently as described herein.

In some embodiments, the present disclosure provides a linker-payload compound of Formula (II-11a):

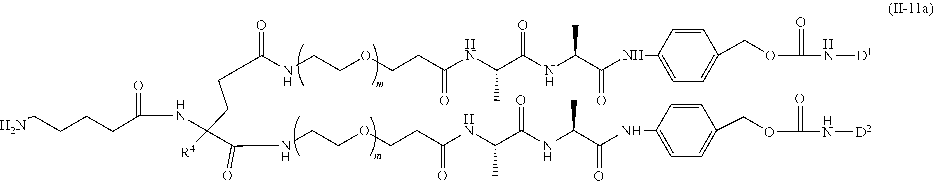

or a pharmaceutically acceptable salt thereof, wherein each variable is independently as described herein.

In some embodiments, the present disclosure provides a linker-payload compound of Formula (II-11b):

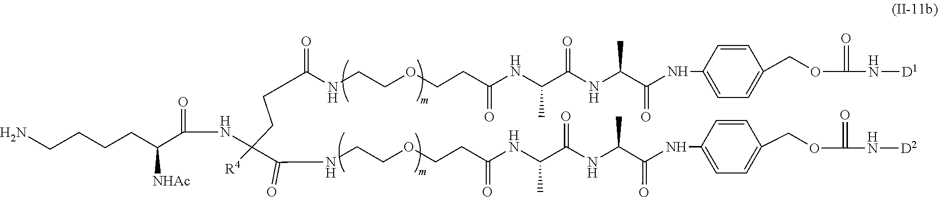

or a pharmaceutically acceptable salt thereof, wherein each variable is independently as described herein.

In some embodiments, the present disclosure provides a linker-payload compound of Formula (II-12):

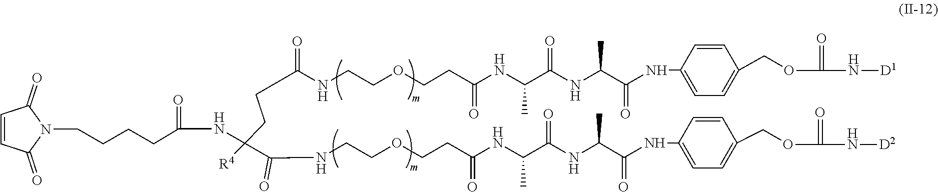

or a pharmaceutically acceptable salt thereof, wherein each variable is independently as described herein.

In some embodiments, the present disclosure provides a linker-payload compound of Formula (II-13):

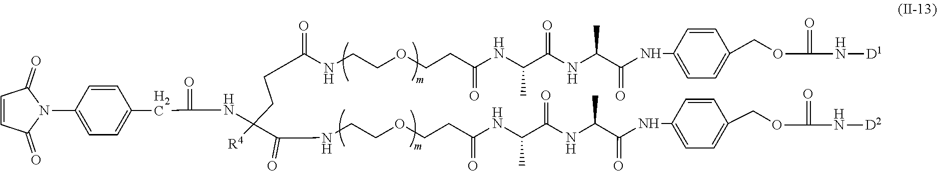

or a pharmaceutically acceptable salt thereof, wherein each variable is independently as described herein.

In some embodiments, the present disclosure provides a linker-payload compound selected from Formulae (II-14)-(II-28):

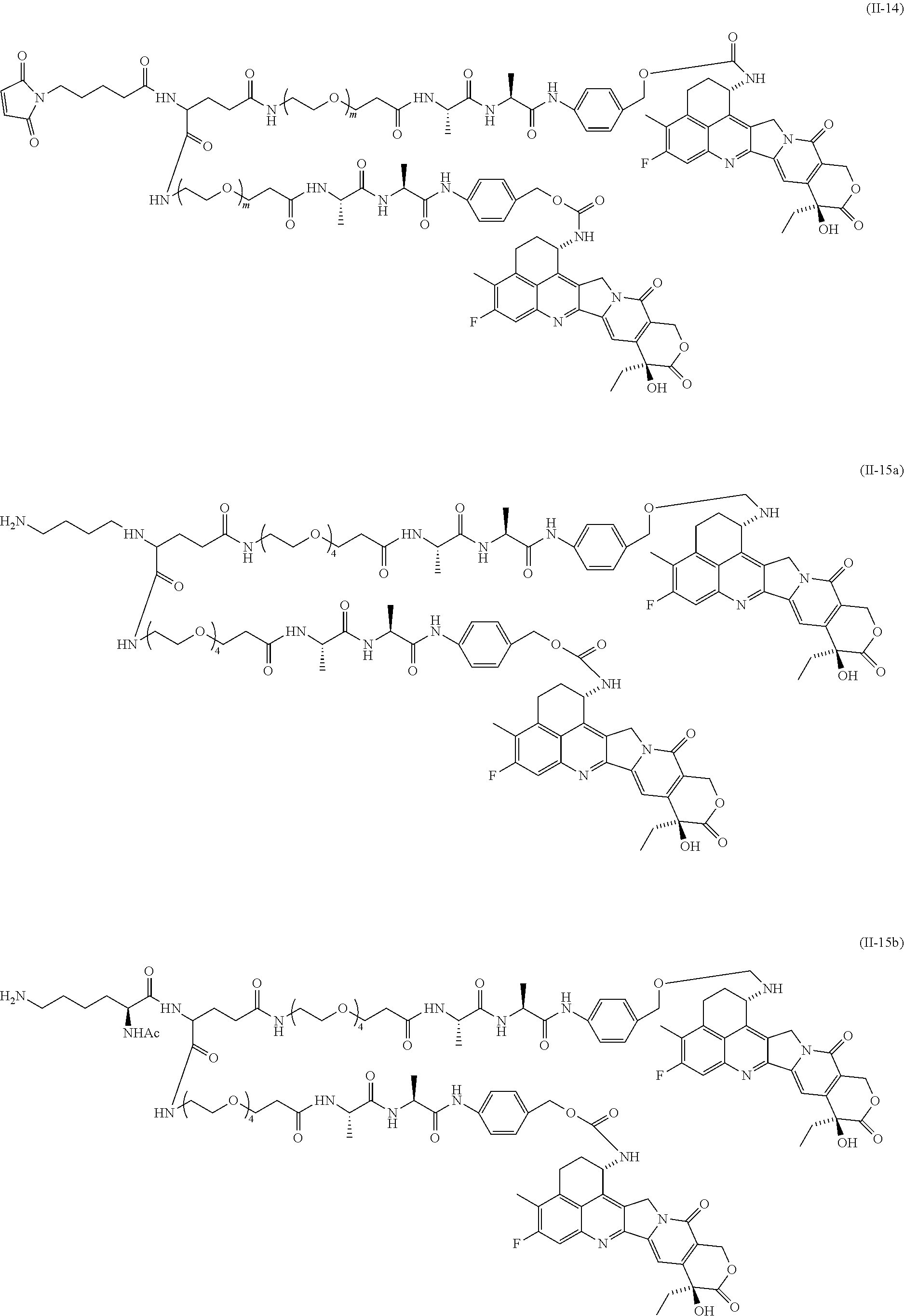

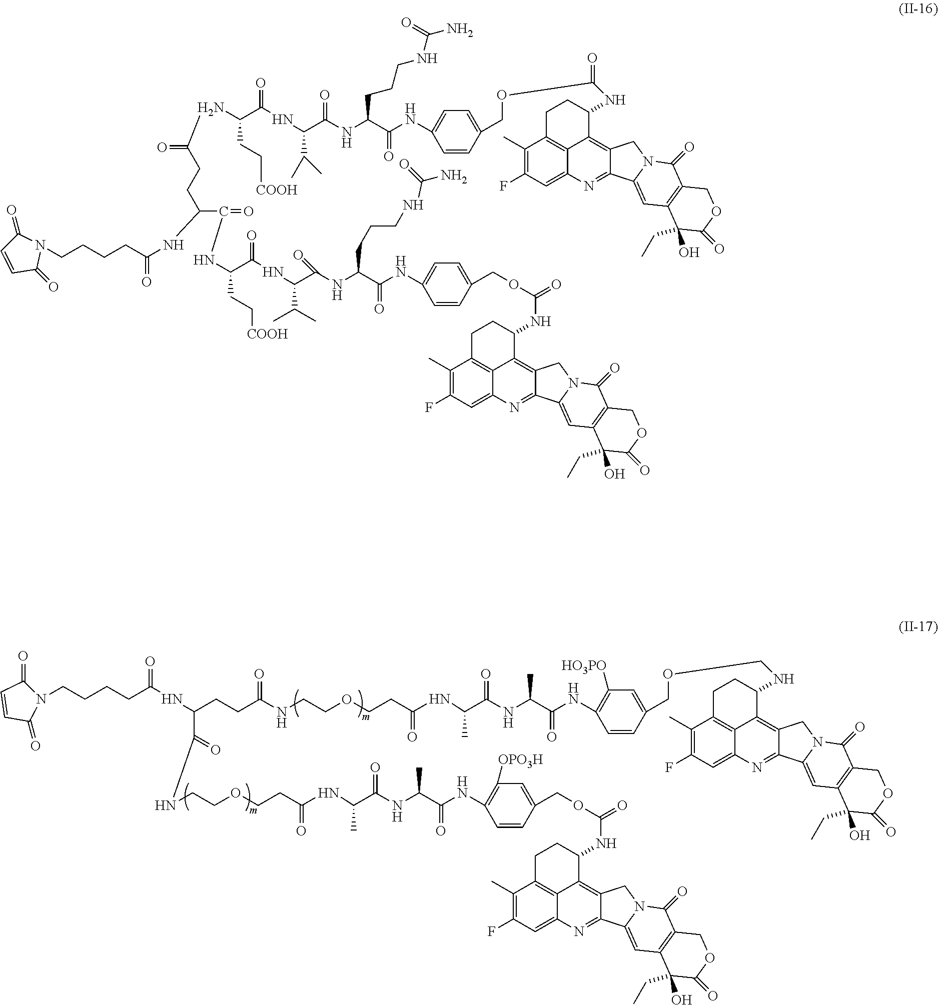

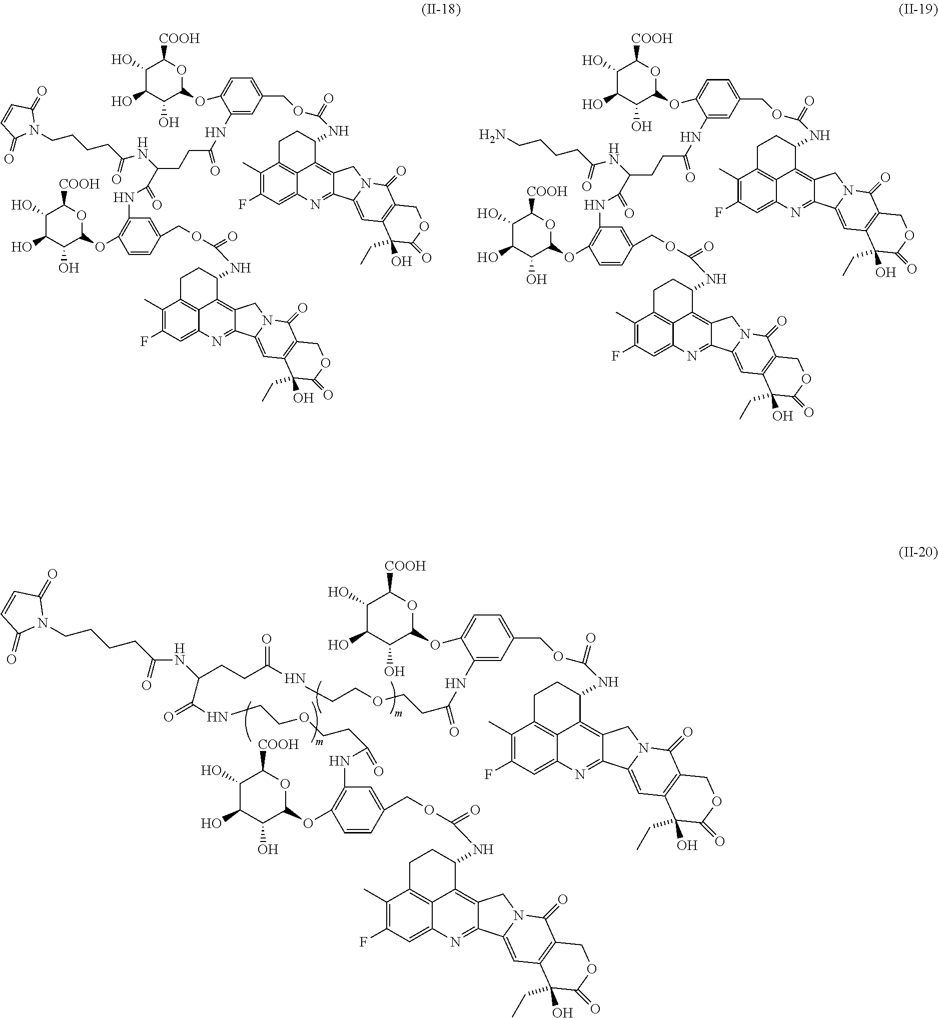

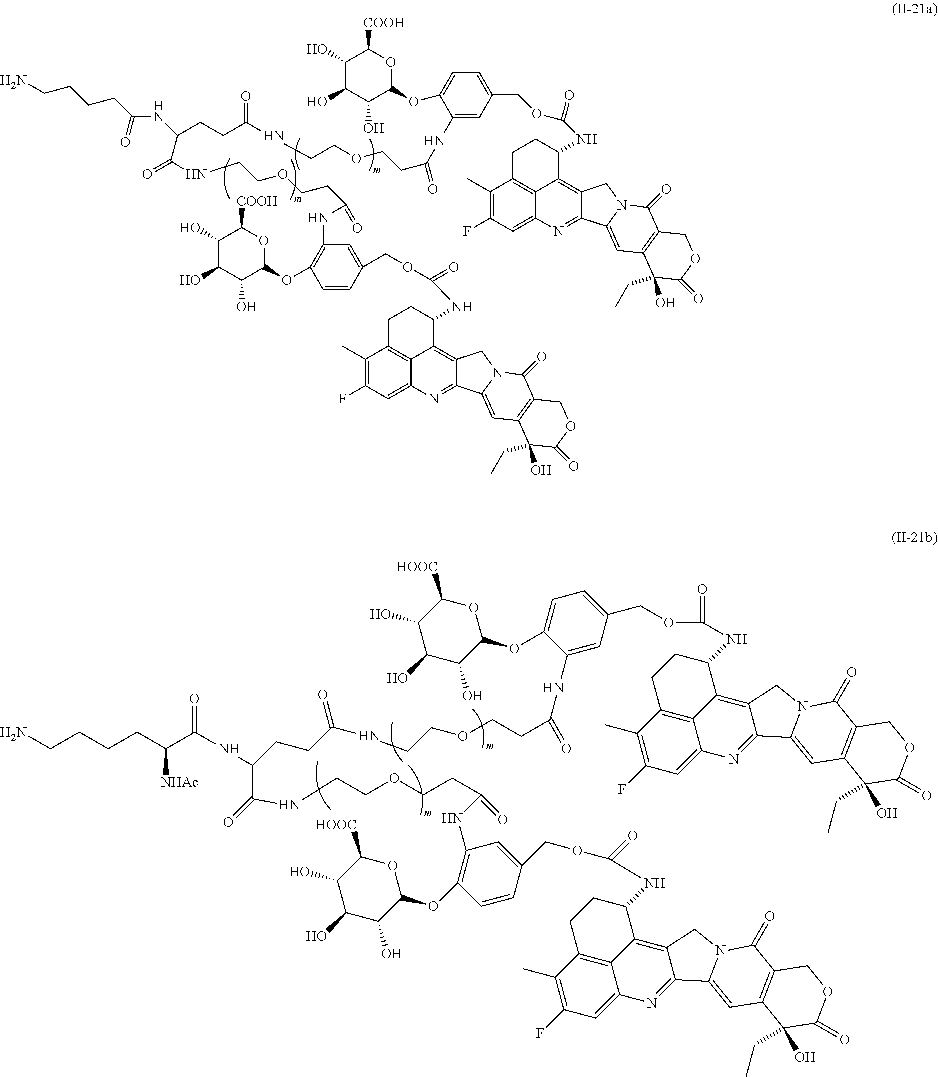

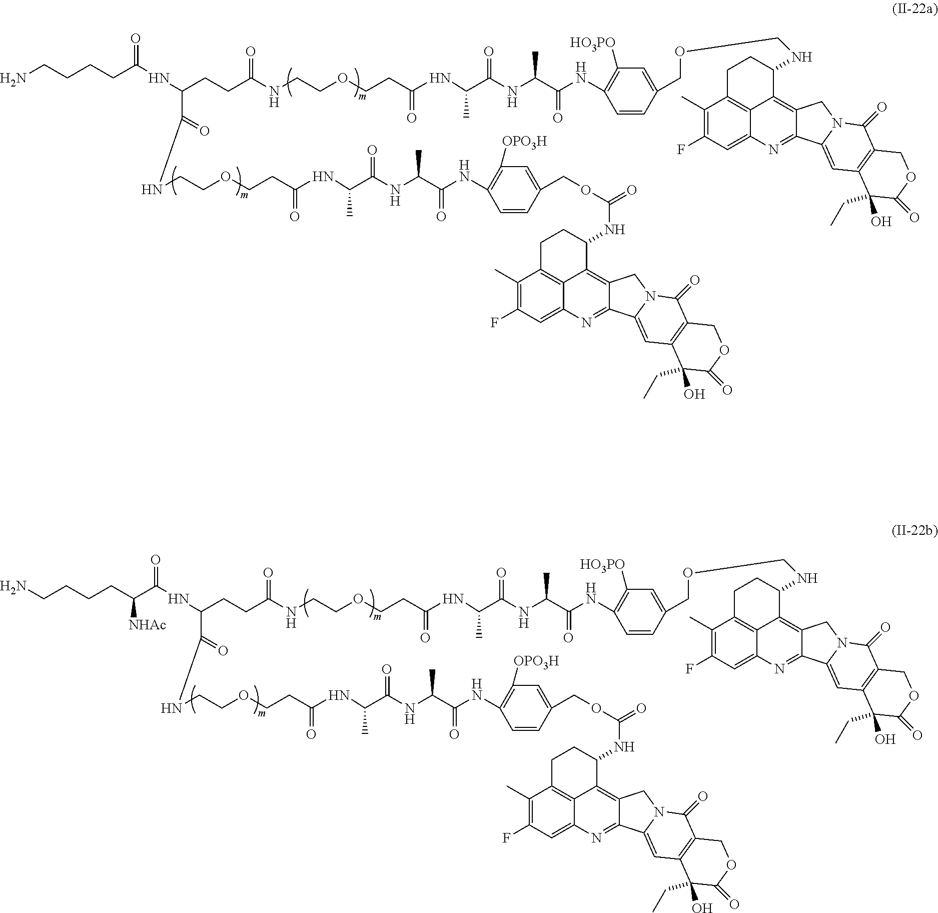

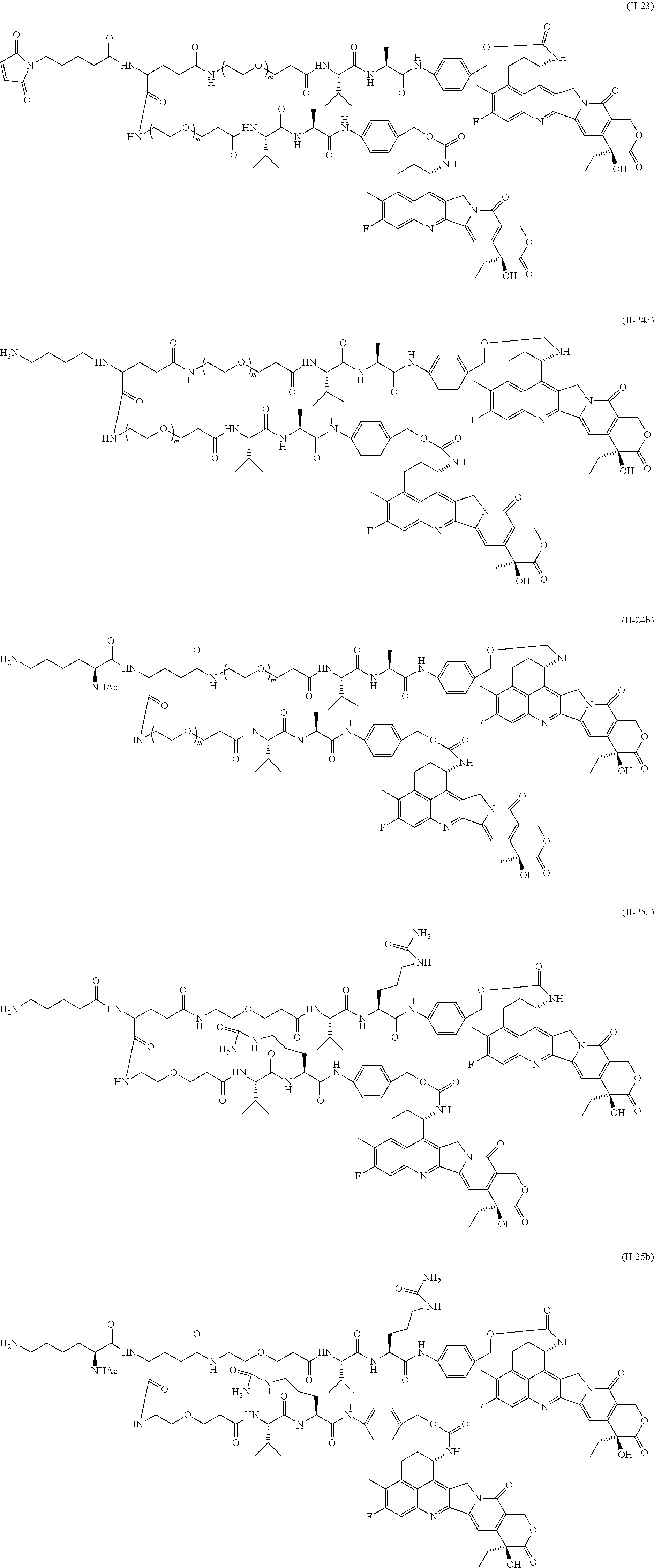

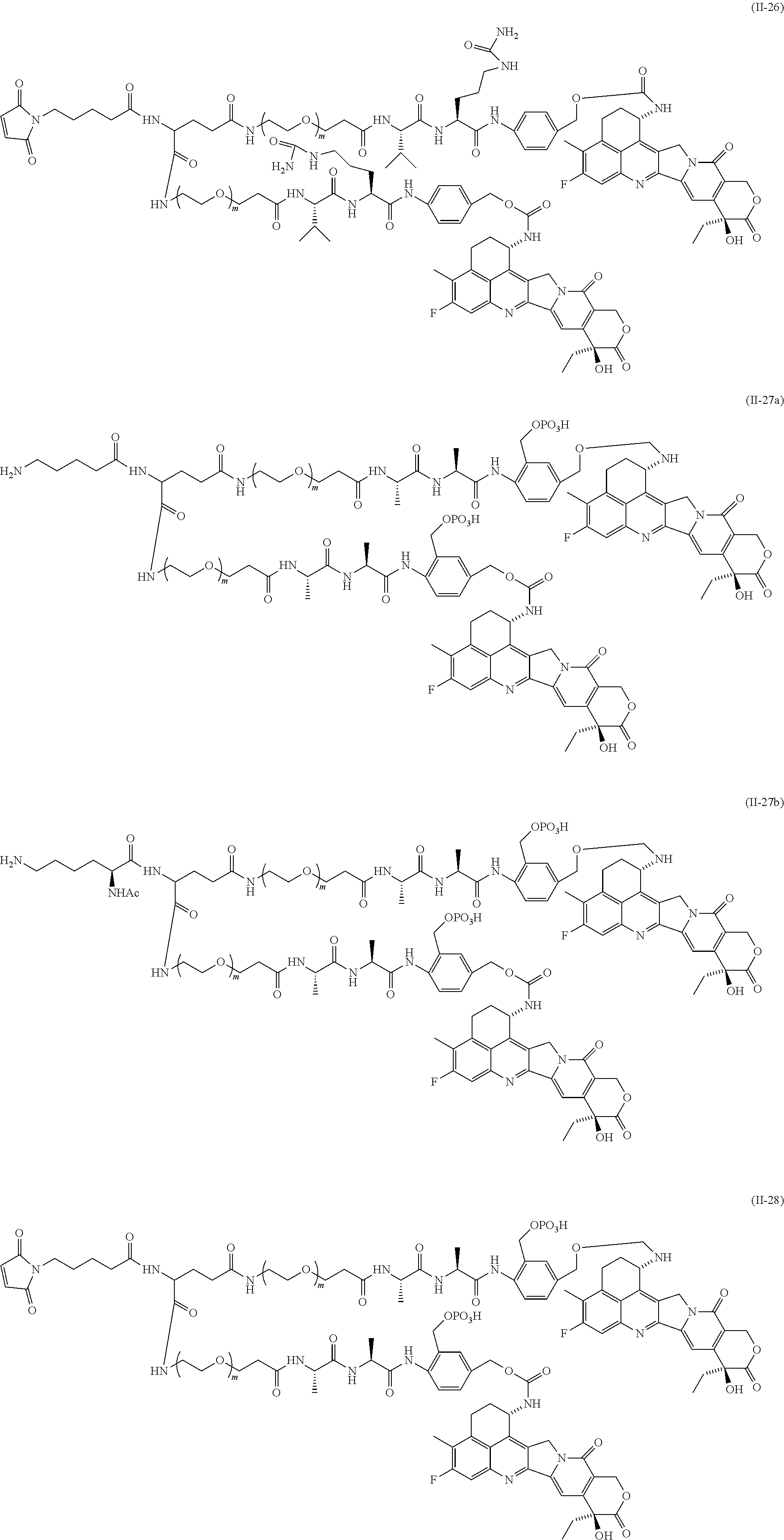

or a pharmaceutically acceptable salt thereof, wherein each variable is independently as described herein.

In some embodiments, the instant disclosure provides a linker-payload compound selected from Formulae (IV-2)-(IV-17):

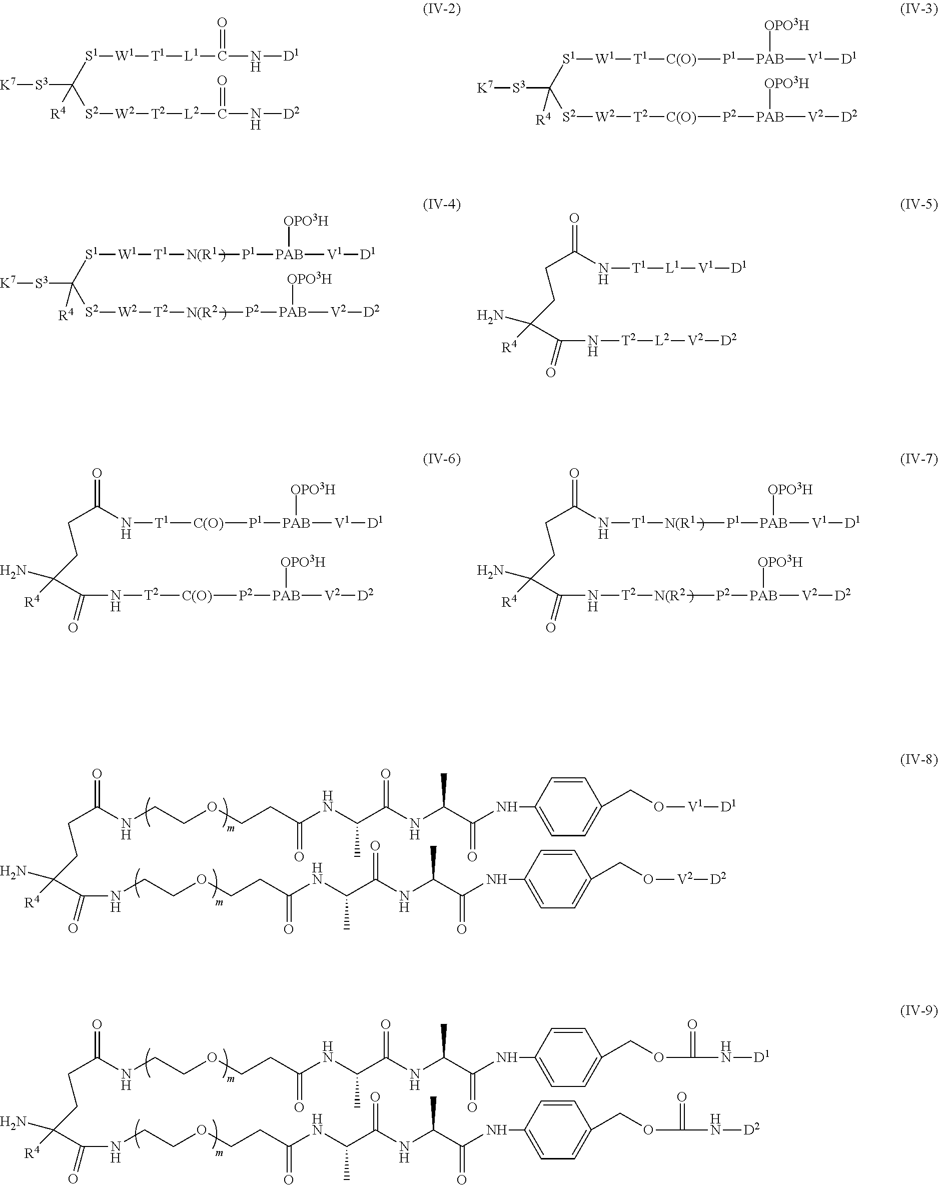

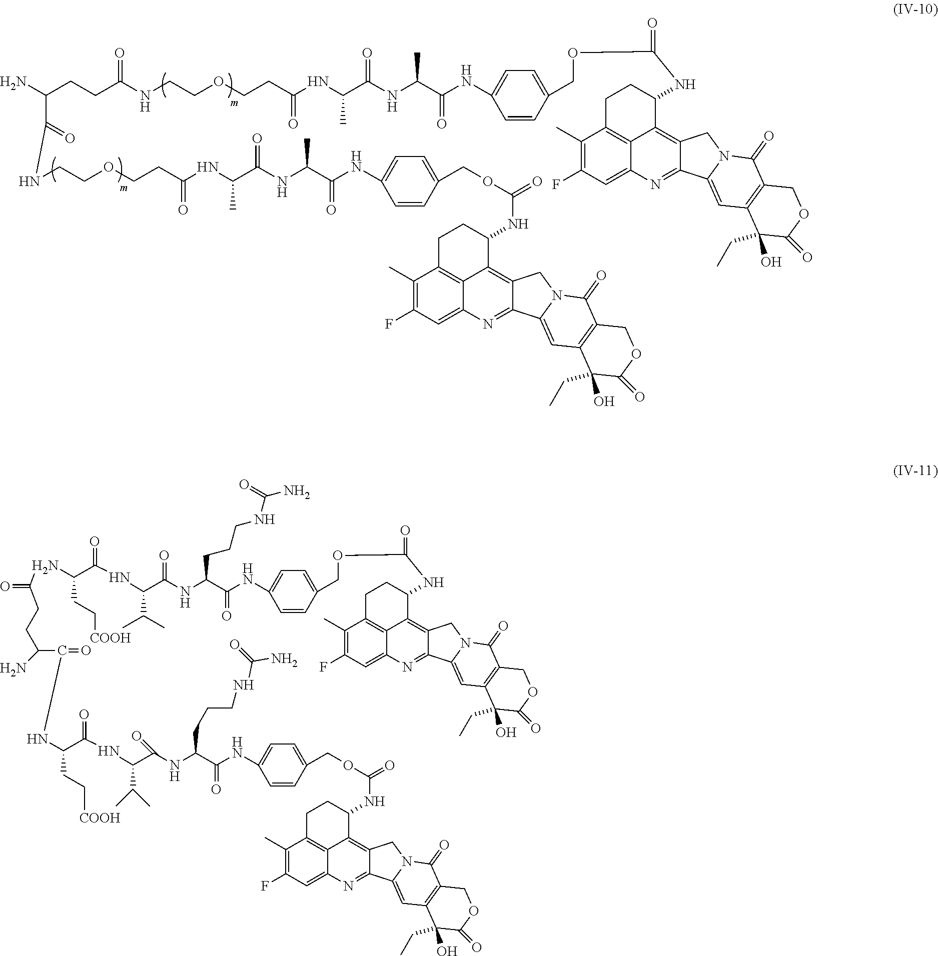

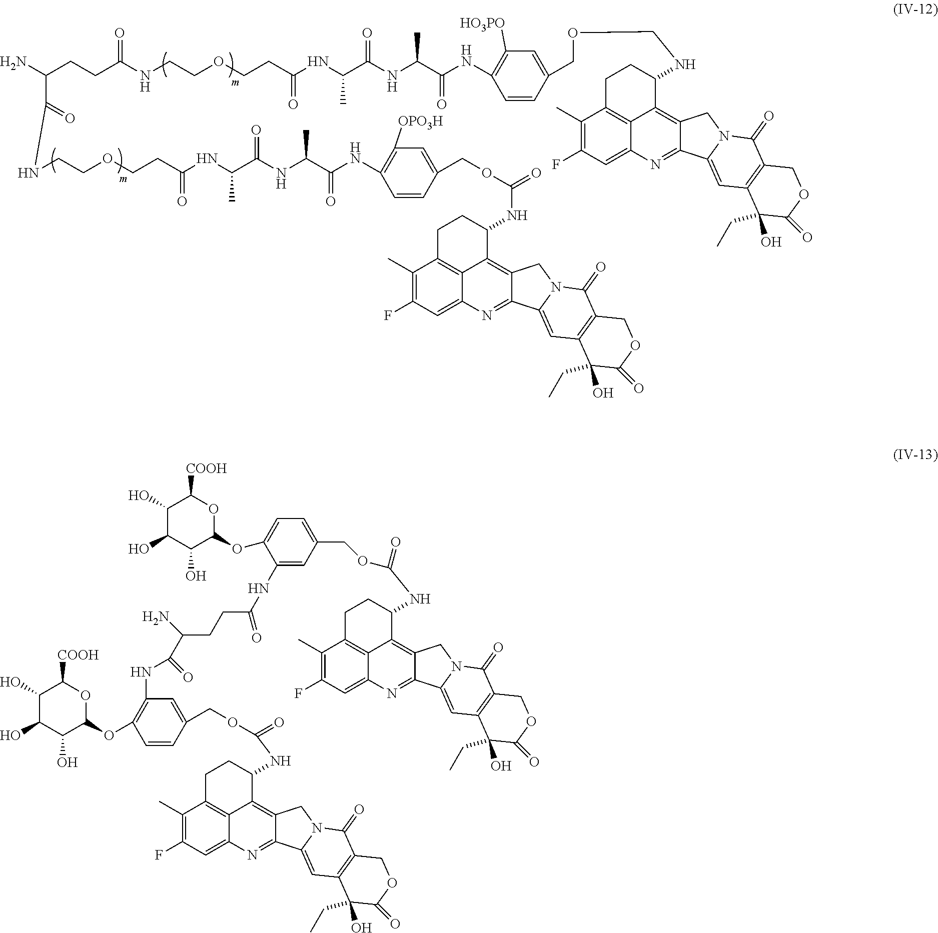

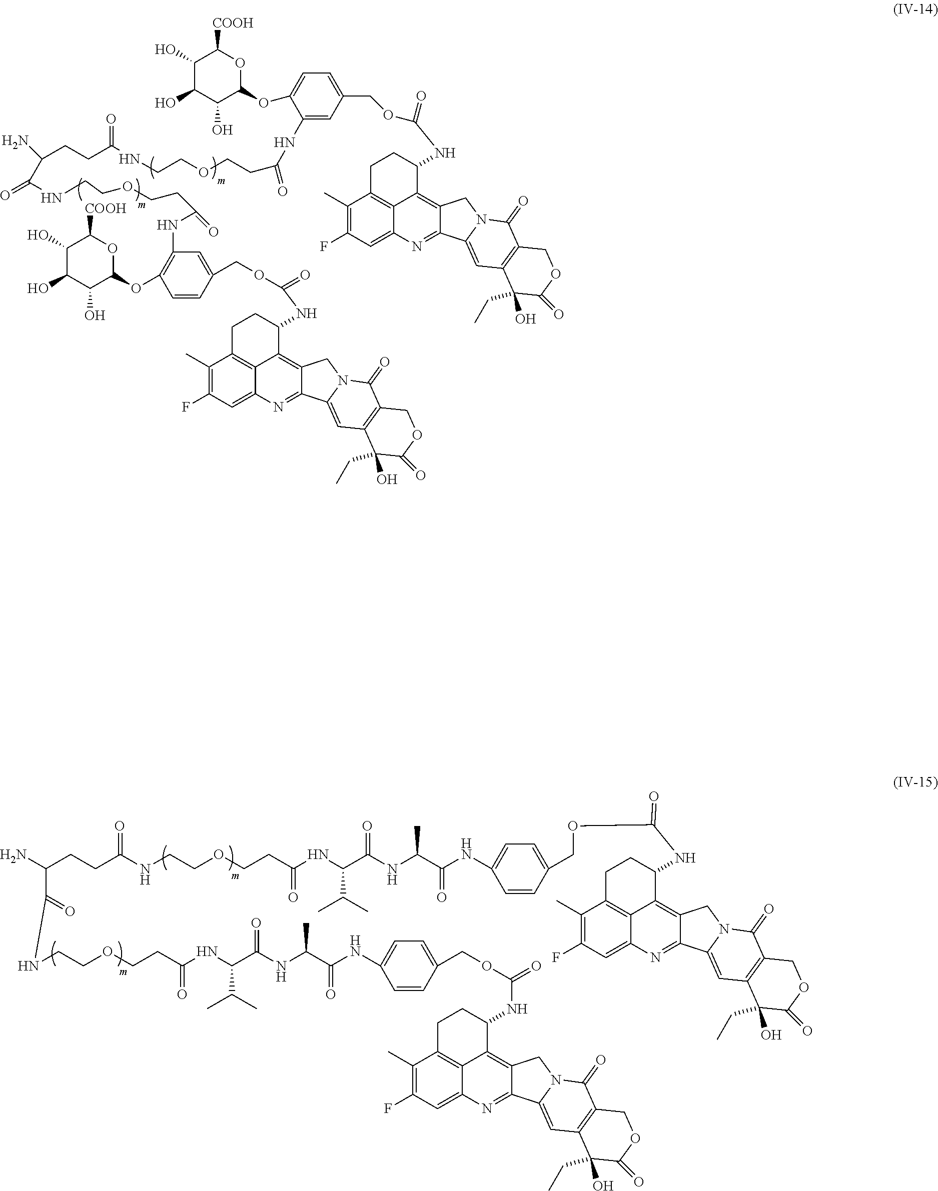

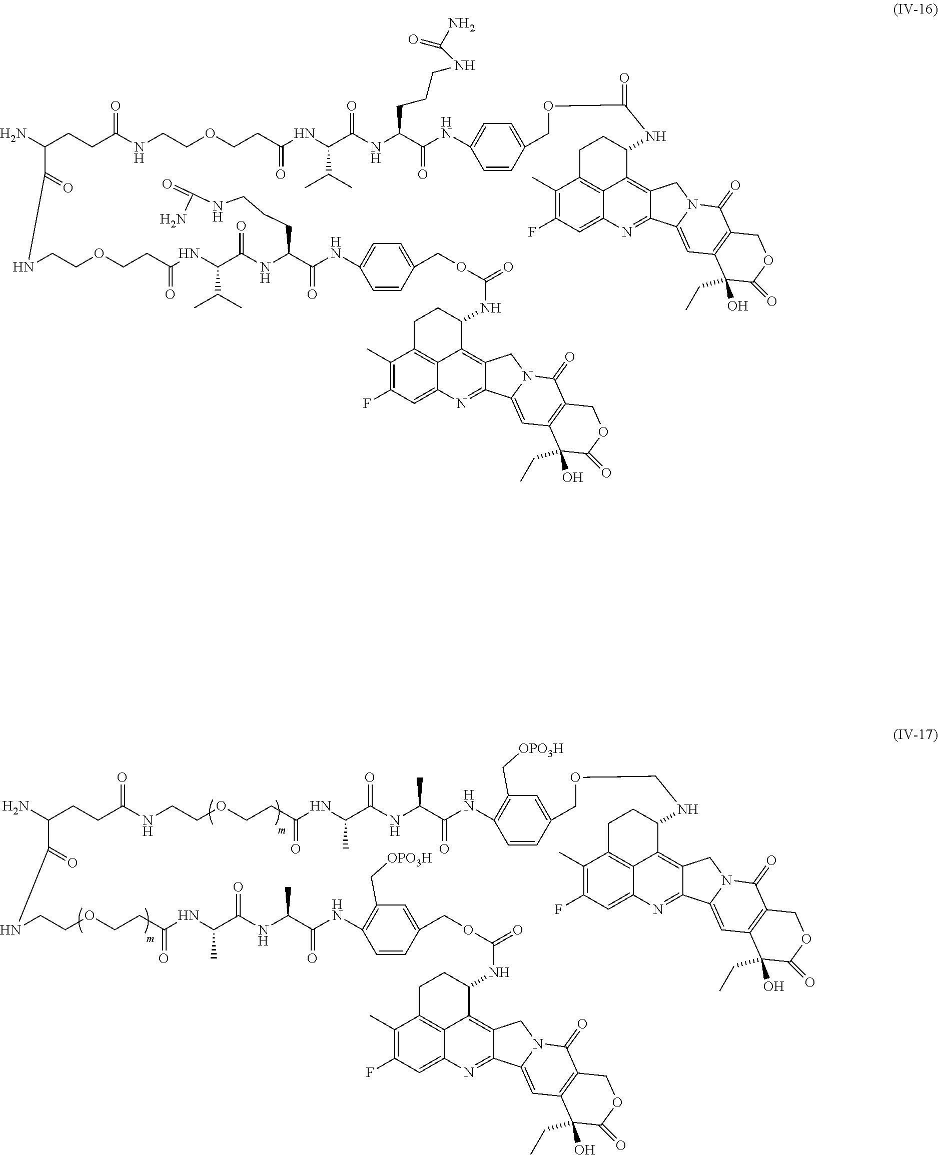

or a pharmaceutically acceptable salt thereof, wherein each variable is independently as described herein.

In some embodiments, the instant disclosure provides a linker selected from Formulae (VI-2)-(VI-8):

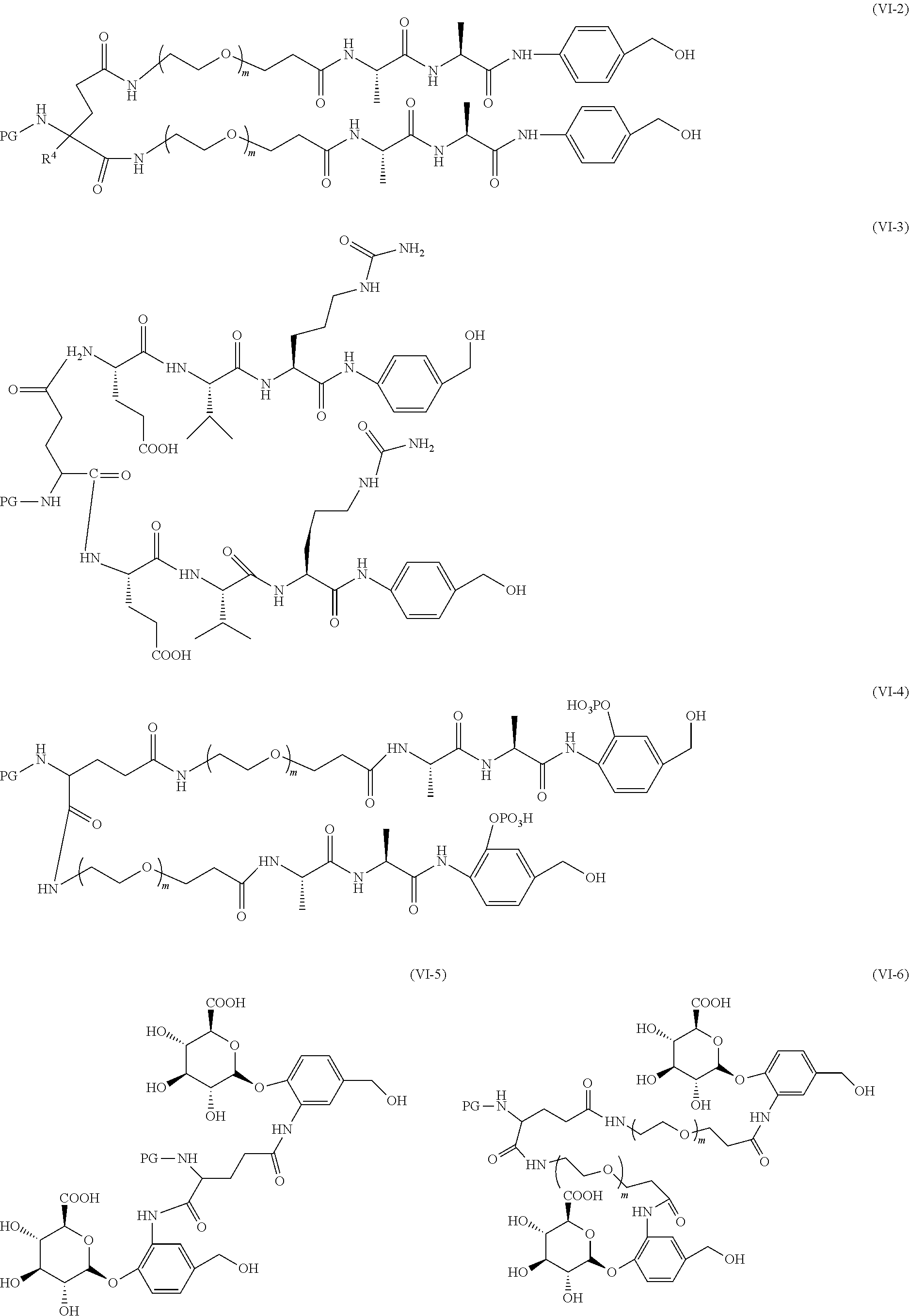

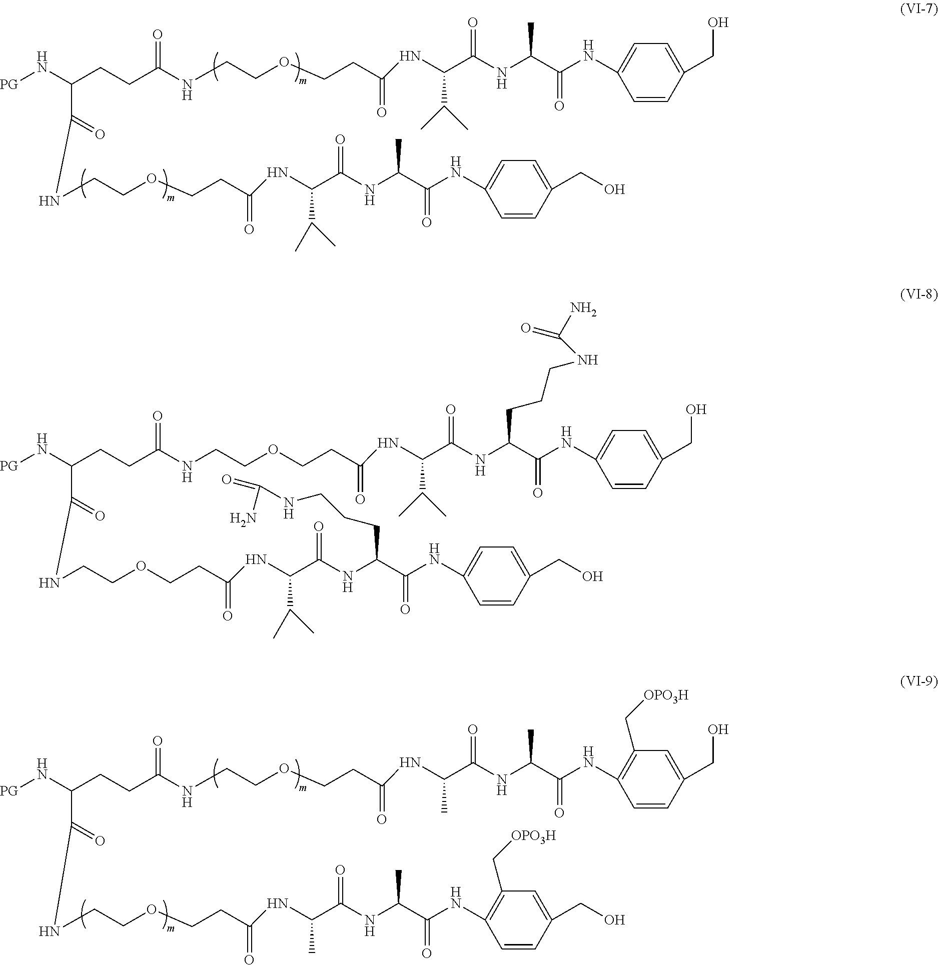

or a pharmaceutically acceptable salt thereof, wherein each variable is independently as described herein.

In one aspect, the instant invention provides a linker-payload compound of formula (II*):

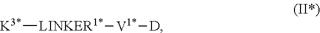

or a pharmaceutically acceptable salt thereof, wherein K³* is a functional group capable of forming a linker moiety by a coupling reaction with a different functional group, and each of LINKER*, V¹*, and D is independently as defined and described herein.

As defined generally above, each K³* is independently a functional group capable of forming a linker moiety by a coupling reaction with a different functional group. In some embodiments, each K³* is independently a functional group selected from those listed in Table 2.

In some embodiments, K³* is a functional group capable of forming a linker moiety by a Michael addition with a different functional group. In some embodiments, K³* is a Michael acceptor. In some embodiments, K³* is a maleimide group

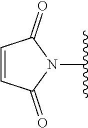 [SMILES: *N1C(=O)C=CC1=O]

attached to LINKER¹*. In some embodiments, K³* is a N-aryl maleimide group

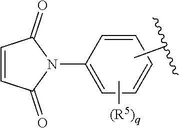

attached to LINKER¹*, wherein each of R⁵* and q is independently as described herein. In some embodiments, K³* is a N-aryl maleimide group

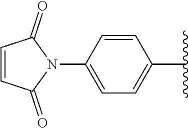 [SMILES: *c1ccc(N2C(=O)C=CC2=O)cc1]

attached to LINKER¹*. In some embodiments, K³* is a maleimide group

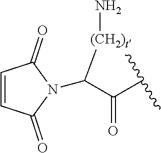

attached to LINKER¹*, wherein each t′ is independently as described herein. In some embodiments, K³* is a maleimide group

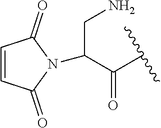 [SMILES: *C(=O)C(CN)N1C(=O)C=CC1=O]

attached to LINKER¹*. In some embodiments, K³* is a Michael donor. In some embodiments, K³* is a thiol group —SH attached to LINKER*.

In some embodiments, K³* is a functional group capable of forming a linker moiety by a leaving group displacement reaction with a different functional group. In some embodiments, K³* is an amine group or an amine donor attached to LINKER¹*. In some embodiments, K³* is —NH₂. In some embodiments, K³* is a leaving group attached to LINKER¹*. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K³* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K³* is an activated ester selected from N-hydroxysuccinimde, pentafluorophenyl, and p-nitrophenyl esters.

In some embodiments, K³* is a functional group capable of forming a linker moiety by an azide-alkyne cycloaddition. In some embodiments, K³* is an azide group —N₃ attached to LINKER¹*. In some embodiments, K³* is an alkyne group

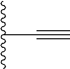 [SMILES: *C#C]

attached to LINKER¹*. In some embodiments, K³* is a cyclooctyne group

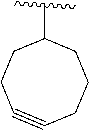 [SMILES: *C1CCC#CCCC1]

attached to LINKER¹*.

In some embodiments, K³* is a functional group capable of forming a linker moiety by a transglutaminase mediated acyl addition. In some embodiments, K³* is an acyl acceptor and the anime donor. In some embodiments, K³* is —NH₂. In some embodiments, K³* is an acyl donor. In some embodiments, K³* is the γ-carboxamide group (—(C═O)NH₂) of a glutamine residue of LINKER¹*.

In one aspect, the instant invention provides a ligan of formula (III*):

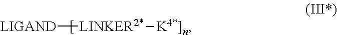

or a pharmaceutically acceptable salt thereof, wherein each K⁴* is independently a functional group capable of forming a linker moiety by a coupling reaction with a different functional group, and each of LIGAND, LINKER²*, and n is independently as defined and described herein.

As defined generally above, each K⁴* is independently a functional group capable of forming a linker moiety by a coupling reaction with a different functional group. In some embodiments, each K⁴* is independently a functional group selected from those listed in Table 2.

In some embodiments, K⁴* is a functional group capable of forming a linker moiety by a Michael addition with a different functional group. In some embodiments, K⁴* is a Michael acceptor. In some embodiments, K⁴* is a maleimide group

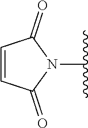 [SMILES: *N1C(=O)C=CC1=O]

attached to LINKER²*. In some embodiments, K⁴* is a N-aryl maleimide group

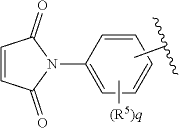

attached to LINKER²*, wherein each of R⁵* and q is independently as described herein. In some embodiments, K⁴* is a N-aryl maleimide group

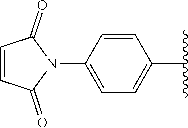 [SMILES: *c1ccc(N2C(=O)C=CC2=O)cc1]

attached to LINKER²*. In some embodiments, K⁴* is a maleimide group

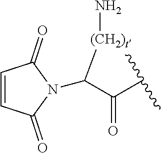

attached to LINKER²*, wherein each t′ is independently as described herein. In some embodiments, K⁴* is a maleimide group

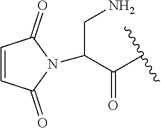 [SMILES: *C(=O)C(CN)N1C(=O)C=CC1=O]

attached to LINKER²*. In some embodiments, K⁴* is a Michael donor. In some embodiments, K⁴* is a thiol group —SH attached to LINKER²*.

In some embodiments, K⁴* is a functional group capable of forming a linker moiety by a leaving group displacement reaction with a different functional group. In some embodiments, K⁴* is an amine group or an amine donor attached to LINKER²*, In some embodiments, K⁴* is —NH₂. In some embodiments, K⁴* is a leaving group attached to LINKER²*. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁴* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁴* is an activated ester selected from N-hydroxysuccinimde, pentafluorophenyl, and p-nitrophenyl esters.

In some embodiments, K⁴* is a functional group capable of forming a linker moiety by an azide-alkyne cycloaddition. In some embodiments, K⁴* is an azide group —N₃ attached to LINKER²*. In some embodiments, K⁴* is an alkyne group

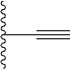 [SMILES: *C#C]

attached to LINKER²*. In some embodiments, K⁴* is a cyclooctyne group

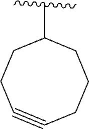 [SMILES: *C1CCC#CCCC1]

attached LINKER²*.

In some embodiments, K⁴* is a functional group capable of forming a linker moiety by a transglutaminase mediated acyl addition. In some embodiments, K⁴* is an acyl acceptor and the anime donor. In some embodiments, K⁴* is an acyl donor. In some embodiments, K⁴* is the γ-carboxamide group (—(C═O)NH₂) of a glutamine residue of LINKER²*.

In one aspect, the present invention provides a method for synthesizing a ligand-payload conjugate of Formula (I*):

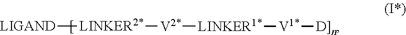

by a coupling reaction between a linker-payload compound of formula (II*): K³*-LINKER¹-V¹*-D and a ligand of formula (III*):

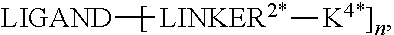

wherein each variable is independently as defined and described herein, and the functional groups K³* and K⁴* form a linker moiety V²* by a coupling reaction.

In some embodiments, K³* and K⁴* form a linker moiety V²* by a Michael addition, wherein K³* is a maleimide group

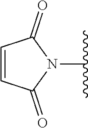 [SMILES: *N1C(=O)C=CC1=O]

and K⁴* is a thiol group —SH, or wherein K³* is a thiol group —SH, and K⁴* is a maleimide group

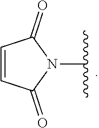 [SMILES: *N1C(=O)C=CC1=O]

In some embodiments, K³* and K⁴* form a linker moiety V²* by a Michael addition, wherein K³* is a maleimide group

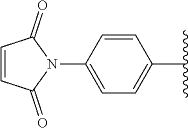 [SMILES: *c1ccc(N2C(=O)C=CC2=O)cc1]

and K⁴* is a thiol group —SH, or wherein K³* is a thiol group —SH, and K⁴* is a maleimide group

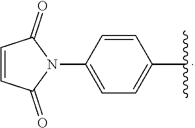 [SMILES: *c1ccc(N2C(=O)C=CC2=O)cc1]

In some embodiments, K³* and K⁴* form a linker moiety V²* by a Michael addition, wherein K³* is a maleimide group

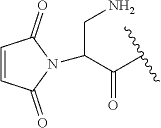 [SMILES: *C(=O)C(CN)N1C(=O)C=CC1=O]

and K⁴* is a thiol group —SH, or wherein K³* is a thiol group —SH, and K⁴* is a maleimide group

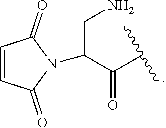 [SMILES: *C(=O)C(CN)N1C(=O)C=CC1=O]

In some embodiments, K³* and K⁴* form a linker moiety V²* by a leaving group displacement reaction, wherein K³* is —NH₂ and K⁴* is a leaving group, or wherein K³* is a leaving group, and K⁴* is —NH₂. In some embodiments, K³* and K⁴* form a linker moiety V²* by a leaving group displacement reaction, wherein K³* is —NH₂ and K⁴* is an activated ester —C(O)-LG, or wherein K³* is an activated ester —C(O)-LG, and K⁴* is —NH₂.

In some embodiments, K³* and K⁴* form a linker moiety V²* by an azide-alkyne cycloaddition, wherein K³* is —N₃ and K⁴* is

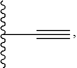 [SMILES: *C#C]

or wherein K³* is

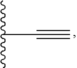 [SMILES: *C#C]

and K⁴* is —N₃. In some embodiments, K³* and K⁴* form a linker moiety V²* by an azide-alkyne cycloaddition, wherein K³* is —N₃ and K⁴* is

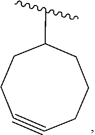 [SMILES: *C1CCC#CCCC1]

or wherein K³* is

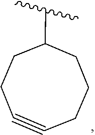 [SMILES: *C1CCC#CCCC1]

and K⁴* is —N₃.

In some embodiments, K³* and K⁴* form a linker moiety V²* by a transglutaminase mediated acyl addition, wherein K³* is —NH₂ and K⁴* is γ-carboxamide group (—(C═O)NH₂) of a glutamine residue of LINKER²*, or K³* is γ-carboxamide group (—(C═O)NH₂) of a glutamine residue of LINKER¹* and K⁴* is —NH₂.

In one aspect, the instant invention provides a linker-payload compound of formula (IV-1*):

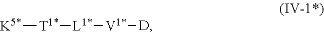

or a pharmaceutically acceptable salt thereof, wherein each K⁵* is independently a functional group capable of forming a carboxamide linker moiety by a coupling reaction with a different functional group, each K⁵* optionally comprising a protecting group, and each of T¹*, L¹*, V¹*, and D is independently as defined and described herein.

As defined generally above, each K⁵* is independently a functional group capable of forming a carboxamide linker moiety by a coupling reaction with a different functional group. In some embodiments, each K⁵* is independently a functional group selected from those listed in Table 2.

In some embodiments, K⁵* is a functional group capable of forming a carboxamide linker moiety by a leaving group displacement reaction with a different functional group. In some embodiments, K⁵* is an amine group or an amine. In some embodiments, K⁵* is —NH₂. In some embodiments, K⁵* comprises a leaving group. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁵* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁵* is an activated ester selected from N-hydroxysuccinimde, pentafluorophenyl, and p-nitrophenyl esters.

In some embodiments, K⁵* is a functional group capable of forming a carboxamide linker moiety by an acyl addition. In some embodiments, K⁵* is an acyl acceptor and the anime donor. In some embodiments, K⁵* is —NH₂. In some embodiments, K⁵* is an acyl donor. In some embodiments, K⁵* is —COOH.

In some embodiments, K⁵* is —NH-PG, wherein PG is a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the nitrogen to which it is attached, include, but are not limited to, aralkylamines, carbamates, allyl amines, amides, and the like. In some embodiments, a suitable amino protecting group PG is selected from t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

In one aspect, the instant invention provides a linker of formula (V*):

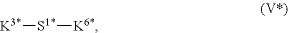

or a pharmaceutically acceptable salt thereof, wherein each K⁶* is independently a functional group capable of forming a carboxamide linker moiety by a coupling reaction with a different functional group, and each of K³* and S¹* is independently as defined and described herein.

As defined generally above, each K⁶* is independently a functional group capable of forming a carboxamide linker moiety by a coupling reaction with a different functional group. In some embodiments, each K⁶* is independently a functional group selected from those listed in Table 2.

In some embodiments, K⁶* is a functional group capable of forming a carboxamide linker moiety by a leaving group displacement reaction with a different functional group. In some embodiments, K⁶* is an amine group or an amine. In some embodiments, K⁶* is —NH₂. In some embodiments, K⁶* comprises a leaving group. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K⁶* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K⁶* is an activated ester selected from N-hydroxysuccinimde, pentafluorophenyl, and p-nitrophenyl esters.

In some embodiments, K⁶* is a functional group capable of forming a carboxamide linker moiety by an acyl addition. In some embodiments, K⁶* is an acyl acceptor and the anime donor. In some embodiments, K⁶* is —NH₂. In some embodiments, K⁶* is an acyl donor. In some embodiments, K⁶* is —COOH.

In one aspect, the present invention provides a method for synthesizing a linker-payload compound of Formula (II-1*):

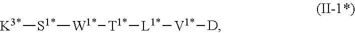

by a coupling reaction between a linker-payload compound of formula (IV-1*): K⁵*-T¹*-L¹*-V¹*-D and a linker of formula (V*): K³*-S¹*-K⁶*, wherein each variable is independently as defined and described herein, and the functional groups K⁵* and K⁶* form a linker moiety W¹* by a coupling reaction.

In some embodiments, the functional groups K⁵* and K⁶* form a carboxamide linker W¹* by a coupling reaction.

In some embodiments, K⁵* is an amine group or an amine, and K⁶* comprises a leaving group as described herein. In some embodiments, K⁵* comprises a leaving group as described herein, and K⁶* is an amine group or an amine. In some embodiments, K⁵* is —NH₂, and K⁶* is an activated ester —C(O)-LG as described herein. In some embodiments, K⁵* is an activated ester —C(O)-LG as described herein, and K⁶* is —NH₂. In some embodiments, K⁵* is —NH₂, and K⁶* is —COOH. In some embodiments, K⁵* is —COOH, and K⁶* is —NH₂.

In one aspect, the instant invention provides a linker of formula (VI-1*):

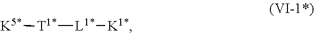

or a pharmaceutically acceptable salt thereof, wherein each K¹* is independently a functional group capable of forming a linker moiety by a coupling reaction with a different functional group, and each of K⁵*, T¹*, and L¹* is independently as defined and described herein.

As defined generally above, each K¹* is independently a functional group capable of forming a linker moiety by a coupling reaction with a different functional group. In some embodiments, each K¹* is independently a functional group selected from those listed in Table 2.

In some embodiments, K¹* is a functional group capable of forming a linker moiety by a Michael addition with a different functional group. In some embodiments, K¹* is a Michael acceptor. In some embodiments, K¹* is a maleimide group

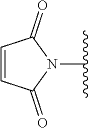 [SMILES: *N1C(=O)C=CC1=O]

attached to LINKER¹*. In some embodiments, K¹* is a N-aryl maleimide group

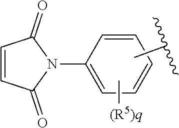

attached to LINKER*, wherein each of R⁵* and q is independently as described herein. In some embodiments, K¹* is a N-aryl maleimide group

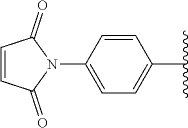 [SMILES: *c1ccc(N2C(=O)C=CC2=O)cc1]

attached to LINKER¹*. In some embodiments, K¹* is a maleimide group

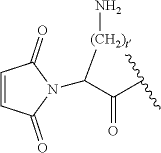

attached to LINKER¹*, wherein each t′ is independently as described herein. In some embodiments, K¹* is a maleimide group

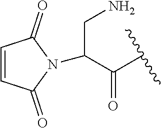 [SMILES: *C(=O)C(CN)N1C(=O)C=CC1=O]

attached to LINKER¹*. In some embodiments, K¹* is a Michael donor. In some embodiments, K¹* is a thiol group —SH attached to LINKER¹*.

In some embodiments, K¹* is a functional group capable of forming a linker moiety by a leaving group displacement reaction with a different functional group. In some embodiments, K¹* is an amine group or an amine donor attached to LINKER*, In some embodiments, K¹* is —NH₂. In some embodiments, K¹* is a leaving group attached to LINKER*. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K¹* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K¹* is an activated ester selected from N-hydroxysuccinimde, pentafluorophenyl, and p-nitrophenyl esters.

In some embodiments, K¹* is a functional group capable of forming a linker moiety by an azide-alkyne cycloaddition. In some embodiments, K¹* is an azide group —N₃ attached to LINKER¹*. In some embodiments, K¹* is an alkyne group

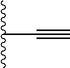 [SMILES: *C#C]

attached to LINKER¹*. In some embodiments, K¹* is a cyclooctyne group

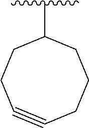 [SMILES: *C1CCC#CCCC1]

attached to LINKER¹*.

In some embodiments, K¹* is a functional group capable of forming a linker moiety by an acyl addition. In some embodiments, K¹* is an acyl acceptor and the anime donor. In some embodiments, K¹* is —NH₂. In some embodiments, K¹* is an acyl donor. In some embodiments, K¹* is —COOH.

In another aspect, the instant invention provides a payload of formula (VII):

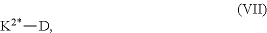

or a pharmaceutically acceptable salt thereof, wherein each K²* is a functional group capable of forming a linker moiety by a coupling reaction with a different functional group, and each D is independently as defined and described herein.

As defined generally above, each K²* is independently a functional group capable of forming a linker moiety by a coupling reaction with a different functional group. In some embodiments, each K²* is independently a functional group selected from those listed in Table 2.

In some embodiments, K²* is a functional group capable of forming a linker moiety by a Michael addition with a different functional group. In some embodiments, K²* is a Michael acceptor. In some embodiments, K²* is a maleimide group

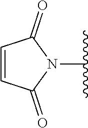 [SMILES: *N1C(=O)C=CC1=O]

attached to D. In some embodiments, K²* is a N-aryl maleimide group

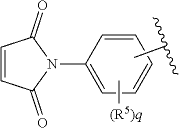

attached to D, wherein each of R⁵* and q is independently as described herein. In some embodiments, K²* is a N-aryl maleimide group

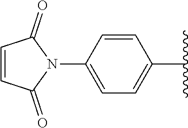 [SMILES: *c1ccc(N2C(=O)C=CC2=O)cc1]

attached to D. In some embodiments, K²* is a maleimide group

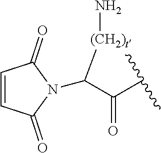

attached to D, wherein each t′ is independently as described herein. In some embodiments, K²* is a maleimide group

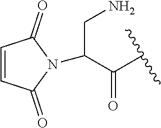 [SMILES: *C(=O)C(CN)N1C(=O)C=CC1=O]

attached to D. In some embodiments, K²* is a Michael donor. In some embodiments, K²* is a thiol group —SH attached to D.

In some embodiments, K²* is a functional group capable of forming a linker moiety by a leaving group displacement reaction with a different functional group. In some embodiments, K²* is an amine group or an amine donor attached to D. In some embodiments, K²* is —NH₂. In some embodiments, K²* is a leaving group attached to D. A leaving group (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like. In some embodiments, K²* is an activated ester —C(O)-LG. An activated ester includes, but is not limited to, acyl halides (e.g. acyl fluoride, acyl chloride, acyl bromide, acyl iodide), N-succinimidyl esters, uronium esters (e.g. 1-hydroxy-7azabenzotriazole, —OAt), and the like. In some embodiments, K²* is an activated ester selected from N-hydroxysuccinimde, pentafluorophenyl, and p-nitrophenyl esters.

In some embodiments, K²* is a functional group capable of forming a linker moiety by an azide-alkyne cycloaddition. In some embodiments, K²* is an azide group —N₃ attached to D. In some embodiments, K²* is an alkyne group

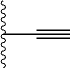 [SMILES: *C#C]

attached to D. In some embodiments, K²* is a cyclooctyne group

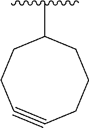 [SMILES: *C1CCC#CCCC1]

attached to D.

In some embodiments, K²* is a functional group capable of forming a linker moiety by an acyl addition. In some embodiments, K²* is an acyl acceptor and the anime donor. In some embodiments, K²* is —NH₂. In some embodiments, K²* is an acyl donor. In some embodiments, K²* is —COOH.

In one aspect, the present invention provides a method for synthesizing a linker-payload of Formula (IV-1):

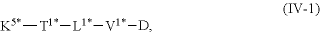

by a coupling reaction between a linker of formula (VI-1): K⁵*-T¹*-L¹*-K¹* and a payload of formula (VII): K²*-D, wherein the functional groups K¹* and K²* form a linker moiety V¹* by a coupling reaction, and each variable is as defined and described herein.

In some embodiments, K¹* and K²* form a linker moiety V¹* by a Michael addition, wherein K¹* is a maleimide group

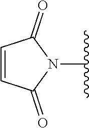 [SMILES: *N1C(=O)C=CC1=O]

and K²* is a thiol group —SH, or wherein K¹* is a thiol group —SH, and K²* is a maleimide group

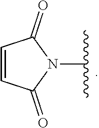 [SMILES: *N1C(=O)C=CC1=O]

In some embodiments, K¹* and K²* form a linker moiety V¹* by a Michael addition, wherein K¹* is a maleimide group

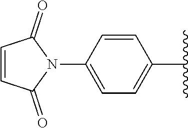 [SMILES: *c1ccc(N2C(=O)C=CC2=O)cc1]

and K²* is a thiol group —SH, or wherein K¹* is a thiol group —SH, and K²* is a maleimide group

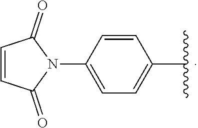 [SMILES: *c1ccc(N2C(=O)C=CC2=O)cc1]

some embodiments, K¹* and K²* form a linker moiety V¹* by a Michael addition, wherein K¹* is a maleimide group

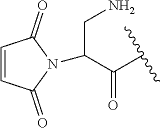 [SMILES: *C(=O)C(CN)N1C(=O)C=CC1=O]

and K²* is a thiol group —SH, or wherein K¹* is a thiol group —SH, and K²* is a maleimide group

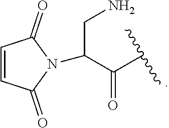 [SMILES: *C(=O)C(CN)N1C(=O)C=CC1=O]

In some embodiments, K¹* and K²* form a linker moiety V¹* by a leaving group displacement reaction, wherein K¹* is —NH₂ and K²* is a leaving group, or wherein K¹* is a leaving group, and K²* is —NH₂. In some embodiments, K¹* and K²* form a linker moiety V¹* by a leaving group displacement reaction, wherein K¹* is —NH₂ and K²* is an activated ester —C(O)-LG, or wherein K¹* is an activated ester —C(O)-LG, and K²* is —NH₂.

In some embodiments, K¹* and K²* form a linker moiety V¹* by an azide-alkyne cycloaddition, wherein K¹* is —N₃ and K²* is

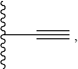 [SMILES: *C#C]

or wherein K¹* is

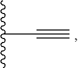 [SMILES: *C#C]

and K²* is —N₃. In some embodiments, K¹* and K²* form a linker moiety V¹* by an azide-alkyne cycloaddition, wherein K¹* is —N₃ and K²* is

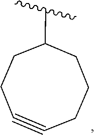 [SMILES: *C1CCC#CCCC1]

or wherein K¹* is

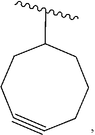 [SMILES: *C1CCC#CCCC1]

and K²* is —N₃.

In some embodiments, K¹* and K²* form a linker moiety V¹* by an acyl addition, wherein K¹* is —NH₂ and K²* is —COOH, or K¹* is —COOH and K²* is —NH₂.

In some embodiments, the instant invention provides a linker-payload compound of formula (II-1*):

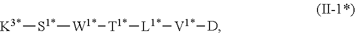

or a pharmaceutically acceptable salt thereof, wherein each variable is independently as described herein.

In some embodiments, the instant invention provides a linker-payload compound of formula (II-2*):

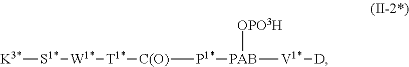

or a pharmaceutically acceptable salt thereof, wherein each variable is independently as described herein.

In some embodiments, the instant invention provides a linker-payload compound of formula (II-3*):

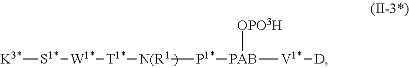

or a pharmaceutically acceptable salt thereof, wherein each variable is independently as described herein.

In some embodiments, the present invention provides a linker-payload compound of Formula (II-4*):

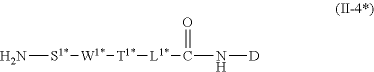

or a pharmaceutically acceptable salt thereof, wherein each variable is independently as described herein.

In some embodiments, the present invention provides a linker-payload compound of Formula (II-5*):

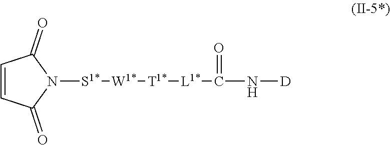

or a pharmaceutically acceptable salt thereof, wherein each variable is independently as described herein.

In some embodiments, the present invention provides a linker-payload compound of Formula (II-6*):

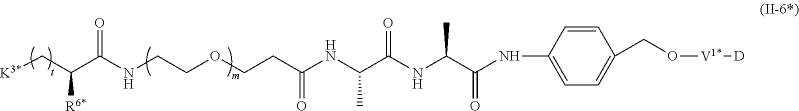

or a pharmaceutically acceptable salt thereof, wherein each variable is independently as described herein.

In some embodiments, the present invention provides a linker-payload compound of Formula (II-7*):

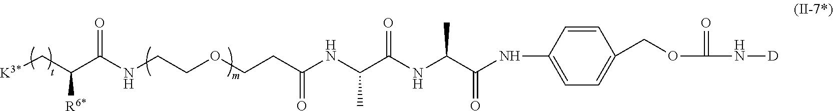

or a pharmaceutically acceptable salt thereof, wherein each variable is independently as described herein.

In some embodiments, the present invention provides a linker-payload compound of Formula (II-8*):

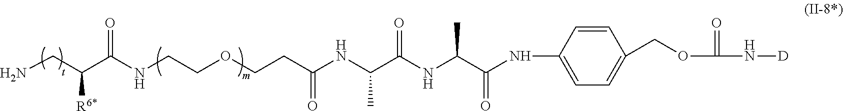

or a pharmaceutically acceptable salt thereof, wherein each variable is independently as described herein.

In some embodiments, the present invention provides a linker-payload compound of Formula II-9*):

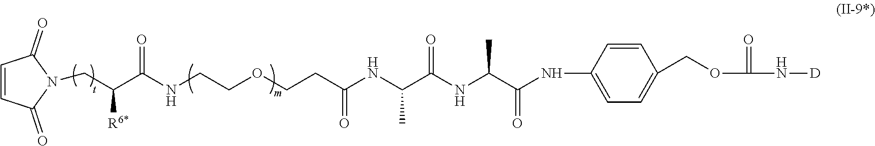

or a pharmaceutically acceptable salt thereof, wherein each variable is independently as described herein.

In some embodiments, the present invention provides a linker-payload compound of Formula (II-10*):

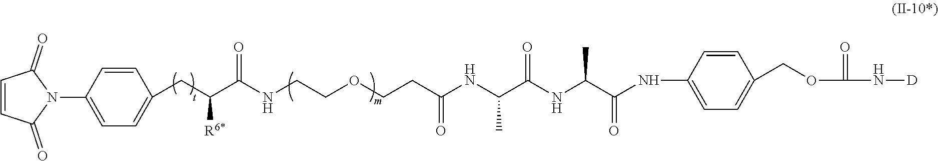

or a pharmaceutically acceptable salt thereof, wherein each variable is independently as described herein.

In some embodiments, the present invention provides a linker-payload compound selected from Formulae (II-11*)-(II-19*):

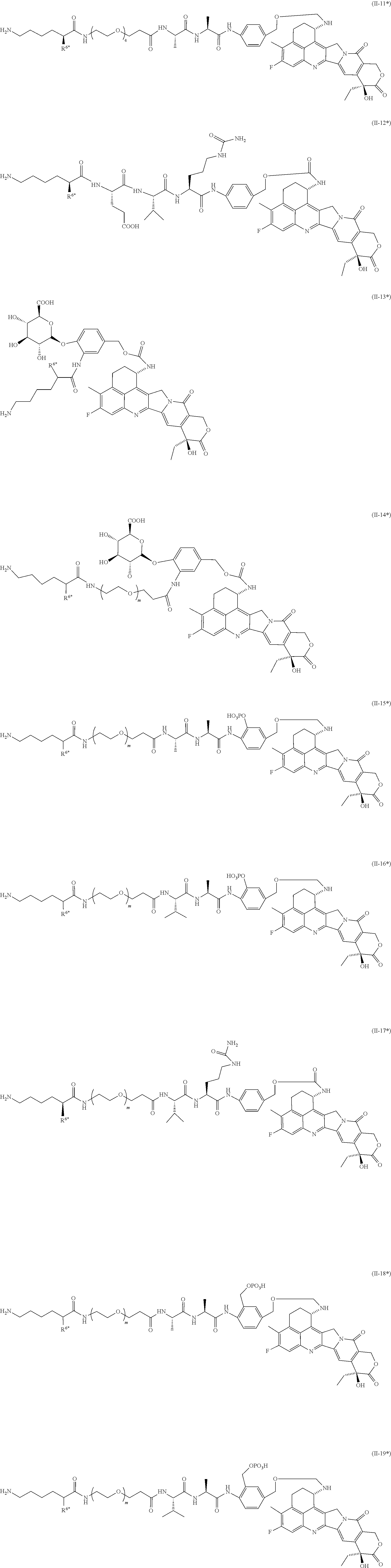

or a pharmaceutically acceptable salt thereof, wherein each variable is independently as described herein.

In some embodiments, the instant invention provides a linker-payload compound selected from Formulae (IV-2*)-(IV-14*):

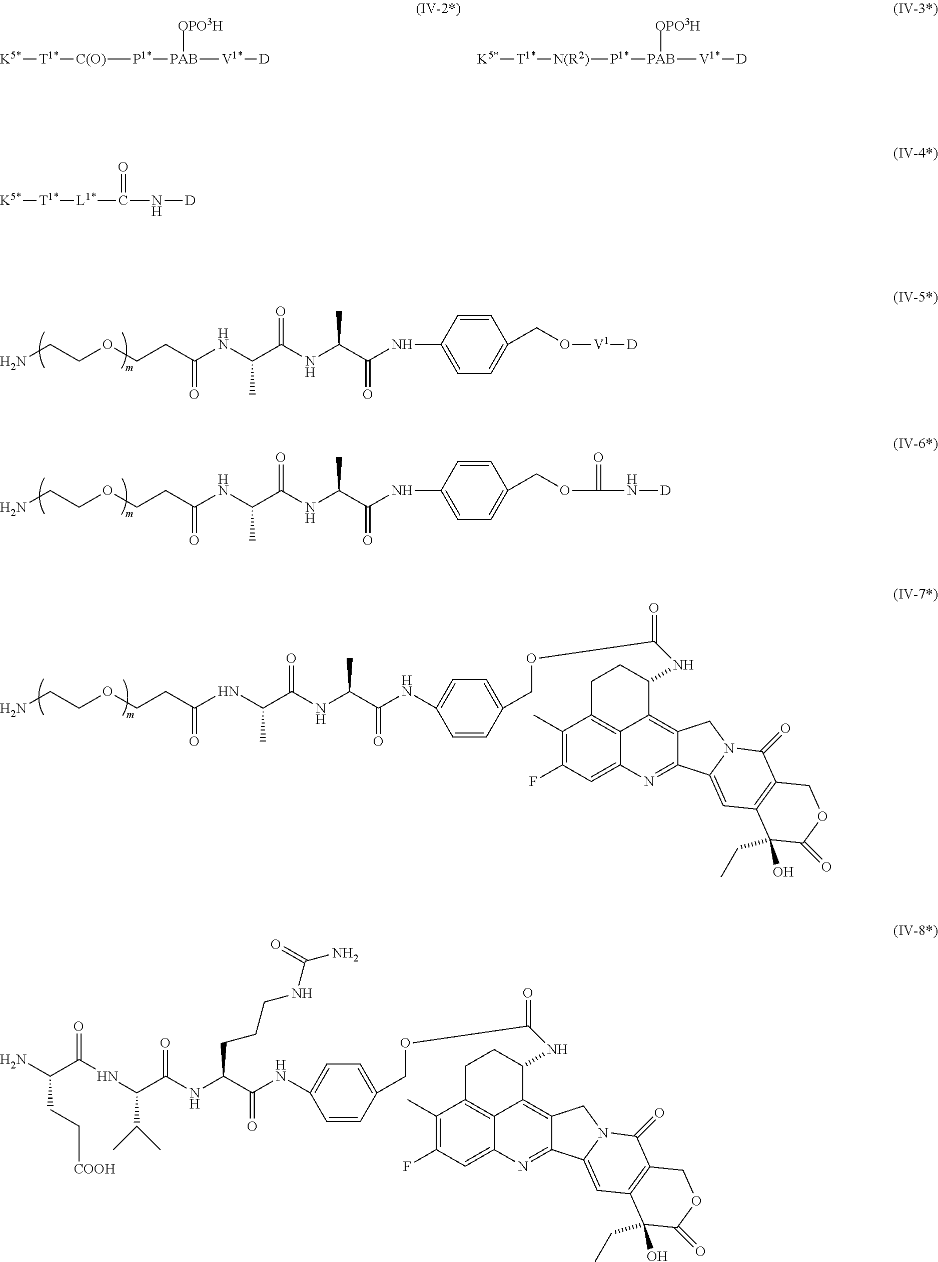

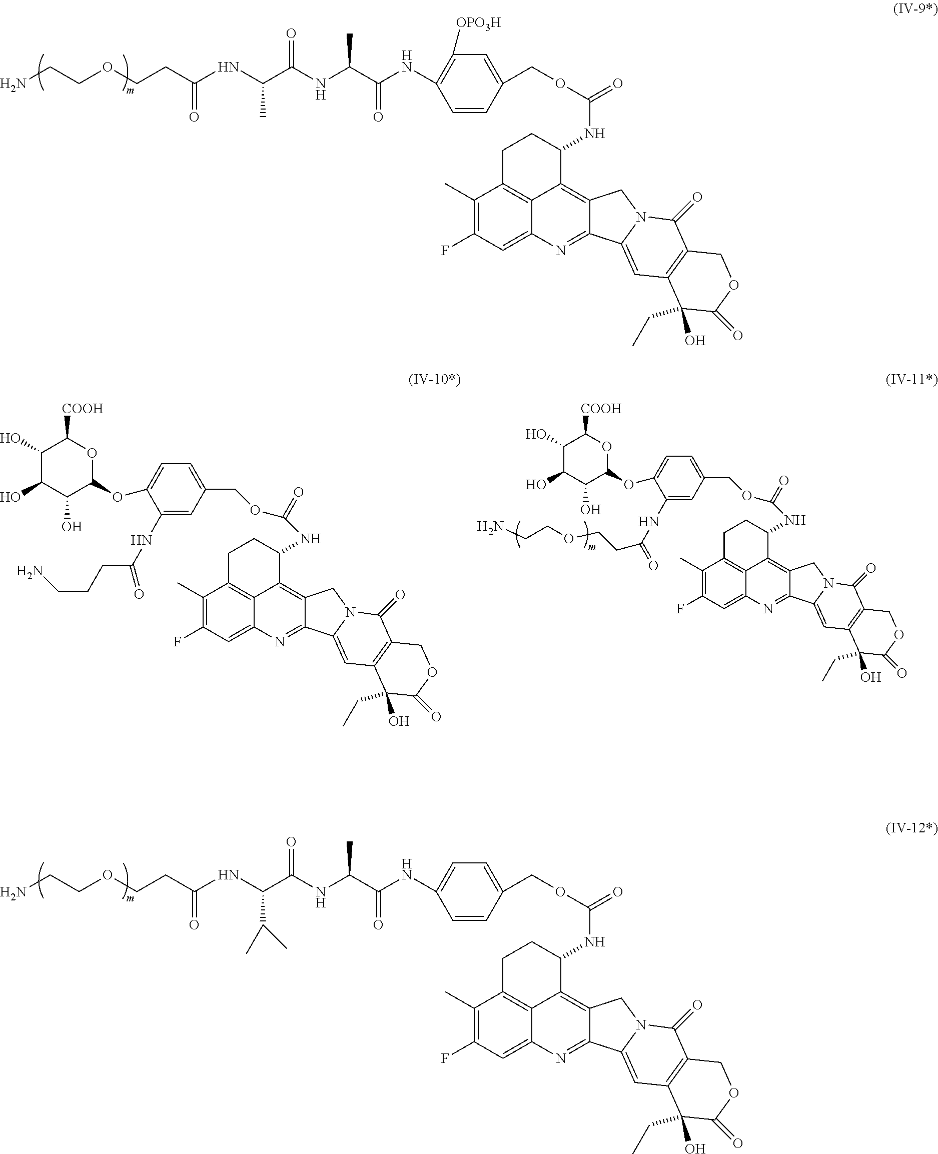

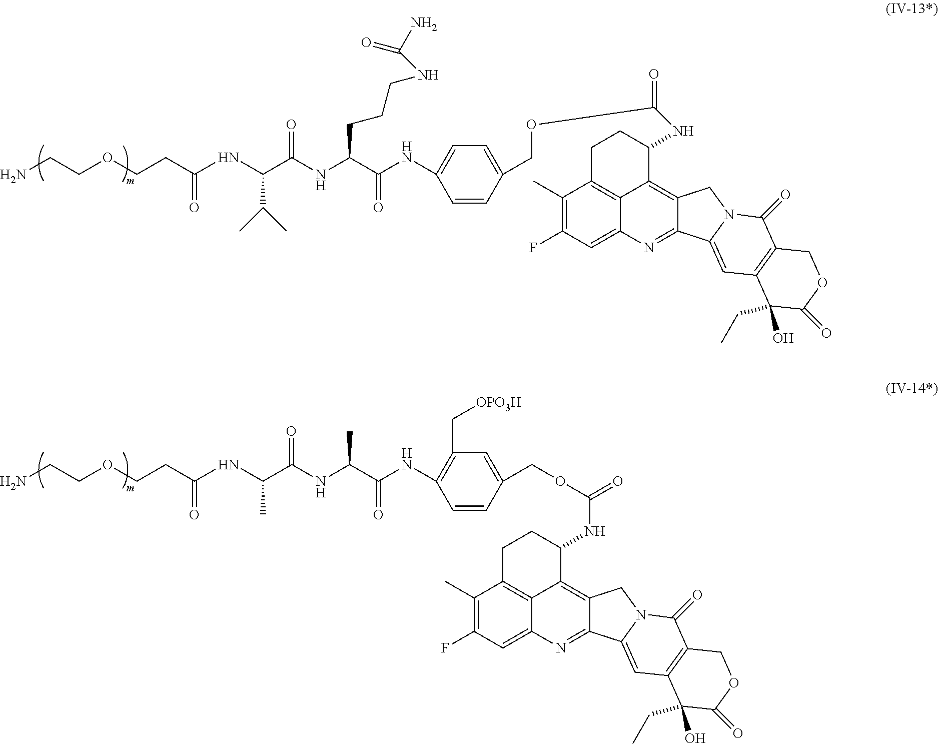

or a pharmaceutically acceptable salt thereof, wherein each variable is independently as described herein.

In some embodiments, the instant invention provides a linker selected from Formulae (VI-2)-(VI-10):

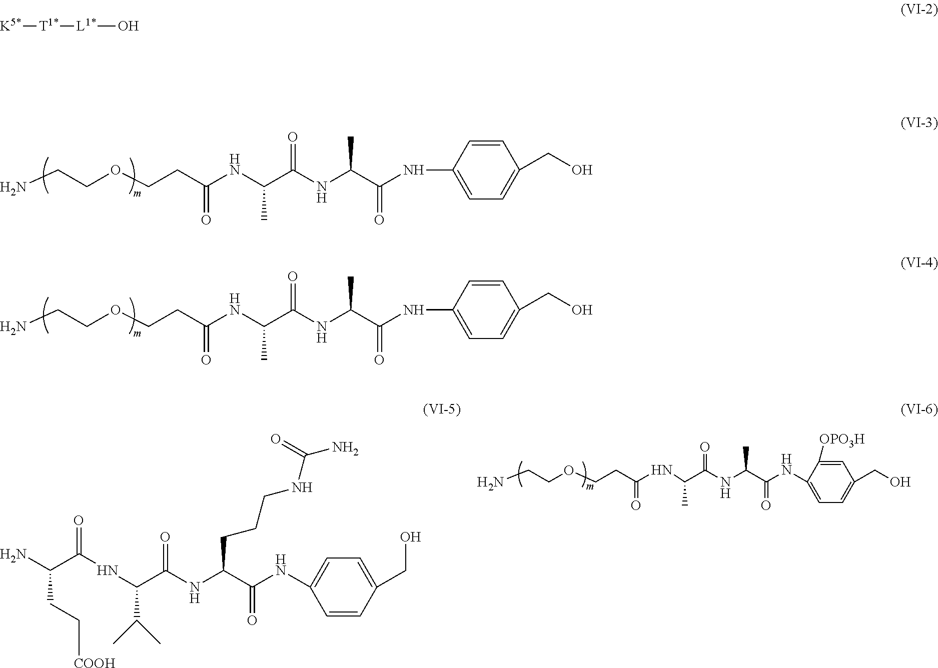

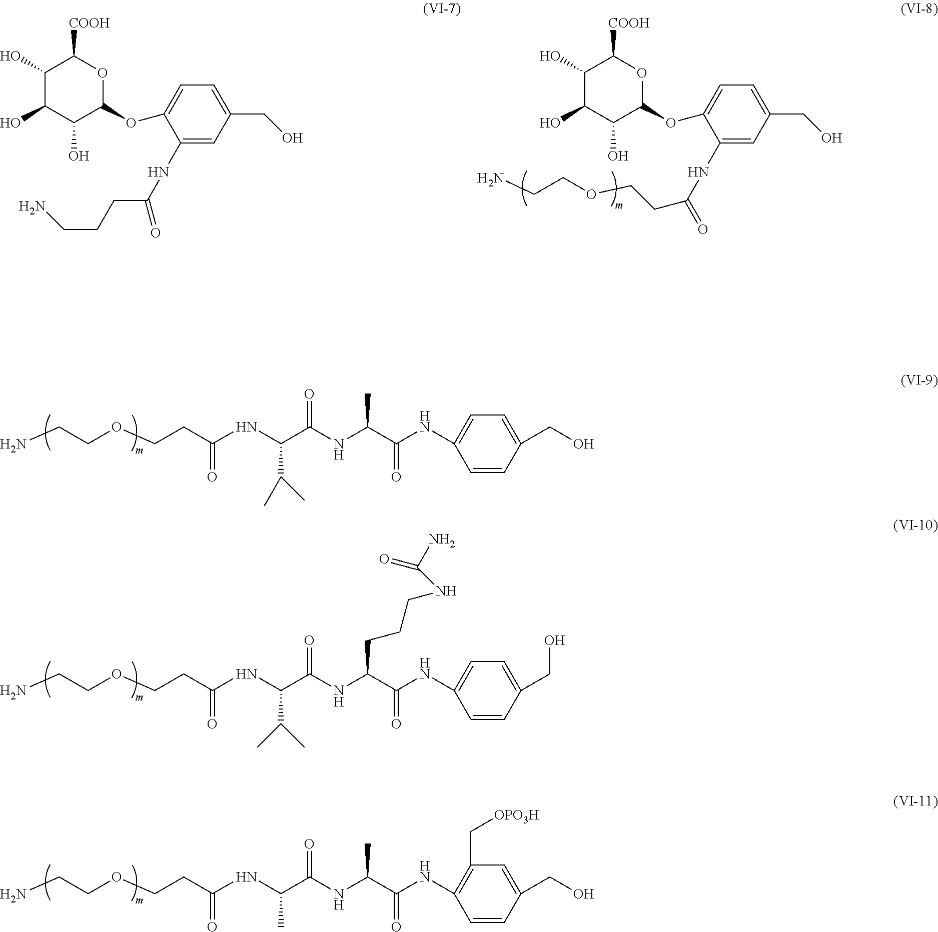

or a pharmaceutically acceptable salt thereof, wherein each variable is independently as described herein.

### 4. Uses, Formulation and Administration Pharmaceutically Acceptable Compositions

According to another embodiment, the disclosure provides a pharmaceutical composition comprising a ligand-payload conjugate of this disclosure, or a pharmaceutically acceptable derivative thereof, and a pharmaceutically acceptable carrier, adjuvant, and/or vehicle. In some embodiments, a composition of this disclosure is formulated for intravenous administration to a patient.

As used herein, the terms “subject” and “patient” are used interchangeably and refer to organisms to be treated by the methods of the present disclosure. Such organisms preferably include, but are not limited to, mammals (e.g., murines, simians, equines, bovines, porcines, canines, felines, and the like), and, most preferably, humans.

The term “pharmaceutically acceptable carrier, adjuvant, and/or vehicle” refers to a non-toxic carrier, adjuvant, and/or vehicle that does not destroy the pharmacological activity of the compound with which it is formulated. Pharmaceutically acceptable carriers, adjuvants, and/or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

Compositions of the present disclosure can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure can be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Alternatively, pharmaceutically acceptable compositions of this disclosure can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, provided pharmaceutically acceptable compositions can be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions can be formulated in an ointment such as petrolatum.

Pharmaceutically acceptable compositions of this disclosure can also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that can be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient depends upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition also depends upon the particular compound in the composition.

### Uses of Compounds and Pharmaceutically Acceptable Compositions

Accordingly, in some aspects and embodiments, the present disclosure provides a method for treating one or more disorders, diseases, and/or conditions, comprising administering to a patient in need thereof a ligand-payload conjugate as described herein, or a pharmaceutical salt or composition thereof. In some embodiments, a disorder, disease, or condition is a cellular proliferative disorder, such as a cancer.

In some embodiments, a disorder, disease, or condition is an autoimmune disease. In some embodiments, an autoimmune disease is selected from Th2-lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

In some embodiments, an autoimmune disease is selected from Active Chronic Hepatitis, Addison's Disease, Allergic Alveolitis, Allergic Reaction Allergic Rhinitis, Alport's Syndrome, Anaphlaxis, Ankylosing Spondylitis, Anti-phosholipid Syndrome Arthritis, Ascariasis, Aspergillosis Atopic Allergy, Axropic Dermatitis, Atropic Rhinitis, Behcet's Disease Bird-Fancier's Lung, Bronchial Asthma, Caplan's Syndrome, Cardiomyopathy, Celiac Disease Chagas' Disease, Chronic Glomeralonephritis, Cogan's Syndrome, Cold Agglutinin Disease, Congenital Rubella Infection CREST Syndrome, Crohn's Disease, Cryoglobulinemia, Cushing's Syndrome, Dermatomyositis Discoid Lupus, Dressier's Syndrome, Eaton-Lambert Syndrome, Echoviras Infection, Encephalomyelitis Endocrine opthalmopathy, Epstein-Barr Viras Infection, Equine Heaves, Erythematosis, Evan's Syndrome Felty's Syndrome, Fibromyalgia, Fuch's Cyclitis, Gastric Atrophy, Gastrointestinal Allergy Giant Cell Arteritis, Glomeralonephritis, Goodpasture's Syndrome, Graft v. Host Disease, Graves' Disease Guillain-Barre Disease, Hashimoto's Thyroiditis, Hemolytic Anemia, Henoch-Schonlein Purpura, Idiopathic Adrenal Atrophy Idiopathic Pulmonary Fibritis, IgA Nephropathy, Inflammatory Bowel Diseases, Insulin-dependent Diabetes Mellitus, Juvenile Arthritis Juvenile Diabetes Mellitus (Type I), Lambert-Eaton Syndrome Laminitis, Lichen Planus, Lupoid Hepatitis, Lupus Lymphopenia, Meniere's Disease, Mixed Connective Tissue Disease, Multiple Sclerosis, Myasthenia Gravis Pernicious Anemia, Polyglandular Syndromes, Presenile Dementia, Primary Agammaglobulinemia, Primary Biliary Cirrhosis Psoriasis, Psoriatic Arthritis, Raynauds Phenomenon, Recurrent Abortion, Reiter's Syndrome Rheumatic Fever, Rheumatoid Arthritis, Sampter's Syndrome, Schistosomiasis, Schmidt's Syndrome Scleroderma, Shulman's Syndrome, Sjorgen's Syndrome, Stiff-Man Syndrome, Sympathetic Ophthalmia Systemic Lupus Erythematosis, Takayasu's Arteritis, Temporal Arteritis, Thyroiditis, Thrombocytopenia Thyrotoxicosis, Toxic Epidermal Necrolysis, Type B Insulin Resistance, Type I Diabetes Mellitus, Ulcerative Colitis Uveitis, Vitiligo, Waldenstrom's Macro globulemia, and Wegener's Granulomatosis.

In some embodiments, a disorder, disease, or condition is an infectious disease. In some embodiments, an infectious disease is selected from infections of pathogenic strains of bacteria (Streptococcus pyogenes, Streptococcus pneumoniae, Neisseria gonorrheae, Neisseria meningitidis, Corynebacterium diphtheriae, Clostridium botulinum, Clostridium perfringens, Clostridium tetani, Hemophilus influenzae, Klebsiella pneumoniae, Klebsiella ozaenas, Klebsiella rhinoscleromotis, Staphylococcus aureus, Vibrio colerae, Escherichia coli, Pseudomonas aeraginosa, Campylobacter (Vibrio) fetus, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Pneumocystis carinii, Francisella tularensis, Bracella abortus, Bracella suis, Bracella melitensis, Mycoplasma spp., Rickettsia prowazeki, Rickettsia tsutsugumushi, Chlamydia spp.); pathogenic fungi (Coccidioides immitis, Aspergillus fumigatus, Candida albicans, Blastomyces dermatitidis, Cryptococcus neoformans, Histoplasma capsulatum); protozoa (Entomoeba histolytica, Toxoplasma gondii, Trichomonas tenas, Trichomonas hominis, Trichomonas vaginalis, Tryoanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminiths (Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms).

In some embodiments, an infectious disease is selected from infections of pathogenic viruses, including and not limited to: Poxviridae, Herpesviridae, Herpes Simplex viras 1, Herpes Simplex virus 2, Adenoviridae, Papovaviridae, Enteroviridae, Picornaviridae, Parvoviridae, Reoviridae, Retroviridae, influenza virases, parainfluenza viruses, mumps, measles, respiratory syncytial viras, rubella, Arboviridae, Rhabdoviridae, Arenaviridae, Hepatitis A virus, Hepatitis B viras, Hepatitis C viras, Hepatitis E viras, Non-A/Non-B Hepatitis viras, Rhinoviridae, Coronaviridae, Rotoviridae, and Human Immunodeficiency Virus.

### Cancer

In some embodiments, the present disclosure provides a method for treating or preventing or reducing the risk of a cancer in patient comprising administering to the patient a compound of the disclosure, or a pharmaceutically acceptable salt thereof, or a pharmaceutical composition thereof.

A “cancer,” as used herein, refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The cancer or proliferative disorder or tumor to be treated using the compounds and methods and uses described herein include, but are not limited to, a hematological cancer, a lymphoma, a myeloma, a leukemia, a neurological cancer, skin cancer, breast cancer, a prostate cancer, a colorectal cancer, a lung cancer, a head and neck cancer, a gastrointestinal cancer, a liver cancer, a pancreatic cancer, a genitourinary cancer, a bone cancer, renal cancer, and a vascular cancer. In some embodiments, the lung cancer is selected from the group consisting of non-small cell lung cancer, small cell lung cancer, and lung carcinoid tumor.

In some embodiments of the methods and uses described herein, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma. In some embodiments, a cancer includes, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, a cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, a cancer is acoustic neuroma, astrocytoma (e.g., Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the methods or uses described herein inhibit or reduce or arrest or ameliorate the growth or spread of a cancer or tumor. In some embodiments, the tumor is treated by arresting, reducing, or inhibiting further growth of the cancer or tumor. In some embodiments, the methods or uses described herein increase or potentiate or activate one or more immune responses to inhibit or reduce or arrest or ameliorate the growth or spread of a cancer or tumor. In some embodiments, the cancer or tumor is treated by reducing the size (e.g., volume or mass) of the cancer or tumor by at least 5%, at least 10%, at least 25%, at least 50%, at least 75%, at least 90%, at least 95%, at least 96%, at least 97%, at least 98%, or at least 99% relative to the size of the cancer or tumor prior to treatment. In some embodiments, cancers or tumors are treated by reducing the quantity of the cancers or tumors in the patient by at least 5%, at least 10%, at least 25%, at least 50%, at least 75%, at least 90%, at least 95%, at least 96%, at least 97%, at least 98%, or at least 99% relative to the quantity of cancers or tumors prior to treatment.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the treatment is initiated. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after the treatment is initiated.

In some embodiments, a patient treated using the methods or uses described herein exhibits an objective response rate (ORR) of at least about 15%, at least about 20%, at least about 25%, at least about 30%, about 35%, about 40%, about 45%, about 50%, about 55%, about 60%, about 65%, about 70%, about 75%, about 80%, about 85%, about 90%, about 95%, or about 100%.

The compounds and compositions, according to the method of the present disclosure, may be administered using any amount and any route of administration effective for treating or lessening the severity of a disease, for example, as those described herein. The exact amount required will vary from subject to subject, depending on the species, age, and general condition of the subject, the severity of the disease or condition, the particular agent, its mode of administration, and the like. Compounds of the disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Co-Administration with One or More Other Therapeutic Agent(s)

Depending upon the particular condition, or disease, to be treated, additional therapeutic agents that are normally administered to treat that condition, can also be present in the compositions of this disclosure. As used herein, additional therapeutic agents that are normally administered to treat a particular disease, or condition, are known as “appropriate for the disease, or condition, being treated.”

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds.

One or more other therapeutic agent(s) can be administered separately from a compound or composition of the disclosure, as part of a multiple dosage regimen. Alternatively, one or more other therapeutic agent(s) may be part of a single dosage form, mixed together with a compound of this disclosure in a single composition. If administered as a multiple dosage regime, one or more other therapeutic agent(s) and a compound or composition of the disclosure can be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent(s) and a compound or composition of the disclosure are administered as a multiple dosage regimen within greater than 24 hours apart.

As used herein, the term “combination,” “combined,” and related terms refers to the simultaneous or sequential administration of therapeutic agents in accordance with this disclosure. For example, a compound of the present disclosure can be administered with one or more other therapeutic agent(s) simultaneously or sequentially in separate unit dosage forms or together in a single unit dosage form. Accordingly, the present disclosure provides a single unit dosage form comprising a compound of the current disclosure, one or more other therapeutic agent(s), and a pharmaceutically acceptable carrier, adjuvant, and/or vehicle.

The amount of a compound of the disclosure and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the disclosure should be formulated so that a dosage of between 0.01-100 mg/kg body weight/day of a compound of the disclosure can be administered.

In those compositions which comprise one or more other therapeutic agent(s), the one or more other therapeutic agent(s) and a compound of the disclosure can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this disclosure may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this disclosure, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

### Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells.

In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF®, Taiho Oncology).

In some embodiments, one or more other therapeutic agent is a kinase inhibitor or VEGF-R antagonist. Approved VEGF inhibitors and kinase inhibitors useful in the present disclosure include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present disclosure include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present disclosure include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present disclosure include olaratumab (LARTRUVO®; Eli Lilly). Approved EGFR antagonists which may be used in the present disclosure include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present disclosure include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present disclosure include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present disclosure include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present disclosure include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present disclosure include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present disclosure include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present disclosure include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present disclosure include irinotecan (ONIVYDE®, Merrimack Pharmaceuticals); topotecan (HYCAMTIN®, GlaxoSmithKline). Topoisomerase inhibitors being studied which may be used in the present disclosure include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present disclosure include venetoclax (VENCLEXTA®, AbbVie/Genentech); and blinatumomab (BLINCYTO®, Amgen). Other therapeutic agents targeting apoptotic proteins which have undergone clinical testing and may be used in the present disclosure include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present disclosure include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON®, Ferring Pharmaceuticals).

In some embodiments, one or more other therapeutic agent is a selective estrogen receptor modulator (SERM), which interferes with the synthesis or activity of estrogens. Approved SERMs useful in the present disclosure include raloxifene (EVISTA®, Eli Lilly).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present disclosure include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGF-0). Inhibitors of TGF-beta proteins being studied which may be used in the present disclosure include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARIMIDEX™. Letrozole is marketed under the trade names FEMARA™ or FEMAr™. Aminoglutethimide is marketed under the trade name ORIMETEN™. A combination of the disclosure comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™. Raloxifene hydrochloride is marketed under the trade name EVISTA™. Fulvestrant can be administered under the trade name FASLODEX™ Fulvestrant can be administered under the trade name Faslodex™. A combination of the disclosure comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™.

The term “topoisomerase I inhibitor” as used herein includes, but is not limited to exatecan, topotecan, gimatecan, irinotecan, camptothecin and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™.

The term “topoisomerase II inhibitor” as used herein includes, but is not limited to the anthracyclines such as doxorubicin (including liposomal formulation, such as CAELYX™), daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™. Teniposide is marketed under the trade name VM 26-Bristol Doxorubicin is marketed under the trade name ACRIBLASTIN™ or ADRIAMYCIN™. Epirubicin is marketed under the trade name FARMORUBICIN™. Idarubicin is marketed. under the trade name ZAVEDOS™ Mitoxantrone is marketed under the trade name NOVANTRON™.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™.

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™.

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g., under the trademark ELOXATIN™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™) Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this disclosure include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), 5-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this disclosure can be found in WO2008039218 and WO2011090760, the entirety of which are incorporated herein by reference.

Further examples of SYK inhibitory compounds, and conditions treatable by such compounds in combination with compounds of this disclosure can be found in WO2003063794, WO2005007623, and WO2006078846, the entirety of which are incorporated herein by reference.

Further examples of PI3K inhibitory compounds, and conditions treatable by such compounds in combination with compounds of this disclosure can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this disclosure can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the disclosure include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g., inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (

CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™. Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE®), everolimus (CERTICAN™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™) rituximab (RITUXAN®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current disclosure can be used in combination with standard leukemia therapies, especially in combination with therapies used for the treatment of AML. In particular, compounds of the current disclosure can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgG1 antibody, Angiozyme (RPI 4610) and Bevacizumab (AVASTIN™).

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the disclosure has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTOR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the disclosure and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present disclosure include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase− (TK−) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-lh68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present disclosure include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present disclosure include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present disclosure include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present disclosure include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present disclosure include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present disclosure include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present disclosure include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that may be used in the present disclosure include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present disclosure include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present disclosure include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present disclosure include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present disclosure include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present disclosure include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present disclosure include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present disclosure include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present disclosure include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXAMPLES

The following examples are intended to illustrate the disclosure and are not to be construed as being limitations thereon.

### Example 1: Synthesis of Certain Intermediates and Ligand-Payload Conjugates

### 1.1. Synthesis of Linkers of Formula (VI)

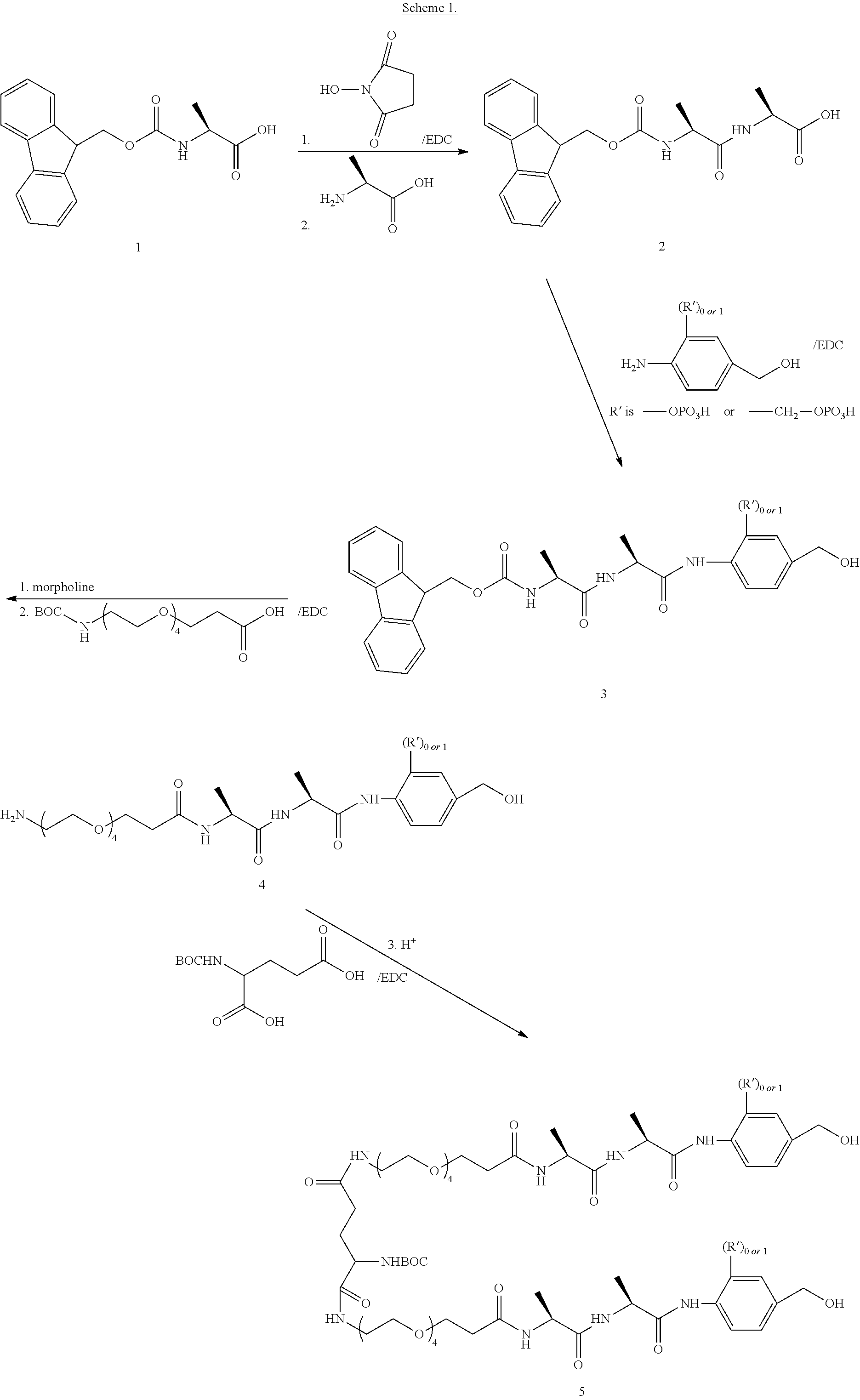

A linker of Formula (VI), Compound 5, is synthesized as shown in Scheme 1. A Fmoc protected L-alanine is converted to a NHS ester, followed by a coupling reaction with a second L-alanine to afford a Fmoc protected dipeptide, Compound 2. Compound 2 is then couple with p-aminobenzyl alcohol via an acid base condensation to afford Compound 3. The Fmoc protecting group on Compound 3 is removed to afford an amine group, followed by a coupling reaction with a PEG linker to afford a linear linker Compound 4. Two equivalents of Compound 4 are coupled with one equivalent of N-protected glutamic acid to afford linker Compound 5.

### 1.2. Synthesis of Linker-Payload Compounds of Formula (IV)

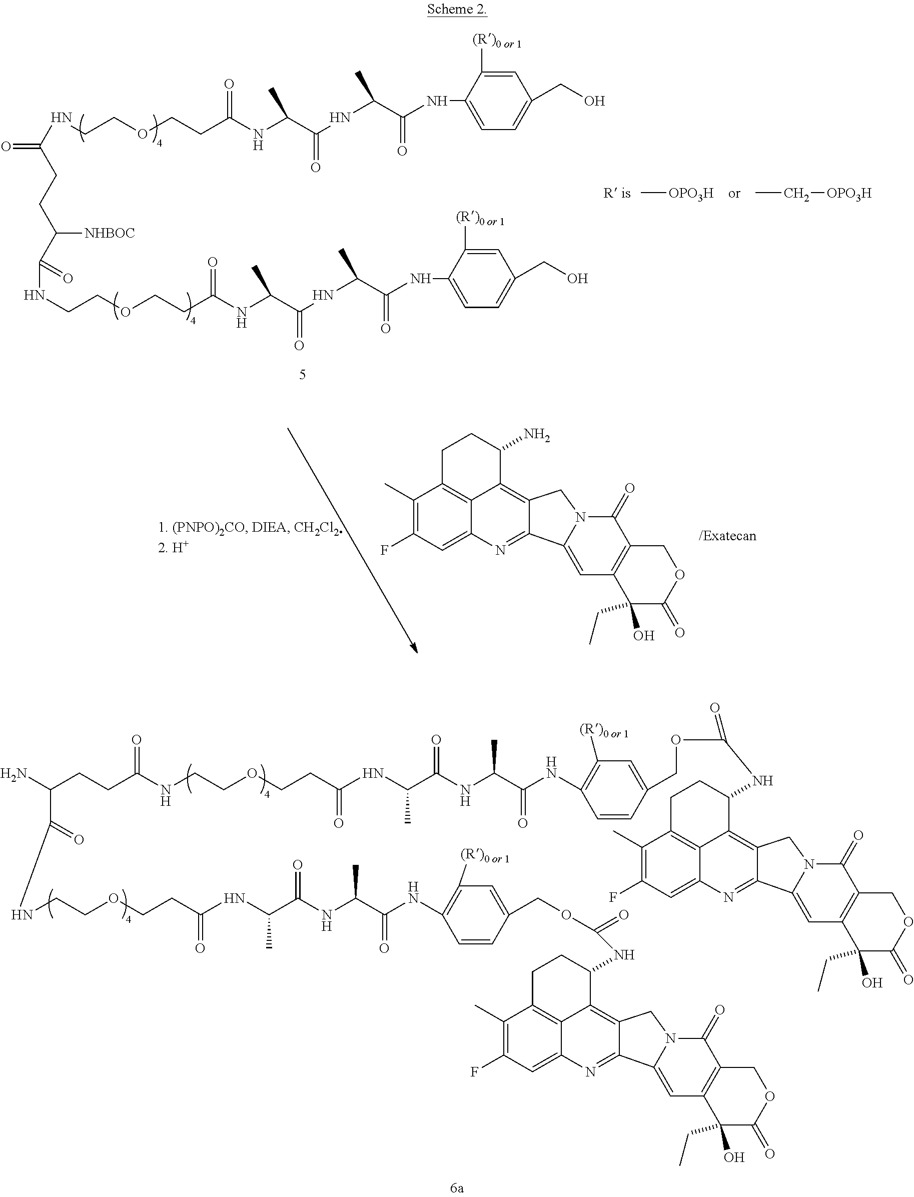

Two equivalents of exatecan are coupled with one equivalent of linker Compound 5, followed by a deprotection to remove the BOC protecting group to form linker-payload compound 6a.

### 1.3. Synthesis of Linker-Payload Compounds of Formula (II)

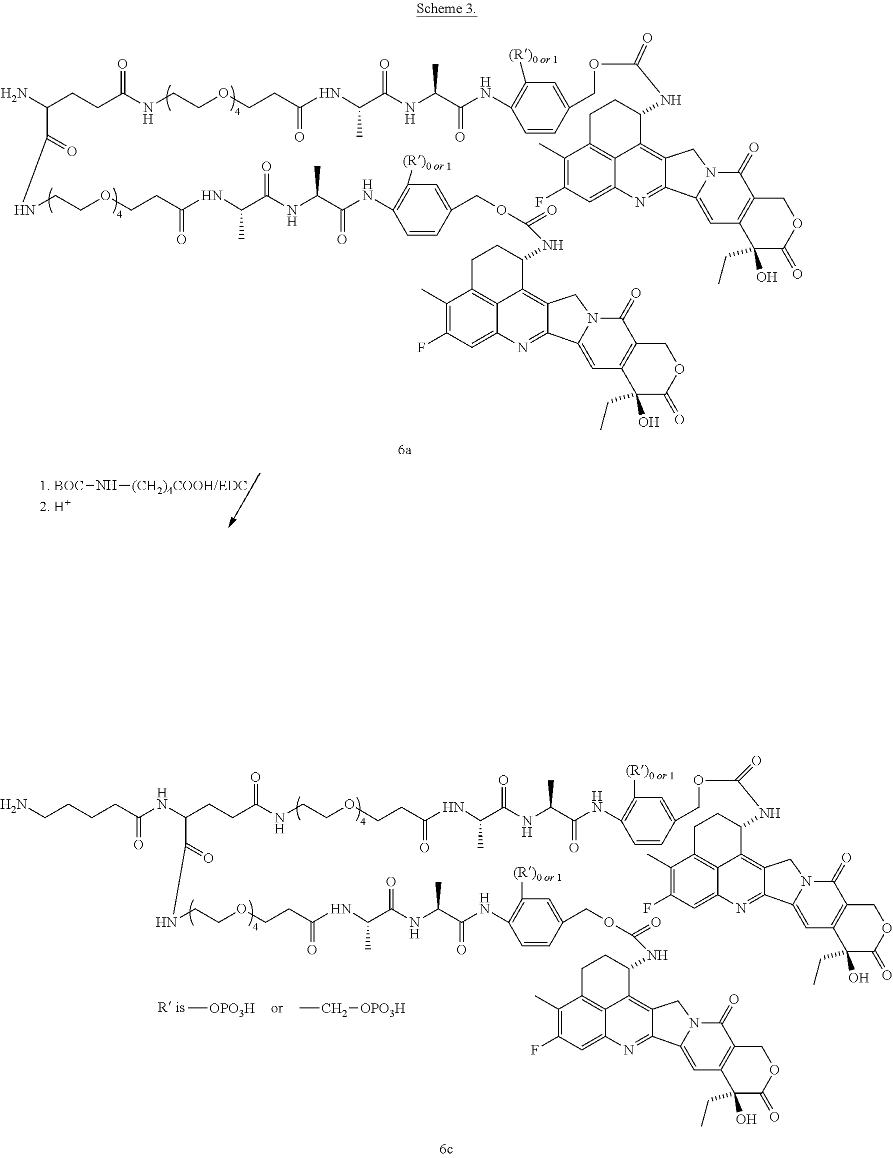

Linker-payload compound 6a and N-protected 5-aminopentanoic acid are coupled via an acid base condensation, followed by a deprotection to remove the BOC protecting group to form linker-payload compound 6c.

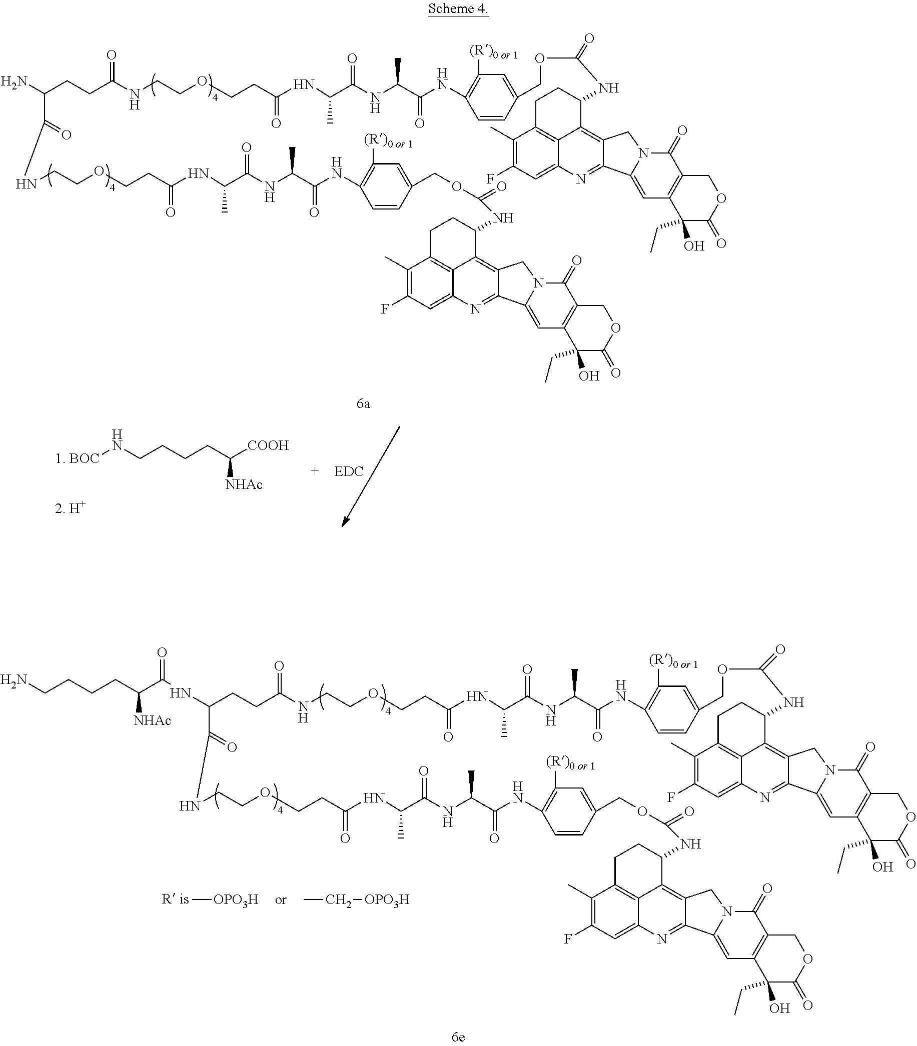

Linker-payload compound 6a and N-protected lysine are coupled via an acid base condensation, followed by a deprotection to remove the BOC protecting group to form linker-payload compound 6e.

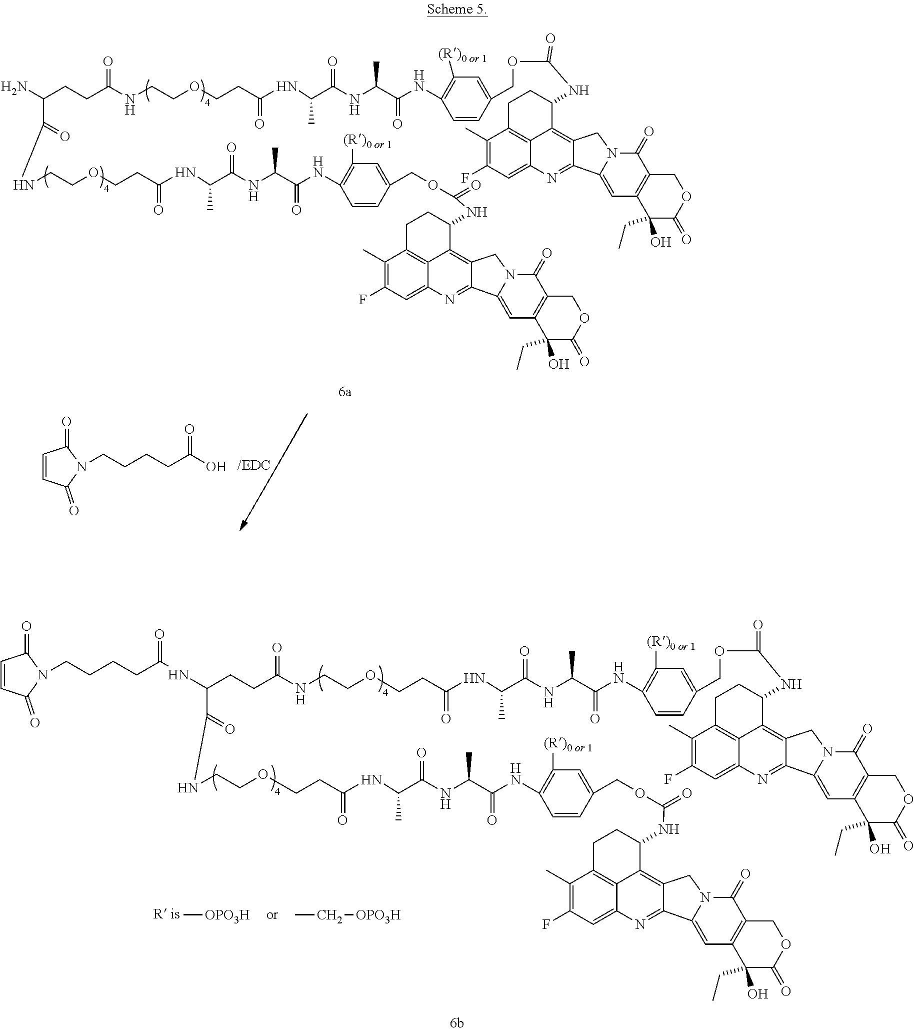

Linker-payload compound 6a and 5-(2,5-dioxo-2,5-dihydro-1H-pyrrol-1-yl)pentanoic acid are coupled via an acid base condensation to form linker-payload compound 6b.

### 1.4. Synthesis of Ligand-Payload Compounds of Formula (I)

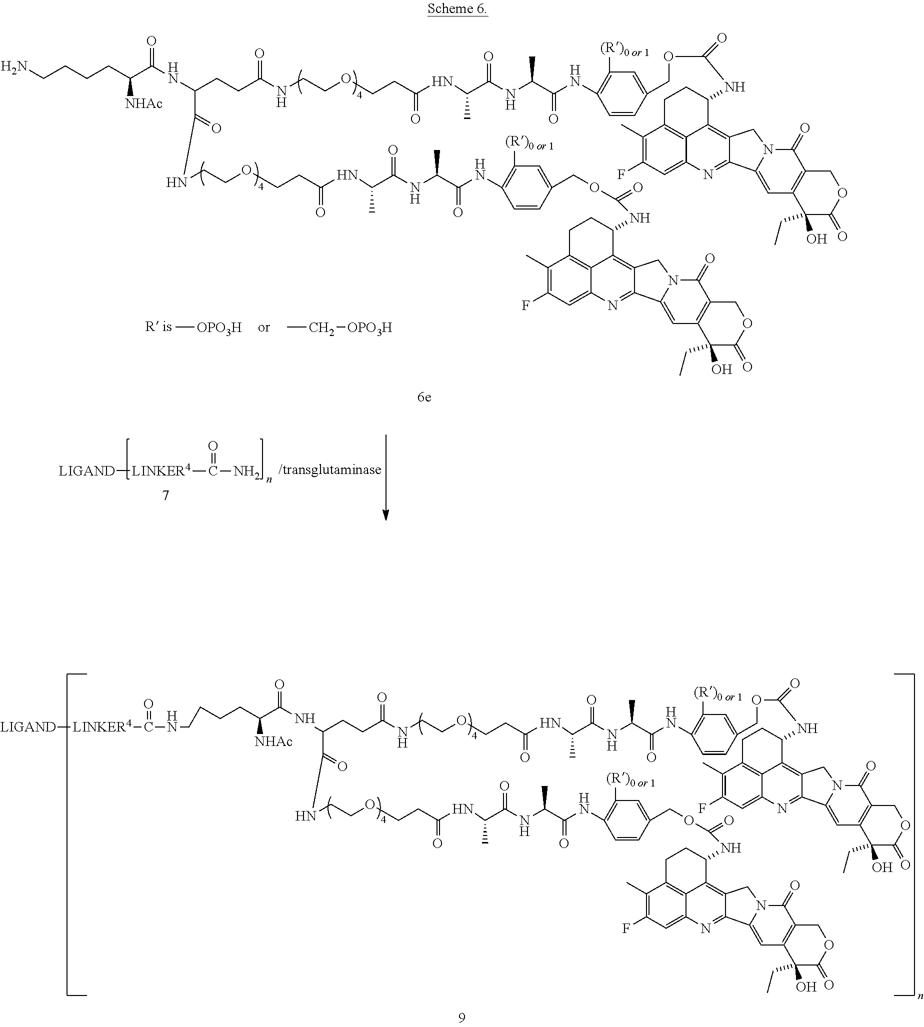

Linker-payload compound 6e and ligand 7 are coupled via an acyl addition to form ligand-payload compound 9.

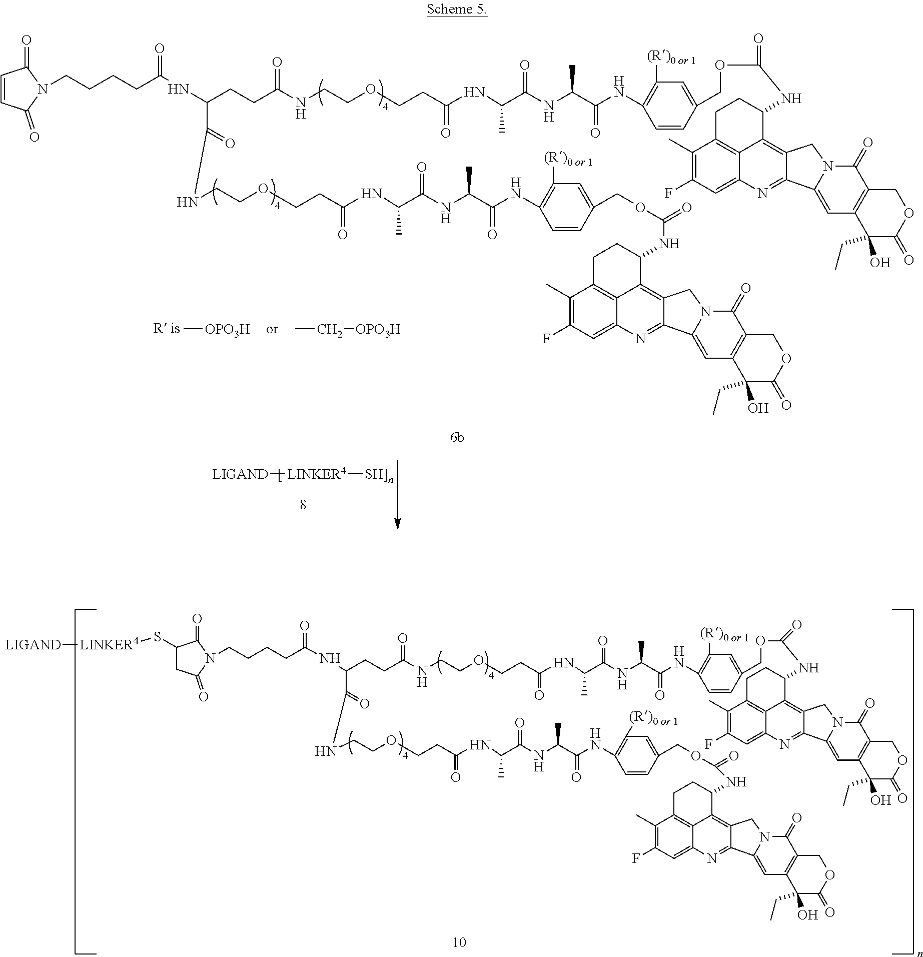

Linker-payload compound 6b and ligand 8 are coupled via a Michael addition to form ligand-payload compound 10.

### Example 2: Synthesis of Certain Intermediates and Ligand-Payload Conjugates

### 2.1. Synthesis of Linker-Payload of Formula (IV)

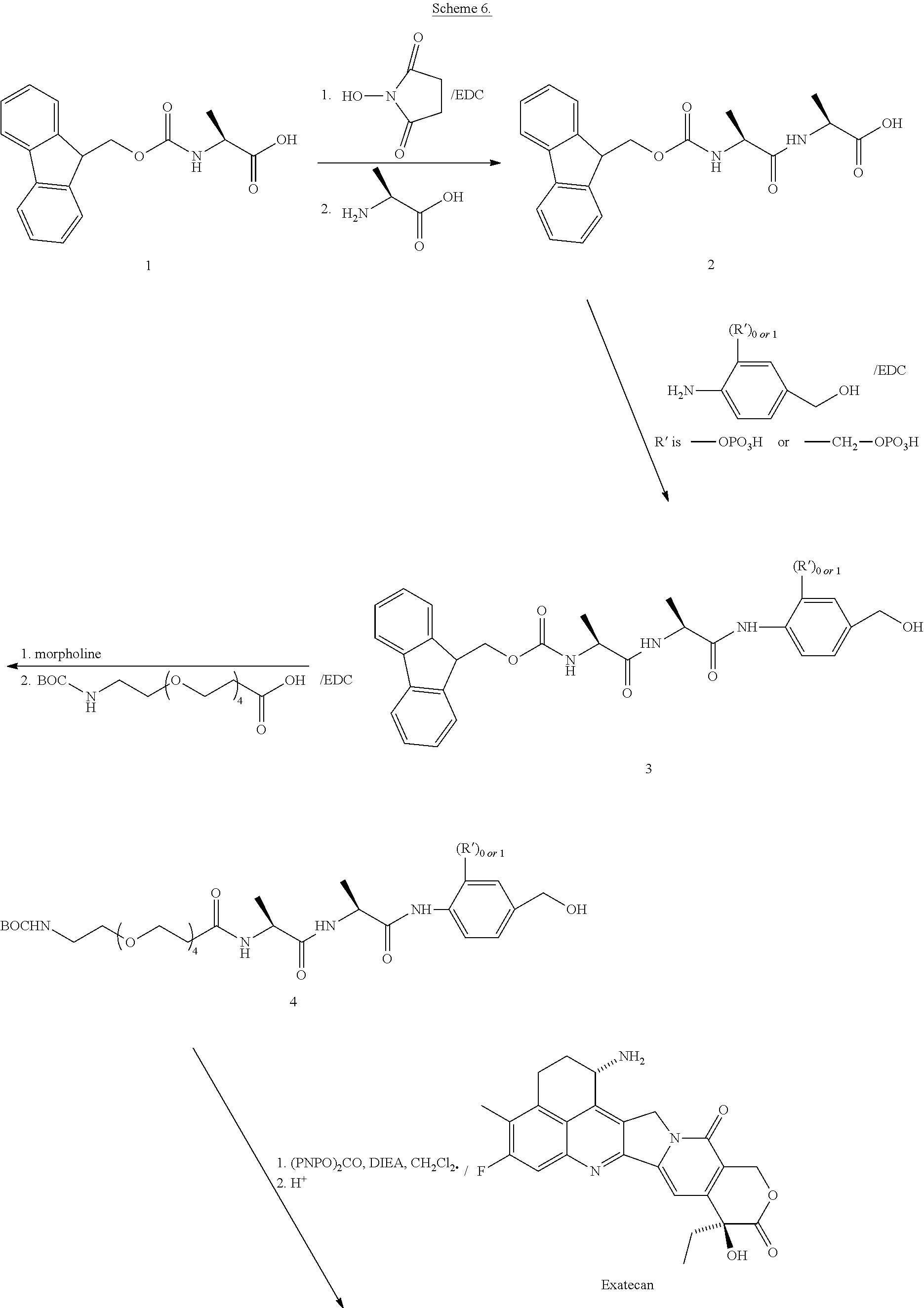

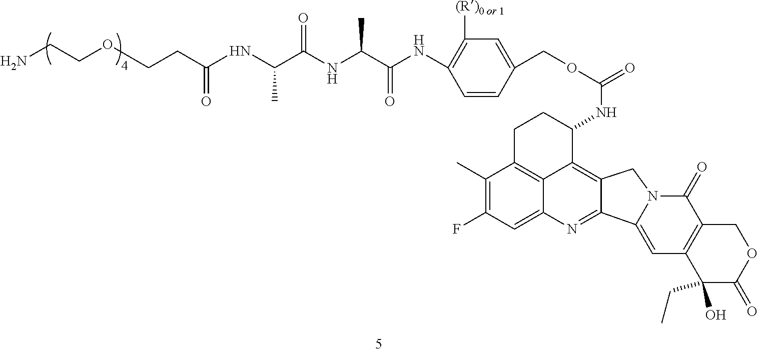

A linker-payload of Formula (IV), Compound 5, is synthesized as shown in Scheme 6. A Fmoc protected L-alanine is converted to a NHS ester, followed by a coupling reaction with a second L-alanine to afford a Fmoc protected dipeptide, Compound 2. Compound 2 is then coupled with p-aminobenzyl alcohol via an acid base condensation to afford Compound 3. The Fmoc protecting group on Compound 3 is removed to afford an amine group, followed by a coupling reaction with a PEG linker to afford a linear linker Compound 4. Compound 4 is coupled with exatecan, followed by a deprotection to afford linker-payload Compound 5.

### 2.2. Synthesis of Linker-Payload Compounds of Formula (II*)

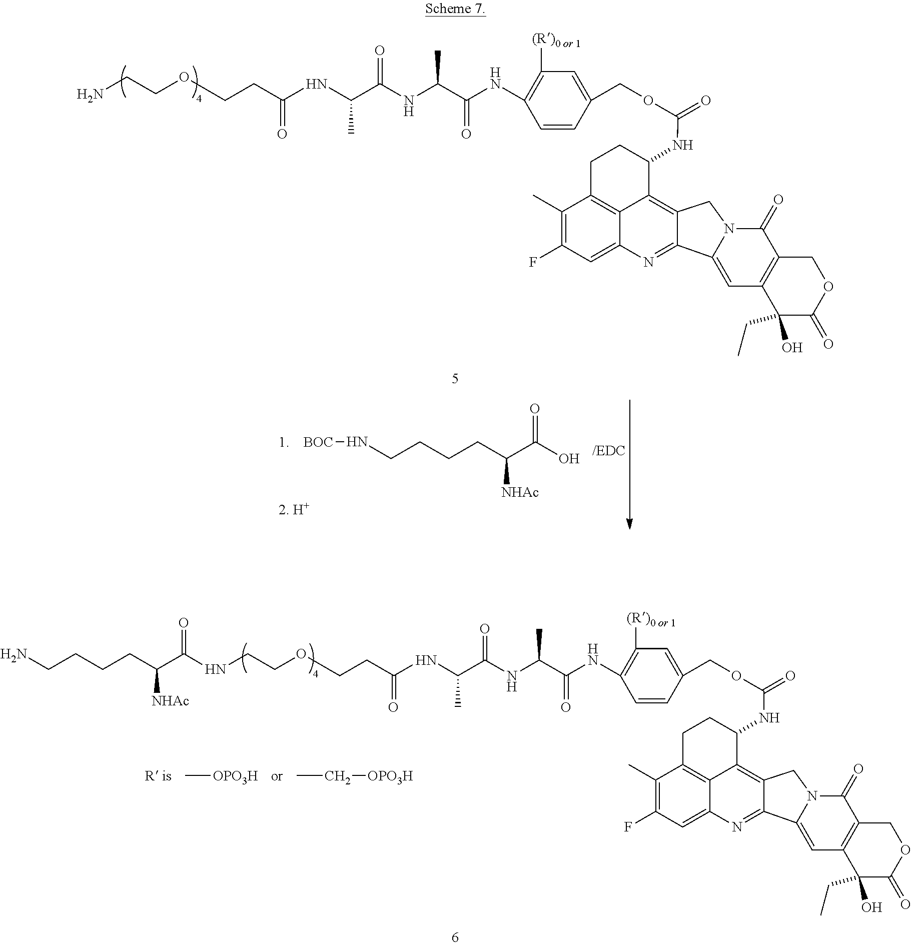

Linker-payload compound 5 and N-protected L-lysine are coupled via an acid base condensation, followed by a deprotection to remove the BOC protecting group to form linker-payload compound 6.

### 2.3. Synthesis of Ligand-Payload Compounds of Formula (I*)

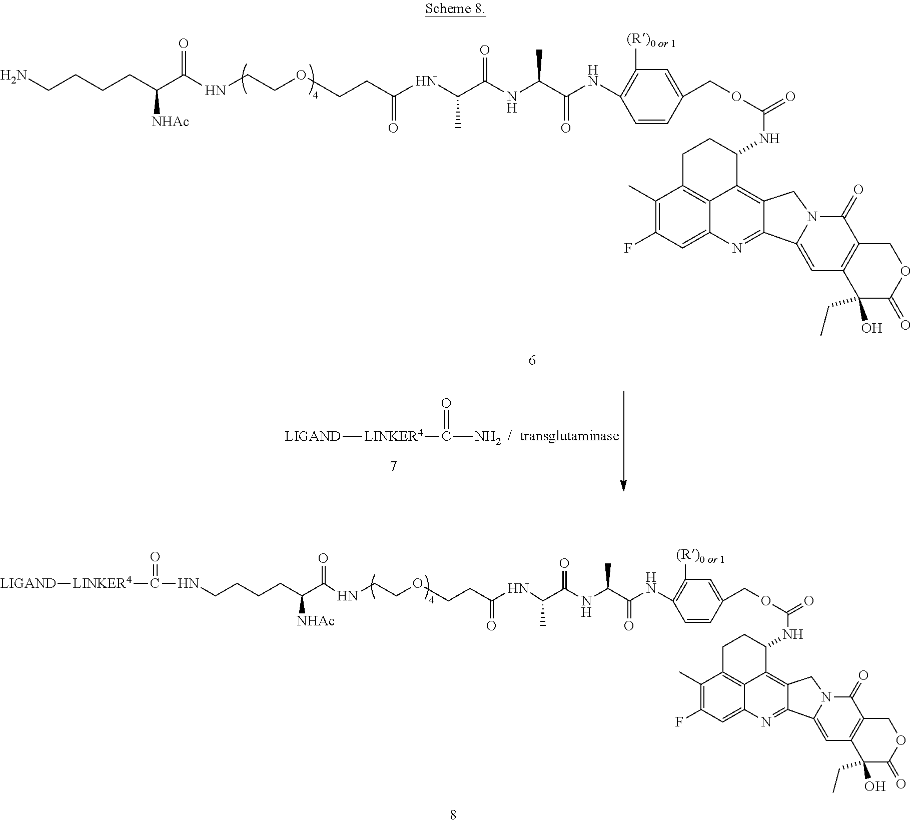

Linker-payload compound 6 and ligand 7 are coupled via an acyl addition to form ligand-payload compound 8.

### Example 3: In Vitro Studies

### Selected Linker-Payload Compounds

The following branched linker-payload compounds, which can be conjugated to antibodies using for example maleimide conjugation chemistry, were prepared according to the methods described herein:

### Branched Linker (6b): Two (PEG)₄AAPABC-Exatecan

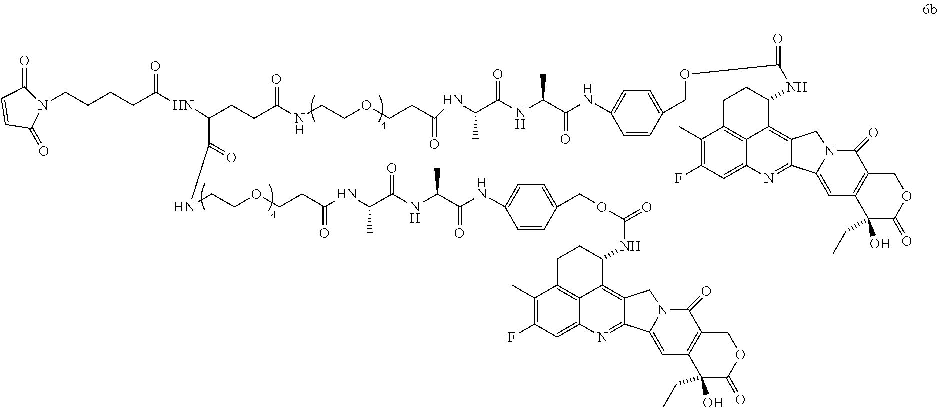

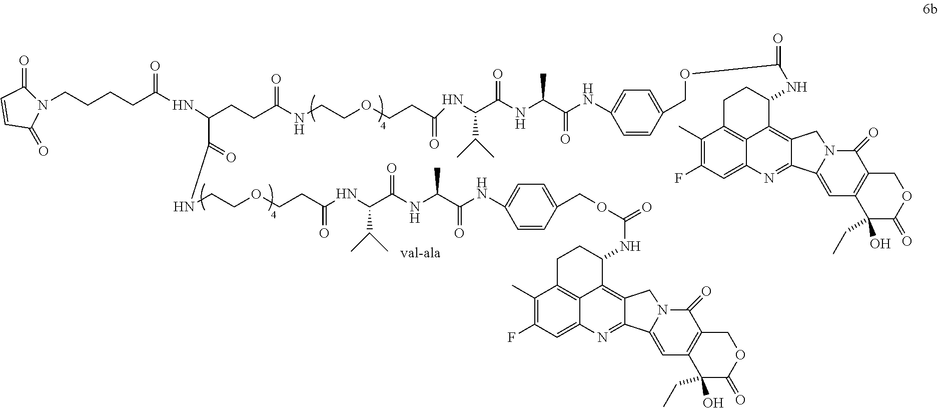

### Branched Linker (6b′): Two (PEG)₄VAPABC-Exatecan

The following linear linker-payload compounds, which can be conjugated to antibodies using for example maleimide conjugation chemistry, were prepared according to the methods described herein:

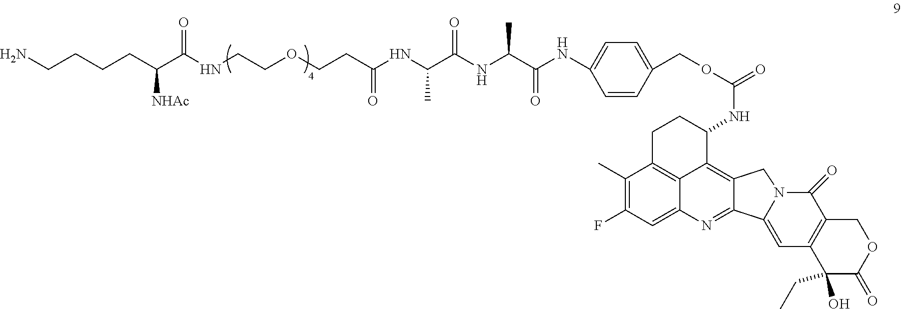

### Linear Linker (9): AcLys(PEG)₄AAPABC-Exatecan

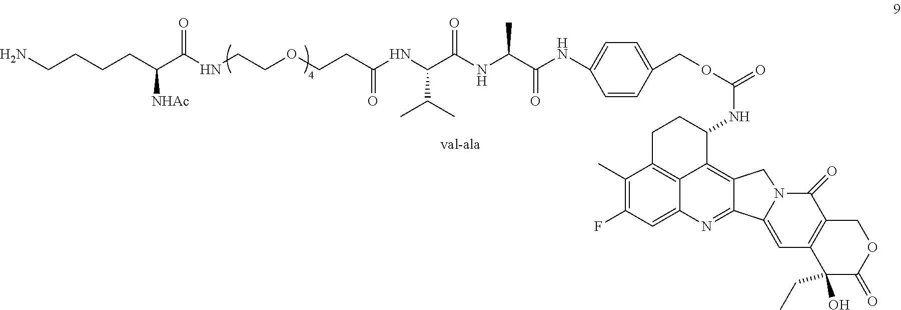

### Linear Linker (9′): AcLys(PEG)₄VAPABC-Exatecan

As described elsewhere herein, branched linker 6b was used to prepare ADC1 and ADC2.

As described elsewhere herein, branched linker 6b′ was used to prepare ADC10.

As described elsewhere herein, linear linker 9 was used to prepare ADC3 and ADC4.

As described elsewhere herein, linear linker 9′ was used to prepare ADC11.

The ADCs prepared are illustrated in Table 3.

ENHERTU® (Trastuzumab deruxtecan), which comprises the humanized monoclonal antibody trastuzumab (HERCEPTIN®; CAS Number 180288-69-1) covalently linked to the topoisomerase I inhibitor deruxtecan through reducing interchain disulfides, was used as a positive control ADC. Trastuzumab binds to and blocks signaling through epidermal growth factor receptor 2 (HER2/neu) on cancers that rely on it for growth. Additionally, once bound to HER2 receptors, the ADC is internalized by the cell and the bound deruxtecan is released by protease cleavage. Then, the drug causes DNA damage during replication and thus kills the cell.

The amino acid sequence for Trastuzumab (as present in ENHERTU®) is as follows:

LC sequence (SEQ ID NO: 34):
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
HC sequence (SEQ ID NO: 35):
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSREE
MTKNQVSLTC LVKGFYPSDI AVEWESNGOP ENNYKTTPPV LDSDGSFFLY SKLTVDKSRW
QQGNVFSCSV MHEALHNHYT QKSLSLSPGK

The amino acid sequence for the Trastuzumab variant present in ADC1 is as follows:

| LC sequence (SEQ ID NO: 34): | |
|---|---|
| DIQMTQSPSS LSASVGDRVT ITCRASQDVN TAVAWYQQKP GKAPKLLIYS ASFLYSGVPS | 60 |
| RFSGSRSGTD FTLTISSLQP EDFATYYCQQ HYTTPPTFGQ GTKVEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | |
| HC sequence [K290C (EU) or K307C (Kabat); bold underlined] (SEQ ID NO: 36): | |
| EVQLVESGGG LVQPGGSLRL SCAASGFNIK DTYIHWVRQA PGKGLEWVAR IYPTNGYTRY | 60 |
| ADSVKGRFTI SADTSKNTAY LQMNSLRAED TAVYYCSRWG GDGFYAMDYW GQGTLVTVSS | 120 |
| ASTKGPSVFP LAPSSKSTSG GTAALGCLVK DYFPEPVTVS WNSGALTSGV HTFPAVLQSS | 180 |
| GLYSLSSVVT VPSSSLGTQT YICNVNHKPS NTKVDKKVEP KSCDKTHTCP PCPAPELLGG | 240 |
| PSVFLFPPKP KDTLMISRTP EVTCVVVDVS HEDPEVKFNW YVDGVEVHNA KTCPREEQYN | 300 |
| STYRVVSVLT VLHQDWLNGK EYKCKVSNKA LPAPIEKTIS KAKGQPREPQ VYTLPPSREE | 360 |
| MTKNQVSLTC LVKGFYPSDI AVEWESNGQP ENNYKTTPPV LDSDGSFFLY SKLTVDKSRW | 420 |
| QQGNVFSCSV MHEALHNHYT QKSLSLSPGK | |

The amino acid sequence for the Trastuzumab variant present in ADC2 and ADC10 is as follows:

| LC sequence [K183C, Kabat&EU; bold underlined] (SEQ ID NO: 37): | |
|---|---|
| DIQMTQSPSS LSASVGDRVT ITCRASQDVN TAVAWYQQKP GKAPKLLIYS ASFLYSGVPS | |
| RFSGSRSGTD FTLTISSLQP EDFATYYCQQ HYTTPPTFGQ GTKVEIKRTV AAPSVFIFPP | |
| SDEQLKSGTA SVVCLLNNFY PREAKVQWKV DNALQSGNSQ ESVTEQDSKD STYSLSSTLT | |
| LSCADYEKHK VYACEVTHQG LSSPVTKSFN RGEC | |
| HC sequence [K290C (EU) or K307C (Kabat); bold underlined] (SEQ ID NO: 36): | |
| EVQLVESGGG LVQPGGSLRL SCAASGFNIK DTYIHWVRQA PGKGLEWVAR IYPTNGYTRY | 60 |
| ADSVKGRFTI SADTSKNTAY LQMNSLRAED TAVYYCSRWG GDGFYAMDYW GQGTLVTVSS | 120 |
| ASTKGPSVFP LAPSSKSTSG GTAALGCLVK DYFPEPVTVS WNSGALTSGV HTFPAVLQSS | 180 |
| GLYSLSSVVT VPSSSLGTQT YICNVNHKPS NTKVDKKVEP KSCDKTHTCP PCPAPELLGG | 240 |
| PSVFLFPPKP KDTLMISRTP EVTCVVVDVS HEDPEVKFNW YVDGVEVHNA KTCPREEQYN | 300 |
| STYRVVSVLT VLHQDWLNGK EYKCKVSNKA LPAPIEKTIS KAKGQPREPQ VYTLPPSREE | 360 |
| MTKNQVSLTC LVKGFYPSDI AVEWESNGQP ENNYKTTPPV LDSDGSFFLY SKLTVDKSRW | 420 |
| QQGNVFSCSV MHEALHNHYT QKSLSLSPGK | |

The amino acid sequence for the Trastuzumab variant present in ADC3 and ADC11 is as follows:

LC sequence (SEQ ID NO: 34):
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG ISSPVIKSFN RGEC

HC sequence [site-specific transglutaminase at HC only, two sites Q295 and Q297 (EU numbering scheme) or Q298 and Q300 (sequential); bold underline] (SEQ ID NO:38):

EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYQ
STYRVVSVLT VLHQDWLNGK EYKCKVSNKA LPAPIEKTIS KAKGQPREPQ VYTLPPSREE
MTKNQVSLTC LVKGFYPSDI AVEWESNGQP ENNYKTTPPV LDSDGSFFLY SKLTVDKSRW
QQGNVFSCSV MHEALHNHYT QKSLSLSPGK

The amino acid sequence for the Trastuzumab variant present in ADC4 is as follows:

LC sequence [site-specific transglutaminase mutations at C-terminal; bold
underlined] (SEQ ID NO: 39):
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGECGGLLQGA
HC sequence [site-specific transglutaminase mutations at positions 295 and 297
(EU numbering scheme) or Q298 and Q300 (sequential); bold underlined]
(SEQ ID NO: 38):
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYQ
STYRVVSVLT VLHQDWLNGK EYKCKVSNKA LPAPIEKTIS KAKGQPREPQ VYTLPPSREE
MTKNQVSLTC LVKGFYPSDI AVEWESNGQP ENNYKTTPPV LDSDGSFFLY SKLTVDKSRW
QQGNVFSCSV MHEALHNHYT QKSLSLSPGK

IgG1 isotype was used as the negative control.

TABLE 3

| | | | | | |
|---|---|---|---|---|---|
| | | | | | Target |
| ADC# | mAb | LP | DAR | MS-DAR | DAR % |

Topo branched linker-payload; Maleimide

| | | | | | |
|---|---|---|---|---|---|
| ADC-1 | 290C | Branched 6b - | 4 | 4.08 | 90.41 |
| ADC-2 | Double mutant | 2 × (PEG)₄AAPABC- | 8 | 7.98 | 73.61 |
| | 183C/290C | Exatecan | | | |
| ADC-10 | Double mutant | Branched 6b′ - | 8 | 7.67 | 73.42 |
| | 183C/290C | 2 × (PEG)₄VAPABC- | | | |
| | | Exatecan | | | |

Topo linear linker-payload; Transglutaminase

| | | | | | |
|---|---|---|---|---|---|
| ADC-3 | N297Q, native Q295 | Linear linker 9 - | 4 | 3.90 | 88.41 |
| ADC-4 | N297Q, native Q295, | AcLys(PEG)₄AAPABC- | 6 | 5.91 | 87.42 |
| | LC C-terminus | Exatecan | | | |
| | GGLLQGA | | | | |
| ADC-11 | N297Q, native Q295 | Linear linker 9′ - | 4 | 3.88 | 85.24 |
| | | AcLys(PEG)₄VAPABC- | | | |
| | | Exatecan | | | |
| ADC-8 | native interchain | GGFG-Deruxtecan (Dxd) | 8 | 7.96 | |
| (ENHERTU ®) | cysteines | | | | |

### Study of ADCs in Cell Lines

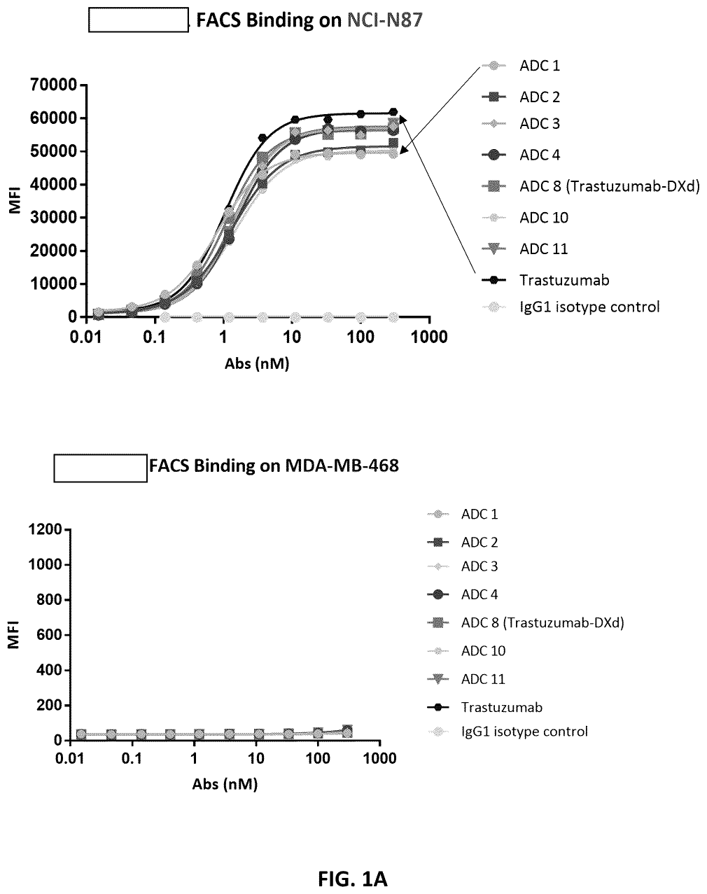

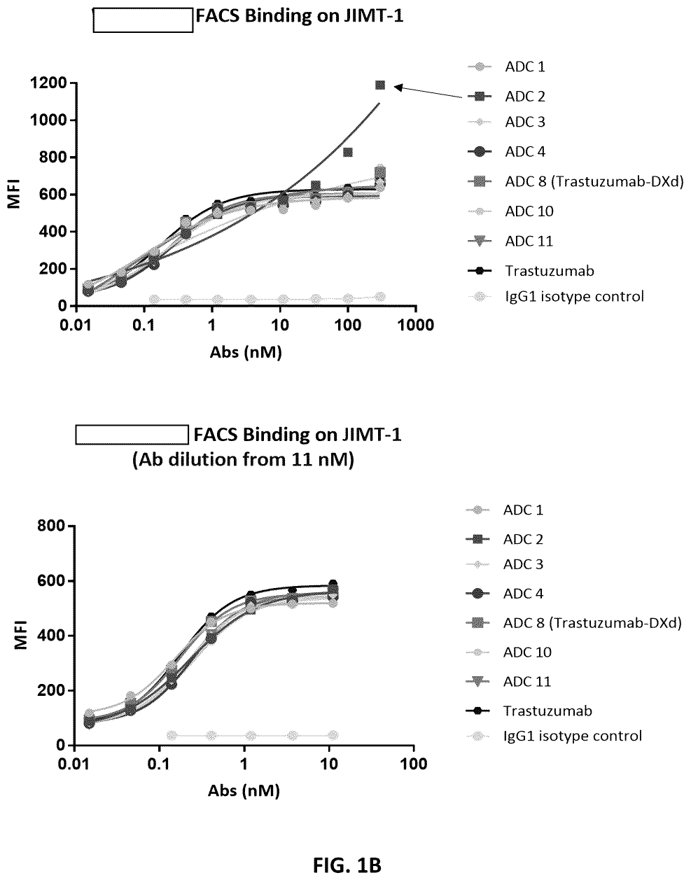

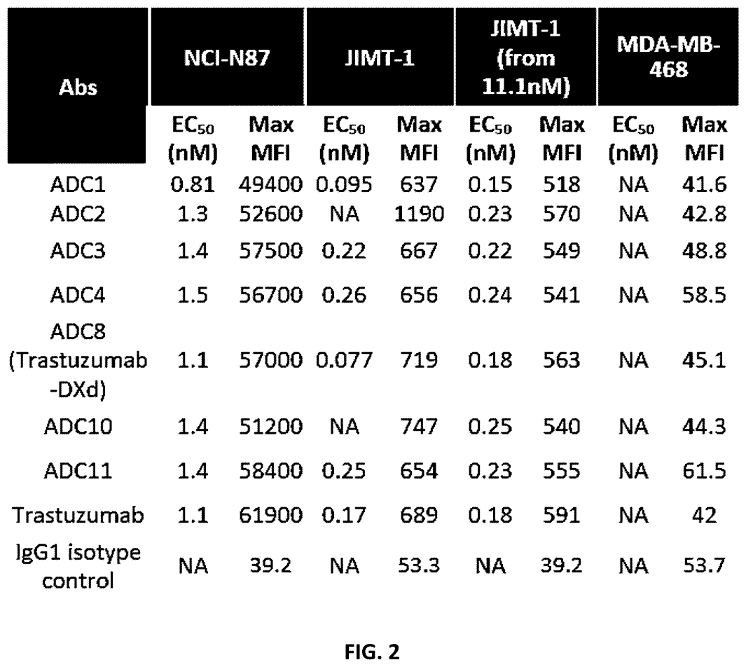

The effect of illustrative ADCs in cell lines with various HER2 expression levels were studied. The cell lines were NCI-N87 (gastric carcinoma cell line, which has high HER2 levels); MDA-MB-468 (breast cancer cell line, which is HER2 negative), and JIMT-1 (breast cancer cell line, which has low HER2 levels). Binding data for the ADCs to existing HER2 in each cell line is exemplified by FACS binding curves in FIGS. 1A-1B. Selected results obtained are summarized in FIG. 2. The ADCs tested showed comparable affinities in this study.

### Plasma Stability Studies of ADCs

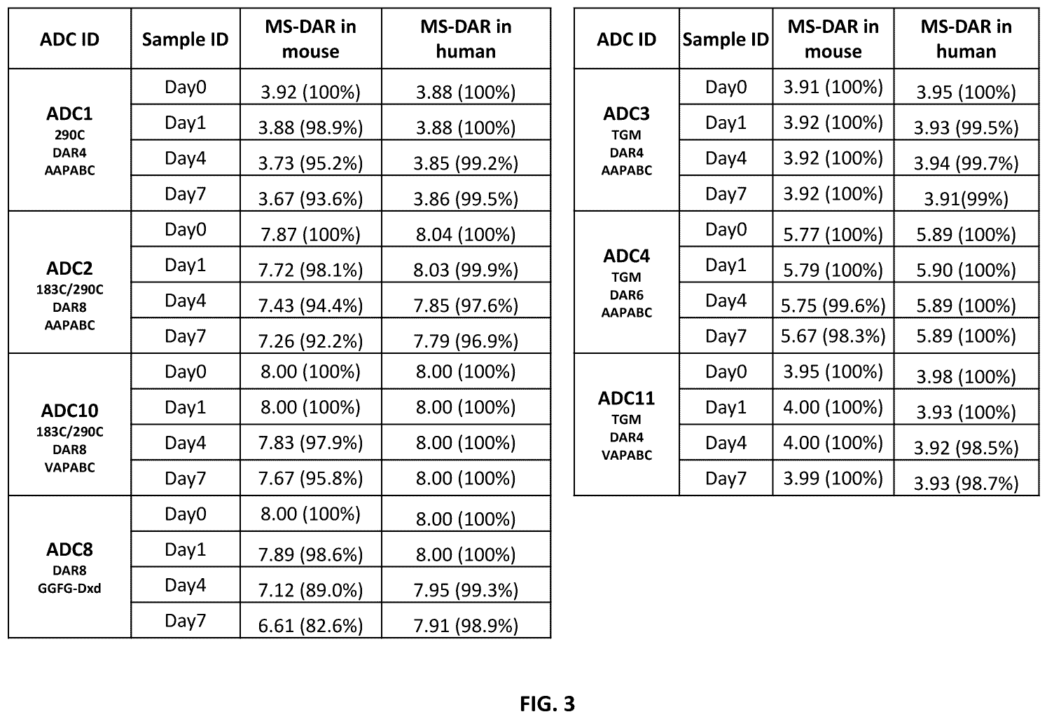

The plasma stability of illustrative ADCs was studied. For such studies, the ADC of interest and plasma (either mouse or human) were mixed at a 1:9 (v/v) ratio. The final concentration of the ADC in these mixtures was set at 0.2 mg/mL. The ADC-plasma mixture was incubated at 37° C. for 0, 1, 4, or 7 days. The ADC was then separated from the plasma using microbeads, and the DAR for the ADC was analyzed using mass spectrometry. Selected results of these experiments are summarized in FIG. 3.

All ADCs were found to be stable in human plasma for at least 7 days.

After incubation in mouse plasma, DAR values slightly declined after 7 days for ADC1, ADC2, ADC8 and ADC10. Interestingly, ADC8 (Trastuzumab deruxtecan) showed higher instability than all other ADCs in mouse plasma.

The results showed that transglutaminase conjugation is more stable than maleimide conjugation. Further, the VAPABC linker was found to be more stable than the AAPABC linker.

### Cytotoxicity Studies of ADCs

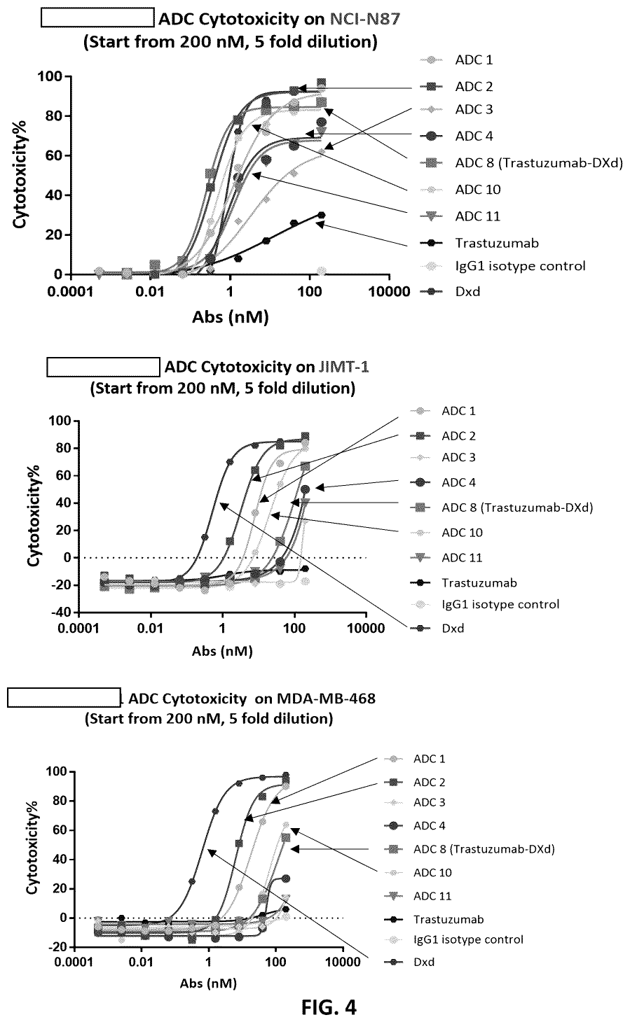

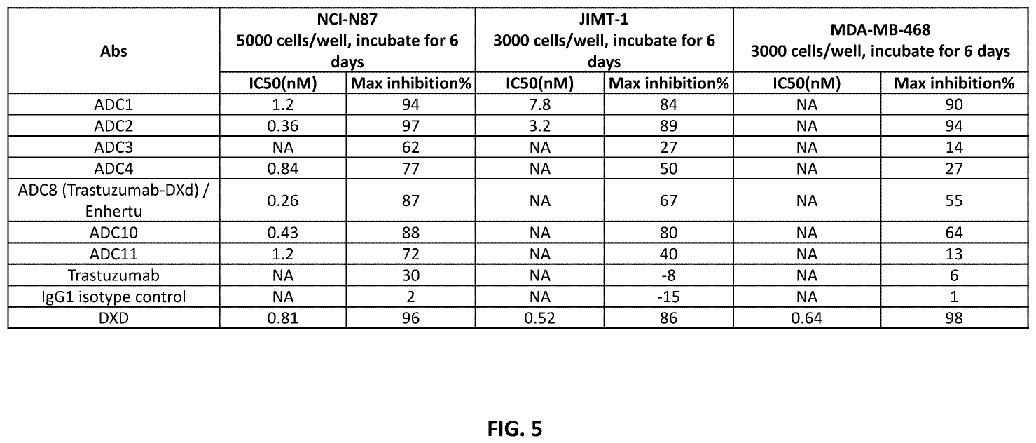

The cytotoxicity of illustrative ADCs was investigated in cell lines with various HER2 expression levels: NCI-N87 (gastric carcinoma cell line, which has high HER2 levels); MDA-MB-468 (breast cancer cell line, which is HER2 negative), and JIMT-1 (breast cancer cell line, which has low HER2 levels). The resulting cytotoxicity curves are illustrated in FIG. 4, and selected results are illustrated in FIG. 5.

The illustrative ADCs showed potent cytotoxicity on Her2^{high} N87 cells and lower cytotoxicity on Her21^{low} JIMT-1 cells by internalizing the ADC. Without wishing to be limited by any theory, cytotoxicity on Her2 negative MDA-MB-468 can be caused by slow release of payload from ADCs in cell culture. Cytotoxicity decreased in the following order: ADC1 & ADC2>ADC8 (Trastuzumab deruxtecan) & ADC10>ADC3, ADC4, ADC11 (which was very low). Without wishing to be limited by any theory, these results are consistent with mouse plasma stability test. Without wishing to be limited by any theory, cytotoxicity of ADC1, ADC2, ADC8 & ADC10 to MBA-MB-468 is caused by release of payload from ADCs.

The results indicated that VAPABC linker had better efficacy than the AAPABC linker.

Compared with ADC8 (Trastuzumab deruxtecan), ADC10 showed comparable potent cytotoxicity on N87, JIMT-1, and MDA-MB-468. ADC10 (DAR8) & ADC11 (DAR4) are selected for further in vivo efficacy study along with ADC8.

### Example 4: In Vitro Efficacy Studies

### JIMT-1 in CB-17 SCID Female Model

ADC8, ADC10, and ADC11 were used in this study. The mouse strain was CB-17 SCID, and the cell line was JIMT-1. 5×10⁶ cells/0.2 mL DPBS (Dulbecco's Phosphate-Buffered Saline) with 1:1 Matrigel were administered subcutaneous (S.C.) to the animals. There were 8 animals per group, and the tumors were allowed to grow to about 147 mm³. The ADC was then administered i.v. at day 0 and 7, at dosages of 5 mg/kg, 10 mg/kg, or 20 mg/kg. The animals were measured twice a week (BIW) for tumor volume and body weight. Data analysis was performed using GraphPad and two-way ANOVA.

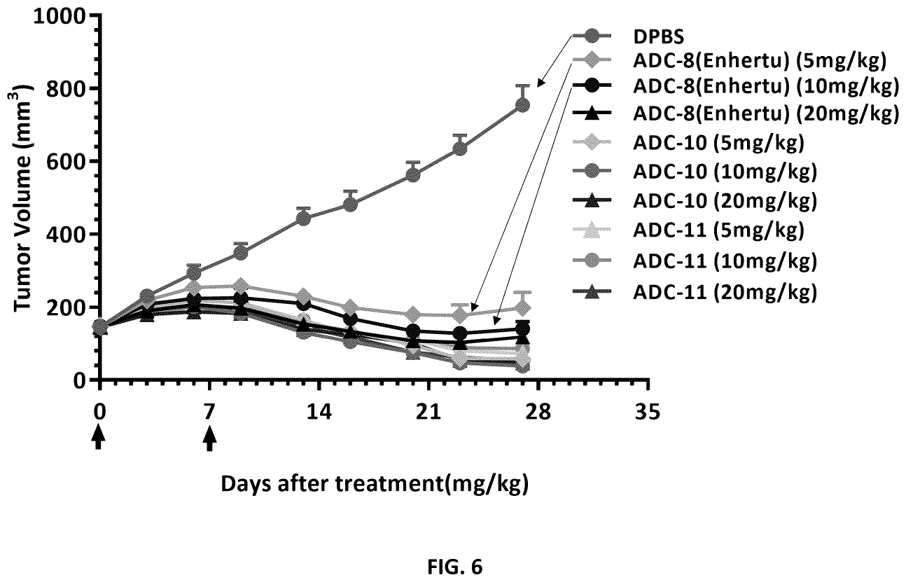

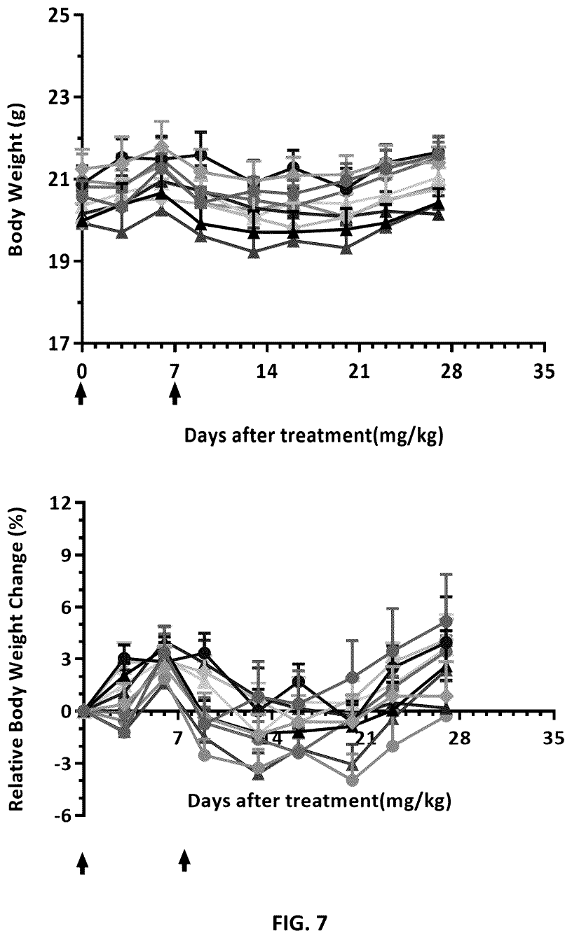

Tumor volume results are illustrated in FIG. 6. Body weight results are illustrated in FIG. 7.

Tumor Growth Inhibition (TGI_{TV}) is defined as:

TGI_{TV} (%)=[1−(Tᵢ−T₀)/(Vᵢ−V₀)]×100%

Tᵢ refers to the mean tumor volume of treatment group measured at each indicated time points following treatment; T₀ refers to the tumor volume of treatment group when grouping; Vᵢ refers to the mean tumor volume of control group measured at each indicated time points following treatment; V₀ refers to the tumor volume of control group when grouping. Results are illustrated in Table 4. ADCs of the disclosure consistently showed better TGI_{TV} values than ADC8 at equivalent doses.

TABLE 4

| | TGI (%) | V.S. ADC-8 (Enhertu) | | |
|---|---|---|---|---|
| Group (mg/kg) | (Day 27) | 5 mpk | 10 mpk | 20 mpk |
| DPBS | — | — | — | — |
| ADC-8(Enhertu) (5 mg/kg) | 91.48* | — | — | — |
| ADC-8(Enhertu) (10 mg/kg) | 101.02** | — | — | — |
| ADC-8(Enhertu) (20 mg/kg) | 104.81* | — | — | — |
| ADC-10 (5 mg/kg) | 115.00** | **(<0.001) | — | — |
| ADC-10 (10 mg/kg) | 118.08** | — | **(<0.001) | — |
| ADC-10 (20 mg/kg) | 116.19** | — | — | **(<0.001) |
| ADC-11 (5 mg/kg) | 112.71** | **(<0.001) | — | — |
| ADC-11 (10 mg/kg) | 110.08** | — | **(0.008) | — |
| ADC-11 (20 mg/kg) | 115.66** | — | — | **(<0.001) |

| | |
|---|---|
| test group | test group vs ADC-8 |
| vs DPBS | *P < 0.05, **P < 0.01 |

### NCI-N87 in Balb/c Nude Female Model

ADC8, ADC10, and ADC11 were used in this study. The mouse strain was Balb/c nude mice, and the cell line was NCI-N87. 5×10⁶ cells/0.2 mL DPBS with 1:1 Matrigel were administered subcutaneous (S.C.) to the animals. There were 8 animals per group, and the tumors were allowed to grow to about 184 mm³. The ADC was then administered i.v., single dose at day 0, at dosages of 0.5 mg/kg, 1 mg/kg, 2 mg/kg, or 4 mg/kg. The animals were measured twice a week (BIW) for tumor volume and body weight. Data analysis was performed using GraphPad and two-way ANOVA.

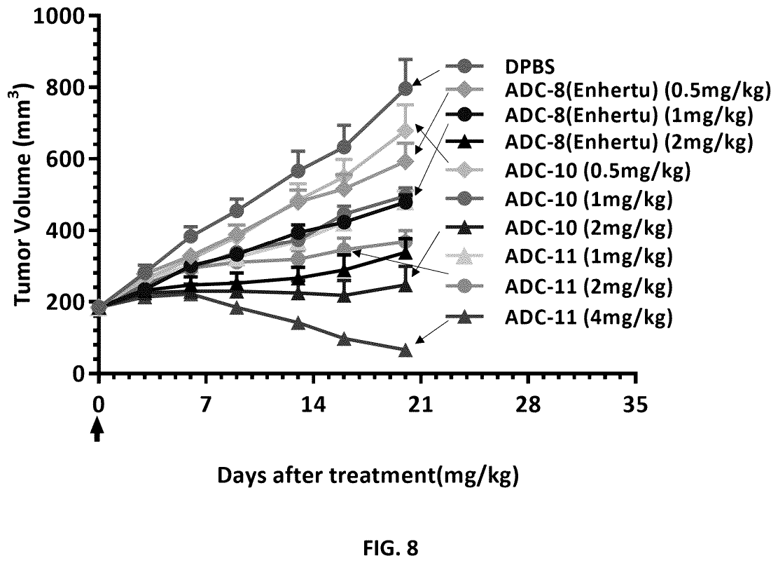

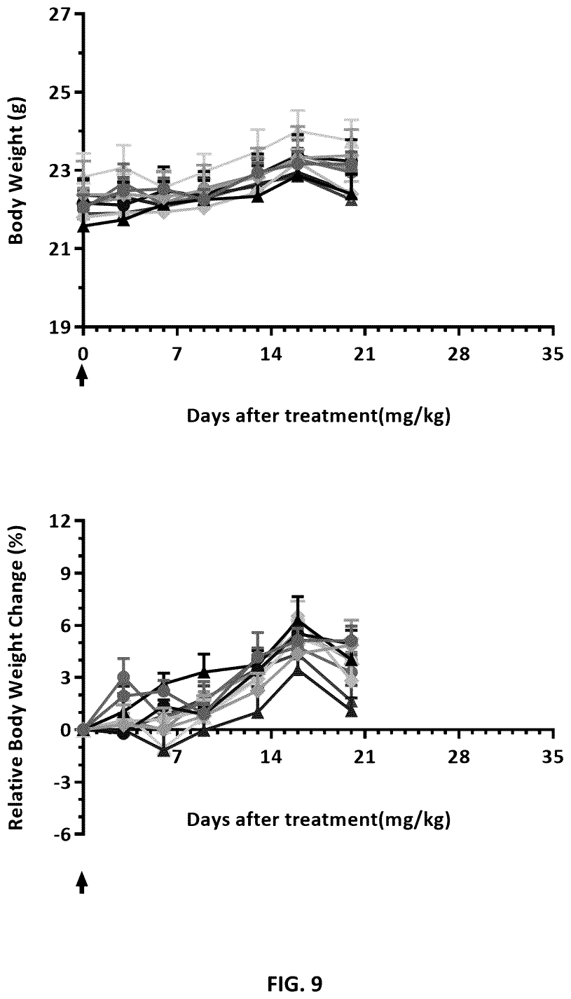

Tumor volume results are illustrated in FIG. 8. Body weight results are illustrated in FIG. 9.

Tumor Growth Inhibition (TGI_{TV}) are illustrated in Table 5. ADCs of the disclosure consistently showed better TGI_{TV} values than ADC8 at equivalent doses.

TABLE 5

| | TGI (%) | V.S. ADC-8 (Enhertu) | | |
|---|---|---|---|---|
| Group (mg/kg) | (Day 20) | 0.5 mpk | 1 mpk | 2 mpk |
| DPBS | — | — | — | — |
| ADC-8(Enhertu) (0.5 mg/kg) | 33.21** | — | — | — |
| ADC-8(Enhertu) (1 mg/kg) | 51.90** | — | — | — |
| ADC-8(Enhertu) (2 mg/kg) | 74.86* | — | — | — |
| ADC-10 (0.5 mg/kg) | 19.18* | (0.104) | — | — |
| ADC-10 (1 mg/kg) | 49.00* | — | (0.745) | — |
| ADC-10 (2 mg/kg) | 89.81** | — | — | *(0.021) |
| ADC-11 (1 mg/kg) | 51.49** | *(0.025) | — | — |
| ADC-11 (2 mg/kg) | 70.13* | — | **(<0.001) | — |
| ADC-11 (4 mg/kg) | 119.44** | — | — | **(<0.001) |

| | |
|---|---|
| test group | test group vs ADC-8 |
| vs DPBS | *P < 0.05, **P < 0.01 |

### ENUMERATED EMBODIMENTS

The following exemplary embodiments are provided, the numbering of which is not to be construed as designating levels of importance:

Embodiment 1: A ligiid-payload conjugate of formula (I):

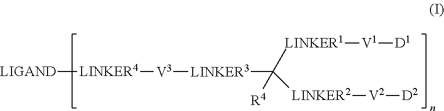

or a pharmaceutically acceptable salt thereof, wherein:
LIGAND is a ligand;
each of V¹, V², and V³ is independently a linker moiety formed by a coupling reaction; each of LINKER¹, LINKER², LINKER³, and LINKER⁴ is independently a bivalent linker; each of D¹ and D² is independently a payload;
each R⁴ is independently R, —N(R)₂, or —N(R)C(O)—R;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur; and
n is 2, 3, 4, 5, or 6.

Embodiment 2: A ligand-payload conjugate of formula (I*):

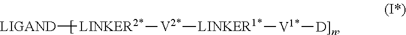

or a pharmaceutically acceptable salt thereof, wherein:
LIGAND is a ligand;
each of V¹* and V²* is independently a linker moiety formed by a coupling reaction;
each of LINKER¹* and LINKER²* is independently a bivalent linker;
each D is independently a payload;
n is 4, 5, 6, 7, 8, 9, 10, 11, or 12.

Embodiment 3: The ligand-payload conjugate of any one of Embodiments 1-2, wherein the LIGAND is an antibody.

Embodiment 4: The ligand-payload conjugate of Embodiment 3, wherein the antibody is selected from the group consisting of Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96, and Glembatumumab, or antigen-binding fragments thereof.

Embodiment 5: The ligand-payload conjugate of any one of Embodiments 3-4, wherein the LIGAND is Trastuzumab.

Embodiment 6: The ligand-payload conjugate of any one of Embodiments 3-5, wherein the light chain of the antibody comprises the amino acid sequence of any of SEQ ID Nos: 34, 37, or 39, and/or wherein the heavy chain of the antibody comprises the amino acid sequence of any of SEQ ID Nos: 35, 36, and 38.

Embodiment 7: The ligand-payload conjugate of any one of Embodiments 1 and 3-6,
wherein each LINKER⁴ is S⁴, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

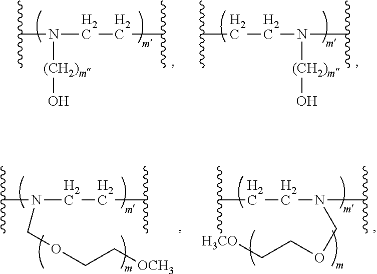

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein:
each -Cy- is independently an optionally substituted 3-8 membered bivalent saturated, partially unsaturated, or aromatic ring having 0-4 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

Embodiment 8: The ligand-payload conjugate of any one of Embodiments 1 and 3-7,
wherein each V³ is independently selected from the group consisting of: —C(O)—NH—, —NH—C(O)—,

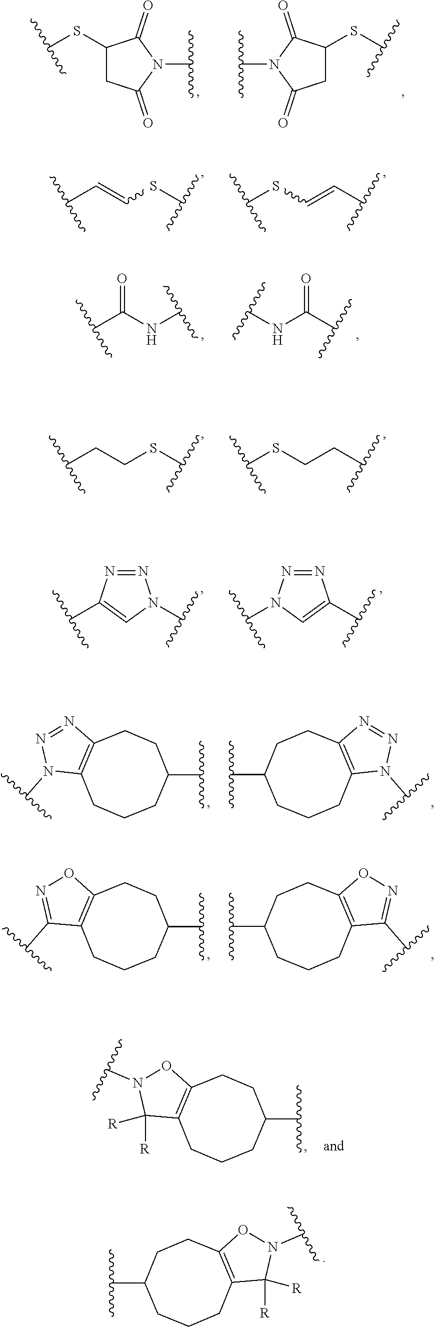

Embodiment 9: The ligand-payload conjugate of any one of Embodiments 1 and 3-8,
wherein each V³ is independently selected from the group consisting of:
—C(O)—NH—, —NH—C(O)—,

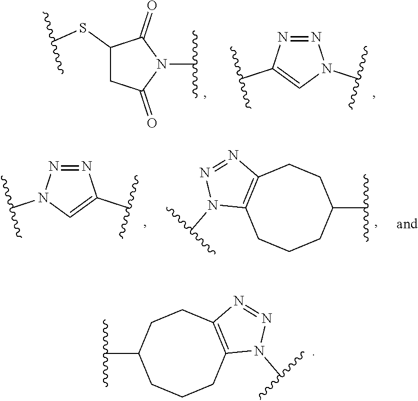 [SMILES: *SC1CC(=O)N(*)C1=O ; *c1cn(*)nn1 ; *c1cn(*)nn1 ; *C1CCCc2c(nnn2*)CC1 ; *C1CCCc2c(nnn2*)CC1]

Embodiment 10: The ligand-payload conjugate of any one of Embodiments 1 and 3-9,
wherein each LINKER³ is independently

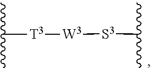

wherein:
each of S³ and T³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

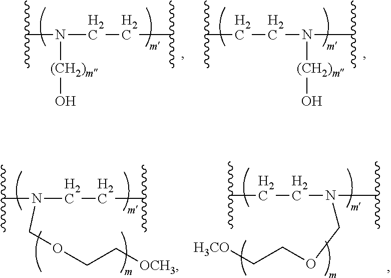

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—; each W³ is independently —C(O)—N(R³)— or —N(R³)—C(O)—;
each -Cy- is independently an optionally substituted 3-8 membered bivalent saturated, partially unsaturated, or aromatic ring having 0-4 heteroatoms independently selected from nitrogen, oxygen, or sulfur;
each R³ is independently H or optionally substituted C₁₋₆ aliphatic;
each m″ is independently 1, 2, or 3; and
each of m and m′ is independently an integer from 0-24.

Embodiment 11: The ligand-payload conjugate of Embodiment 10, wherein T³ is

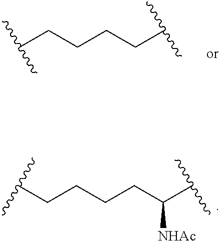 [SMILES: *CCCC* ; *CCCC[C@@H](*)NC(C)=O]

Embodiment 12: The ligand-payload conjugate of any one of Embodiments 10-11, wherein W³ is —C(O)—NH—.

Embodiment 13: The ligand-payload conjugate of any one of Embodiments 10-12, wherein S³ is a covalent bond.

Embodiment 14: The ligand-payload conjugate of any one of Embodiments 1 and 3-13,
wherein each LINKER¹ is independently

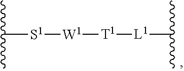

wherein:
each S¹ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

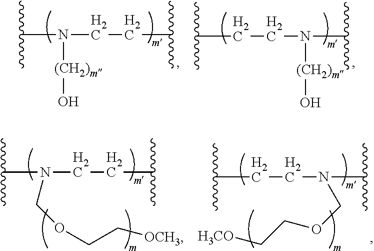

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

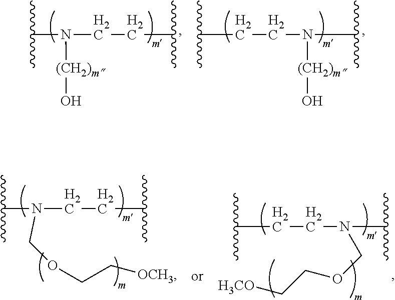

or W¹ and T¹ are absent;
each L¹ is independently —C(O)-P¹-X¹— or —N(R¹)-P¹-X¹—, wherein each P¹ is absent or a peptide comprising 1-20 amino acids, and each X¹ is a cleavable linker;
each of R¹ and R³ is independently H or optionally substituted C₁₋₆ aliphatic;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

Embodiment 15: The ligand-payload conjugate of Embodiment 14, wherein S¹ is —CH₂CH₂—.

Embodiment 16: The ligand-payload conjugate of any one of Embodiments 14-15, wherein W¹ is —C(O)—NH—.

Embodiment 17: The ligand-payload conjugate of any one of Embodiments 14-16, wherein T¹ is

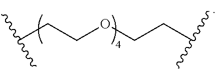

Embodiment 18: The ligand-payload conjugate of any one of Embodiments 14-17, wherein L¹ is —C(O)-P¹-X¹—.

Embodiment 19: The ligand-payload conjugate of any one of Embodiments 14-18, wherein P¹ is a peptide having 2 amino acids (a dipeptide).

Embodiment 20: The ligand-payload conjugate of any one of Embodiments 14-19, wherein P¹ is a dipeptide selected from -(L-ala)-(L-ala)-, -Val-Cit-, and -Val-Ala-, or a tripeptide selected from -AcLys-Val-Cit- and -Glu-Val-Cit-, or a tetrapeptide -Gly-Gly-Phe-Gly-.

Embodiment 21: The ligand-payload conjugate of any one of Embodiments 14-20, wherein X¹ is a PAB group

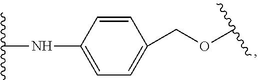 [SMILES: *Nc1ccc(CO*)cc1]

which is linked to P¹ via the amino nitrogen atom of the PAB group, and linked to V¹ via the oxygen atom of the PAB group.

Embodiment 22: The ligand-payload conjugate of any one of Embodiments 1 and 3-21,
wherein each LINKER² is independently

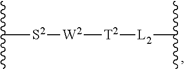

wherein:
S² is a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

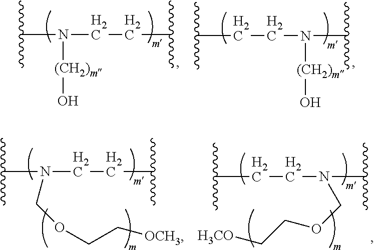

—S—, —N(R)—, —O—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(CH₂) m (CH₂)ₘ,

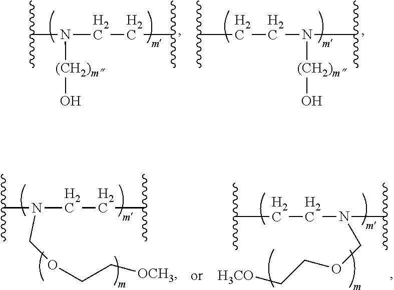

or W² and T² are absent;
L² is —C(O)-P²-X²— or —N(R²)-P²-X²—, wherein each P² is absent or a peptide comprising 1-20 amino acids, and each X² is a cleavable linker;
each of R² and R³ is independently H or optionally substituted C₁₋₆ aliphatic;
each m″ is independently 1, 2, or 3; and
each of m and m′ is independently an integer from 0-24.

Embodiment 23: The ligand-payload conjugate of Embodiment 22, wherein S² is a covalent bond.

Embodiment 24: The ligand-payload conjugate of any one of Embodiments 22-23, wherein W² is —C(O)—NH—.

Embodiment 25: The ligand-payload conjugate of any one of Embodiments 22-24, wherein T² is

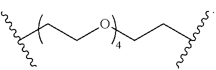

Embodiment 26: The ligand-payload conjugate of any one of Embodiments 22-25, wherein L² is —C(O)-P²-X²—.

Embodiment 27: The ligand-payload conjugate of any one of Embodiments 22-26, wherein P² is a peptide having 2 amino acids (a dipeptide).

Embodiment 28: The ligand-payload conjugate of any one of Embodiments 22-27, wherein P² is a dipeptide selected from -(L-ala)-(L-ala)-, -Val-Cit-, and -Val-Ala-.

Embodiment 29: The ligand-payload conjugate of any one of Embodiments 22-28,
wherein X² is a PAB group

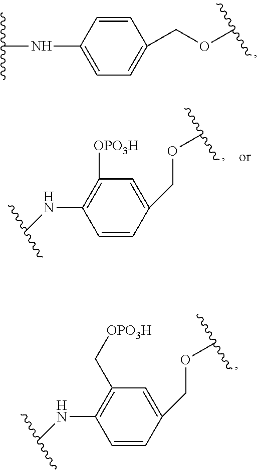

which is linked to P² via the amino nitrogen atom of the PAB group, and linked to V² via the oxygen atom of the PAB group.

Embodiment 30: The ligand-payload conjugate of any one of Embodiments 1 and 3-29, wherein each of D¹ and D² is independently selected from anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, and a pladienolide, or stereoisomers, isosteres, analogs, or derivatives thereof.

Embodiment 31: The ligand-payload conjugate of any one of Embodiments 1 and 3-30, wherein D¹ and D² are exatecan

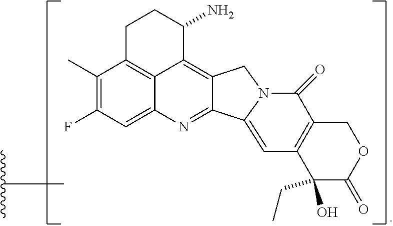

Embodiment 32: The ligand-payload conjugate of any one of Embodiments 1 and 3-31, wherein n is 2, 3, or 4.

Embodiment 33: The ligand-payload conjugate of any one of Embodiments 1 and 3-32, which is a compound of Formula (I-11):

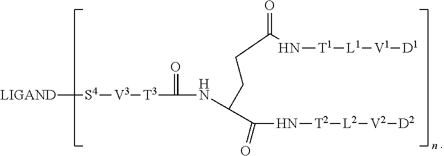

Embodiment 34: The ligand-payload conjugate of any one of Embodiments 1 and 3-33, which is a compound of Formula (I-14) or (I-15):

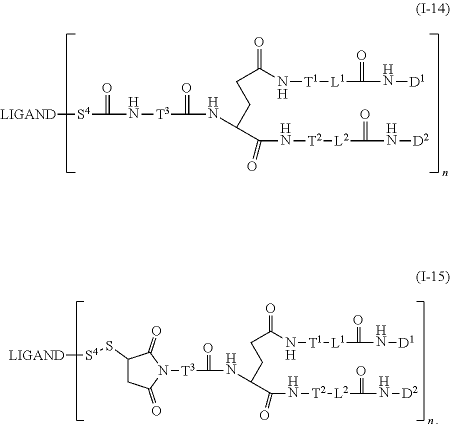

Embodiment 35: The ligand-payload conjugate of any one of Embodiments 2-6, wherein each LINKER²* is S²*, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

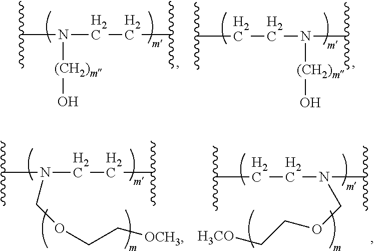

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein:
each -Cy- is independently an optionally substituted 3-8 membered bivalent saturated, partially unsaturated, or aromatic ring having 0-4 heteroatoms independently selected from nitrogen, oxygen, or sulfur;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

Embodiment 36: The ligand-payload conjugate of any one of Embodiments 2-6 and 35,
wherein each V²* is independently selected from: —C(O)—NH—, —NH—C(O)—,

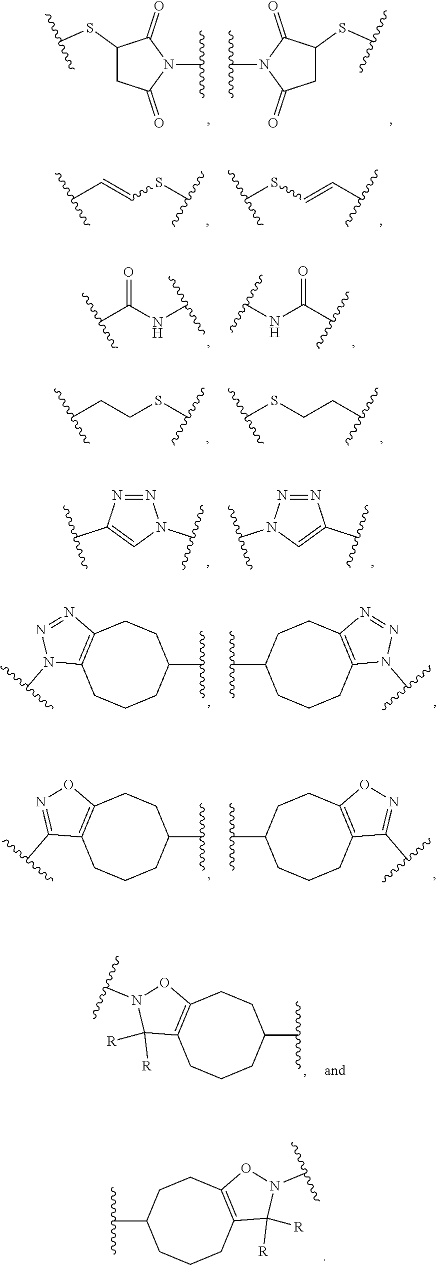

Embodiment 37: The ligand-payload conjugate of any one of Embodiments 2-6 and 35-36,
wherein each V²* is independently selected from:
C(O)—NH—, —NH—C(O)—,

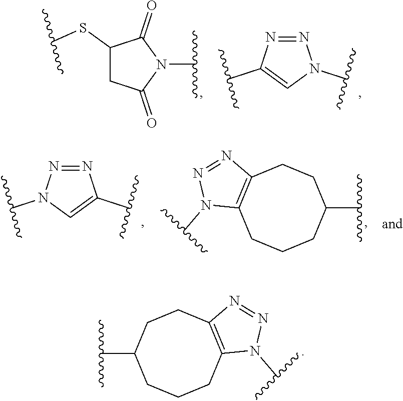

Embodiment 38: The ligand-payload conjugate of any one of Embodiments 2-6 and 35-37,
wherein each LINKER¹* is independently

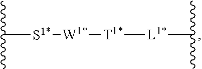

wherein:
each S¹* is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

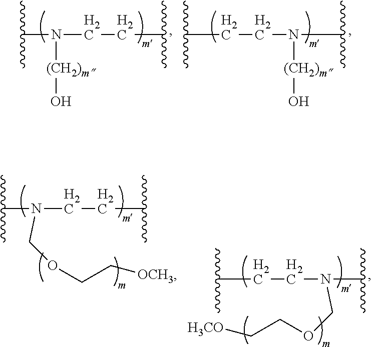

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹* is independently —C(O)—N(R³*)— or —N(R³*)—C(O)—, and T¹* is independently a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

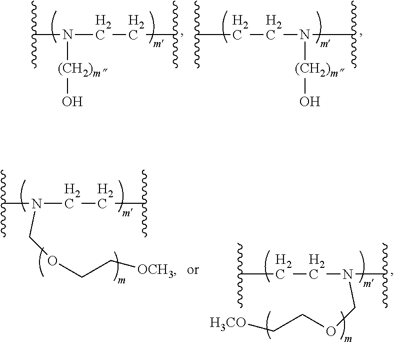

or W¹* and T¹* are absent;
each L¹* is independently —C(O)-P¹*-X¹*— or —N(R¹*)-P¹*-X¹*—, wherein each P¹* is absent or a peptide comprising 1-20 amino acids, and each X¹* is a cleavable linker;
each of R¹* and R³* is independently H or optionally substituted C₁₋₆ aliphatic;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

Embodiment 39: The ligand-payload conjugate of Embodiment 38, wherein S¹* is

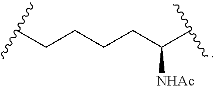 [SMILES: *CCCC[C@@H](*)NC(C)=O]

Embodiment 40: The ligand-payload conjugate of any one of Embodiments 38-39, wherein W¹* is —C(O)—NH—.

Embodiment 41: The ligand-payload conjugate of any one of Embodiments 38-40, wherein T¹* is

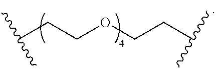

Embodiment 42: The ligand-payload conjugate of any one of Embodiments 38-41, wherein L¹* is —C(O)-P¹*-X¹*—.

Embodiment 43: The ligand-payload conjugate of any one of Embodiments 38-42, wherein P¹* is a peptide having 2 amino acids (a dipeptide).

Embodiment 44: The ligand-payload conjugate of any one of Embodiments 38-43, wherein P¹* is a dipeptide selected from -(L-ala)-(L-ala)-, -Val-Cit-, and -Val-Ala-, or a tripeptide selected from -AcLys-Val-Cit- and -Glu-Val-Cit-, or a tetrapeptide -Gly-Gly-Phe-Gly-.

Embodiment 45: The ligand-payload conjugate of any one of Embodiments 38-44, wherein X¹* is a PAB group

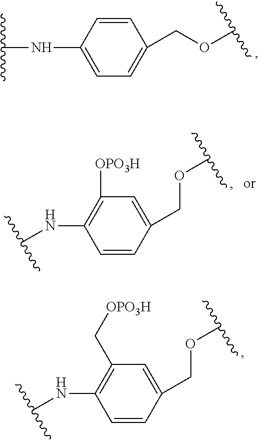

which is linked to P¹* via the amino nitrogen atom of the PAB group, and linked to V¹* via the oxygen atom of the PAB group.

Embodiment 46: The ligand-payload conjugate of any one of Embodiments 2-6 and 35-45, wherein each D is independently selected from anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, and a pladienolide, or stereoisomers, isosteres, analogs, or derivatives thereof.

Embodiment 47: The ligand-payload conjugate of any one of Embodiments 2-6 and 35-46, wherein each D is exatecan

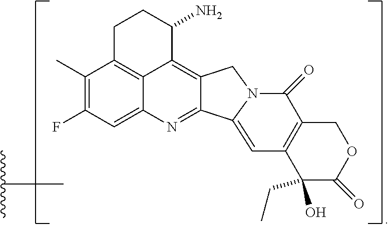

Embodiment 48: The ligand-payload conjugate of any one of Embodiments 2-6 and 35-47, wherein n is 4, 6, or 8.

Embodiment 49: The ligand-payload conjugate of any one of Embodiments 2-6 and 35-48, which is a compound of Formula (I-6*):

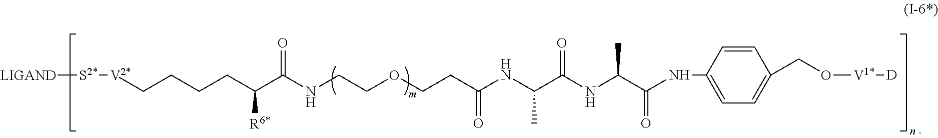

Embodiment 50: The ligand-payload conjugate of any one of Embodiments 2-6 and 35-49, which is a compound of Formula (I-6*):

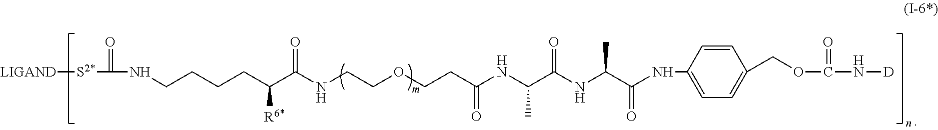

Embodiment 51: The ligand-payload conjugate of any one of Embodiments 1-50, which is selected from the group consisting of ADC1, ADC2, ADC3, ADC4, ADC10, and ADC11.

Embodiment 52: A linker-payload compound selected from the group consisting of:
(a) a linker-payload compound of formula (II):

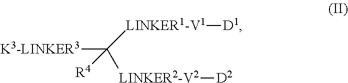

or a pharmaceutically acceptable salt thereof,
wherein:
K³ is a functional group capable of forming a linker moiety by a coupling reaction with a different functional group;
each of V¹ and V² is independently a linker moiety formed by a coupling reaction; each of LINKER¹, LINKER², and LINKER³ is independently a bivalent linker; each of D¹ and D² is independently a payload;
each R⁴ is independently R, —N(R)₂, or —N(R)C(O)—R; and
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
(b) a linker-payload compound of formula (II*):

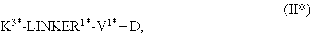

or a pharmaceutically acceptable salt thereof, wherein:
K³* is a functional group capable of forming a linker moiety by a coupling reaction with a different functional group;
V¹* is a linker moiety formed by a coupling reaction;
LINKER¹* is independently a bivalent linker; and
D is independently a payload.

Embodiment 53: A linker-payload compound selected from the group consisting of:
(a) a linker-payload compound of formula (IV-1):

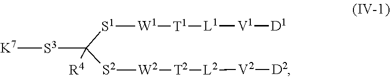

or a pharmaceutically acceptable salt thereof, wherein:
each K⁷ is independently a functional group capable of forming a carboxamide linker moiety by a coupling reaction with a different functional group;
each of S¹, S², and S³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

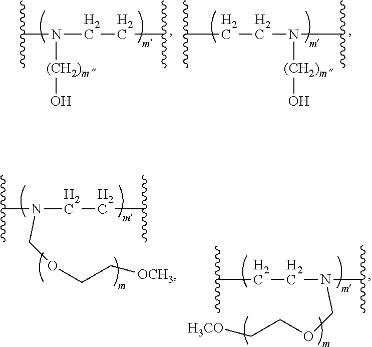

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ,

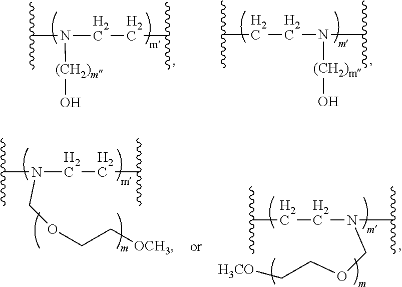

or W¹ and T¹ are absent;
each W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

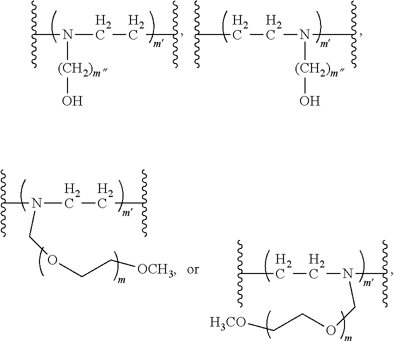

or W² and T² are absent;
each L¹ is independently —C(O)-P¹-X¹— or —N(R¹)-P¹-X¹—, wherein each P¹ is absent or a peptide comprising 1-20 amino acids, and each X¹ is a cleavable linker; each L² is independently —C(O)-P²-X²— or —N(R²)_P²-X²—, wherein each P² is absent or a peptide comprising 1-20 amino acids, and each X² is a cleavable linker; each of V¹ and V² is independently a linker moiety formed by a coupling reaction; each of R¹, R², and R³ is independently H or optionally substituted C₁₋₆ aliphatic; each R⁴ is independently R, —N(R)₂, or —N(R)C(O)—R;
each of D¹ and D² is independently a payload;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently an integer from 0-24;
(b) a linker-payload compound of formula (IV-1*):

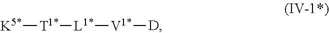

or a pharmaceutically acceptable salt thereof, wherein:
K⁵* is a functional group capable of forming a carboxamide linker moiety by a coupling reaction with a different functional group;
T¹* is a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

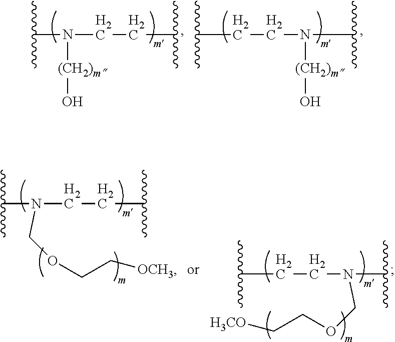

each -Cy- is independently an optionally substituted 3-8 membered bivalent saturated, partially unsaturated, or aromatic ring having 0-4 heteroatoms independently selected from nitrogen, oxygen, or sulfur;
L¹* is —C(O)-P¹*-X¹*— or —N(R¹*)-P¹*-X¹*—, wherein each P¹* is absent or a peptide comprising 1-20 amino acids, and each X¹* is a cleavable linker;
V¹* is a linker moiety formed by a coupling reaction;
each R¹* is independently H or optionally substituted C₁₋₆ aliphatic;
each D is a payload;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2 3, 4, 5, 6, 7, 8, 9, 10, 11, 12, 13, 14, 15, 16, 17, 18, 19, 20, 21, 22, 23, or 24.

Embodiment 54: A linker-payload compound selected from the group consisting of:
(a) a linker-payload compound of formula (VI-1):

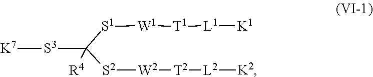

or a pharmaceutically acceptable salt thereof, wherein:
each of K¹ and K² is independently a functional group capable of forming a linker moiety by a coupling reaction with a different functional group;
each K⁷ is independently a functional group capable of forming a carboxamide linker moiety by a coupling reaction with a different functional group;
each of S¹, S², and S³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—,

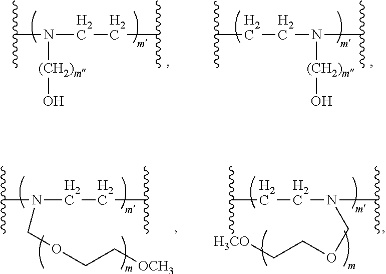

—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

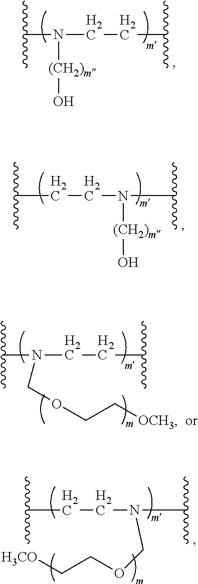

or W¹ and T¹ are absent;
each W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

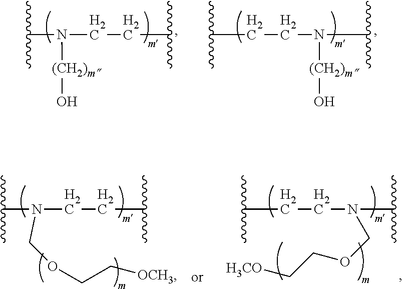

or W² and T² are absent;
each L¹ is independently —C(O)-P¹-X¹— or —N(R¹)-P¹-X¹—, wherein each P¹ is absent or a peptide comprising 1-20 amino acids, and each X¹ is a cleavable linker; each L² is independently —C(O)-P²-X²— or —N(R²)_P²-X²—, wherein each P² is absent or a peptide comprising 1-20 amino acids, and each X² is a cleavable linker;
each of R¹, R², and R³ is independently H or optionally substituted C₁₋₆ aliphatic;
each R⁴ is independently R, —N(R)₂, or —N(R)C(O)—R;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2 3, 4, 5, 6, 7, 8, 9, 10, 11, 12, 13, 14, 15, 16, 17, 18, 19, 20, 21, 22, 23, or 24;
(b) a linker-payload compound of formula (VI-1*):

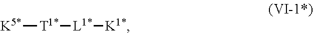

or a pharmaceutically acceptable salt thereof, wherein:
K¹* is a functional group capable of forming a linker moiety by a coupling reaction with a different functional group;
K⁵* is a functional group capable of forming a carboxamide linker moiety by a coupling reaction with a different functional group;
T¹* is a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—,

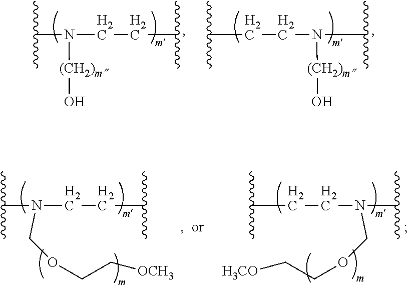

each -Cy- is independently an optionally substituted 3-8 membered bivalent saturated, partially unsaturated, or aromatic ring having 0-4 heteroatoms independently selected from nitrogen, oxygen, or sulfur;
each L¹* is independently —C(O)-P¹*-X¹*— or —N(R¹*)-P¹*-X¹*—, wherein each P¹* is absent or a peptide comprising 1-20 amino acids, and each X¹* is a cleavable linker;
each R¹ is independently H or optionally substituted C₁₋₆ aliphatic;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2 3, 4, 5, 6, 7, 8, 9, 10, 11, 12, 13, 14, 15, 16, 17, 18, 19, 20, 21, 22, 23, or 24.

Embodiment 55: A method of treating, ameliorating, and/or preventing a disorder, disease, or condition in a patient in need thereof, the method comprising administering to the patient a therapeutically effective amount of the ligand-payload conjugate of any one of Embodiments 1-54, or a pharmaceutically acceptable salt thereof.

Embodiment 56: The method of Embodiment 55, wherein the disorder, disease, or condition is at least one of a cancer, an autoimmune disease, and an infectious disease.

Embodiment 57: The method of any one of Embodiments 55-56, wherein the ligand-payload conjugate is administered to the patient as a pharmaceutical composition that further comprises at least one pharmaceutically acceptable carrier, adjuvant, and/or vehicle.

Embodiment 58: The method of any one of Embodiments 55-57, wherein the subject is further administered at least one additional agent to treat, ameliorate, and/or prevent the disorder, disease, or condition.

Embodiment 59: The method of Embodiment 58, wherein the ligand-payload conjugate and the at least one agent are co-administered.

Embodiment 60: The method of Embodiment 59, wherein the ligand-payload conjugate and the at least one agent are co-formulated.

While a number of embodiments of this disclosure is described, it is apparent that the basic examples may be altered to provide other embodiments that utilize the compounds and methods of this disclosure. Therefore, it will be appreciated that the scope of this disclosure is to be defined by the application and appended claims rather than by the specific embodiments that have been represented by way of example.

A number of references and publications have been cited, each of which is incorporated herein by reference in its entirety.

What is claimed is:

1-60. (canceled)

61. A ligand-payload conjugate of formula (I): 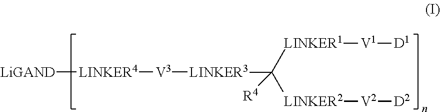
or a pharmaceutically acceptable salt thereof,
wherein:
LIGAND is a ligand;
each of V¹, V², and V³ is independently a linker moiety formed by a coupling reaction;
each of LINKER¹, LINKER², LINKER³, and LINKER⁴ is independently a bivalent linker;
each of D¹ and D² is independently a payload;
each R⁴ is independently R, —N(R)₂, or —N(R)C(O)—R;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur; and
n is 2, 3, 4, 5, or 6.

62. The ligand-payload conjugate of claim 61, wherein the LIGAND is an antibody.

63. The ligand-payload conjugate of any one of claim 61,
wherein each LINKER⁴ is S⁴, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ, (CH₂CH₂O)ₘ—, 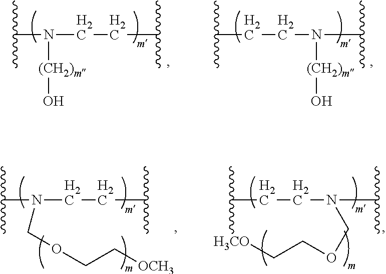
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)— wherein:
each -Cy- is independently an optionally substituted 3-8 membered bivalent saturated, partially unsaturated, or aromatic ring having 0-4 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

64. The ligand-payload conjugate of any one of claim 61,
wherein each V³ is independently selected from the group consisting of:
—C(O)—NH—, —NH—C(O)—,

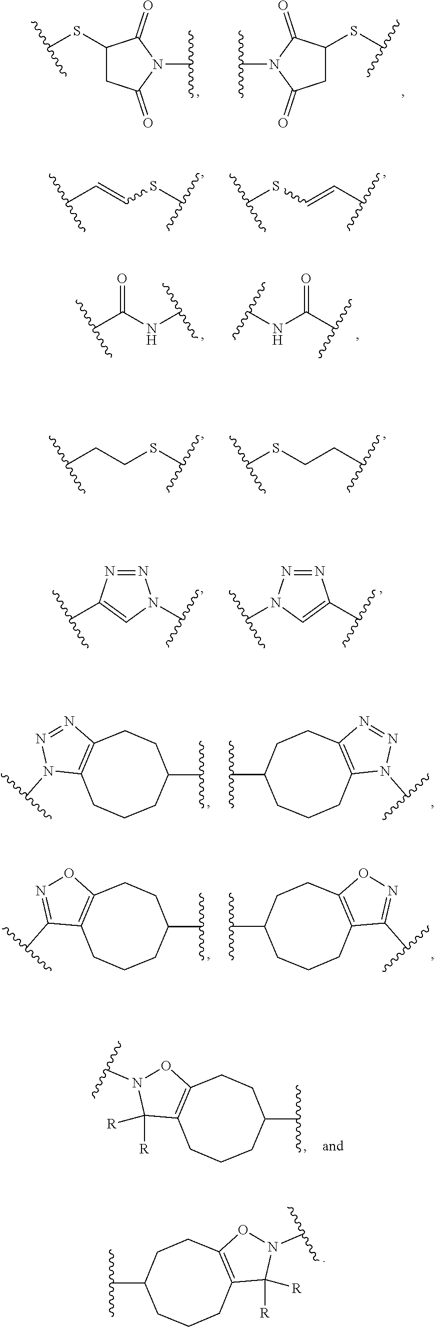

65. The ligand-pay load conjugate of claim 61,
wherein each LINKER³ is independently 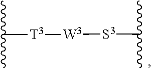
wherein:
each of S³ and T³ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—, 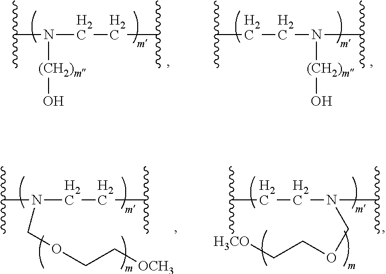
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each W³ is independently —C(O)—N(R³)— or —N(R³)—C(O)—;
each -Cy- is independently an optionally substituted 3-8 membered bivalent saturated, partially unsaturated, or aromatic ring having 0-4 heteroatoms independently selected from nitrogen, oxygen, or sulfur;
each R³ is independently H or optionally substituted C₁₋₆ aliphatic;
each m″ is independently 1, 2, or 3; and
each of m and m′ is independently an integer from 0-24.

66. The ligand-payload conjugate of claim 65, wherein:
(a) T³ is or 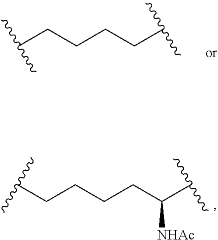 [SMILES: *CCCC* ; *CCCC[C@@H](*)NC(C)=O]
(b) W³ is —C(O)—NH—, or
(c) S³ is a covalent bond.

67. The ligand-payload conjugate of claim 61,
wherein each LINKER¹ is independently 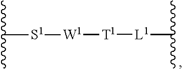
wherein:
each S¹ is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—, 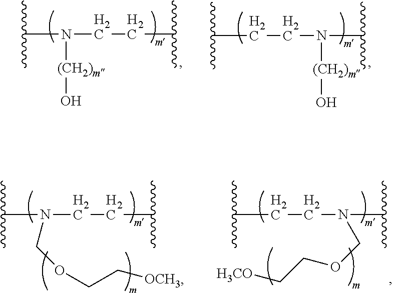
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹ is independently —C(O)—N(R³)— or —N(R³)—C(O)—, and T¹ is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —CH₂CH₂O)ₘ—, 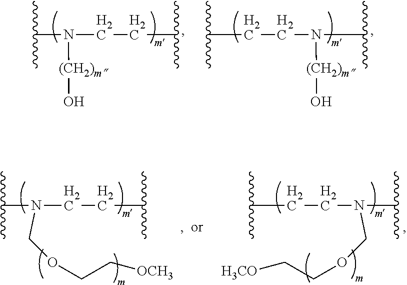
or W¹ and T¹ are absent;
each L¹ is independently —C(O)-P¹-X¹— or —N(R¹)-P¹-X¹—, wherein each P¹ is absent or a peptide comprising 1-20 amino acids, and each X¹ is a cleavable linker;
each of R¹ and R³ is independently H or optionally substituted C₁₋₆ aliphatic;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24.

68. The ligand-pay load conjugate of claim 67, wherein:
(a) S¹ is —CH₂CH₂—,
(b) W¹ is —C(O)—NH—,
(c) T¹ is 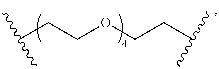
or
(d) L¹ is —C(O)-P¹-X¹—.

69. The ligand-payload conjugate of claim 67, wherein P¹ is a dipeptide selected from -(L-ala)-(L-ala)-, -Val-Cit-, and -Val-Ala-, or a tripeptide selected from -AcLys-Val-Cit- and -Glu-Val-Cit-, or a tetrapeptide -Gly-Gly-Phe-Gly-, and
wherein X¹ is a PAB group 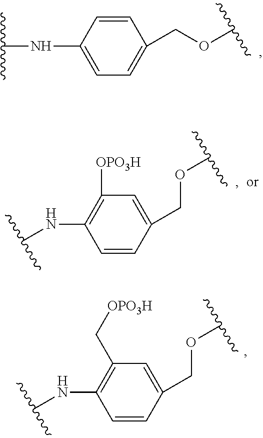
which is linked to P¹ via the amino nitrogen atom of the PAB group, and linked to V¹ via the oxygen atom of the PAB group.

70. The ligand-payload conjugate of claim 61,
wherein each LINKER² is independently 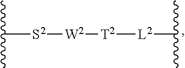
wherein:
S² is a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—, 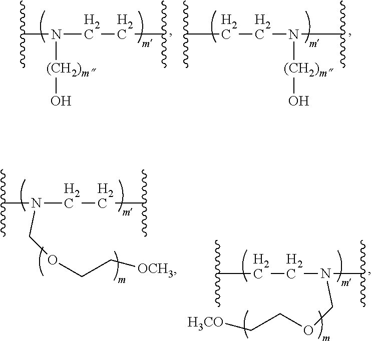
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
W² is independently —C(O)—N(R³)— or —N(R³)—C(O)—, and T² is independently a covalent bond or a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—, 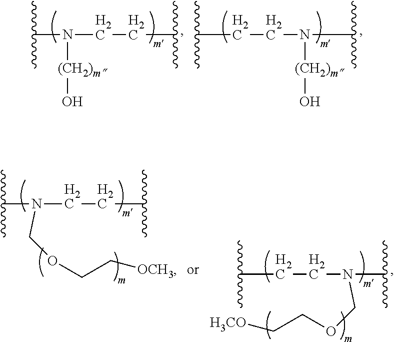
or W² and T² are absent;
L² is —C(O)-P²-X²— or —N(R²)-P²-X²—, wherein each P² is absent or a peptide comprising 1-20 amino acids, and each X² is a cleavable linker;
each of R² and R³ is independently H or optionally substituted C₁₋₆ aliphatic;
each m″ is independently 1, 2, or 3; and
each of m and m′ is independently an integer from 0-24.

71. The ligand-payload conjugate of claim 70, wherein:
(a) S² is a covalent bond,
(b) W² is —C(O)—NH—,
(c) T² is 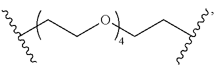
or
(d) L² is —C(O)-P²-X²—.

72. The ligand-payload conjugate of claim 70, wherein P² is a dipeptide selected from -(L-ala)-(L-ala)-, -Val-Cit-, and -Val-Ala-, and wherein X² is a PAB group 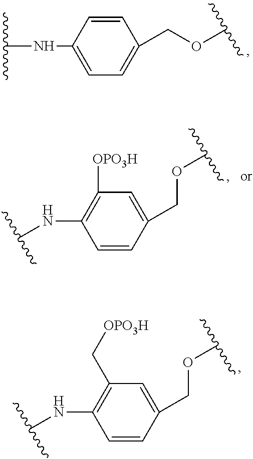
which is linked to P² via the amino nitrogen atom of the PAB group, and linked to V² via the oxygen atom of the PAB group.

73. The ligand-payload conjugate of claim 61, wherein each of D¹ and D² is independently selected from anthracycline, an auristatin or dolastatin, including but not limited to, auristatin E, auristatin EB, AEFP, MMAD, MMAF, MMAE, AEVB, and PF06380101, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, including but not limited to, topotecan, irinotecan, and SN-38, a tubulysin, a hemiasterlin, a spliceostatin, and a pladienolide, or stereoisomers, isosteres, analogs, or derivatives thereof.

74. The ligand-payload conjugate of claim 61, wherein D¹ and D² are exatecan

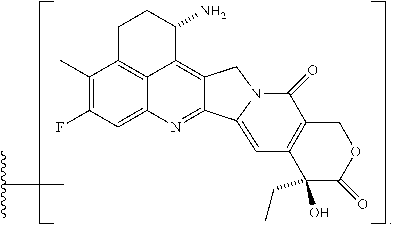

75. The ligand-payload conjugate of claim 61, wherein n is 2, 3, or 4.

76. The ligand-payload conjugate of claim 61, which is a compound of Formula (I-11):

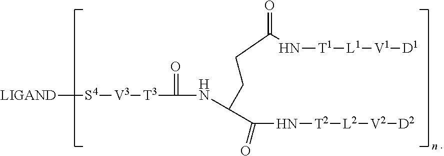

77. The ligand-payload conjugate of claim 61, which is a compound of Formula (I-14) or (I-15):

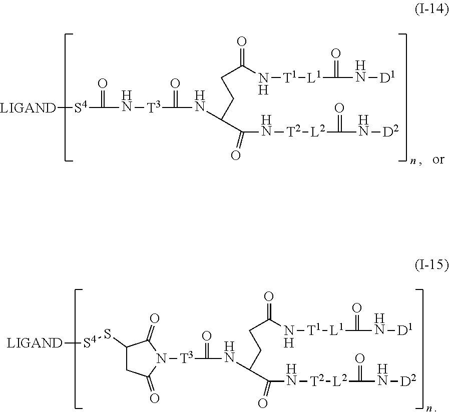

78. The ligand-payload conjugate of claim 61, which is a compound of Formula (I-16), (I-17) or (I-18):

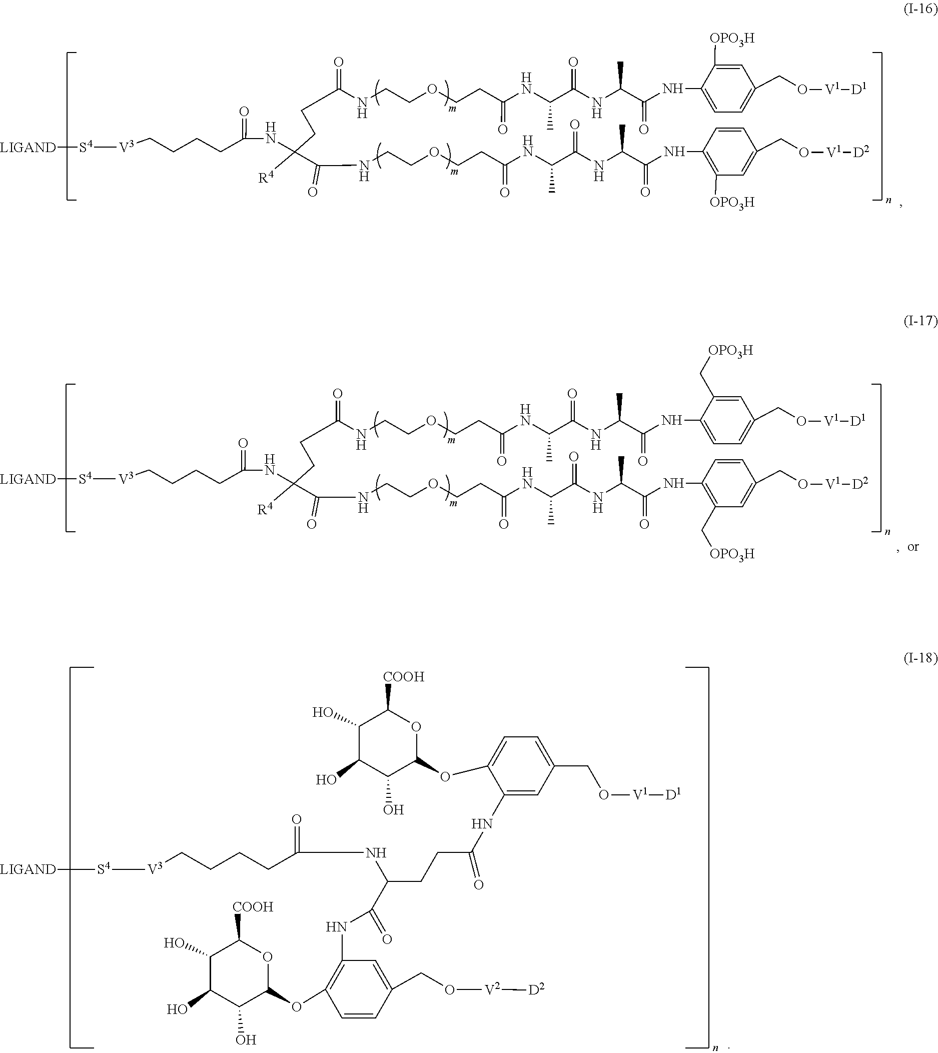

79. A ligand-payload conjugate of formula (I*): 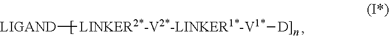
or a pharmaceutically acceptable salt thereof, wherein:
LIGAND is a ligand;
each of V¹* and V²* is independently a linker moiety formed by a coupling reaction;
each of LINKER¹* and LINKER²* is independently a bivalent linker;
each D is independently a payload;
n is 4, 5, 6, 7, 8, 9, 10, 11, or 12; wherein
(a) each LINKER²* is S²*, which is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ, —(CH₂CH₂O)ₘ—, 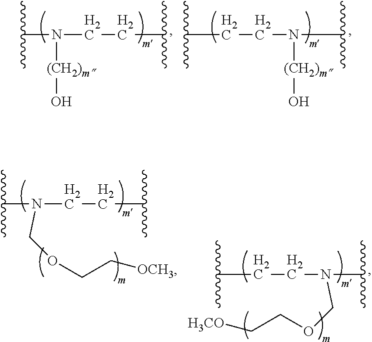
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, or —N(R)CH₂C(O)—, wherein:
each -Cy- is independently an optionally substituted 3-8 membered bivalent saturated, partially unsaturated, or aromatic ring having 0-4 heteroatoms independently selected from nitrogen, oxygen, or sulfur;
each R is independently H, or an optionally substituted group selected from C₁₋₆ aliphatic, a 3-8 membered saturated or partially unsaturated monocyclic carbocyclic ring, a 3-8 membered saturated or partially unsaturated monocyclic heterocyclic ring having 1-4 heteroatoms independently selected from nitrogen, oxygen, or sulfur, phenyl, a 5-6 membered monocyclic heteroaromatic ring having 1-4 heteroatoms independently selected from nitrogen, oxygen, or sulfur, an 8-10 membered bicyclic aromatic carbocyclic ring, and an 8-10 membered bicyclic heteroaromatic ring having 1-5 heteroatoms independently selected from nitrogen, oxygen, or sulfur;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24;
(b) each V²* is independently selected from: —C(O)—NH—, —NH—C(O)—, 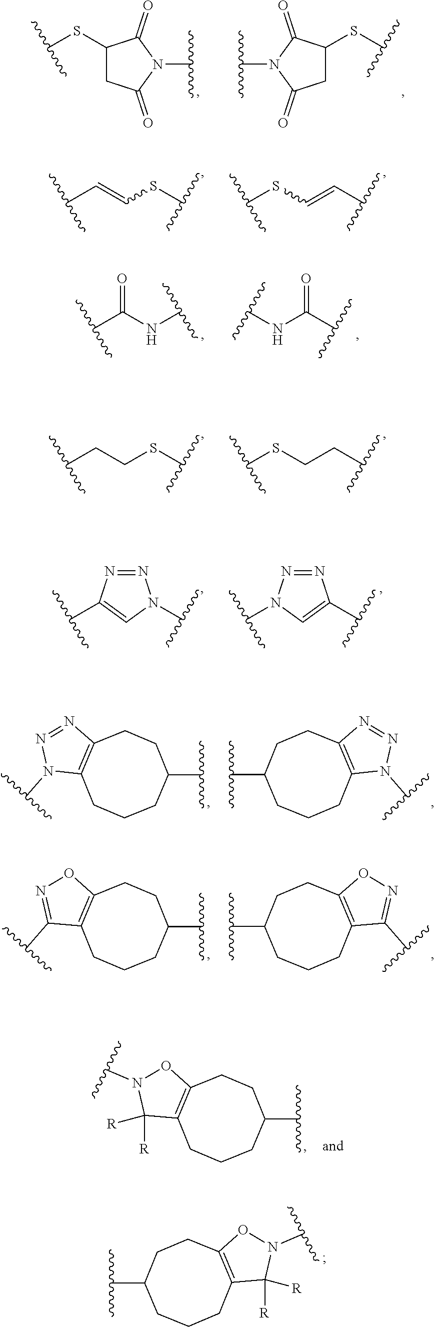
(c) each LINKER is independently 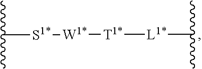
wherein:
each S¹* is independently a covalent bond or a bivalent C₁₋₈ saturated or unsaturated, straight or branched, hydrocarbon chain wherein one, two or three methylene units of the chain are optionally and independently replaced by -Cy-, —C(R)₂—, —(OCH₂CH₂)ₘ—, —(CH₂CH₂O)ₘ—, 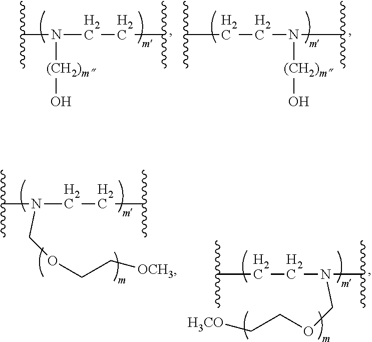
—S—, —N(R)—, —O—, —C(O)—, —OC(O)—, —C(O)O—, —C(O)N(R)—, —N(R)C(O)—, —S(O)—, —S(O)₂—, —C(O)CH₂N(R)—, —N(R)CH₂C(O)—;
each -Cy- is independently an optionally substituted 3-8 membered bivalent saturated, partially unsaturated, or aromatic ring having 0-4 heteroatoms independently selected from nitrogen, oxygen, or sulfur;
each W¹* is independently —C(O)—N(R³*)— or —N(R³*)—C(O)—, and T¹* is independently a bivalent C₁₋₈ saturated or unsaturated, straight or branched, hydrocarbon chain wherein one, two or three methylene units of the chain are optionally and independently replaced by —(OCH₂CH₂)ₘ—, —(CH₂CH₂O)ₘ—, 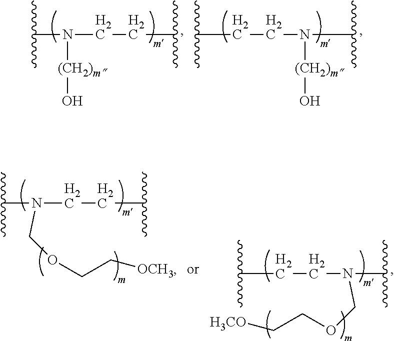
or W¹* and T¹* are absent;
each L¹* is independently —C(O)—P¹*-X¹*— or —N(R¹*)-P¹*-X¹*—, wherein each P¹* is absent or a peptide comprising 1-20 amino acids, and each X¹* is a cleavable linker;
each of R¹* and R³* is independently H or optionally substituted C₁₋₆ aliphatic;
each m″ is independently 1, 2, or 3; and
each of m and m′ is independently 0, 1, 2, 3, 4, 5, 6, 7, 8, 9, 10, 11, 12, 13, 14, 15, 16, 17, 18, 19, 20, 21, 22, 23, or 24; and
(d) each V¹* is independently selected from: —C(O)—NH—, —NH—C(O)—, 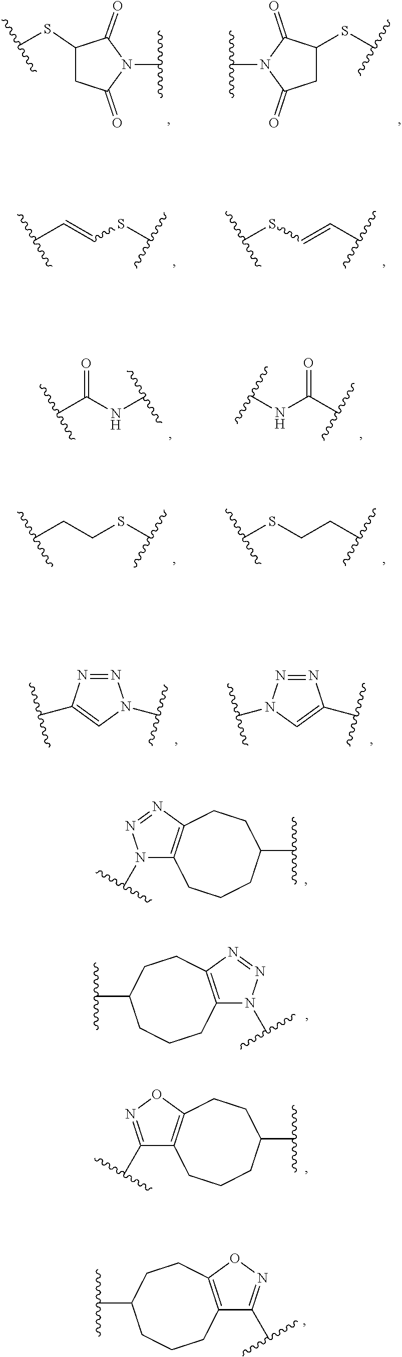 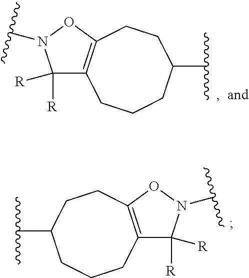
and
(e) each D is independently selected from anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, and a pladienolide, or stereoisomers, isosteres, analogs, or derivatives thereof.

80. The ligand-payload conjugate of claim 79, wherein: each X¹* is a PAB group 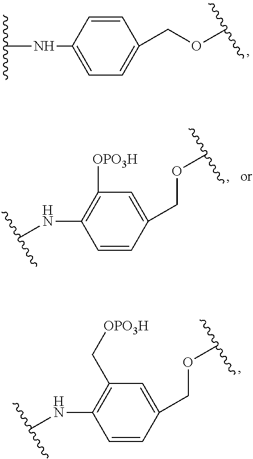
which is linked to P¹* via the amino nitrogen atom of the PAB group, and linked to V¹* via the oxygen atom of the PAB group.


SEQUENCE LISTING (39 sequences)

SEQ ID NO: 1 - protein, 5 aa, synthetic construct
LLQGG

SEQ ID NO: 2 - protein, 4 aa, synthetic construct
LLQG

SEQ ID NO: 3 - protein, 6 aa, synthetic construct
LSLSQG

SEQ ID NO: 4 - protein, 8 aa, synthetic construct
GGGLLQGG

SEQ ID NO: 5 - protein, 5 aa, synthetic construct
GLLQG

SEQ ID NO: 6 - protein, 10 aa, synthetic construct
GSPLAQSHGG

SEQ ID NO: 7 - protein, 7 aa, synthetic construct
GLLQGGG

SEQ ID NO: 8 - protein, 6 aa, synthetic construct
GLLQGG

SEQ ID NO: 9 - protein, 4 aa, synthetic construct
GLLQ

SEQ ID NO: 10 - protein, 8 aa, synthetic construct
LLQLLQGA

SEQ ID NO: 11 - protein, 5 aa, synthetic construct
LLQGA

SEQ ID NO: 12 - protein, 7 aa, synthetic construct
LLQYQGA

SEQ ID NO: 13 - protein, 6 aa, synthetic construct
LLQGSG

SEQ ID NO: 14 - protein, 6 aa, synthetic construct
LLQYQG

SEQ ID NO: 15 - protein, 7 aa, synthetic construct
LLQLLQG

SEQ ID NO: 16 - protein, 5 aa, synthetic construct
SLLQG

SEQ ID NO: 17 - protein, 5 aa, synthetic construct
LLQLQ

SEQ ID NO: 18 - protein, 6 aa, synthetic construct
LLQLLQ

SEQ ID NO: 19 - protein, 5 aa, synthetic construct
LLQGR

SEQ ID NO: 20 - protein, 6 aa, synthetic construct
LLQGPP

SEQ ID NO: 21 - protein, 6 aa, synthetic construct
LLQGPA

SEQ ID NO: 22 - protein, 8 aa, synthetic construct
GGLLQGPP

SEQ ID NO: 23 - protein, 7 aa, synthetic construct
GGLLQGA

SEQ ID NO: 24 - skipped

SEQ ID NO: 25 - protein, 7 aa, synthetic construct
LLQGPGK

SEQ ID NO: 26 - protein, 6 aa, synthetic construct
LLQGPG

SEQ ID NO: 27 - protein, 5 aa, synthetic construct
LLQGP

SEQ ID NO: 28 - protein, 4 aa, synthetic construct
LLQP

SEQ ID NO: 29 - protein, 6 aa, synthetic construct
LLQPGK

SEQ ID NO: 30 - protein, 7 aa, synthetic construct
LLQAPGK

SEQ ID NO: 31 - protein, 7 aa, synthetic construct
LLQGAPG

SEQ ID NO: 32 - protein, 6 aa, synthetic construct
LLQGAP

SEQ ID NO: 33 - protein, 6 aa, synthetic construct
LLQLQG

SEQ ID NO: 34 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 38 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 221 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGLLQGA